SEQUENCE LISTING (17 sequences)

SEQ ID NO: 1 - DNA, 18 bp, Artificial Sequence
  DNA gapmer
ggggactcgctgacatgg

SEQ ID NO: 2 - DNA, 18 bp, Artificial Sequence
  DNA gamper
tgggtgtagcgagaatcc

SEQ ID NO: 3 - DNA, 18 bp, Artificial Sequence
  DNA gamper
gggtgcactagtttatag

SEQ ID NO: 4 - DNA, 18 bp, Artificial Sequence
  DNA gapmer
ggggtcttctaatatcct

SEQ ID NO: 5 - DNA, 18 bp, Artificial Sequence
  DNA gapmer
aggttctcgctatatcgc

SEQ ID NO: 6 - DNA, 18 bp, Artificial Sequence
  DNA gapmer
gagttagaagctttgact

SEQ ID NO: 7 - DNA, 18 bp, Artificial Sequence
  DNA gapmer
gcagatgacccttagaca

SEQ ID NO: 8 - DNA, 18 bp, Artificial Sequence
  DNA gapmer
caaacctgtcacacccga

SEQ ID NO: 9 - DNA, 18 bp, Artificial Sequence
  DNA gapmer
ttaaaccccatagacata

SEQ ID NO: 10 - DNA, 18 bp, Artificial Sequence
  DNA gapmer
gaggcccaaatgatcaca

SEQ ID NO: 11 - DNA, 18 bp, Artificial Sequence
  DNA gapmer
tggatttagcagtagggt

SEQ ID NO: 12 - DNA, 18 bp, Artificial Sequence
  DNA gapmer
agcagatgacccttagac

SEQ ID NO: 13 - DNA, 18 bp, Artificial Sequence
  DNA gapmer
agccggcatacagtatat

SEQ ID NO: 14 - DNA, 18 bp, Artificial Sequence
  DNA gapmer
tgtgctctttatggatgg

SEQ ID NO: 15 - DNA, 18 bp, Artificial Sequence
  DNA gapmer
ggatttagcagtagggtg

SEQ ID NO: 16 - DNA, 18 bp, Artificial Sequence
  DNA gapmer
ccccatgactacagtgtg

SEQ ID NO: 17 - DNA, 18 bp, Artificial Sequence
  DNA gapmer
gcttttgtgaccagggac

(19) United States
(12) Patent Application Publication
(10) Pub. No.: US 2024/0060068 A1
(43) Pub. Date: Feb. 22, 2024

(54) POLY-MORPHOLINO OLIGONUCLEOTIDE GAPMERS

(71) Applicants: Francis G. FANG, Andover, MA (US); Dai-Shik KIM, Andover, MA (US); Hyeong Wook CHOI, Andover, MA (US); Yoshinori TAKAHASHI, Tsukuba-shi, Ibaraki (JP); Kenji KIKUTA, Tsukuba-shi, Ibaraki (JP); Hikaru KAWASHIMA, Tsukuba-shi, Ibaraki (JP); Toshiki KUROKAWA, Tsukuba-shi, Ibaraki (JP); Tamaki HOSHIKAWA, Tsukuba-shi, Ibaraki (JP); Mingde SHAN, Andover, MA (US); John WANG, Andover, MA (US); Eisai R&D Management Co., Ltd., Bunkyo-ku, Tokyo (JP)

(72) Inventors: Francis G. Fang, Andover, MA (US); Dae-Shik Kim, Andover, MA (US); Hyeong Wook Choi, Andover, MA (US); Yoshinori Takahashi, Tsukuba-shi, Ibaraki (JP); Kenji Kikuta, Tsukuba-shi, Ibaraki (JP); Hikaru Kawashima, Tsukuba-shi, Ibaraki (JP); Wataru Itano, Tsukuba-shi, Ibaraki (JP); Toshiki Kurokawa, Tsukuba-shi, Ibaraki (JP); Tamaki Hoshikawa, Tsukuba-shi, Ibaraki (JP); Mingde SHAN, Andover, MA (US); John Wang, Andover, MA (US)

(73) Assignee: Eisai R&D Management Co., Ltd., Bunkyo-ku, Tokyo (JP)

(21) Appl. No.: 18/256,428

(22) PCT Filed: Dec. 10, 2021
(86) PCT No.: PCT/US2021/062952

§ 371 Date: Jun. 8, 2023

Related U.S. Application Data
(60) Provisional application No. 63/124,471, filed on Dec. 11, 2020.

(51) Int. Cl. C12N 15/113; C07H 21/04

(52) U.S. Cl. CPC C12N 15/113; C07H 21/04; C12N 2310/341; C12N 2310/3233; C12N 2310/315

(57) ABSTRACT

Gapmers or pharmaceutically acceptable salt of the gapmers and methods of making the gapmers are provided. The gapmers include a gap region that contains deoxyribonucleosides linked to each other by phosphorothioate bonds, a 5′ wing region positioned at the 5′ end of the gap region that contains morpholino monomers linked to each other by phosphorodiamidate bonds, and a 3′ wing region positioned at the 3′ end of the gap region that contains morpholino monomers linked to each other by phosphorodiamidate bonds. Antisense oligonucleotides are also provided. These antisense oligonucleotides are useful in the preparation of gapmers for inhibition of Tau mRNA transcription.

### CROSS-REFERENCE TO RELATED APPLICATIONS

This patent application is the United States National Phase under 35 U.S.C. § 371 of PCT International Patent Application No. PCT/US2021/062952, filed Dec. 10, 2021, which claims the benefit of U.S. Provisional Patent Application No. 63/124,471, filed on Dec. 11, 2020. These applications are incorporated by reference as if fully rewritten herein.

### FIELD

The present disclosure relates to stereorandom and stereodefined poly-morpholino oligonucleotide gapmer embodiments and methods of their synthesis.

### BACKGROUND

Neurodegenerative disorders are a group of disorders characterized by the decline of central nervous system and peripheral nervous system structure and function. While neurodegenerative disorders exhibit heterogeneous symptoms, they can share similar features. One neurodegenerative disease, Alzheimer's disease, is a neurodegenerative disorder characterized by buildup of amyloid beta plaques and neurofibrillary tangles. It is also the leading cause of dementia. Although some cases of rare familial Alzheimer's disease involve autosomal dominant mutations to the amyloid beta precursor protein, the majority of cases are late-onset Alzheimer's disease (LOAD), which do not follow Mendelian inheritance patterns. While the mechanics of LOAD are not completely understood, genome-wide association studies have identified genetic risk factors for LOAD. Scientists have shown the ability of these genes to impact the production, aggregation, or clearance of amyloid beta plaques.

One reported pathological indicator of Alzheimer's disease is the presence of intracellular neurofibrillary tangles composed of hyperphosphorylated Tau. See Chong, et al., “Tau Proteins and Tauopathies in Alzheimer's Disease,” Cell Mol. Neurobiol. 2018 July; 38(5):965-980. Research has reported that modulation of Tau mRNA and Tau protein expression may be useful in ameliorating the effects of Tau-related neurodegenerative diseases including Alzheimer's disease and primary tauopathies.

Antisense oligonucleotides (ASO) are used in the modulation of gene expression in a sequence-specific manner. They have been developed for target validation and therapeutic purposes. Antisense technology has the potential to cure disease caused by the expression of harmful genes, including diseases caused by viral infections, cancer growth, and inflammatory diseases. Optimized antisense oligonucleotides (ASOs) such as gapmers can be used to target primary gene transcripts, mRNA product(s), spliced and unspliced coding and noncoding RNAs.

ASOs modulate RNA function by two broad mechanisms. A steric blocking mechanism that could lead to splicing modulation, non-sense mediated decay (NMD), translation blocking, RNase H-mediated degradation that results in cleavage of the target RNA by making an RNA-ASO heteroduplex.

A gapmer is a chimeric antisense oligonucleotide containing a deoxynucleotide gap region flanked with wing regions of modified oligonucleotides. The gap region of deoxynucleotide monomers is sufficiently long to induce RNase H-mediated cleavage. The wing regions are blocks of 2′-modified ribonucleotides or other artificially modified ribonucleotide monomers that protect the internal block from nuclease degradation and increase binding affinity to the target RNA. Modified DNA analogs such as 2′-MOE, 2′-OMe, LNA and cEt have been examined as wing regions due to their stability in biological fluids and increased binding affinity to RNA.

Phosphorodiamidate morpholino oligomers (PMO) are short single-stranded DNA analogs that contain a backbone of morpholine rings connected by phosphorodiamidate linkages. PMO are generally uncharged nucleic acid analogs that bind to complementary sequences of target RNA by Watson-Crick base pairing to block protein translation. PMO are resistant to a variety of enzymes present in biologic fluids, a property that makes them useful for in vivo applications.

### BRIEF SUMMARY

One aspect of the present disclosure is directed to embodiments of a gapmer or a pharmaceutically acceptable salt of the gapmer. A gapmer or pharmaceutically acceptable salt of a gapmer contains a gap region and wing regions. In preferred embodiments, the gap region is flanked by the wing regions.

In some embodiments, the gapmer or pharmaceutically acceptable salt of the gapmer possesses a gap region that may contain 6 to 12 (i.e., each of 6, 7, 8, 9, 10, 11 or 12) deoxyribonucleosides linked to each other by phosphorothioate bonds.

In other embodiments, the gapmer or pharmaceutically acceptable salt of the gapmer possess a 5′ wing region positioned at the 5′ end of the gap region, wherein the 5′ end wing region contains 3 to 7 (i.e., each of 3, 4, 5, 6 or 7) morpholino monomers linked to each other by phosphorodiamidate bonds.

In some embodiments, the gapmer or pharmaceutically acceptable salt of the gapmer possess a 3′ wing region positioned at the 3′ end of the gap region, wherein the 3′ end wing region contains 3 to 7 (i.e., each of 3, 4, 5, 6 or 7) morpholino monomers linked to each other by phosphorodiamidate bonds.

The deoxyribonucleosides of the gap region of the gapmers or pharmaceutically acceptable salts of the gapmers may be comprised of the following structure:

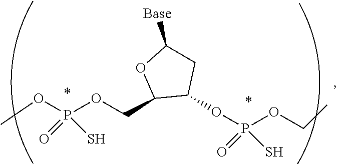

wherein P* represents a stereocenter that may either be in an R (Rₚ) or S (Sₚ) configuration.

The morpholino monomers in the wing regions of the gapmers or pharmaceutically acceptable salts of the gapmers may be comprised of the following structure:

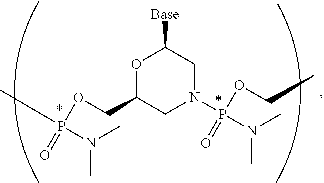

wherein P* represents a stereocenter that may either be in an R (Rₚ) or S (Sₚ) configuration.

Each base moiety (B) recited in each of the deoxyribonucleosides and morpholino oligomer structures may be independently selected from the groups included within Formula I:

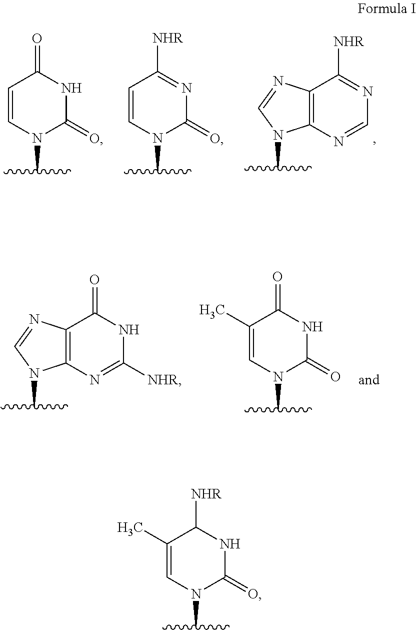

wherein R is selected from H, C(O)R₁ or C(O)OR₁; R₁ is selected from C₁-C₆ alkyl or aryl; and the aryl is unsubstituted or is substituted with a substituent selected from the group that includes halogen, nitro and methoxy.

In some embodiments, each phosphorus in the phosphorothioate and phosphorodiamidate bonds of the gapmer may be independently in an R or S configuration. Each R or S configuration is at least 90% pure, at least 95% pure, or at least 99% pure. When referring to configurations as “pure” we intend to state that at least the given percentage of gapmer will include that orientation at each position that is given.

In other embodiments, the 5′ and 3′ wing regions each include five morpholino monomers linked to each other by phosphorodiamidate bonds. In some embodiments, the 5′ and 3′ wing regions each include 4 morpholino monomers linked to each other by phosphorodiamidate bonds.

In some embodiments, the gap region includes ten deoxyribonucleosides linked to each other by phosphorothioate bonds. In other embodiments, the gap region includes eight deoxyribonucleosides linked to each other by phosphorothioate bonds.

In other embodiments, each phosphorus in the 5′ and 3′ wing regions has an S configuration. Each S configuration is at least 90% pure, at least 95% pure, or at least 99% pure.

In some embodiments, each phosphorus in the gap region has an S configuration. Each S configuration is at least 90% pure, at least 95% pure, or at least 99% pure.

In other embodiments, the phosphorus in the gap region have a mix of R and S configurations. Each phosphorus has an R or S configuration that is at least 90% pure, at least 95% pure, or at least 99% pure.

In some embodiments, the phosphorus in the gap regions, the phosphorus in the wing regions, or the phosphorus in both regions are stereorandom.

In other embodiments, the gapmers or a pharmaceutically acceptable salts of the gapmers may be conjugated to a lipid. In some embodiments, the lipid is a palmitoyl lipid or a cholesterol. The lipid may be conjugated at either the 3′ end and/or the 5′ end of the gapmers. The lipid may be conjugated to the gapmers through the use of a linker at the 3′ and/or 5′ end of the gapmers. In preferred embodiments, the linker may be a PEG or hexylamino linker.

Another aspect of the present disclosure is directed to a pharmaceutical composition that includes a gapmer or a pharmaceutically acceptable salt of a gapmer. The gapmer or a pharmaceutically acceptable salt of a gapmer may be any embodiment as discussed within the present application.

In other embodiments, gapmers are provided that may include one or two phosphodiester bonds in the DNA gap region of the gapmer.

Gapmers may be useful for treatment of a number of diseases and disorders. For example, they may be useful as antisense oligonucleotides for in vitro targeting of human microtubule-associated protein tau (MAPT) gene transcripts for the treatment of Tau-related neurodegenerative diseases including Alzheimer's disease and primary tauopathies.

In some embodiments, the antisense oligonucleotide or pharmaceutically acceptable salt thereof is a gapmer that is between 12 to 24 (i.e., each of 12, 13, 14, 15, 16, 17, 18, 19, 20, 21, 22, 23 or 24) nucleobases in length that comprises a nucleotide sequence selected from the group consisting of SEQ ID NO: 1 through SEQ ID NO: 17. In other embodiments the gapmer may be 12 to 26 (i.e., each of 12, 13, 14, 15, 16, 17, 18, 19, 20, 21, 22, 23, 24, 25 or 26) nucleobases in length comprising an nucleotide sequence selected from the group consisting of SEQ ID NO: 1 through SEQ ID NO: 17. The antisense oligonucleotide may be a chimeric oligonucleotide. The chimeric oligonucleotide may be designed to be a gapmer disclosed herein.

In other embodiments, the gapmers disclosed herein may consist of or comprise a nucleotide sequence selected from the group consisting of SEQ ID NO: 1 through SEQ ID NO: 17. In further embodiments the gapmers have at least one modified internucleoside linkage, sugar moiety, or nucleobase. In yet further embodiments the modified internucleoside linkage is a phosphorodiamidate morpholino nucleoside linkage and/or a phosphorothioate linkage.

Another aspect of the present disclosure relates to methods of inhibiting expression of Tau in a patient in need of Tau inhibition, wherein the method comprises contacting a cell or tissue of the patient with an antisense oligonucleotide, gapmer or a pharmaceutically acceptable salt of an antisense oligonucleotide and/or gapmer as disclosed herein.

Other aspects and advantages of the discussed embodiments will be apparent from the following description, drawings, and the appended claims.

### BRIEF DESCRIPTION OF THE DRAWINGS

FIG. 1A and FIG. 1B illustrate a schematic representation of a solid phase synthesis of the oligonucleotides and the synthesis cycles of the coupling reactions in the solid-phase synthesis.

FIG. 2A and FIG. 2B depict a representative synthesis of a PMO-gapmer according to a solution phase synthesis method.

FIG. 3 displays examples of general SEQ ID NO: 7 as 5-8-5 PMO-gapmers (SEQ ID NO: 7) (bold nucleotides are those present in the wing regions). “R” and “S” indicate phosphorus stereochemistry of each linkage.

FIG. 4 displays examples of general SEQ ID NO: 12 as stereodefined 4-10-4 PMO-gapmers (SEQ ID NO: 12) (bold nucleotides are those present in the wing regions). “R” and “S” indicate phosphorus stereochemistry of each linkage.

FIG. 5 shows structures of 5-8-5 and 4-10-4 PMO-gapmers.

FIG. 6 shows the sequence and phosphorus stereochemistry of compounds 123 and 132a to 132n in Table 13a and 13b (SEQ ID NO. 12). The first and last four nucleotides are wing region nucleotides. “R” and “S” indicate phosphorus stereochemistry of each linkage. “M” means a mixture of R configuration and S configuration, ^{m}C means 5-methylcytosine, and C means cytosine.

### DETAILED DESCRIPTION

An aspect of the present disclosure is directed to embodiments of a gapmer or a pharmaceutically acceptable salt of a gapmer that is comprised of gap regions and wing regions. Preferably, the gap regions are flanked by the wing regions.

Therefore, a typical utility of the disclosed gapmers is that they may be functionalized against selective gene transcripts and act as translation inhibitors. Gene transcripts of interest are those which have been identified to aid in the onset and progression of deleterious diseases.

While the terms used herein are believed to be well understood by one of ordinary skill in the art, definitions are set forth herein to facilitate explanation of the subject matter disclosed herein.

Unless defined otherwise, all technical and scientific terms used herein have the same meaning as commonly understood by one of ordinary skill in the art to which the subject matter disclosed herein belongs. Although any methods, devices, and materials similar or equivalent to those described herein can be used in the practice or testing of the presently disclosed subject matter, representative methods, devices, and materials are described herein.

All combinations of method or process steps as used herein can be performed in any order, unless otherwise specified or clearly implied to the contrary by the context in which the referenced combination is made.

The methods and devices of the present disclosure, including components thereof, can comprise, consist of, or consist essentially of the essential elements and limitations of the embodiments described herein, as well as any additional or optional components or limitations described herein or otherwise useful. For example, gapmers that are listed as “comprising” certain sequences may, in other embodiments, consist of those sequences or consist essentially of those sequences.

Unless otherwise indicated, all numbers expressing physical dimensions, quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject matter.

“Gapmer” as used herein refers to a chimeric antisense oligonucleotide that contains a central block of deoxynucleotide monomers sufficiently long to induce RNase H cleavage. A “stereorandom gapmer” is a gapmer that possesses a mixture of (R) or (S) configurations at each of its stereocenters. In some embodiments a stereorandom gapmer is a product from elongation reactions with morpholino or deoxyribonucleoside monomers. A “stereodefined gapmer” is a gapmer that possesses (R) or (S) stereochemical configurations at each of its stereocenters, wherein the configurations are controlled. A stereodefined gapmer may be a product from stereospecific elongation reactions with stereopure morpholino or deoxyribonucleoside monomers, wherein the phosphorus stereochemistry of the gapmer is controlled as a sequence of defined stereochemical (R) or (S) configurations.

A “PMO-gapmer” is a gapmer including wing regions comprising morpholino monomers linked to each other by phosphorodiamidate bonds.

“Stereorandom” when referring to a reaction means that a reaction has been conducted without preference for a resulting stereochemistry.

“R” and “S” as terms describing isomers are descriptors of the stereochemical configuration at asymmetrically substituted atoms, including but not limited to: carbon, sulfur, phosphorus and quaternary nitrogen. The designation of asymmetrically substituted atoms as “R” or “S” is done by application of the Cahn-Ingold-Prelog priority rules, as are well known to those skilled in the art, and described in the International Union of Pure and Applied Chemistry (IUPAC) Rules for the Nomenclature of Organic Chemistry. Section E, Stereochemistry.

“Pharmaceutically acceptable salt” as used herein refers to acid addition salts or base addition salts of the compounds in the present disclosure. A pharmaceutically acceptable salt is any salt which retains the activity of the parent compound and does not impart any unduly deleterious or undesirable effect on a subject to whom it is administered and in the context in which it is administered. Pharmaceutically acceptable salts include, but are not limited to, metal complexes and salts of both inorganic and carboxylic acids. Pharmaceutically acceptable salts also include metal salts such as aluminum, calcium, iron, magnesium, manganese, sodium and complex salts. In addition, pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycolic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methyl sulfuric, mucic, muconic, napsylic, nitric, oxalic, p nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like.

The term “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1% to 99.9% (more preferably, 0.5 to 90%) of active ingredient in combination with a pharmaceutically acceptable carrier.

The compounds described herein can be combined with a pharmaceutically acceptable carrier according to conventional pharmaceutical compounding techniques. As used herein, “pharmaceutically acceptable carrier” may include any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds such as by producing any undesirable biological effect or otherwise interacting in a deleterious manner with any other component(s) of the pharmaceutical composition, its use is contemplated to be within the scope of this invention.

Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; water-based solutions such as PBS or saline; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Furthermore, the carrier may take a wide variety of forms depending on the form of the preparation desired for administration, e.g., oral, nasal, rectal, vaginal, intrathecal, parenteral (including intravenous injections or infusions). In preparing compositions for oral dosage form any of the usual pharmaceutical media may be employed. Usual pharmaceutical media include, for example, water, glycols, oils, alcohols, flavoring agents, preservatives, coloring agents, and the like in the case of oral liquid preparations (such as for example, suspensions, solutions, emulsions and elixirs); aerosols; or carriers such as starches, sugars, microcrystalline cellulose, diluents, granulating agents, lubricants, binders, disintegrating agents and the like, in the case of oral solid preparations (such as for example, powders, capsules, and tablets).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, tocopherols, and the like; and metal chelating agents, such as citric acid, ethylenediamine tetraacetic acid (EDTA), sorbitol, tartaric acid, phosphoric acid, and the like.

Pharmaceutical compositions comprising the compounds may be formulated to have any concentration desired. In some embodiments, the composition is formulated such that it comprises at least a therapeutically effective amount. In some embodiments, the composition is formulated such that it comprises an amount that would not cause one or more unwanted side effects.

Pharmaceutical compositions include those suitable for oral, sublingual, nasal, rectal, vaginal, topical, buccal, intrathecal and/or parenteral (including subcutaneous, intramuscular, and intravenous) administration, although the most suitable route will depend on the nature and severity of the condition being treated. The compositions may be conveniently presented in unit dosage form, and prepared by any of the methods well known in the art of pharmacy. In certain embodiments, the pharmaceutical composition is formulated for oral administration in the form of a pill, capsule, lozenge or tablet. In other embodiments, the pharmaceutical composition is in the form of a suspension.

The term “alkyl” includes branched, straight chain and cyclic, substituted or unsubstituted saturated aliphatic hydrocarbon groups. Examples of C₁-C₆ alkyl groups include, but are not limited to, methyl, ethyl, propyl, isopropyl, butyl, sec-butyl, tert-butyl, pentyl, isopentyl, neopentyl, hexyl, isohexyl, cyclohexyl, cyclohexylmethyl, cyclopropylmethyl and neohexyl radicals.

The term “aryl” includes a 6- to 14-membered (i.e., each of 6, 7, 8, 9, 10, 11, 12, 13 or 14 membered) monocyclic, bicyclic or tricyclic aromatic hydrocarbon ring system. Examples of an aryl group include phenyl and naphthyl.

The halogen can be F, Cl, Br or I.

This application describes, in detail, improvements to conventional gapmers. A conventional gapmer can be represented by the following diagram:

One improvement is directed to the use of phosphorodiamidate morpholino oligomers (PMOs) in the wing regions. These PMOs have higher RNA binding affinity than DNA, and are resistant to nucleases.

A second improvement is directed to the linking together of the deoxyribonucleosides by phosphorothioate bonds in the gap region. These phosphorothioate bonds render the internucleotide linkage resistant to nuclease degradation.

In some embodiments, each of the 5′ and 3′ wing regions is connected to the gap region by one of a phosphorothioate or phosphorodiamidate linkage.

A general structure of the improved gapmers can be represented by the following diagram:

In some embodiments, the gapmers or pharmaceutically acceptable salt of the gapmers possess phosphorothioate and phosphorodiamidate bonds, wherein the bonds each possess a phosphorus that is independently in an R or S configuration, and wherein each R or S configuration is at least 90% pure.

In other embodiments, the gapmers or pharmaceutically acceptable salt of the gapmers possess phosphorothioate and phosphorodiamidate bonds, wherein the bonds each possess a phosphorus that is independently in an R or S configuration, and wherein each R or S configuration is at least 95% pure.

In some embodiments, the gapmers or pharmaceutically acceptable salt of the gapmers possess phosphorothioate and phosphorodiamidate bonds, wherein the bonds each possess a phosphorus that is independently in an R or S configuration, and wherein each R or S configuration is at least 99% pure.

In some embodiments, the gapmers or pharmaceutically acceptable salt of the gapmers possess a gap region containing 6-12 (i.e. each of 6, 7, 8, 9, 10, 11, or 12) deoxyribonucleosides linked to each other by phosphorothioate bonds.

In preferred embodiments, the gapmers or pharmaceutically acceptable salt of the gapmers further possess a gap region containing 8-10 (i.e. each of 8, 9, or 10) deoxyribonucleosides linked to each other by phosphorothioate bonds.

In some embodiments, the gapmers or pharmaceutically acceptable salt of the gapmers possess 5′ and 3′ wing regions, wherein the 5′ and 3′ wing regions may each consist of 3-7 (i.e. each of 3, 4, 5, 6, or 7) morpholino monomers linked to each other by phosphorodiamidate bonds. In preferred embodiments, the 5′ and 3′ wing regions each consist of 4 or 5 morpholino monomers linked to each other by phosphorodiamidate bonds.

In other embodiments, the gapmers or pharmaceutically acceptable salt of the gapmers possess phosphorodiamidate bonds, wherein all the phosphorodiamidate bonds of the 5′ and 3′ wing regions possess a phosphorus atom having an S configuration, and wherein each S configuration is at least 90% pure.

In some embodiments, the gapmers or pharmaceutically acceptable salt of the gapmers possess phosphorodiamidate bonds, wherein all the phosphorodiamidate bonds of the 5′ and 3′ wing regions possess a phosphorus atom having an S configuration, and wherein each S configuration is at least 95% pure.

In other embodiments, the gapmers or pharmaceutically acceptable salt of the gapmers possess phosphorodiamidate bonds, wherein all the phosphorodiamidate bonds of the 5′ and 3′ wing regions possess a phosphorus atom having an S configuration, and wherein each S configuration is at least 99% pure.

In other embodiments, the gapmers or pharmaceutically acceptable salt of the gapmers possess phosphorothioate bonds, wherein the phosphorothioate bonds in the gap region have a mix of R and S phosphorus configurations, and wherein each R and S configuration is at least 90% pure.

In some embodiments, the gapmers or pharmaceutically acceptable salt of the gapmers possess phosphorothioate bonds, wherein the phosphorothioate bonds in the gap region have a mix of R and S phosphorus configurations, and wherein each R and S configuration is at least 95% pure.

In other embodiments, the gapmers or pharmaceutically acceptable salt of the gapmers possess phosphorothioate bonds, wherein the phosphorothioate bonds in the gap region have a mix of R and S phosphorus configurations, and wherein each R and S configuration is at least 99% pure.

In some embodiments, the gapmers or pharmaceutically acceptable salt of the gapmers possess phosphorothioate bonds in the gap region, wherein at least one phosphorus of the phosphorothioate bonds has an R configuration. In other embodiments, the gapmers or pharmaceutically acceptable salt of the gapmers possess phosphorothioate bonds in the gap region, wherein one phosphorus of the phosphorothioate bonds has an R configuration.

In some embodiments, the gapmers or pharmaceutically acceptable salt of the gapmers possess phosphorothioate bonds in the gap region, wherein at least two phosphorus atoms of the phosphorothioate bonds have an R configuration. In other embodiments, the gapmers or pharmaceutically acceptable salt of the gapmers possess phosphorothioate bonds in the gap region, wherein two phosphorus atoms of the phosphorothioate bonds have an R configuration.

In some embodiments, the gapmers or pharmaceutically acceptable salt of the gapmers possess phosphorothioate bonds in the gap region, wherein all the phosphorothioate bonds have a S phosphorothioate configuration.

In some embodiments, the gapmers or pharmaceutically acceptable salt of the gapmers possess phosphorothioate bonds in the gap region, wherein all the phosphorothioate bonds have an R phosphorothioate configuration.

In some embodiments, the gapmers or pharmaceutically acceptable salt of the gapmers possess phosphorothioate bonds and the phosphorodiamidate bonds, wherein all the phosphorus atoms in the bonds are stereorandom.

In other embodiments, the gapmers or pharmaceutically acceptable salt of the gapmers are conjugated to a lipid, a cell-penetrating peptide, or multiple N-acetylgalactosamines (GalNAc). The lipid may be, for example, a tocopherol, a cholesterol, a palmitoyl lipid, or a docosahexaenoic acid (DHA) lipid.

In some embodiments, the gapmers or pharmaceutically acceptable salt of the gapmers are conjugated to a lipid, a cell-penetrating peptide or multiple GalNAc, wherein the lipid, a cell-penetrating peptide or multiple GalNAc is conjugated to the gapmers via a linker.

In preferred embodiments, the gapmers or pharmaceutically acceptable salt of the gapmers are conjugated to a lipid with a PEG linker or a hexylamino linker.

In other embodiments, the gapmers or pharmaceutically acceptable salt of the gapmers are conjugated to a lipid, a cell-penetrating peptide or multiple GalNAc, wherein the lipid, a cell-penetrating peptide or multiple GalNAc is conjugated at the 3′ end of the gapmers.

In some embodiments, the gapmers or pharmaceutically acceptable salt of the gapmers are conjugated to a lipid, a cell-penetrating peptide or multiple GalNAc, wherein the lipid, a cell-penetrating peptide or multiple GalNAc is conjugated at the 5′ end of the gapmers.

In other embodiments, the gapmers or pharmaceutically acceptable salt of the gapmers are conjugated to a lipid, a cell-penetrating peptide or multiple GalNAc, wherein the phosphorothioate bonds and the phosphorodiamidate bonds all possess phosphorus atoms that are stereorandom.

In some embodiments, the gapmers or pharmaceutically acceptable salt of the gapmers are conjugated to a lipid, a cell-penetrating peptide or multiple GalNAc, wherein all the phosphorodiamidate bonds of the 5′ and 3′ wing regions possess a phosphorus atom having an S configuration, and wherein each S configuration is at least 90% pure.

In some embodiments, the gapmers or pharmaceutically acceptable salt of the gapmers are conjugated to a lipid, a cell-penetrating peptide or multiple GalNAc, wherein the phosphorothioate bonds in the gap region have a mix of R and S phosphorus configurations, and wherein each R and S configuration is at least 90% pure.

In other embodiments, the gapmers or pharmaceutically acceptable salts of the gapmers comprise nucleotide sequences representing oligonucleotides useful in or as antisense oligonucleotides for modulation of Tau mRNA and expression of Tau protein. These sequences are shown in Table 1:

TABLE 1

| Sequence | SEQ ID NO: |
|---|---|
| GGGGACTCGCTGACATGG | (SEQ ID NO: 1) |
| TGGGTGTAGCGAGAATCC | (SEQ ID NO: 2) |
| GGGTGCACTAGTTTATAG | (SEQ ID NO: 3) |
| GGGGTCTTCTAATATCCT | (SEQ ID NO: 4) |
| AGGTTCTCGCTATATCGC | (SEQ ID NO: 5) |
| GAGTTAGAAGCTTTGACT | (SEQ ID NO: 6) |
| GCAGATGACCCTTAGACA | (SEQ ID NO: 7) |
| CAAACCTGTCACACCCGA | (SEQ ID NO: 8) |
| TTAAACCCCATAGACATA | (SEQ ID NO: 9) |
| GAGGCCCAAATGATCACA | (SEQ ID NO: 10) |
| TGGATTTAGCAGTAGGGT | (SEQ ID NO: 11) |
| AGCAGATGACCCTTAGAC | (SEQ ID NO: 12) |
| AGCCGGCATACAGTATAT | (SEQ ID NO: 13) |
| TGTGCTCTTTATGGATGG | (SEQ ID NO: 14) |
| GGATTTAGCAGTAGGGTG | (SEQ ID NO: 15) |
| CCCCATGACTACAGTGTG | (SEQ ID NO: 16) |
| GCTTTTGTGACCAGGGAC | (SEQ ID NO: 17) |

The sequences presented in Table 1 are in a 5′ to 3′ orientation.

In some embodiments, the nucleotide sequences presented in Table 1 may exist as a 5-8-5 gapmer as disclosed herein, which means that they possess an 8 oligonucleotide antisense gap region which is flanked by two 5 oligonucleotide wing regions. For example, if SEQ ID NO: 7 is a 5-8-5 gapmer, then it would possess the following sequence:

(SEQ ID NO: 7)
GCAGATGACCCTTAGACA,

wherein the underlined portion represents the deoxyribonucleosides present within the gap region of the gapmer, which are linked to one another by phosphorothioate bonds. The non-underlined portions represent the morpholino monomers present within the wing regions, which are linked to one another by phosphorodiamidate bonds. In preferred embodiments, the 5-8-5 gapmers are PMO-gapmers.

The nucleotide sequences presented in Table 1 may also exist as either stereo-random or stereodefined 5-8-5 gapmers.

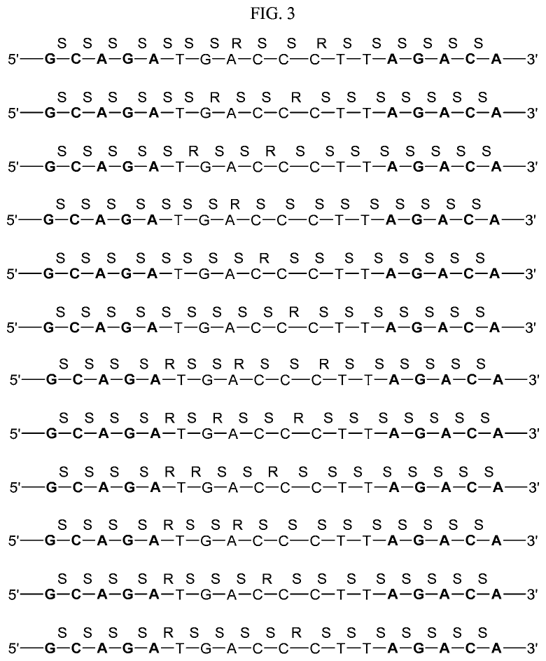

The gapmers in Table 1 may be stereodefined 5-8-5 gapmers. FIG. 3 depicts stereodefined 5-8-5 gapmers of general SEQ ID NO. 7.

In other embodiments, the nucleotide sequences presented in Table 1 may exist a 4-10-4 gapmer as disclosed herein, which means that they possess a 10 oligonucleotide antisense gap region which is flanked by two 4 oligonucleotide wing regions. For example, if general SEQ ID NO: 12 is a 4-10-4 gapmer, then it would possess the following sequence:

(SEQ ID NO: 12)
AGCAGATGACCCTTAGAC

wherein the underline portions represents the deoxyribonucleosides present within the gap region of the gapmer, which are linked to one another by phosphorothioate bonds. The non-underlined portions represent the morpholino monomers present within the wing regions, which are linked to one another by phosphorodiamidate bonds. In preferred embodiments, the 4-10-4 gapmers are PMO-gapmers.

The nucleotide sequences presented in Table 1 may also exist as either stereo-random or stereodefined 4-10-4 gapmers.

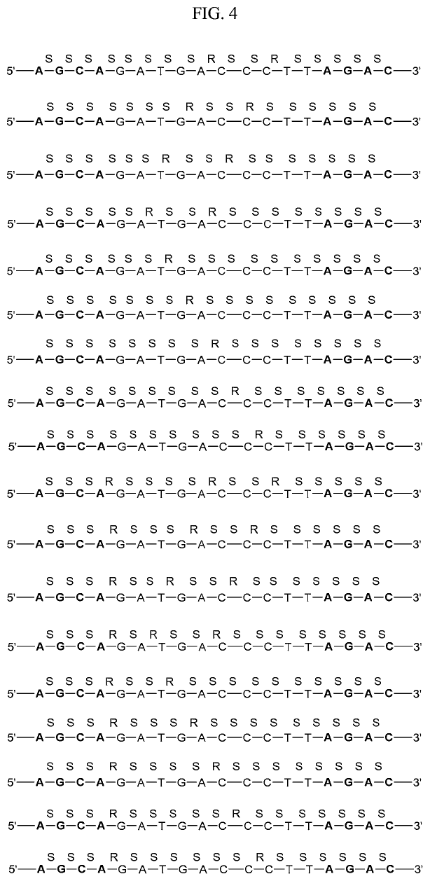

The gapmers in Table 1 may be stereodefined 4-10-4 gapmers. FIG. 4 depicts stereodefined 4-10-4 gapmers of general SEQ ID NO. 12.

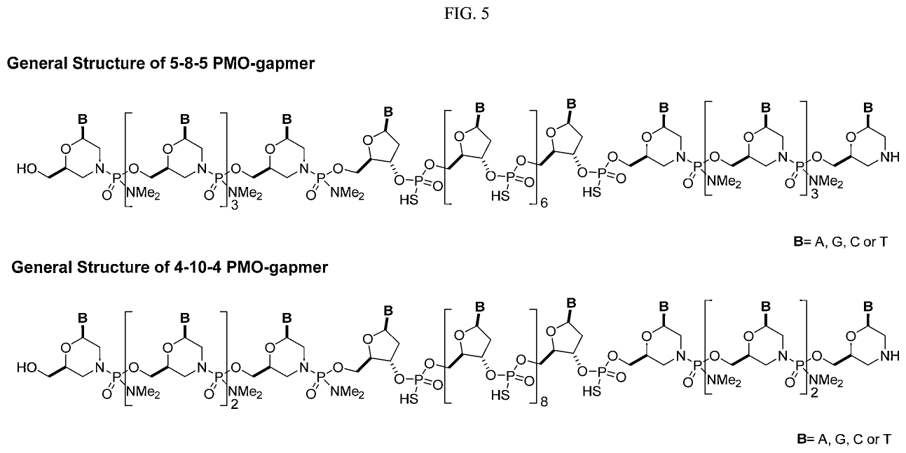

The general structures of 5-8-5 and 4-10-4 PMO-gapmers are shown in FIG. 5.

In a particular embodiment the morpholino monomers in the wing regions are linked by phosphorodiamidate bonds, and the deoxyribonucleosides in the gap region are linked by phosphorothioate bonds. In other embodiments the gap region is linked to the wing regions by either a phosphorothioate bond and/or a phosphorodiamidate bond.

The nucleotide sequences presented within Table 1 may exist as gapmers disclosed herein, wherein each phosphorus in the phosphorothioate and phosphorodiamidate bonds of the gapmers may be independently in an R or S configuration. Each R or S configuration is at least 90% pure, at least 95% pure, or at least 99% pure.

Those of skill in the art will appreciate that single nucleotide substitutions may be made in the gapmers, and that in some instances this will not affect activity.

Therefore, a utility of the disclosed gapmers is that they may be functionalized against selective gene transcripts and act as translation inhibitors, in particular translation inhibitors of Tau mRNA. Gene transcripts of interest are those which have been identified to aid in the onset and progression of deleterious diseases. In particular embodiments those deleterious diseases are associated with Tau expression.

The present disclosure also includes methods for the solid-phase synthesis of the disclosed PMO-gapmers.

In some embodiments, the PMO-gapmers are synthesized via solid-phase synthesis methods, wherein the solid-phase synthesis methods further comprise attaching a morpholino monomer onto a solid support. In preferred embodiments, the solid support is an aminomethyl polystyrene resin.

In other embodiments, the solid-phase synthesis method further comprises elongating the 5′-wing region by coupling a morpholino- or reverse DNA-dimethylphosphoramidochloridate to a morpholino monomer on a solid support.

In some embodiments, the solid-phase synthesis method further comprises elongating the DNA gap region by coupling a reverse DNA- or morpholino-phosphoramidite to the PMO on a solid support.

In other methods, the solid-phase synthesis method further comprises elongating the 3′-wing region by coupling a morpholino- or reverse DNA-dimethylphosphoramidochloridate to a PMO-DNA chimera on a solid support.

In some embodiments, elongating the 5′ PMO-gapmer wing region via a solid-phase synthesis method further may comprise a detritylation step. The detritylation step may comprises treating the elongating 5′-wing region in a mixture of 3 wt/v % TCA in CH₂Cl₂.

In other embodiments, elongating the 5′-wing region via a solid-phase synthesis method further comprises neutralizing the elongating 5′-wing region. The neutralization may comprise washing the elongating 5′-wing region with a mixture of iPr₂NEt, DMI and CH₂Cl₂ in a ratio of 10:45:45.

In some embodiments, the solid-phase synthesis method further comprises elongating the 5′-wing region by coupling a morpholino- or reverse DNA-dimethylphosphoramidochloridate to a morpholino monomer in the presence of 1,2,2,6,6-pentamethylpiperidine (PMP) in DMI.

In some embodiments, elongating the 5′-wing region via a solid-phase synthesis method further comprises capping the elongating 5′-wing region. The capping may further comprise mixing the elongating 5′-wing region with a mixture of tetrahydrofuran (THF), 2,6-lutidine and Ac20. The capping of the elongating 5′-wing region may also further comprise mixing the elongating 5′-wing region with a mixture of 16% 1-methylimidazole and THF. In some embodiments, the capping of the elongating 5′-wing region may comprise mixing the elongating 5′-wing region with both of the above mentioned mixtures.

In other embodiments, elongating the 5′-wing region via a solid-phase synthesis method further comprises removing Ac₂O from the elongating 5′-wing region. The removal of Ac₂O may further comprise mixing the elongating 5′-wing region with a 0.4M solution of morpholin in DMI.

The detritylation step, neutralization step, coupling step, the capping step and Ac₂O removal step may be repeated until a 5′-wing region possessing a desired amount of morpholino monomers have been linked.

In other embodiments, elongating the DNA gap region via a solid-phase synthesis method further may comprise a detritylation step. The detritylation step may comprises treating the elongating PMO-gapmer in a mixture of 3 wt/v % TCA in CH₂Cl₂.

In other embodiments, the solid-phase synthesis method further comprises elongating the DNA gap region by coupling a reverse DNA- or morpholino-phosphoramidite to the 5′-PMO wing region in a mixture of amidites and 5-(ethylthio)-1H-tetrazole (ETT) in acetonitrile.

In some embodiments, elongating the DNA gap region via a solid-phase synthesis method further may comprise a sulfurization step. The sulfurization step may comprises treating the elongating PMO-gapmer in a mixture of ((dimethylamino-methylidene)amino)-3H-1,2,4-dithiazoline-3-thione (DDTT) in pyridine and acetonitrile, wherein the ratio of pyridine and acetonitrile may be 2/3.

In other embodiments, elongating the DNA gap region via a solid-phase synthesis method further comprises a capping step. The capping may further comprise mixing and elongating the DNA gap region with a mixture of 10 vol % acetic anhydride in THF. The capping of the elongating DNA gap region may also further comprise mixing the elongating DNA gap region with a mixture of 1-methylimidazole-THF-Pyridine in a ratio of 10:80:10 (w/w/w). In some embodiments, the capping of the elongating DNA gap region may comprise mixing the elongating DNA gap region with both of the above mentioned mixtures.

The detritylation step, coupling step, sulfurization step and capping step may be repeated until a DNA gap region possessing the desired number of deoxyribonucleosides have been linked.

In some embodiments, elongating the 3′-PMO wing region via a solid-phase synthesis method further may comprise a detritylation step. The detritylation step may comprises washing the elongating 3′ PMO-gapmer wing region in a mixture of 3 wt/v % TCA in CH₂Cl₂.

In other embodiments, elongating the 3′ PMO-gapmer wing region via a solid-phase synthesis method further comprises neutralizing the elongating 3′ PMO-gapmer wing region. The neutralization may comprise washing the elongating 3′ PMO-gapmer wing region with iPr₂NEt in DMI and CH₂Cl₂ in a ratio of 10:45:45.

In some embodiments, the solid-phase synthesis method further comprises elongating the 3′ PMO-gapmer wing region by coupling a morpholino- or a reverse DNA-dimethylphosphoramidochloridate to a morpholino monomer in the presence of PMP in DMI.

In some embodiments, elongating the 3′ PMO-gapmer wing region via a solid-phase synthesis method further comprises capping the elongating 3′ PMO-gapmer wing region. The capping may further comprise mixing the elongating 3′ PMO-gapmer wing region with a mixture of THF, 2,6-lutidine and Ac₂O. The capping of the elongating 3′ PMO-gapmer wing region may also further comprise mixing the elongating 3′ PMO-gapmer wing region with a mixture of 16% 1-methylimidazole and THF. In some embodiments, the capping of the elongating 3′ PMO-gapmer wing region may comprise mixing the PMO-gapmer with both of the above mentioned mixtures.

In other embodiments, elongating the 3′ PMO-gapmer wing region via a solid-phase synthesis method further comprises removing Ac₂O from the elongating 3′ PMO-gapmer wing region. The removal of Ac₂O may further comprise mixing the elongating 3′ PMO-gapmer wing region with a 0.4M solution of morpholin in DMI.

In some embodiments, elongating the 3′ PMO-gapmer wing region via a solid-phase synthesis method further comprises washing the elongating 3′ PMO-gapmer wing region with CH₂Cl₂. The elongating 3′ PMO-gapmer wing region may be washed with CH₂Cl₂ after the removal of Ac₂O step, after the detritylation step, after the neutralization step, after the coupling step, and/or after the capping step.

The detritylation step, neutralization step, coupling step, the capping step and Ac₂O removal step may be repeated until a 3′ PMO-gapmer wing region possessing the desired number of morpholino monomers have been linked.

In some embodiments, the solid-phase synthesis method of forming the disclosed PMO-gapmers may further comprise cleaving the fully elongated PMO-gapmer from the solid support. The cleavage step may comprise mixing the fully elongated PMO-gapmer attached to the solid support with a mixture of 20 vol % diethylamine in CH₃CN. The cleavage step may further comprise mixing the fully elongated PMO-gapmer attached to the solid support with a mixture of 28% NH₄OH and EtOH in a 3:1 ratio.

In other embodiments, the solid-phase synthesis method of forming the disclosed PMO-gapmers further comprises purifying the PMO-gapmers by reverse-phase liquid chromatography. In preferred embodiments, the PMO-gapmers are purified by reverse-phase high-performance liquid chromatography.

In some embodiments, the solid-phase synthesis method of forming the disclosed PMO-gapmers further comprises purifying the PMO-gapmers by either a desalting step, an anion exchange step, a concentration step or any combination of the three steps.

Another aspect of the present disclosure relates to solution-phase synthesis methods to produce a stereodefined PMO-gapmer.

In some embodiments, the stereodefined PMO-gapmers are produced by a coupling of stereodefined 5′-fragment and stereodefined 3′-fragment in the solution-phase synthesis methods.

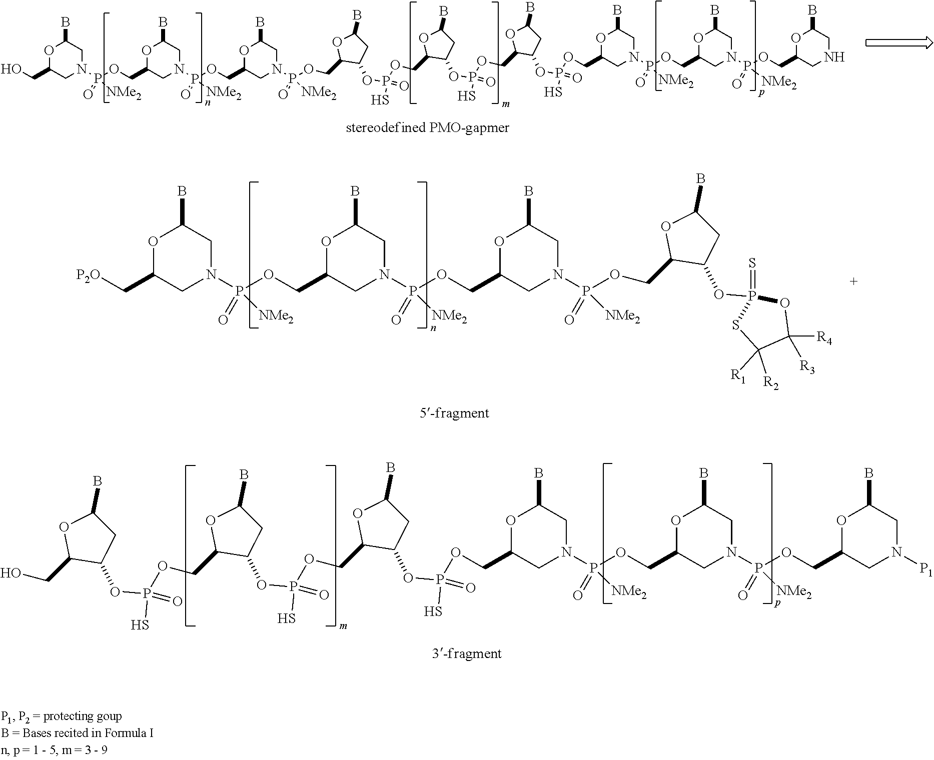

In other embodiments, the coupling step of the solution-phase synthesis methods comprises a coupling between a 12-mer stereodefined 3′-fragment and a 6-mer stereodefined 5′-fragment.

In some embodiments, the coupling step of the solution-phase synthesis methods comprises a coupling between a 13-mer stereodefined 3′-fragment and a 5-mer stereodefined 5′-fragment.

In some embodiments, the coupling step of the solution-phase synthesis methods comprises a coupling between a 14-mer stereodefined 3′-fragment and a 6-mer stereodefined 5′-fragment.

The 12-mer, 13-mer and 14-mer stereodefined 3′-fragments may further include phosphorodiamidate-linked morpholino monomers and/or phosphorothioate-linked deoxyribonucleosides.

The 5-mer and 6-mer stereodefined 5′-fragments may contain phosphorodiamidate-linked morpholino monomers and/or phosphorothioate-linked deoxyribonucleosides.

In some embodiments, synthesis of stereodefined PMO-gapmers requires a deprotection step. The deprotection step may comprise mixing a stereodefined PMO-Gapmer intermediate in a solution of methanol, 28% ammonium hydroxide and/or DL-dithiothreitol. A mixture of acetonitrile and EtOAc may further be added to the solution.

In other embodiments, synthesis of stereodefined PMO-gapmers requires a purification step. The purification step may comprise filtering a precipitate, washing a precipitate, drying a precipitate, purifying a solution with silica gel chromatography, filtering a slurry, centrifuging a slurry or solution, purifying a solution with RP-HPLC, purifying a solution with IEX-HPLC, de-salting a solution, freeze-drying a solution and/or combinations thereof.

In some embodiments, synthesis of 5′-fragment comprise a coupling step, a Tr deprotection step, an activation step or combinations thereof. The solution-phase synthesis methods may further comprise a series of these steps which can be repeated until a stereodefined 5′-fragment of a desired length is synthesized.

The coupling step of the solution-phase synthesis methods may further comprise coupling a morpholino- or reverse DNA-dimethylphosphoramidochloridate to a PMO. Other embodiments may include coupling a morpholino- or reverse DNA-dimethylphosphoramidochloridate to a 1-mer morpholino.

In other embodiments, the coupling step of the solution-phase synthesis methods may further comprise mixing a morpholino- or a reverse DNA-dimethylphosphoramidochloridate in 1,3-dimethyl-2-imidazolidinone and in the presence of 1,2,2,6,6-pentamethylpiperidine (PMP).

In some embodiments, the coupling step of the solution-phase synthesis methods may further comprise adding EtOAc, methyl tert-butyl ether and/or n-heptane to the coupling reaction mixture once the coupling is completed until the target product is precipitated out.

In other embodiments, the coupling step of the solution-phase synthesis methods may further comprise adding morpholine once the coupling is completed.

In some embodiments, the Tr deprotection step of 5′-fragment synthesis may comprise mixing a stereodefined PMO in a solution of DCM, ethanol and trifluoroacetic acid (TFA). A further embodiment may include use of a solution of 4-cyanopyridine/TFA in DCM/TFA/ethanol The deprotection step may further comprise adding EtOAc, methyl tert-butyl ether, and/or n-heptane to the mixture until the target is precipitated out. The precipitate may be collected and further washed with EtOAc, DCM, methyl tert-butyl ether, ethanol, methanol and/or combinations thereof.

The precipitate in this process would be a TFA salt of the desired product. The free base of the product may be formed by dissolving the TFA salt in DCM, optionally with MeOH, and treating it with PMP. Subsequently one would add EtOAc, MTBE, and/or n-heptane to precipitate out the product.

In some embodiments, the activation step of 5′-fragment synthesis may comprise mixing a 5-mer or 6-mer stereodefined PMO-gapmer intermediate comprising a PMO and deoxyribonucleoside with (2S,3aS,6R,7aS)-3a-Methyl-2-((perfluorophenyl)thio)-6-(prop-1-en-2-yl)hexahydrobenzo[d][1,3,2]oxathiaphosphole 2-sulfide ((−)-PSI reagent) or (2R,3aR,6S,7aR)-3a-Methyl-2-((perfluorophenyl)thio)-6-(prop-1-en-2-yl)hexahydrobenzo[d][1,3,2]oxathiaphosphole 2-sulfide ((+)-PSI reagent). The reaction mixture may further comprise 4 Å molecular sieves, DBU, DMI, DCM and/or THF. The solution may further be flushed with nitrogen before the addition of DBU. EtOAc, methyl tert-butyl ether and/or n-heptane may also be added to the solution until the target product is precipitated out. The precipitate may be washed with EtOAc and/or methyl tert-butyl ether.

In some embodiments, activation of a 5′-fragment may be conducted with 2-chloro-“spiro”-4,4-pentamethylene-1,3,2-oxathiaphospholane. The activation process may further comprise diisopropylethylamine, THF and DCM in the reaction mixture, as well as addition of elemental sulfur.

In some embodiments, synthesis of 3′-fragment comprise synthesis of a stereodefined PMO, a deprotection of base protecting groups, a N-protecting step, a deprotection of 5′-O-protecting group, a coupling step, a DMT deprotection step or combinations thereof. The solution-phase synthesis methods may further comprise a series of these steps which can be repeated until a stereodefined 3′-fragment of a desired length is synthesized.

In other embodiments, the deprotection step of base protecting groups for 3′-fragment synthesis may comprise mixing a stereodefined PMO in a solution of methanol and/or 28% ammonium hydroxide. The deprotection step may further comprise adding EtOAc, MeCN, and/or methyl tert-butyl ether to the solution until the target product is precipitated out. The precipitate may be washed with EtOAc, DCM, methyl tert-butyl ether, ethanol, methanol and/or combinations thereof.

In other embodiments, the N-protection step of the solution-phase synthesis methods may comprise mixing a deprotected stereodefined PMO in a solution of THF, water and methanol. 1,2,2,6,6-pentamethylpiperidine, and 3,5-bis(trifluoromethyl)benzoyl chloride may further be added to the solution. The N-protection step may further comprise adding EtOAc, DCM, methanol and/or combinations thereof until the target product is precipitated out. The precipitate may be washed with EtOAc, DCM and/or combinations thereof.

In some embodiments, the 5′-OTBDPS deprotection step of the solution-phase synthesis methods may comprise mixing a stereodefined PMO in a solution of 1,3-dimethyl-2-imidazolidinone, methoxytrimethylsilane, pyridine, TEA, methanol and/or TEA-3HF. The deprotection step may further comprise adding EtOAc to the solution until the target product is precipitated out. The precipitate may be collected and further washed with EtOAc, DCM, methyl tert-butyl ether, ethanol, methanol and/or combinations thereof.

In other embodiments, synthesis of 3′-fragment comprises coupling a chiral P(V) activated nucleoside to either a deoxyribonucleotide comprising stereodefined phosphorothioate linkages or a stereodefined PMO.

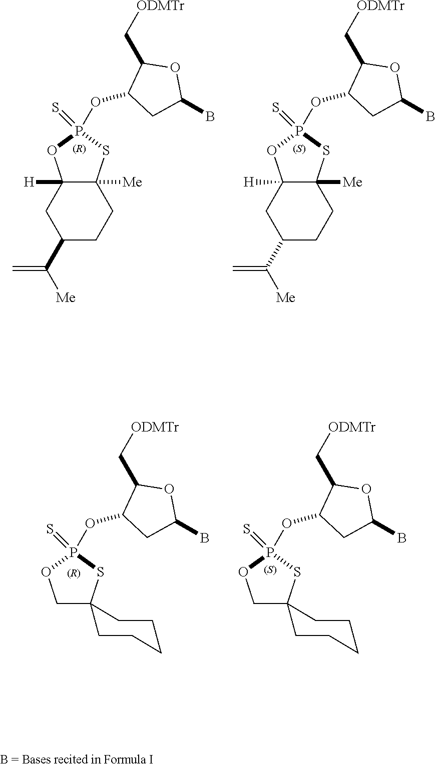

### Chiral P(V) Activated Nucleosides

In other embodiments, the coupling step of the solution-phase synthesis methods may further comprise coupling a (+)- or (−)-PSI-conjugated nucleoside to a stereodefined PMO-gapmer intermediate comprising stereodefined phosphorothioate linkages or a stereodefined PMO. The coupling of the (+)- or (−)-PSI-conjugated nucleoside to either a stereodefined PMO or a stereodefined PMO-gapmer intermediate may occur in a solution of 1,3-dimethyl-2-imidazolidinone. The reaction mixture may further comprise 4 Å molecular sieves and/or 1,8-Diazabicyclo[5.4.0]undec-7-ene (DBU). The solution may also be azeotroped with toluene between one to three times before the addition of the 4 Å molecular sieves and/or DBU. The solution may also be flushed with nitrogen or argon gas either once or three times and placed under an inert atmosphere before the addition of DBU.

In some embodiments, the coupling step of the solution-phase synthesis methods is performed at room temperature.

This specification includes a required sequence listing, and various compounds indicate the nucleotide sequence that is used in that compound. Those of skill in the art will appreciate that where a sequence is referred to as a “5-8-5 PMO-gapmer” or a “5-8-5” sequence, that in the identified compounds, in the 5′ to 3′ direction, the nucleotides indicated in the sequence listing are linked such that nucleotides 1 through 6 are linked by phosphorodiamidate bonds, nucleotides 6 through 14 are linked by phosphorothioate bonds, and nucleotides 14 through 18 are linked by phosphorodiamidate bonds. Similarly, in a 4-10-4 PMO-gapmer, in the identified compounds, in the 5′ to 3′ direction, the nucleotides indicated in the sequence listing are linked such that nucleotides 1 through 5 are linked by phosphorodiamidate bonds, nucleotides 5 through 15 are linked by phosphorothioate bonds, and nucleotides 15 through 18 are linked by phosphorodiamidate bonds. Further, the stereochemistry of such compounds is as reported in the body of this application.

In other embodiments, the DMT deprotection step of the solution-phase synthesis methods may further comprise mixing a stereodefined PMO-gapmer intermediate in a mixture of 1,1,1,3,3,3-hexafluoro-2-propanol, 2,2,2-trifluoroethanol, DCM and/or triethylsilane. The deprotection step may further comprise adding EtOAc, methyl tert-butyl ether and/or n-heptane to the solution until the target is precipitated out. The precipitate may be collected and further washed with EtOAc, DCM, methyl tert-butyl ether, ethanol, methanol and/or combinations thereof.

### EXAMPLES

### Abbreviations

The following abbreviations may be used throughout the examples.
Bz: benzoyl
iBu: isobutyryl
CE: cyanoethyl

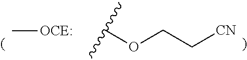

DBU: 1,8-Diazabicyclo[5.4.0]undec-7-ene
DCM: dichloromethane
DIPEA: N,N-Diisopropylethylamine
DMAP: 4-(Dimethylamino)pyridine
DMF: N,N-Dimethylformamide
DMI: 1,3-dimethyl-2-imidazolidinone
DMSO: Dimethyl sulfoxide
DMT: 4,4′-Dimethoxytrityl

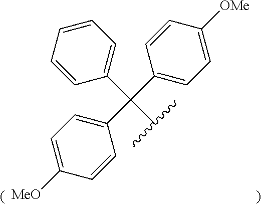 [SMILES: *C(c1ccccc1)(c1ccc(OC)cc1)c1ccc(OC)cc1]

EtOAc: Ethyl acetate
HATU: 1-[Bis(dimethylamino)methylene]-1H-1,2,3-triazolo[4,5-b]pyridinium 3-oxide hexafluorophosphate
MeCN: Acetonitrile
MMT: 4-Methoxytriphenylmethyl
MTBE: Methyl tert-butyl ether
PMP: 1,2,2,6,6-Pentamethylpiperidine
tert-: Tertiary
TEA: Triethylamine
TFA: Trifluoroacetic acid
THF: Tetrahydrofuran
TBDPS: t-butyldiphenylsilyl
Tr: Triphenylmethyl

The chemical names for the compounds in the following examples were created based on the chemical structures using “E-Notebook 2014” version 13 or E-Notebook version 18.1.1.0073 (PerkinElmer Co., Ltd.).

In Examples, flash chromatography separations were performed using SNAP cartridges (Biotage®) or Hi-Flash™ Column Silicagel or Amino (YAMAZENE CORPORATION).

Proton nuclear magnetic resonance (NMR) spectra were recorded on a JEOL JNM-ECZ 400S/L1 or JEOL JNM-ECZ 500R/S1 or Varian Inova 500 MHz or Varian Inova 400 MHz, or Bruker 400 MHz spectrometer. Chemical shifts are reported in the unit of a (ppm) and coupling constants are reported in the unit of Hertz (Hz). Abbreviations for splitting patterns are as follows: s: singlet; d: doublet; t: triplet; m: multiplet; and brs: broad singlet. 31P nuclear magnetic resonance (NMR) spectra were recorded on Varian Inova 400 MHz or Bruker 400 MHz spectrometer. Chemical shifts are reported in the unit of a (ppm). Abbreviation for splitting patterns is as follows: s: singlet.

Mass spectrometry was carried out using an Acquity UPLC and SQD2 (Waters), or a Acquity UPLC and Synapt G2 (Waters), or a Nexera X3 UHPLC (Shimadzu) and a Q Exactive Plus (ThermoFisherScientific).

In Examples, commercially available products were appropriately used as commercially available compounds.

### Example 1: Synthesis of Monomers and Loading of Morpholino Monomer on Solid Support

### Synthesis of ((2R,3S,5R)-3-(bis(4-methoxyphenyl)(phenyl)methoxy)-5-(5-methyl-2,4-dioxo-3,4-dihydropyrimidin-1(2H)-yl)tetrahydrofuran-2-yl)methyl Dimethylphosphoramidochloridate

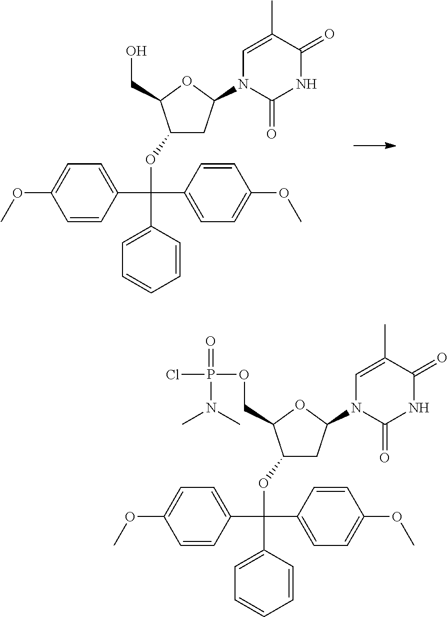 [SMILES: COc1ccc(C(O[C@H]2C[C@H](n3cc(C)c(=O)[nH]c3=O)O[C@@H]2CO)(c2ccccc2)c2ccc(OC)cc2)cc1 ; COc1ccc(C(O[C@H]2C[C@H](n3cc(C)c(=O)[nH]c3=O)O[C@@H]2COP(=O)(Cl)N(C)C)(c2ccccc2)c2ccc(OC)cc2)cc1]

### Method-1

To a solution of 3′-O-[Bis(4-methoxyphenyl)(phenyl)methyl]thymidine (CAS 76054-81-4) (3.00 g, 5.51 mmol) in DCM (20 mL) was added 1-methylimidazole (0.524 mL, 6.61 mmol), 2,6-lutidine (1.60 mL, 13.8 mmol), followed by (dimethylamino)phosphonoyl dichloride (1.63 mL, 13.8 mmol) in one portion with ice-cooling. The resulting solution was stirred for 6 h at room temperature. To 5% citric acid aqueous solution (60 mL) was added the reaction mixture with ice-cooling. The mixture was separated and the aqueous layer was extracted with DCM. The organic layer was washed with brine, dried over Na₂SO₄, filtered and concentrated in vacuo to give the crude. Silica gel column chromatography of the residue using 50% to 80% EtOAc/Heptane to afford the target material (2.71 g).

### Method-2

To a solution of 3′-O-[Bis(4-methoxyphenyl)(phenyl)methyl]thymidine (3.00 g, 5.51 mmol) in CH₃CN (55 mL) and DCM (55 mL) was added lithium bromide (1.58 g, 18.2 mmol) and DBU (2.74 mL, 18.2 mmol), followed by (dimethylamino)phosphonoyl dichloride (0.853 mL, 7.16 mmol) in one portion at 0° C. and stirred at the same temperature for 15 min. The resulting solution was stirred at room temperature for 1 h. To the reaction mixture was added citric acid monohydrate (5.0 g, 23.8 mmol) in water (95 mL) at 0° C. To the mixture was added DCM (50 mL) and the mixture was separated by ISOLUTE™ phase separater (Biotage) and the organic layer was concentrated in vacuo to give the crude. Silica gel column chromatography of the residue using 50% to 100% EtOAc/Heptane to afford the target material (1.18 g).

¹HNMR (396 MHz, CHLOROFORM-d) δ 7.28-7.36 (m, 7H), 7.94 (br s, 1H), 7.42-7.46 (m, 2H), 6.80-6.88 (m, 4H), 6.34-6.45 (m, 1H), 4.26-4.35 (m, 1H), 3.86-4.03 (m, 2H), 3.79 (s, 6H), 3.45-3.57 (m, 1H), 2.59-2.67 (m, 7H), 2.04-2.20 (m, 1H), 1.84-1.91 (m, 3H), 1.61-1.73 (m, 1H).

### Synthesis of ((2R,3S,5R)-5-(4-benzamido-2-oxopyrimidin-1(2H)-yl)-3-(bis(4-methoxyphenyl)(phenyl)methoxy)tetrahydrofuran-2-yl)methyl Dimethylphosphoramidochloridate

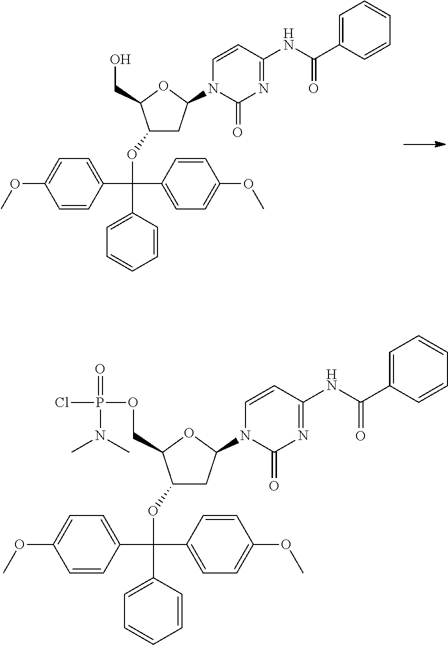 [SMILES: COc1ccc(C(O[C@H]2C[C@H](n3ccc(NC(=O)c4ccccc4)nc3=O)O[C@@H]2CO)(c2ccccc2)c2ccc(OC)cc2)cc1 ; COc1ccc(C(O[C@H]2C[C@H](n3ccc(NC(=O)c4ccccc4)nc3=O)O[C@@H]2COP(=O)(Cl)N(C)C)(c2ccccc2)c2ccc(OC)cc2)cc1]

To a solution of N-Benzoyl-3′-O-[bis(4-methoxyphenyl)(phenyl)methyl]-2′-deoxycytidine (CAS 140712-80-7) (2.00 g, 3.16 mmol) in CH₃CN (20 mL) and DCM (28 mL) was added lithium bromide (0.850 g, 9.78 mmol) and DBU (1.46 mL, 9.78 mmol), followed by (dimethylamino)phosphonoyl dichloride (0.560 mL, 4.73 mmol) in one portion at −10° C. The resulting solution was stirred for 4 h at −10° C. To the reaction mixture was added 5% citric acid aqueous solution (220 mL). The mixture was stirred at −10° C. for 5 min. To the mixture was added DCM and then it was separated. The aqueous layer was extracted with DCM, and the combined organic layer was washed with water, then washed with brine, dried over Na₂SO₄, filtered and concentrated in vacuo to give the crude. Silica gel column chromatography of the residue using 60% to 80% EtOAc/Heptane to afford the target material (1.49 g).

¹H NMR (CHLOROFORM-d, 396 MHz) δ 8.02-8.05 (m, 1H), 7.87 (br d, 2H, J=7.7 Hz), 7.60 (t, 1H, J=7.7 Hz), 7.44-7.52 (m, 5H), 7.28-7.36 (m, 6H), 7.21-7.26 (m, 1H), 6.83-6.85 (m, 4H), 6.38-6.42 (m, 1H), 4.29-4.32 (m, 1H), 3.99-4.04 (m, 0.5H), 3.92-3.93 (m, 0.5H), 3.83-3.87 (m, 1H), 3.79 (s, 6H), 3.44-3.52 (m, 1H), 2.63 (s, 1.5H), 2.63 (s, 1.5H), 2.60 (s, 1.5H), 2.59 (s, 1.5H), 1.63-1.73 (m, 2H). MS (ESI) m/z: [M+H]⁺ calcd for C₃₉H₄₁ClN₄O₈P: 759.235. Found: 759.372.

### Synthesis of ((2R,3S,5R)-5-(6-benzamido-9H-purin-9-yl)-3-(bis(4-methoxyphenyl)(phenyl)methoxy)tetrahydrofuran-2-yl)methyl Dimethylphosphoramidochloridate

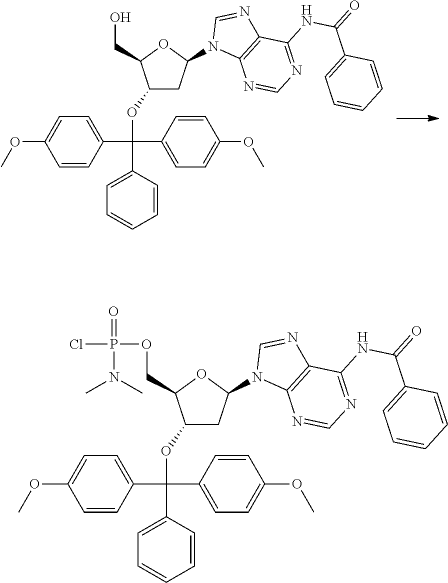 [SMILES: COc1ccc(C(O[C@H]2C[C@H](n3cnc4c(NC(=O)c5ccccc5)ncnc43)O[C@@H]2CO)(c2ccccc2)c2ccc(OC)cc2)cc1 ; COc1ccc(C(O[C@H]2C[C@H](n3cnc4c(NC(=O)c5ccccc5)ncnc43)O[C@@H]2COP(=O)(Cl)N(C)C)(c2ccccc2)c2ccc(OC)cc2)cc1]

To a solution of N-Benzoyl-3′-O-[bis(4-methoxyphenyl)(phenyl)methyl]-2′-deoxyadenosine (CAS 140712-79-4) (3.00 g, 4.56 mmol), 1-methylimidazole (0.434 mL, 5.47 mmol), and 2,6-lutidine (1.32 mL, 11.4 mmol) in DCM (22.6 mL, 351.2 mmol) at 0° C. was added (dimethylamino)phosphonoyl dichloride (1.35 mL, 11.4 mmol). The mixture was gradually warmed to room temperature and stirred at room temperature for 5 h. The reaction mixture was poured into the ice-cold 5% citric acid aqueous solution, then extracted with EtOAc (2 times). The combined organic layers were washed with brine, dried over Na₂SO₄, filtered, and concentrated in vacuo. Silica gel column chromatography of the residue using 20% to 40% to 80% EtOAc/Heptane to afford the target material (2.10 g). 1H NMR (396 MHz, CHLOROFORM-d) δ ppm 8.84-8.95 (m, 1H), 8.78 (s, 1H), 8.13 (m, 1H), 8.00 (m, 2H), 7.58-7.64 (m, 1H), 7.47-7.53 (m, 4H), 7.28-7.42 (m, 6H), 6.79-6.92 (m, 4H), 6.54 (m, 1H), 4.48-4.57 (m, 1H), 4.06-4.17 (m, 2H), 3.94-4.05 (m, 1H), 3.80 (m, 1H), 3.79 (s, 6H), 2.59-2.60 (m, 3H), 2.55-2.56 (m, 3H), 2.33-2.46 (m, 1H), 2.11-2.30 (m, 1H).

MS (ESI) m/z: [M+H]+ Calcd for C₄₀H₄₁ClN₆O₇P: 783.246. Found: 783.368.

### Synthesis of ((2R,3S,5R)-3-(bis(4-methoxyphenyl)(phenyl)methoxy)-5-(2-isobutyramido-6-oxo-1,6-dihydro-9H-purin-9-yl)tetrahydrofuran-2-yl)methyl Dimethylphosphoramidochloridate

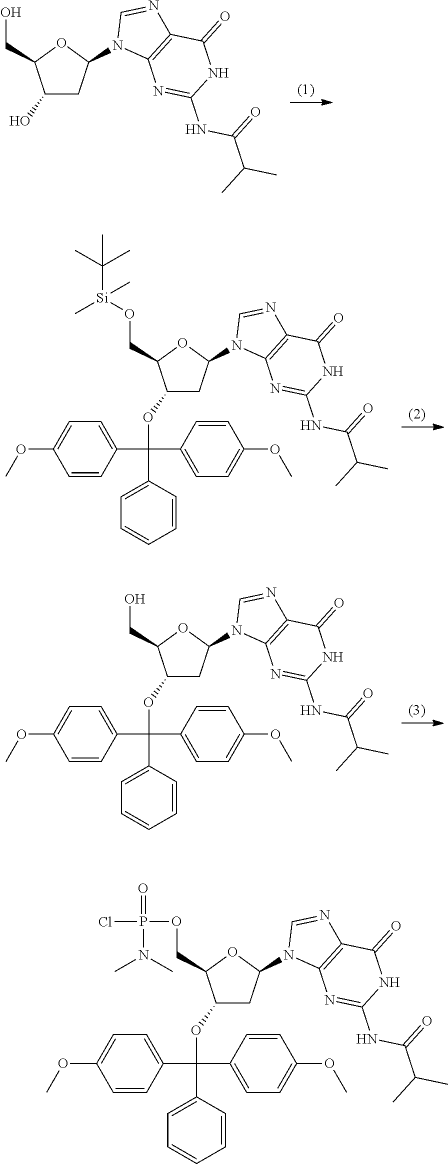

### (1) N-(9-((2R,4S,5R)-4-(bis(4-methoxyphenyl)(phenyl)methoxy)-5-(((tert-butyldimethylsilyl)oxy)methyl)tetrahydrofuran-2-yl)-6-oxo-6,9-dihydro-1H-purin-2-yl)isobutyramide

To a solution of N-(9-((2R,4S,5R)-4-hydroxy-5-(hydroxymethyl)tetrahydrofuran-2-yl)-6-oxo-6,9-dihydro-1H-purin-2-yl)isobutyramide (CAS 68892-42-2) (5.00 g, 14.8 mmol) in pyridine (33.5 mL, 0.414 mol) was added tert-butylchlorodimethylsilane (3.35 g, 22.2 mmol) with ice-cooling. The resulting solution was stirred for 190 min at room temperature. To the solution was added 4,4′-(chloro(phenyl)methylene)bis(methoxybenzene) (8.54 g, 25.2 mmol). The resulting solution was stirred for 2 h at 50° C. To the reaction mixture was added sat. NaHCO₃ aqueous solution (150 mL) and then it was separated. The aqueous layer was extracted with DCM twice, and the combined organic layer was washed with water and brine, then dried over Na₂SO₄, filtered and concentrated in vacuo to give the crude. Silica gel column chromatography of the residue using 33% to 66% EtOAc/Heptane to afford the target material (8.78 g).

¹H NMR (CHLOROFORM-d, 396 MHz) δ 11.87 (s, 1H), 7.98 (s, 1H), 7.80 (s, 1H), 7.45-7.47 (m, 2H), 7.28-7.36 (m, 6H), 7.21-7.24 (m, 1H), 6.82-6.84 (m, 4H), 6.20-6.24 (m, 1H), 4.36-4.38 (m, 1H), 4.05-4.07 (m, 1H), 3.78 (s, 6H), 3.58-3.62 (m, 1H), 3.31-3.35 (m, 1H), 2.54-2.61 (m, 1H), 1.94-2.01 (m, 1H), 1.83-1.88 (m, 1H), 1.27-1.29 (m, 6H), 0.77 (s, 9H), −0.07 (s, 3H), −0.09 (s, 3H). MS (ESI) m/z: [M+H]⁺ Calcd for C₄₁H₅₂N₅O₇Si: 754.363. Found: 754.387.

### (2) N-(9-((2R,4S,5R)-4-(bis(4-methoxyphenyl)(phenyl)methoxy)-5-(hydroxymethyl)tetrahydrofuran-2-yl)-6-oxo-6,9-dihydro-1H-purin-2-yl)isobutyramide

To a solution of N-(942R,4S,5R)-4-(bis(4-methoxyphenyl)(phenyl)methoxy)-5-(((tert-butyldimethylsilyl)oxy)methyl)tetrahydrofuran-2-yl)-6-oxo-6,9-dihydro-1H-purin-2-yl)isobutyramide (4.50 g, 5.97 mmol) in THF (41 mL) was added tetra-n-butylammonium fluoride (1 M THF solution, 6.57 mL, 6.57 mmol). The resulting solution was stirred for 18 h at room temperature. The reaction mixture was diluted with EtOAc (400 mL) and washed with sat. NH₄Cl aqueous solution (200 mL), sat. NaHCO₃ aqueous solution (200 mL) and brine (200 mL). The organic layer was dried over Na₂SO₄, filtered and concentrated in vacuo to give the crude. Silica gel column chromatography of the residue using 0% to 20% MeOH/DCM to afford the mixture containing target material. Further silica gel column chromatography of the mixture using 1% to 5% MeOH/DCM to afford the target material (3.03 g).

¹H NMR (CHLOROFORM-d, 396 MHz) δ 12.00 (br s, 1H), 8.24 (br s, 1H), 7.65 (s, 1H), 7.43-7.46 (m, 2H), 7.28-7.35 (m, 6H), 7.21-7.23 (m, 1H), 6.81-6.85 (m, 4H), 6.15 (dd, 1H, J=5.3, 9.7 Hz), 5.14 (br d, 1H, J=11.0 Hz), 4.50 (d, 1H, J=5.7 Hz), 4.05 (s, 1H), 3.78 (s, 3H), 3.78 (s, 3H), 3.68-3.71 (m, 1H), 3.27 (t, 1H, J=11.0 Hz), 2.55-2.62 (m, 1H), 2.41 (ddd, 1H, J=5.7, 9.7, 13.6 Hz), 1.70 (dd, 1H, J=5.3, 13.6 Hz), 1.22-1.23 (m, 6H). MS (ESI) m/z: [M+H]⁺ Calcd for C₃₅H₃₈N₅O₇: 640.277. Found: 640.615.

(3) ((2R,3S,5R)-3-(bis(4-methoxyphenyl)(phenyl)methoxy)-5-(2-isobutyramido-6-oxo-1,6-dihydro-9H-purin-9-yl)tetrahydrofuran-2-yl)methyl dimethylphosphoramidochloridate

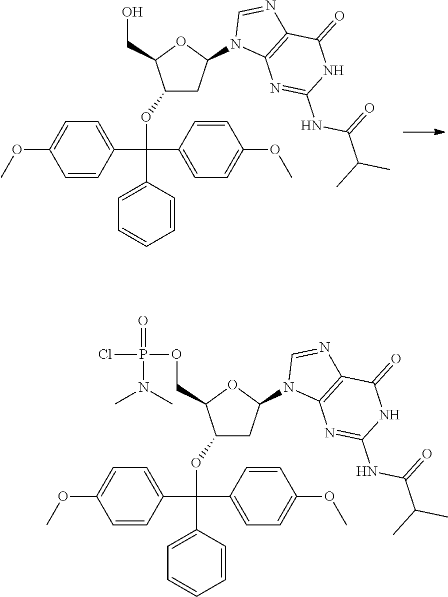

To a solution of N-(9-((2R,4S,5R)-4-(bis(4-methoxyphenyl)(phenyl)methoxy)-5-(hydroxymethyl)tetrahydrofuran-2-yl)-6-oxo-6,9-dihydro-1H-purin-2-yl)isobutyramide (2.38 g, 3.73 mmol) in CH₃CN (32 mL) and DCM (32 mL) was added lithium bromide (1.29 g, 14.9 mmol) and DBU (2.25 mL, 14.9 mmol), followed by (dimethylamino)phosphonoyl dichloride (0.887 mL, 7.45 mmol) in one portion with ice-cooling. The resulting solution was stirred for 45 min with ice-cooling. To the reaction mixture was added 5% citric acid aqueous solution (300 mL). The mixture was stirred with ice-cooling for 5 min. To the mixture was added DCM (270 mL) and then it was separated. The aqueous layer was extracted with DCM twice, and the combined organic layer was washed with water. The water layer was extracted with DCM twice, and the combined organic layer was dried over Na₂SO₄, filtered and concentrated in vacuo to give the crude. Silica gel column chromatography of the residue using 0% to 16% THF/DCM to afford the target material (2.08 g).

¹H NMR (CHLOROFORM-d, 396 MHz) δ 12.15 (s, 0.5H), 12.11 (s, 0.5H), 10.01 (s, 0.5H), 9.93 (s, 0.5H), 7.64 (s, 0.5H), 7.61 (s, 0.5H), 7.44-7.47 (m, 2H), 7.30-7.36 (m, 6H), 7.21-7.24 (m, 1H), 6.83-6.86 (m, 4H), 6.27-6.31 (m, 0.5H), 6.16-6.20 (m, 0.5H), 4.70-4.76 (m, 0.5H), 4.48-4.49 (m, 0.5H), 4.32-4.38 (m, 1H), 4.20-4.25 (m, 1H), 3.99-4.03 (m, 0.5H), 3.85-3.88 (m, 0.5H), 3.78 (s, 3H), 3.78 (s, 3H), 2.65-2.76 (m, 2H), 2.62 (s, 1.5H), 2.61 (s, 1.5H), 2.59 (s, 1.5H), 2.58 (s, 1.5H), 1.94-1.99 (m, 0.5H), 1.67-1.72 (m, 0.5H), 1.16-1.21 (m, 6H). MS (ESI) m/z: [M+H]⁺ Calcd for C₃₇H₄₃ClN₆O₈P: 765.256. Found: 765.383.

### Synthesis of ((2S,6R)-6-(2-isobutyramido-6-oxo-1,6-dihydro-9H-purin-9-yl)-4-tritylmorpholin-2-yl)methyl Dimethylphosphoramidochloridate

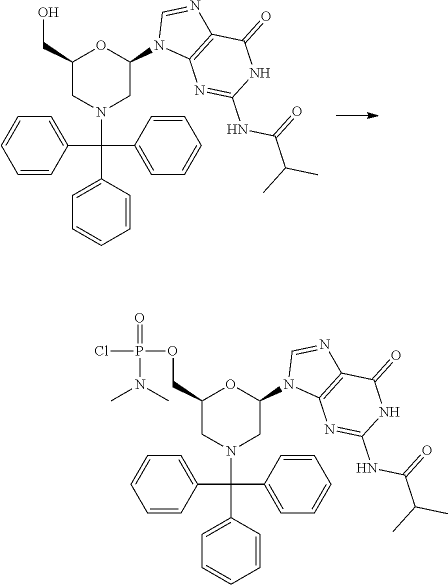 [SMILES: CC(C)C(=O)Nc1nc2c(ncn2[C@H]2CN(C(c3ccccc3)(c3ccccc3)c3ccccc3)C[C@@H](CO)O2)c(=O)[nH]1 ; CC(C)C(=O)Nc1nc2c(ncn2[C@H]2CN(C(c3ccccc3)(c3ccccc3)c3ccccc3)C[C@@H](COP(=O)(Cl)N(C)C)O2)c(=O)[nH]1]

To a solution of N-(9-((2R,6S)-6-(hydroxymethyl)-4-tritylmorpholin-2-yl)-6-oxo-6,9-dihydro-1H-purin-2-yl)isobutyramide (4.00 g, 6.91 mmol) in CH₃CN (59 mL) and DCM (59 mL) was added lithium bromide (2.40 g, 27.6 mmol) and DBU (4.17 mL, 27.6 mmol), followed by (dimethylamino)phosphonoyl dichloride (1.65 mL, 13.8 mmol) in one portion with ice-cooling. The resulting solution was stirred for 35 min with ice bath. To the reaction mixture was added 5% citric acid aqueous solution (220 mL). The mixture was stirred with ice bath for 5 min. To the mixture was added DCM (180 mL) and then it was separated. The aqueous layer was extracted with DCM twice, and the combined organic layer was dried over Na₂SO₄, filtered and concentrated in vacuo to give the crude. Silica gel column chromatography of the residue using 0% to 16% THF/DCM to afford the target material (2.70 g).

1H NMR (CHLOROFORM-d, 396 MHz) δ 11.98 (br s, 0.5H), 11.97 (br s, 0.5H), 8.62 (s, 8.46 (s, 0.5H), 7.58 (s, 0.5H), 7.57 (s, 0.5H), 7.44 (br s, 6H), 7.28-7.31 (m, 6H), 7.17-7.21 (m, 3H), 5.96-6.01 (m, 1H), 4.42-4.47 (m, 1H), 4.02-4.18 (m, 2H), 3.41-3.44 (m, 1H), 3.19-3.23 (m, 1H), 2.66-2.71 (m, 1H), 2.64 (s, 1.5H), 2.63 (s, 1.5H), 2.61 (s, 1.5H), 2.59 (s, 1.5H), 1.69-1.75 (m, 1H), 1.50-1.57 (m, 1H), 1.26-1.31 (m, 6H). MS (ESI) m/z: [M+H]⁺ Calcd for C₃₅H₄₀ClN₇O₅P: 704.251. Found: 704.380.

### Synthesis of ((2S,6R)-6-(6-(2-cyanoethoxy)-2-isobutyramido-9H-purin-9-yl)-4-tritylmorpholin-2-yl)methyl (2-cyanoethyl) diisopropylphosphoramidite

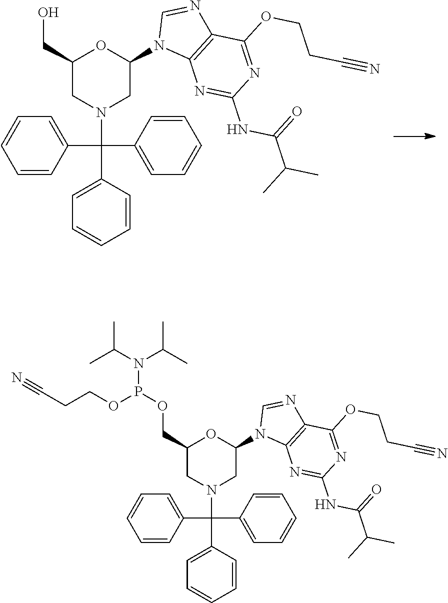 [SMILES: CC(C)C(=O)Nc1nc(OCCC#N)c2ncn([C@H]3CN(C(c4ccccc4)(c4ccccc4)c4ccccc4)C[C@@H](CO)O3)c2n1 ; CC(C)C(=O)Nc1nc(OCCC#N)c2ncn([C@H]3CN(C(c4ccccc4)(c4ccccc4)c4ccccc4)C[C@@H](COP(OCCC#N)N(C(C)C)C(C)C)O3)c2n1]

To a solution of N-(6-(2-cyanoethoxy)-9-((2R,6S)-6-(hydroxymethyl)-4-tritylmorpholin-2-yl)-9H-purin-2-yl)isobutyramide (3.00 g, 4.75 mmol) in DCM (30 mL) was added DIPEA (1.82 mL, 10.5 mmol), followed by 2-CYANOETHYL N,N-DIISOPROPYLCHLOROPHOSPHORAMIDITE (1.17 mL, 5.22 mmol) at 0° C. and the reaction mixture was stirred for 1 h at room temperature. To the mixture was added sat. NaHCO₃ aqueous solution at 0° C. The organic layer was separated by ISOLUTE′ phase separater (Biotage) and the organic layer was concentrated in vacuo to give the crude. Silica gel column chromatography of the residue using 50% to 100% EtOAc/Heptane afforded the target material (1.50 g).

¹H NMR (400 MHz, CHLOROFORM-d) δ 7.76-7.82 (m, 2H), 7.43-7.53 (m, 5H), 7.26-7.32 (m, 6H), 7.15-7.22 (m, 3H), 6.18-6.25 (m, 1H), 4.69-4.83 (m, 2H), 4.32-4.41 (m, 1H), 3.43-3.76 (m, 8H), 3.21-3.33 (m, 1H), 2.93-3.09 (m, 3H), 2.45-2.57 (m, 2H), 1.68-1.81 (m, 1H), 1.32-1.36 (m, 6H), 1.10-1.14 (m, 6H), 0.99-1.06 (m, 6H).

### Synthesis of 4-(((2S,6R)-6-(5-methyl-2,4-dioxo-3,4-dihydropyrimidin-1(2H)-yl)-4-tritylmorpholin-2-yl)methoxy)-4-oxobutanoic Acid Loaded onto Aminomethylpolystyrene Resin

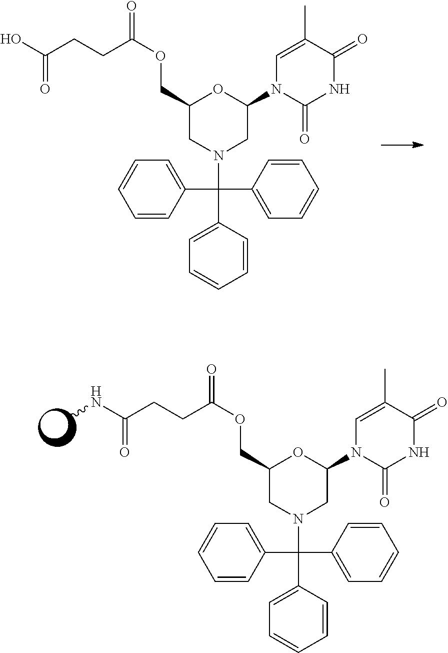

4-(((2S,6R)-6-(5-Methyl-2,4-dioxo-3,4-dihydropyrimidin-1(2H)-yl)-4-tritylmorpholin-2-yl)methoxy)-4-oxobutanoic acid (CAS 1362664-41-2) (360 mg, 0.617 mmol) was dissolved in DMF (15.4 mL). HATU (793 mg, 2.09 mmol) and DIPEA (0.539 mL, 3.08 mmol) were added and then Aminomethyl Polystyrene Resin (Primer Support™ 5G Amino, 29-0999-92, manufactured by GE Healthcare) (2.00 g, amine content: 400 μmol/g) was added to the reaction mixture and gently shaken at room temperature on Bio-shaker (110 rpm) for 12 h. The resin was filtered, washed with DCM, 50% MeOH in CHCl₃, DCM and ether in this order. The resin was dried under vacuum for 1 h. The unreacted amines on the resin were capped by reacting with Cap B Solution-1 (THF/l-Me-imidazole/Pyridine (8:1:1)) (97 mL) and Cap A Solution-1 (10 vol % Ac2O/THF) (65 mL) on Bio-shaker (110 rpm) for 1 h at room temperature. The resin was filtered, washed with DCM, 20% MeOH in DCM, DCM and ether in this order. The resin was dried under high vacuum to afford the target material (1.80 g, loading: 229 μmol/g).

### Synthesis of 4-(((2S,6R)-6-(4-benzamido-2-oxopyrimidin-1(2H)-yl)-4-tritylmorpholin-2-yl)methoxy)-4-oxobutanoic Acid Loaded onto Aminomethylpolystyrene Resin

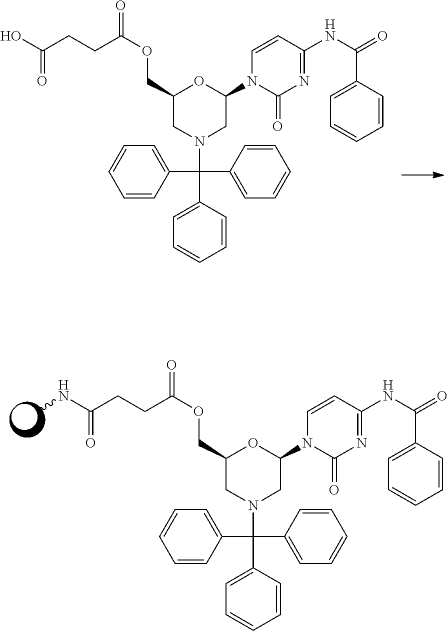

4-(((2S,6R)-6-(4-Benzamido-2-oxopyrimidin-1(2H)-yl)-4-tritylmorpholin-2-yl)methoxy)-4-oxobutanoic acid (CAS 1362664-31-0) (540 mg, 0.803 mmol) was dissolved in DMF (22 mL). HATU (1.03 g, 2.71 mmol) and DIPEA (0.701 mL, 4.01 mmol) were added and then Aminomethyl Polystyrene Resin (Primer Support™ 5G Amino, 29-0999-92, manufactured by GE Healthcare) (2.32 g, amine content: 450 μmol/g) was added to the reaction mixture and gently shaken at room temperature on Bio-shaker (110 rpm) for 12 h. The resin was filtered, washed with DCM, 50% MeOH in CHCl₃, DCM and ether in this order. The resin was dried under vacuum for 1 h. The unreacted amines on the resin were capped by reacting with Cap B Solution-1 (THF/l-Me-imidazole/Pyridine (8:1:1)) (127 mL) and Cap A Solution-1 (10 vol % Ac2O/THF) (84 mL) on Bio-shaker (110 rpm) for 2 h at room temperature. The resin was filtered, washed with DCM, 20% MeOH in DCM, DCM and ether in this order. The resin was dried under high vacuum to afford the target material (2 g, loading: 194 μmol/g).

### Synthesis of 4-(((2S,6R)-6-(6-benzamido-9H-purin-9-yl)-4-tritylmorpholin-2-yl)methoxy)-4-oxobutanoic Acid Loaded onto Aminomethylpolystyrene Resin

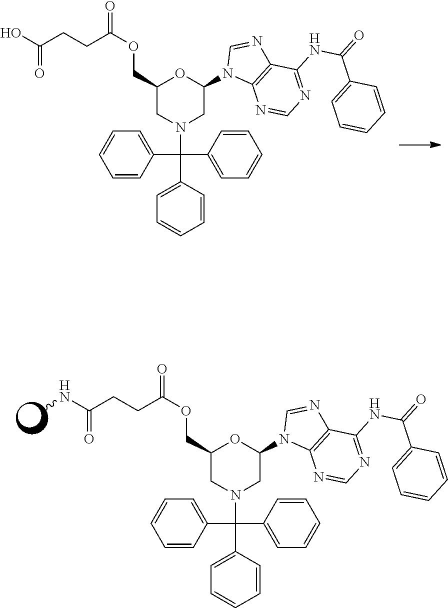

4-(((2S,6R)-6-(6-Benzamido-9H-purin-9-yl)-4-tritylmorpholin-2-yl)methoxy)-4-oxobutanoic acid (CAS 446206-67-2) (174 mg, 0.250 mmol) was dissolved in DMF (6.3 mL). HATU (321 mg, 0.845 mmol) and DIPEA (0.218 mL, 1.25 mmol) were added and then Aminomethyl Polystyrene Resin (Primer Support 5G Amino, 29-0999-92, manufactured by GE Healthcare) (813 mg, amine content: 400 μmol/g) was added to the reaction mixture and gently shaken at room temperature on Bio-shaker (110 rpm) for 12 h. The resin was filtered, washed with DCM, 50% MeOH in CHCl₃, DCM and ether in this order. The resin was dried under vacuum for 1 h. The unreacted amines on the resin were capped by reacting with Cap B Solution-1 (THF/1-Me-imidazole/Pyridine (8:1:1)) (39.4 mL) and Cap A Solution-1 (10 vol % Ac2O/THF) (26.2 mL) on Bio-shaker (110 rpm) for 1 h at room temperature. The resin was filtered, washed with DCM, 20% MeOH in DCM, DCM and ether in this order. The resin was dried under high vacuum to afford target material (827 mg, loading: 196 μmol/g).

### Synthesis of 4-(((2S,6R)-6-(6-(2-cyanoethoxy)-2-isobutyramido-9H-purin-9-yl)-4-tritylmorpholin-2-yl)methoxy)-4-oxobutanoic Acid Loaded onto Aminomethylpolystyrene Resin

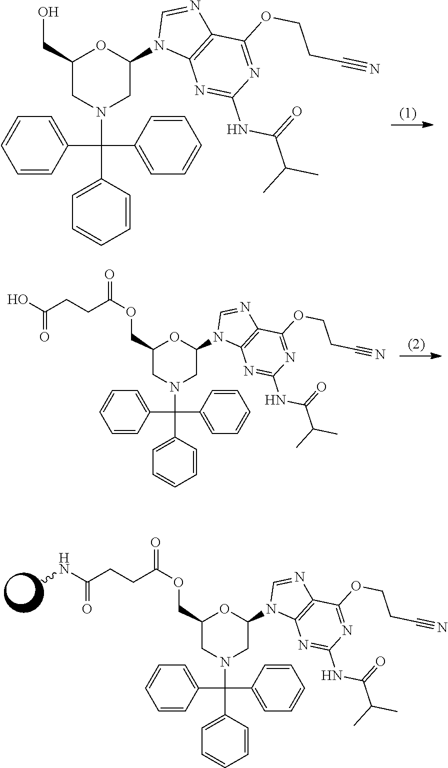

### (1) 4-(((2S,6R)-6-(6-(2-cyanoethoxy)-2-isobutyramido-9H-purin-9-yl)-4-tritylmorpholin-2-yl)methoxy)-4-oxobutanoic Acid

To a solution of N-(6-(2-cyanoethoxy)-9-((2R,6S)-6-(hydroxymethyl)-4-tritylmorpholin-2-yl)-9H-purin-2-yl)isobutyramide (1.50 g, 2.37 mmol) and DMAP (0.87 g, 7.12 mmol) in 1,2-Dichloroethane (15 mL) was added succinic anhydride (0.475 g, 4.75 mmol) at room temperature and stirred for 1.5 h at 45° C. The mixture was cooled to room temperature. MeOH (5 mL) was added and the mixture was evaporated. EtOAc and 0.5M KH₂PO₄ aq (pH-7) was added to the residue, and the organic layer was separated. The aqueous layer was extracted with EtOAc. The combined organic layer was washed with 0.5M KH₂PO₄ aq (acidic), water, then brine, dried over MgSO₄, filtered and concentrated in vacuo to give the 4-(((2S,6R)-6-(6-(2-cyanoethoxy)-2-isobutyramido-9H-purin-9-yl)-4-tritylmorpholin-2-yl)methoxy)-4-oxobutanoic acid (1.51 g).

¹H NMR (396 MHz, CHLOROFORM-d) δ 9.22-9.36 (m, 1H), 7.73-7.79 (m, 1H), 7.43-7.54 (m, 5H), 7.28-7.35 (m, 6H), 7.15-7.23 (m, 4H), 5.95-6.05 (m, 1H), 4.71-4.88 (m, 2H), 4.45-4.56 (m, 1H), 4.30-4.39 (m, 1H), 3.77-3.89 (m, 1H), 3.38-3.46 (m, 1H), 3.13-3.21 (m, 1H), 2.97-3.09 (m, 2H), 2.80-2.92 (m, 2H), 2.47-2.67 (m, 4H), 2.05-2.11 (m, 1H), 1.23-1.30 (m, 6H). MS (ESI) m/z: [M+H]⁺ Calcd for C₄₀H₄₂N₇O₇: 732.314. Found: 732.493.

### (2) 4-(((2S,6R)-6-(6-(2-cyanoethoxy)-2-isobutyramido-9H-purin-9-yl)-4-tritylmorpholin-2-yl)methoxy)-4-oxobutanoic Acid Loaded onto Aminomethylpolystyrene Resin

4-(((2S,6R)-6-(6-(2-Cyanoethoxy)-2-isobutyramido-9H-purin-9-yl)-4-tritylmorpholin-2-yl)methoxy)-4-oxobutanoic acid (183 mg, 0.25 mmol) was dissolved in DMF (7.5 mL). HATU (321 mg, 0.845 mmol) and DIPEA (0.218 mL, 1.25 mmol) were added and then Aminomethyl Polystyrene Resin (Primer Support™ 5G Amino, 29-0999-92, manufactured by GE Healthcare) (813 mg, amine content: 400 μmol/g) was added to the reaction mixture and gently shaken at room temperature on Bio-shaker (110 rpm) for 18 h. The resin was filtered, washed with DCM, 50% MeOH in CHCl₃, DCM and ether in this order. The resin was dried under vacuum for 1 h. The unreacted amines on the resin were capped by reacting with Cap B Solution-1 (THF/l-Me-imidazole/Pyridine (8:1:1)) (39.4 mL) and Cap A Solution-1 (10 vol % Ac₂O/THF) (26.2 mL) on Bio-shaker (110 rpm) for 1 h at room temperature. The resin was filtered, washed with DCM, 20% MeOH in DCM, DCM and ether in this order. The resin was dried under high vacuum to afford target material (750 mg, loading: 208 mol/g).

### 1H-NMR: Proton Nuclear Magnetic Resonance Spectrometry

The chemical shifts of proton nuclear magnetic resonance spectrometry are recorded in δ unit (ppm) from tetramethylsilane. The abbreviations in the patterns are as indicated below:

s: singlet, d: doublet, t: triplet, q: quartet, quin: quintet, m: multiplet, br: broad.

### Silica Gel Column Chromatography

Parallel Prep produced by YAMAZEN Corporation {Hi-Flash Column packing normal silica gel), size; S (16×60 mm), M (20×75 mm), L (26×100 mm), 2 L (26×150 mm), produced by YAMAZEN Corporation} was used.

### Example 2: Overall Synthetic Scheme for Solid-Phase Synthesis of Stereorandom PMO-Gapmers

Oligonucleotides were synthesized on a NTS DNA/RNA synthesizer (NIHON TECHNO SERVICE) and a nS-8II synthesizer (GeneDesign). All syntheses were performed using an empty synthesis column of 1.0 μmol scale (Empty Synthesis Columns-TWIST, Glen Research) packed with a N-Tr-morpholino monomers loaded PrimerSupport (Primer Support™ 5G Amino, GE Healthcare, succinate linker).

Coupling of N-Tr-morpholino (PMO)-dimethylphosphoramidochloridate or 3′-DMT-DNA-5′-dimethylphosphoramidochloridate was performed by NTS DNA/RNA synthesizer. Dimethylphosphoramidochloridate reagents were prepared as 0.20 M solutions in 1,3-dimethyl-2-imidazolidinone (DMI), and 0.3 M solution of 1,2,2,6,6-Pentamethylpiperidine (PMP) in DMI was used as coupling activator. Detritylations were performed using 3% trichloroacetic acid (TCA) in DCM (CH₂Cl₂) and capping was done with Cap Mix A (THF/2,6-Lutidine/Ac₂O, Glen Research) and Cap Mix B (16% 1-Me-imidazole/THF, Glen Research). Neutrizations were performed using DIPEA in DMI and DCM. Remaining Ac₂O in the solid support was removed by 0.4M solution of morpholine in DMI. A stepwise description of the synthesis cycle is described in Table 2.

TABLE 2
Synthesis cycle for the coupling of PMO- or DNA-
dimethylphosphoramidochloridate.

| Step | Reaction | Reagent | Time |
|---|---|---|---|
| 1 | Ac₂O removal | Morpholine in DMI (0.4M) | 540 sec |
| 2 | Wash | DCM | |
| 3 | Detritylation | 3 wt/v % TCA in DCM | 40 sec |
| 4 | Wash | DCM | |
| 5 | Neutrization | DIPEA in DMI and DCM (10:45:45) | 120 sec |
| 6 | Wash | DCM | |
| 7 | Coupling | Dimethylphosphoramidochloridate | 8 h |
| | | in DMI (0.2M) | |
| | | PMP in DMI (0.3M) | |
| | | (final concentration of | |
| | | dimethylphosphoramido- | |
| | | chloridate was 0.1M) | |
| 8 | Wash | DCM | |
| 9 | Capping | Cap Mix A (THF/2,6-Lutidine/Ac₂O) | 60 sec |
| | | Cap Mix B (16% 1-Me-imidazole/THF) | |
| 10 | Wash | DCM | |

Performed by NTS DNA/RNA synthesizer (Nihon-techno service)

Coupling of 3′-DMT-DNA-5′-cyanoethyl phosphoramidites and N-Tr-morpholino cyanoethyl phosphoramidites was performed by nS-8II synthesizer. The phosphoramidites were prepared as 0.20 M or 0.30 M solutions in CH₃CN as shown in Table 2. A 0.40 M solution of 5-(Ethylthio)-1H-tetrazole (ETT) in CH₃CN was used as coupling activator. Detritylations were performed using 3% trichloroacetic acid in DCM and capping was done with Cap A Solution-1 (10 vol % Ac2O/THF, WAKO) and Cap B Solution-1 (THF/1-Me-imidazole/Pyridine, (8:1:1, WAKO). Sulfurizations were carried out with 0.05 M solution of ((dimethylamino-methylidene)amino)-3H-1,2,4-dithiazoline-3-thione (DDTT) in pyridine and CH₃CN (3:2). A stepwise description of the synthesis cycle is described in Table 3.

TABLE 3
Synthesis cycle for the coupling of DNA- or PMO-phosphoramidites.

| Step | Reaction | Reagent | Time |
|---|---|---|---|
| 1 | Detritylation | 3 wt/v % TCA in DCM | 20 sec |
| 2 | Coupling | DNA-amidites in CH₃CN (0.20M) | 5 min |
| | | PMO-amidites in CH₃CN (0.20M for A, | |
| | | T, C and 0.3M for G) | |
| | | ETT in CH3CN (0.4M) | |
| | | (final concentration of amidites was 0.1M | |
| | | except for 0.15M of PMO-G) | |
| 3 | Sulfurization | DDTT in pyridine and CH₃CN(3:2) (0.05M) | 10 min |
| 4 | Capping | Cap A (10 vol % acetic anhydride in THF) | 30 sec |
| | | Cap B (1-Me-imidazole/THF/Pyridine) | |

Performed by nS-8II synthesizer (GeneDesign)

Cleavage and de-protection of oligonucleotides: after completion of the automated synthesis, the solid support was treated with 20 vol % diethylamine in CH₃CN and then allowed to stand still for 1 h. The support was washed with anhydrous CH₃CN and dried with argon. The support was transferred into empty screwcap tube and treated with a solution of 28% NH₄OH and EtOH (3:1, 1 mL) at 60° C. for overnight. The support was filtered with Disc SyringeFilter (Hydrophilic PTEE, 0.45 Shimadzu). The filtrate was dried with N₂ flow. The resultant residue was dissolved in water. (Further filtration was performed when there was a suspension in the solution.) The crude material was analyzed by reverse-phase high-performance liquid chromatography (RP-HPLC) and liquid chromatography mass spectrometry (LCMS).

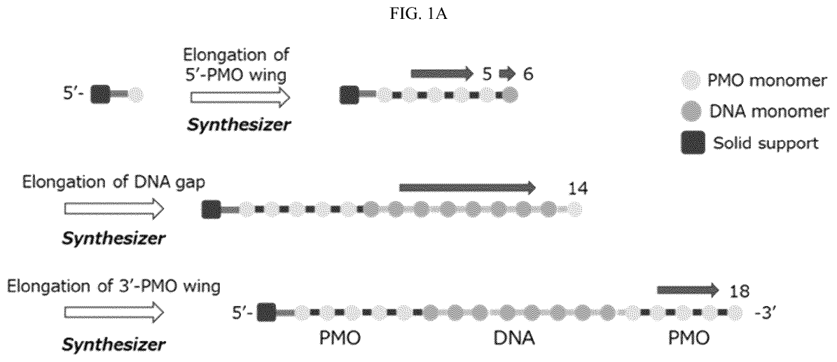

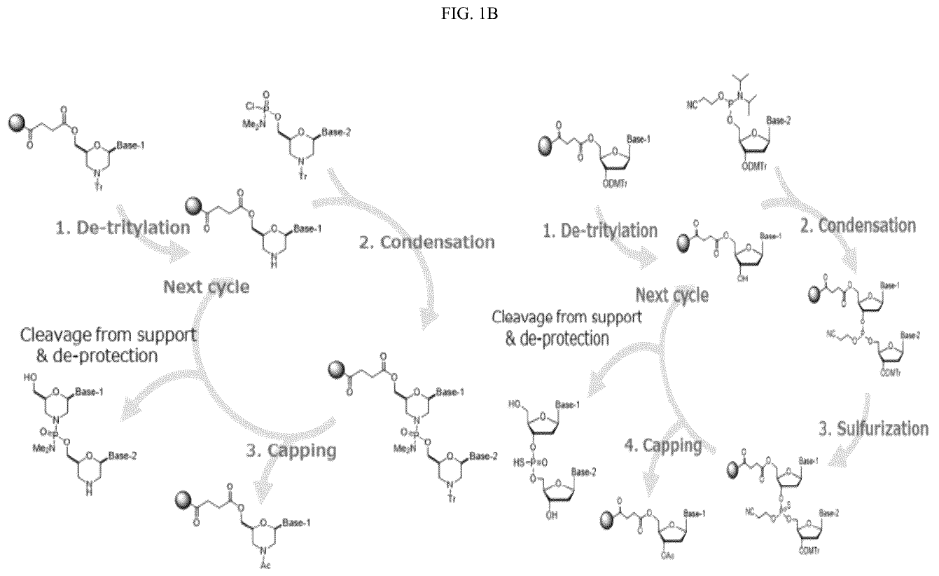

FIG. 1A and FIG. 1B are a schematic representation of the solid phase synthesis of the oligonucleotides and the synthesis cycles of the coupling reactions detailed in this example. 5′-activated DNA monomers were used to overcome the synthetic challenges due to opposite direction of synthesis (i.e. 5′ to 3′ for PMOs and 3′ to 5′ for DNAs).

Purification of N-Tr: the crude material was purified by RP-HPLC with purification condition-1 (small scale) or condition-2 (medium scale). The obtained fractions were collected and dried with N₂ flow.

Purification Condition-1:
Column: XBridge BEH C18 OBD prep (10×150 mm, Particle size 5 μm, Waters)
Detection: 260 nm
Column temperature: 55° C.
Eluent A: 100 mM HFIP, 8.6 mM TEA/water
Eluent B: 100% MeOH
Gradient B: 25% to 56% in 25 min
Flow rate: 3.5 mL/min

Purification Condition-2:
Column: XBridge BEH Prep C18 OBD (19×150 mm, Particle size 5 μm, Waters)
Detection: 260 nm
Column temperature: 55° C.
Eluent A: 100 mM HFIP, 8.6 mM TEA/water
Eluent B: 100% MeOH
Gradient B: 10% to 70% in 20 min
Flow rate: 20 mL/min

Detritylation and purification (for in vitro/in vivo): The solution for detritylation was prepared by mixing TFA (0.17 mL), Et₃N (0.16 mL), EtOH (0.25 mL), 2,2,2-trifluoroethanol (2.5 mL) and DCM (22.25 mL). To the residue of purified N-Tr was added the above solution (excess amount) at 0° C. After several hours at 0° C., 5% DIPEA in DCM was added to the mixture for neutrization. Then the mixture was dried by N2 flow. The residue was dissolved by water and purified by RP-HPLC with purification condition-1 using gradient B: 25% to 35% in 25 min. The obtained fractions were collected and dried with N₂ flow.

Desalting of oligonucleotides (for in vitro): The purified oligonucleotides after detritylation was diluted with water to 2.5 mL of total volume and then desalted by Illustra™ NAP™-25 Columns (GE Healthcare) using water as an equilibration buffer according to the manufacturer's protocol. The obtained solution were dried with N₂ flow.

Ion-exchange of oligonucleotides (for in vivo-1): the purified oligonucleotides after detritylation were diluted with start buffer (0.02 M Na phosphate buffer (pH 8.0), 20% CH₃CN) until the total volume became 1 mL. Anion-exchange was carried out by HiTrapQ HP (1 mL, GE Healthcare) following the manufacturer's protocol using the strat buffer and elution buffer (start buffer with 1.5 M NaCl). The obtained fractions were collected and dried with N2 flow. The residue was diluted with water to 2.5 mL of total volume and then desalted by Illustra™ NAP™-25 Columns (GE Healthcare) using water as an equilibration buffer according to the manufacturer's protocol. The obtained solution were dried with N₂ flow.

Ion-exchange of oligonucleotides (for in vivo-2): anion-exchange was carried out by using centrifugal spin filters (Vivaspin 20, 3,000 molecular weight cut-off, GE Healthcare). The purified oligonucleotides after detritylation were dissolved with NaOAc (0.1 M) up to 14 mL of total volume and then the solution was applied to the spin filter. The sample was concentrated to less than 5 mL with centrifuge. The concenrated solution was diluted with water up to 14 mL of total volume and concentrated to less than 5 mL. This dilution and concentration process was repeated twice. The residue was transferred to empty tube and concentrated with the vacuum concentrator.

Analysis: the obtained residue was dissolved with water and the concentration was determined by the absorbance at 260 nm (measured with Nanodrop) and the factor value (ng·cm/μL).

### Example 3: Determination of Phosphorus Stereochemistry in PMO

Absolute stereochemistry of activated morpholino monomers was determined by X-ray structure of TA PMO dinucleotide (U.S. Pat. No. 10,457,698) and ³¹P NMR chemical shifts. A2 monomer gave TA2 dimer with Sp configuration, which was determined by X-ray crystallography. The stereochemistry of A2 was determined to be Rp based on the inversion of stereochemistry during stereospecific coupling reaction.

A2, T1, C1 and G2 monomers showed a same trend in ³¹P NMR (lower chemical shift than the other corresponding isomer) to suggest A2, T1, C1 and G2 have the same P configuration which was assigned as Rp based on the stereochemistry of A2, and give coupling products with Sp configuration.

Dimers from A2, T1, C1 and G2 showed a same trend in ³¹P NMR: higher chemical shifts than dimers from A1, T2, G1 and C2, respectively.

Table 4 depicts the ³¹P NMR chemical shift and the assigned P stereochemistry for various morpholino monomers and dimers.

TABLE 4
³¹P NMR chemical shift and the assigned P stereochemistry for various
morpholino monomers and dimers.

| | | Assigned | | | Assigned |
|---|---|---|---|---|---|
| Activated Monomer | P NMR (ppm) | stereochemistry* | Dimer | P NMR (ppm) | stereochmistry |
| A1 | 18.416 | S | CT1 | 15.41 | S |
| A2 | 18.069 | R | CT2 | 15.25 | R |
| T1 | 18.016 | R | AT1 | 15.76 | S |
| T2 | 18.355 | S | AT2 | 15.70 | R |
| G1 | 18.466 | S | UT1 | 15.73 | S |
| G2 | 18.119 | R | UT2 | 15.32 | R |
| C1 | 18.027 | R | GT1 | 15.89 | S |
| C2 | 18.435 | S | GT2 | 15.84 | R |
| | | | AC1 | 15.73 | S |
| | | | AC2 | 15.62 | R |
| | | | CA1 | 15.24 | R |
| | | | CA2 | 15.78 | S |
| | | | AG1 | 15.34 | R |
| | | | AG2 | 15.41 | S |

*A1 and A2 mean the early eluting A isomer (A1) and late eluting A isomer (A2) on chiral HPLC conditions for the activated A monomer. Similarly the “1” and “2” designations denote the early and late eluting chiral HPLC conditions for the other activated monomers.

### Example 4: Solution-Phase Synthesis of Stereodefined 5-8-5 PMO-Gapmers

An overall synthetic scheme for the solution phase synthesis of stereodefined PMO-gapmers as an alternative to the scheme in Example 4 is illustrated below:

The stereochemistry of the phosphorus atoms in the phosphorothioate linkages between the deoxyribonucleosides of the PMO-gamers were controlled by using similar methods as those disclosed by Knouse and deGruyter et al. (see Knouse, K. and deGrutyer, J. et al, “Unlocking P(V): Reagents for chiral phosphorothioate synthesis”, Science, 2018, 361(6408): 1234-1238) and Stec et al (see Stec et al, “Deoxyribonucleoside 3¢-O-(2-Thio- and 2-Oxo-“spiro”-4,4-pentamethylene-1,3,2-oxathiaphospholane)s: Monomers for Stereocontrolled Synthesis of Oligo(deoxyribonucleoside phosphorothioate)s and Chimeric PS/PO Oligonucleotides”, J. Am. Chem. Soc. 1998, 120, 7156-7167; Karwowski and Stec et al, “Stereocontrolled synthesis of LNA Dinucleoside phosphorothioate by the oxathiaphospholane approach”, Bioorg. Med. Chem. Lett., 11 (2001) 1001-1003; and Karwowski and Stec et al, “Nucleoside 3′-O-(2-Oxo-“Spiro”-4.4-Pentamethylene-1.3.2-Oxathiaphospholane)S: Monomers For Stereocontrolled Synthesis Of Oligo(Nucleoside Phosphorothioate/Phosphate)S”, Nucleosides & Nucleotides, 17(9-11), 1747-1759 (1998)), which are herein fully incorporated by reference.

The solution phase synthesis of stereodefined PMO-gapmers presented within this example differs from previous solution phase syntheses of antisense oligonucleotides in that the present synthesis utilizes a 12+6 coupling step. Prior solution phase syntheses typically couple one nucleotide at a time until the final product is formed; however, these coupling methods lead to an increased chance that the final product will be contaminated with other species of oligonucleotides of varying lengths. This increased chance of contamination is due to the occurrence of not all of the oligonucleotides having enough time to interact with the next nucleotide added into the solution. Therefore, not only does the final product have an increased chance of containing nucleotides of varying lengths, but also varying nucleotide sequences.

An advantage of performing a 6+12 coupling is that it lowers the number of steps where one nucleotide is added at a time before formation of the final product, hence potentially leading to final products with increased purity and yields.

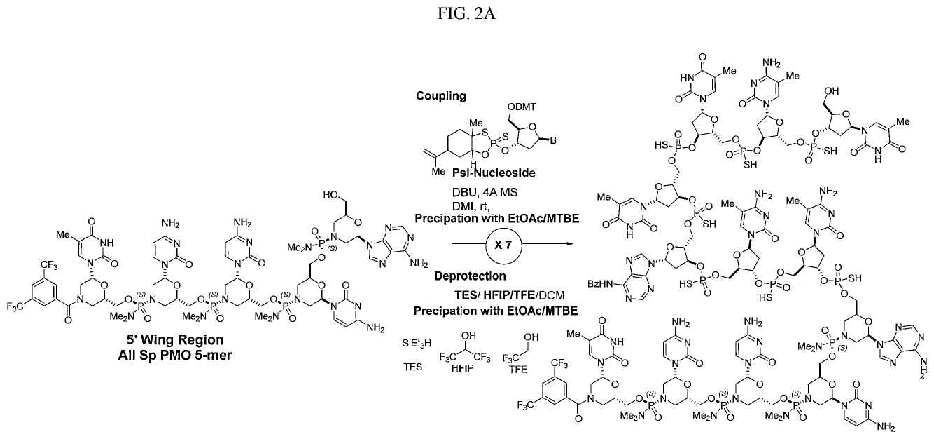

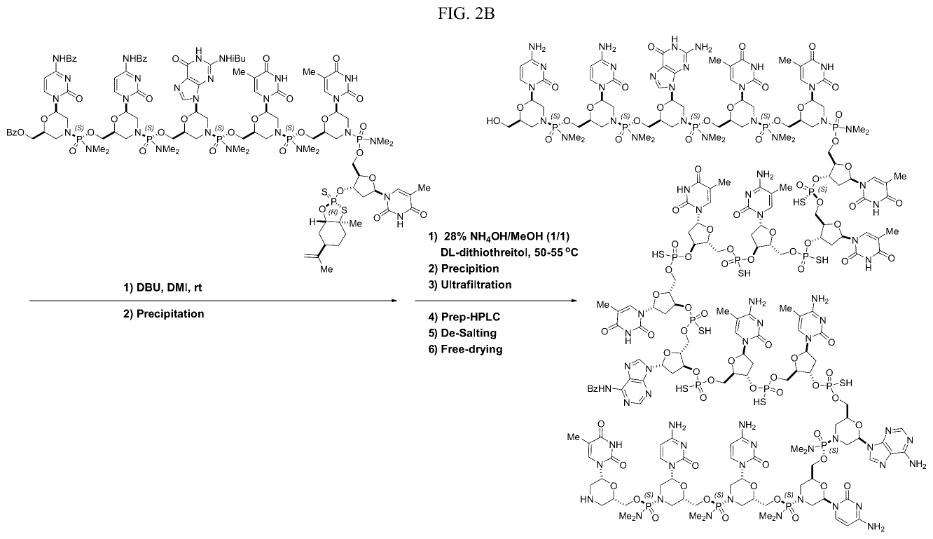

FIG. 2A and FIG. 2B depict a representative synthesis of a PMO-gapmer according to the solution phase synthesis methods detailed in this example.

### Example 4.1: Preparation of 5′-PMO Wing

### 2-Mer of 5′-PMO Wing: Coupling

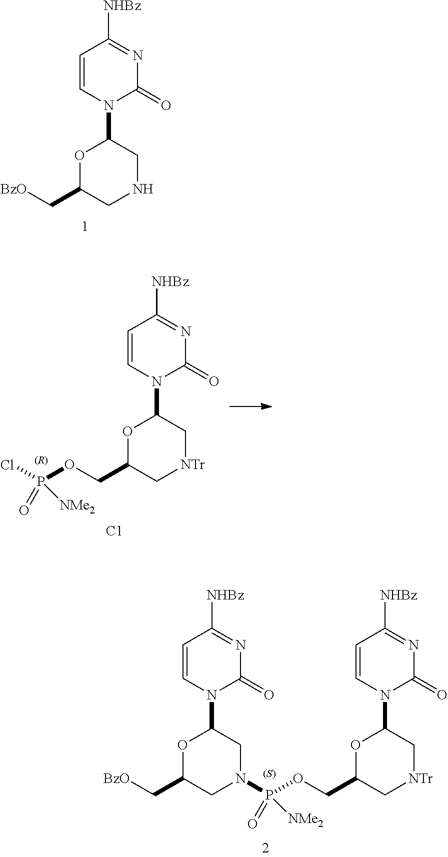

To a solution of starting material 1 (0.500 g, 1.15 mmol) in 1,3-dimethyl-2-imidazolidinone (8.76 mL) was added 1,2,2,6,6-pentamethylpiperidine (0.63 mL) followed by addition of C1 (0.803 g, 1.15 mmol) at room temperature. The solution was stirred till the reaction was completed. Methyl tertiary butyl ether (MTBE) (45 mL) was added slowly, followed by addition of n-heptane (40 mL). The supernatant solution was removed. The solids were dissolved in DCM and purified by silica gel column chromatography using a 0-25% gradient of methanol in DCM as eluents to afford target compound 2 (0.98 g). MS (ESI) m/z: [M+H]⁺ Calcd for C₆₀H₅₉N₉O₁₀P, 1096.41. Found 1096.13.

### 2-Mer of 5′-PMO Wing: Deprotection

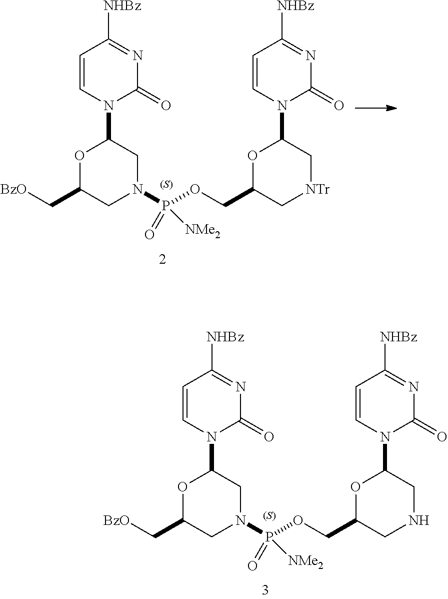

To a solution of starting material 2 (1.2 g, 1.1 mmol) in DCM (12.00 mL) and ethanol (0.64 mL, 11 mmol) was added TFA (0.548 mL, 7.12 mmol) dropwise at room temperature. The reaction was stirred overnight. MTBE (45 mL) was added slowly to the reaction, white precipitate formed (TFA salt). The slurry mixture was stirred for 10-15 min and then filtered. The cake was washed with MTBE (2×10 mL). The TFA salt was dissolved in DCM (12 mL) and treated with 1,2,2,6,6-pentamethylpiperidine (0.991 mL, 5.47 mmol) to form the free base. After the solution was stirred for 10-15 min, MTBE (50 mL) was added slowly to the reaction, leading to white precipitate. The mixture was stirred for 10-15 min and filtered. The cake was washed with MTBE (2×10 mL). 0.74 g of target product 3 was obtained. MS (ESI) m/z: [M+H]⁺ Calcd for C₄₁H₄₅N₉O₁₀P, 854.29. Found 854.20.

### 3-Mer of 5′-PMO Wing: Coupling

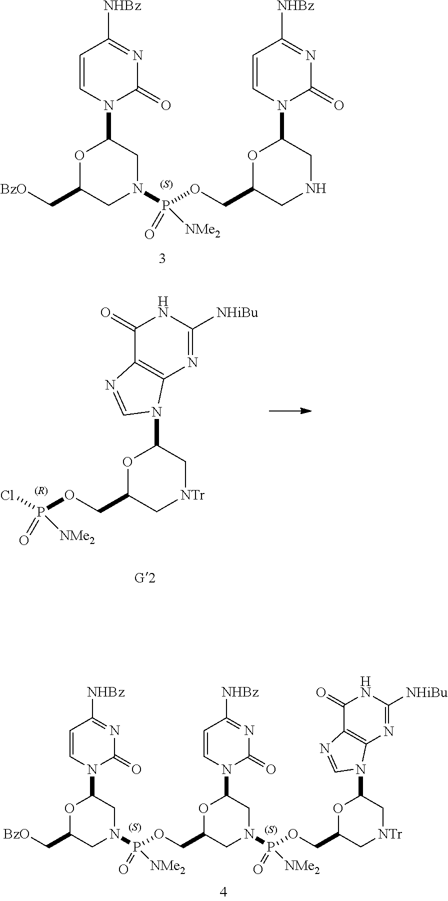

Starting material 3 (0.74 g, 0.87 mmol) was dissolved in 1,3-dimethyl-2-imidazolidinone (8 mL). 1,2,2,6,6-pentamethylpiperidine (0.475 mL, 2.60 mmol) was added followed by addition of G′2 (0.732 g, 1.04 mmol) at room temperature. The mixture was stirred at room temperature for 3-4 h and treated with EtOAc (˜10 mL) and then MTBE (50 mL). The precipitate was collected by filtration and washed with MTBE (2×10 mL). 1.3 g of target product 4 was obtained.

MS (ESI) m/z: [M+H]⁺ Calcd for C₇₆H₈₄N₁₆O₁₅P₂ 1522.56. Found 1522.25.

### 3-Mer of 5′-PMO Wing: Deprotection

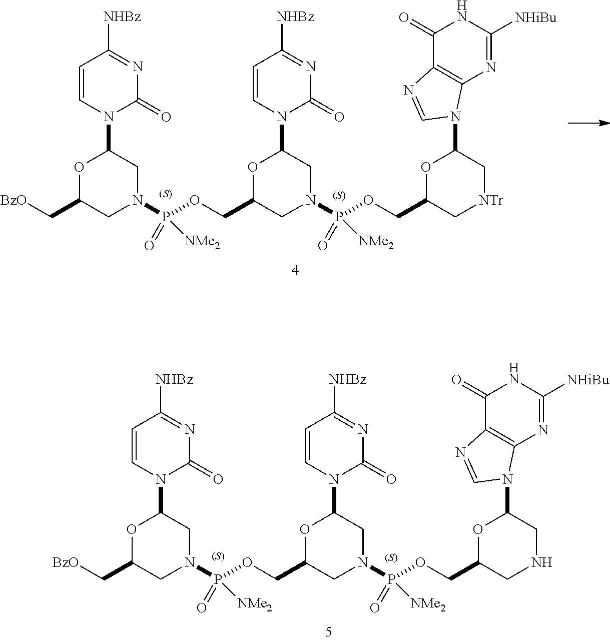

Starting material 4 (1.3 g, 0.85 mmol) was dissolved in DCM (16.8 mL) and ethanol (0.499 mL, 8.54 mmol). TFA (0.329 mL, 4.27 mmol) was added at room temperature. After 2 h, MTBE (55 mL) was added slowly, leading to precipitation. After stirred for 5-10 min, the solids were filtered and washed with MTBE (2×10 mL). The resulting solids were redissolved in 10 mL DCM and treated with 1,2,2,6,6-pentamethylpiperidine (0.780 mL, 4.27 mmol) at room temperature. After the solution was stirred for 10 minutes, MTBE (50 mL) was added slowly, leading to precipitation. After being stirred for 10-15 min, the mixture was filtered, washed with MTBE (2×10 mL) and dried. 1.05 g of target product 5 was obtained. MS (ESI) m/z: [M+H]⁺ Calcd for C₅₇H₆₉N₁₆O₁₅P₂ 1279.46. Found 1279.14.

### 4-Mer of 5′-PMO Wing: Coupling

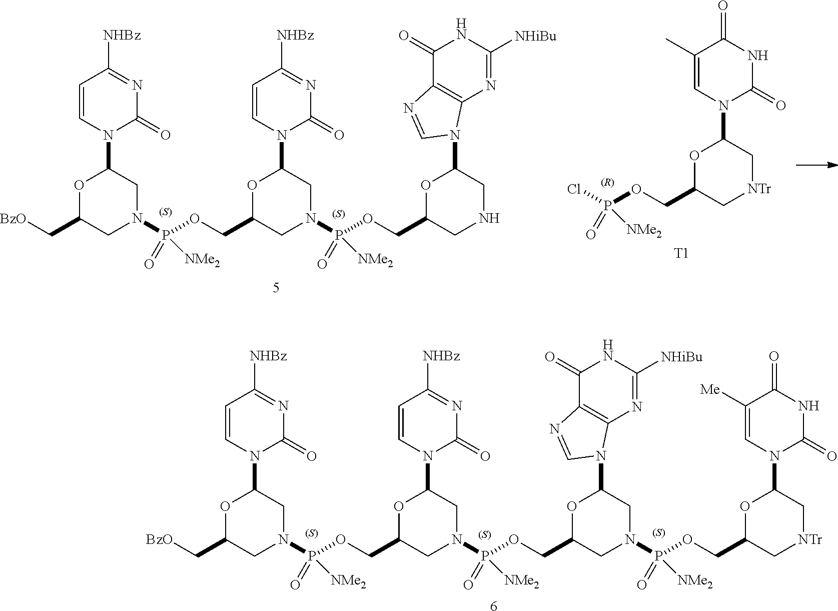

Starting material 5 (1.05 g, 0.821 mmol) was dissolved in 1,3-dimethyl-2-imidazolidinone (10.7 mL). 1,2,2,6,6-pentamethylpiperidine (0.450 mL, 2.46 mmol) was added followed by addition of Ti (0.600 g, 0.985 mmol) at room temperature. The mixture was stirred at room temperature for 2-4 h. 10 mL of EtOAc was added slowly. MTBE (50 mL) was added until a white suspension persisted. The resulting slurry was stirred for 10-15 min and then filtered. The cake was washed with MTBE (2×10 mL) and dried. 1.52 g of target product 6 was obtained.

MS (ESI) m/z: [M+H]⁺ Calcd for C₈₈H₁₀₂N₂₀O₂₀P₃ 1851.68. Found 1852.17.

### 4-Mer of 5′-PMO Wing: Deprotection

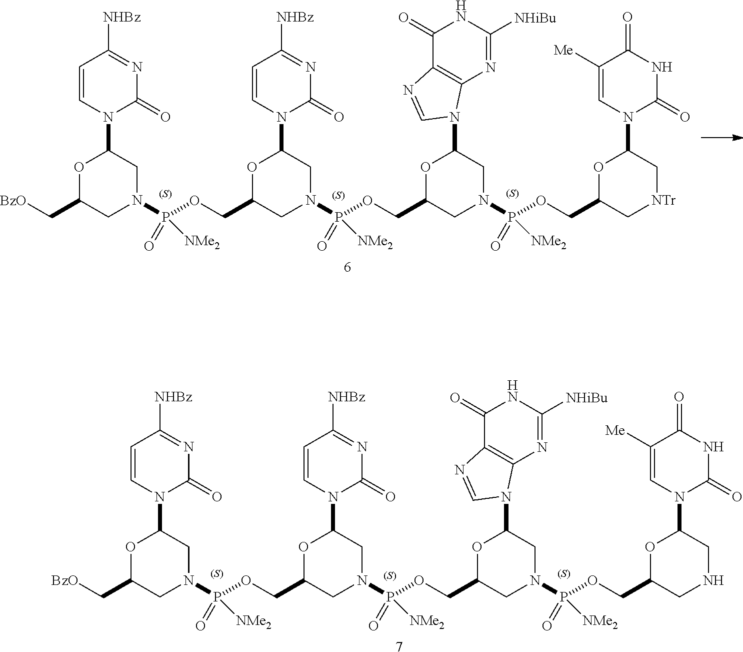

Starting material 6 (1.5 g, 0.81 mmol) was dissolved in DCM (15.9 mL) and ethanol (0.946 mL, 16.2 mmol). TFA (0.478 mL, 6.20 mmol) was added dropwise and the resulting mixture was stirred at room temperature for 2-4 h. EtOAc (10 mL) followed by MTBE (30-mL) was added. White precipitate formed. The slurry was stirred for 10-15 min and filtered. The cake was washed with MTBE (2×10 mL). The precipitate was redissolved in 10 mL DCM and treated with 1,2,2,6,6-pentamethylpiperidine (1.18 mL, 6.48 mmol). After 10 min stirring, EtOAc (30 mL) was added, followed by addition of MTBE (30 mL). The resulting mixture was stirred for 10-15 min and the precipitate was collected by filtration, washed with MTBE (2×10 mL) and dried. 0.96 g of target product 7 was obtained. MS (ESI) m/z: [M+H]⁺ Calcd for C₆₉H₈₈N₂₀O₂₀P₃ 1609.57. Found 1610.21.

### Of 5′-PMO Wing: Coupling

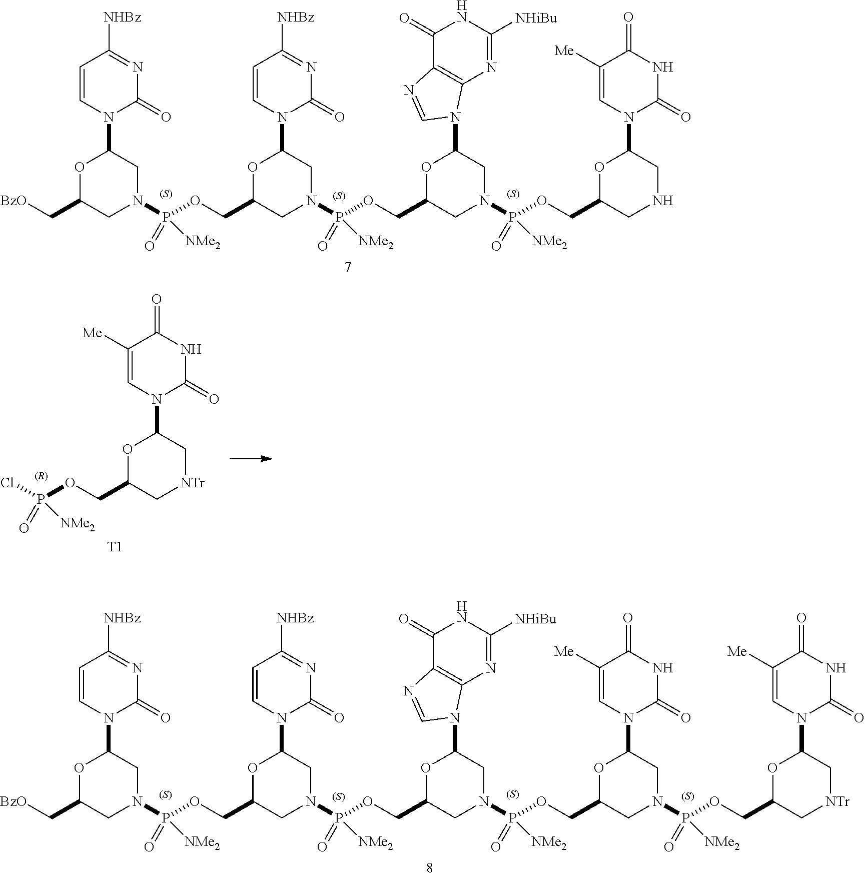

Starting material 7 (0.96 g, 0.60 mmol) was dissolved in 1,3-dimethyl-2-imidazolidinone (9.73 mL). 1,2,2,6,6-pentamethylpiperidine (0.327 mL, 1.789 mmol) was added followed by addition of Ti (0.436 g, 0.716 mmol) at room temperature. The mixture was stirred for 12-16 h. EtOAc (20 mL) was added followed by MTBE (40 mL). The resulting mixture was stirred for 10-15 min and filtered. The cake was washed with EtOAc (2×10 mL) and dried. 1.3 g of target product 8 was obtained.

MS (ESI) m/z: [M+2H]²⁺ Calcd for C₁₀₀H₁₂₁N₂₄O₂₅P₄ 1090.89. Found 1091.55.

### 5-Mer of 5′-PMO Wing: Deprotection

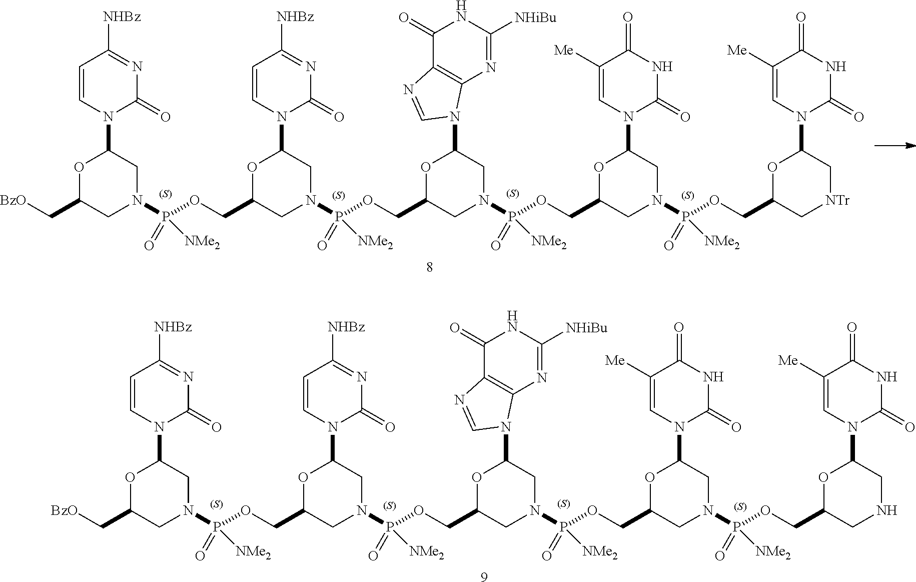

Starting material 8 (1.3 g, 0.60 mmol) was dissolved in DCM (11.7 mL). Ethanol (0.696 mL, 11.9 mmol) followed by TFA (0.275 mL, 3.57 mmol) was added dropwise at room temperature. The resulting mixture was stirred for 2-3 hr at room temperature. EtOAc (40 mL) was added until precipitate formed. The slurry was stirred for 5-10 min and filtered. The cake was washed with EtOAc (2×5 mL). The precipitate was redissolved in DCM 8 mL and 1,2,2,6,6-pentamethylpiperidine (0.871 mL, 4.77 mmol) was added. The resulting solution was stirred at room temperature for 10-15 minutes and treated with EtOAc (10 mL) followed by MTBE (40 mL). The resulting mixture was stirred for 5-10 min and filtered. The cake was washed with MTBE (2×5 mL) and dried. 1.1 g of target product 9. MS (ESI) m/z: [M+H]⁺ Calcd for C₈₁H₁₀₇N₂₄O₂₅P₄ 1939.68. Found 1939.98.

### 6-Mer of 5′-PMO Wing: Coupling

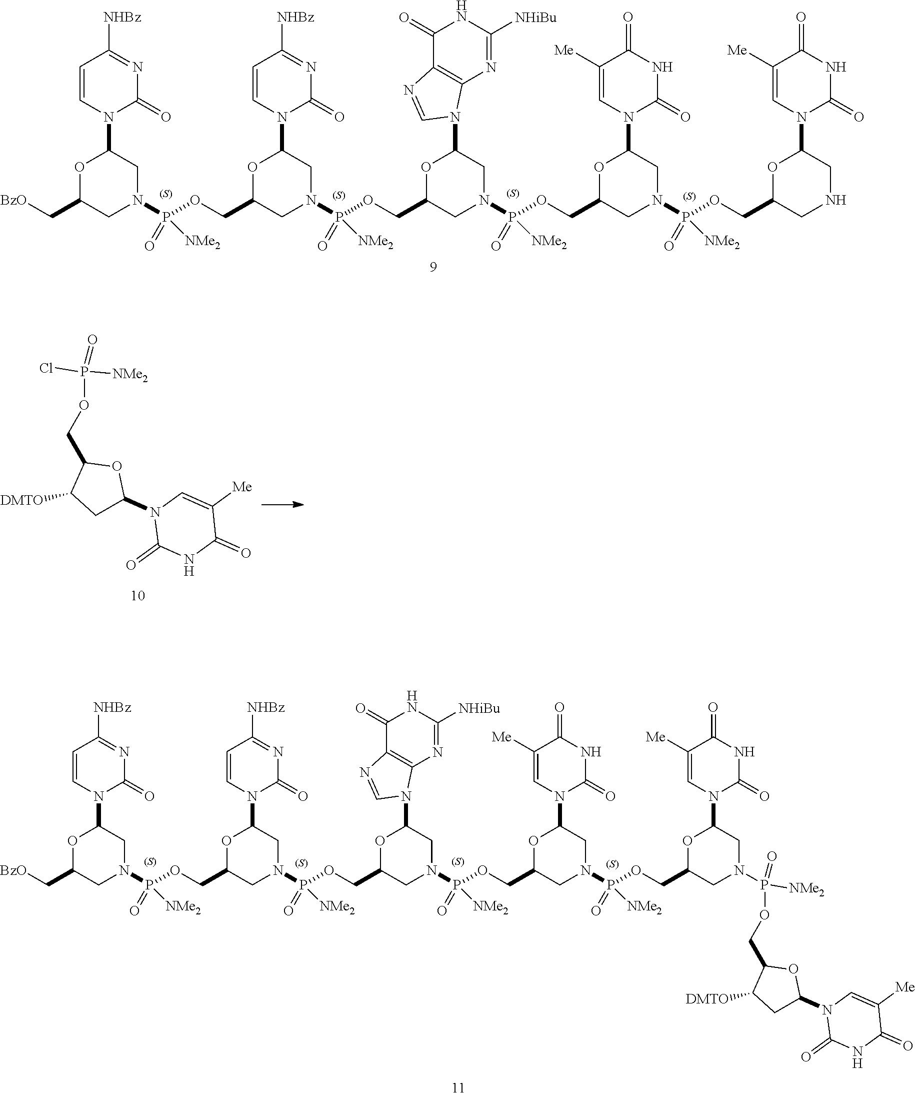

Starting material 9 (1.1 g, 0.567 mmol) was dissolved in 1,3-dimethyl-2-imidazolidinone (12 mL). 1,2,2,6,6-Pentamethylpiperidine (0.411 mL, 2.27 mmol) was added followed by addition of ((2R,3S,5R)-3-(bis(4-methoxyphenyl)(phenyl)methoxy)-5-(5-methyl-2,4-dioxo-3,4-dihydropyrimidin-1(2H)-yl)tetrahydrofuran-2-yl)methyl dimethylphosphoramidochloridate 10 (0.532 g, 0.794 mmol) at room temperature. The mixture was stirred at room temperature overnight. 10 mL EtOAc and 20-30 mL MTBE were added. The resulting slurry was stirred for 10-15 minutes and filtered. The cake was washed with EA (2×10 mL) and dried. 1.45 g of target product 11 was obtained.

MS (ESI) m/z: [M+2H]²⁺ Calcd for C₁₁₄H₁₄₄N₂₇O₃₃P₅ 1286.96. Found 1287.22.

### 6-Mer of 5′-PMO Wing: Deprotection

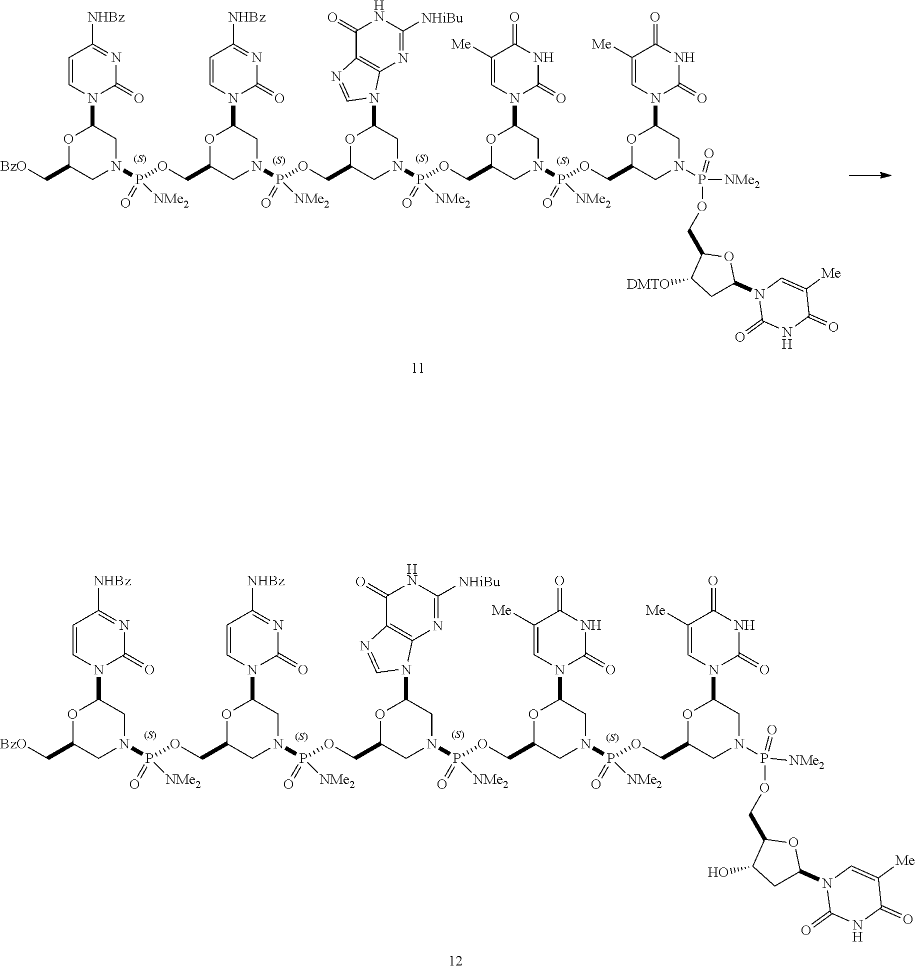

Starting material 11 (1.45 g, 0.563 mmol) was dissolved in DCM (27.2 mL) and ethanol (1.65 mL, 28.2 mmol). Dichloroacetic acid (1.86 mL, 22.5 mmol) was added at room temperature. After 3 h reaction was completed. EtOAc (10 mL) was added followed by MTBE (40-50 mL) until precipitate persisted. The mixture was stirred for 5 min and filtered. The cake was washed with MTBE (2×10 mL) and dried. 1.28 g of target product 12 was obtained.

MS (ESI) m/z: [M+2H]²⁺ Calcd for C₉₃H₁₂₆N₂₇O₃₁P₅ 1135.89. Found 1135.95.

Activation of 5′ 6-Mer with (−)-PSI

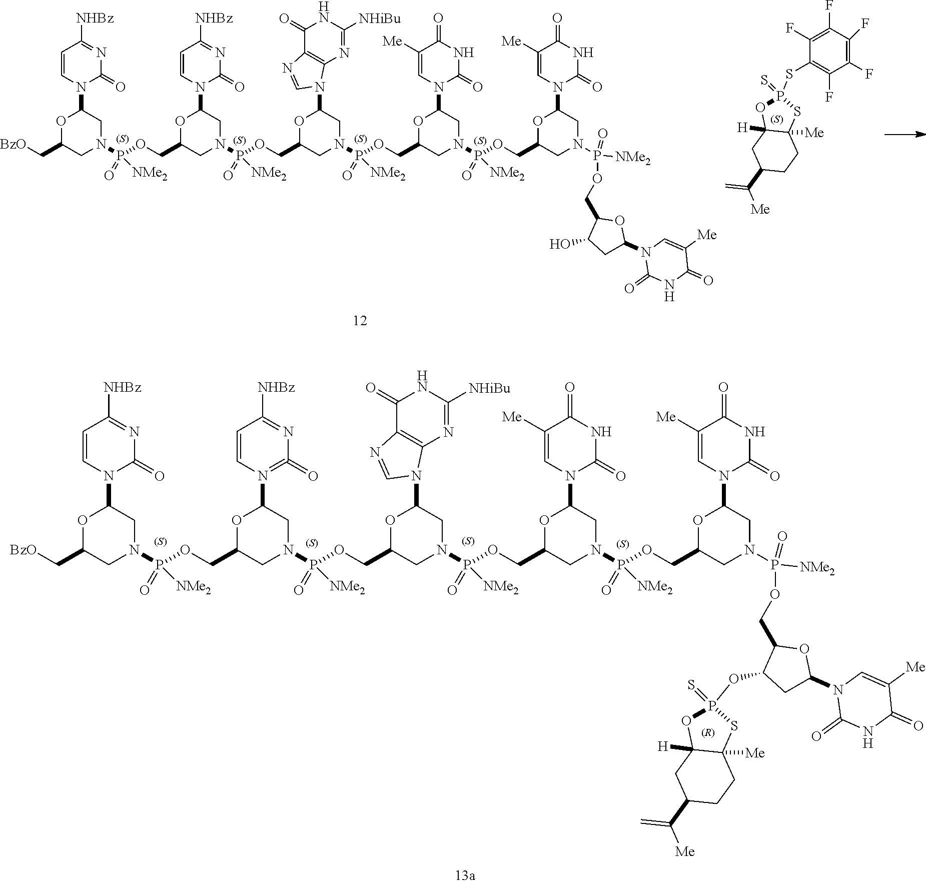

Starting material 12 (1.28 g, 0.564 mmol) and (−)-PSI reagent (Aldrich, CAS: 2245335-70-8, 0.352 g, 0.789 mmol) were added to a reaction flask. 3.8 g 4 Å molecular sieves was added, the reaction mixture was flushed with nitrogen for 10-20 min. DCM (30 mL) and THF (20 mL) were added. The resulting mixture was stirred at room temperature and flushed with N₂ for 30 min. DBU (0.119 mL, 0.789 mmol) was added dropwise. The reaction mixture was stirred for 1-2 h. Once completed, the reaction mixture was filtered into a flask containing MTBE (120 mL). White precipitate formed. The precipitate was stirred for min. The precipitate were filtered, washed with MTBE (2×10 mL) and dried. The precipitate was recovered and dried to give 1.3 g of target product 13a.

MS (ESI) m/z: [M+2H]²⁺ Calcd for C₁₀₃H₁₄₁N₂₇O₃₂P₆S₂ 1258.91. Found 1259.17.

Alternative Route: Activation of 6-Mer with 2-Chloro-“Spiro”-4,4-Pentamethylene-1,3,2-Oxathiaphospholane

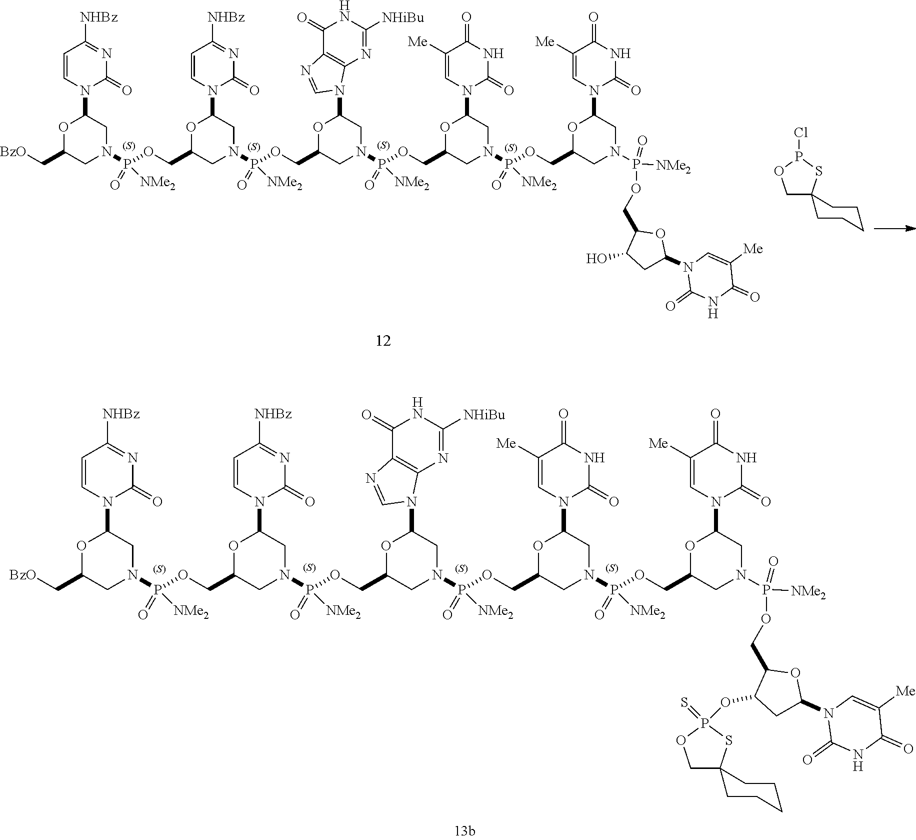

To a magnetically stirred solution of 12 (2.3 g, 1.0 mmol) and 0.19 mL of diisopropylethylamine (1.1 mmol) in THF and DCM, 2-Chloro-“spiro”-4,4-pentamethylene-1,3,2-oxathiaphospholane (1.1 mmol) is added dropwise at room temperature. After the reaction is complete, elemental sulfur (1.5 mmol) is added. Stirring is continued for 12 h. Once completed, the reaction mixture is filtered into a flask containing MTBE. The resulting precipitate is filtered, washed with MTBE and dried in vacuo. The precipitate is recovered and further dried to give target product 13b.

### Example 4.2: Preparation of 3′-PMO Wing

### 2-Mer of 3′-PMO: Coupling

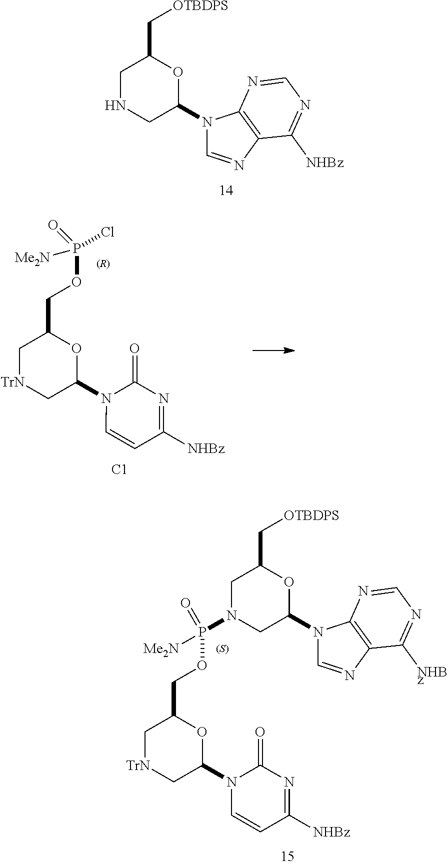

Starting material 14 (100 mg, 0.169 mmol) was chased with MeCN once, then dissolved in DCM (2 mL), followed by addition of 1,2,2,6,6-pentamethylpiperidine (92 mmol). To the mixture was added reactant C1 (144 mg, 0.206 mmol) at room temperature. The reaction mixture was stirred at room temperature overnight. It was then directly subjected to silica gel column chromatography. Elution with 8% MeOH in DCM afforded 216 mg of target product 15.

MS (ESI) m/z: [M+H]⁺ Calcd for C₇₀H₇₃N₁₁O₈PSi 1254.51. Found 1254.43.

### 2-Mer of 3′-PMO: Deprotection

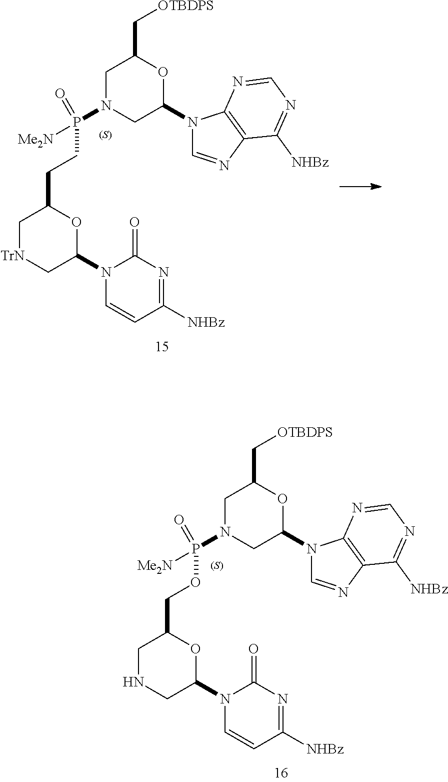

Into a flask charged with starting material 15 (212 mg, 0.169 mmol) was added a solution of TFA (85 μL, 1.1 mmol) in DCM (2.8 mL), followed by addition of ethanol (99 μL, 1.7 mmol). The reaction mixture was stirred at room temperature for 1 h. It was worked up with a saturated aqueous NaHCO₃ solution, and extracted with DCM twice. The DCM layers were combined and washed with half saturated brine, dried over Na₂SO₄, concentrated. The resulting residue was purified with silica gel column chromatography to give 137 mg of target product 16.

MS (ESI) m/z: [M+H]⁺ Calcd for C₅₁H₅₉N₁₁O₈PSi 1012.41. Found 1012.30.

### 3-Mer of 3′-PMO: Coupling

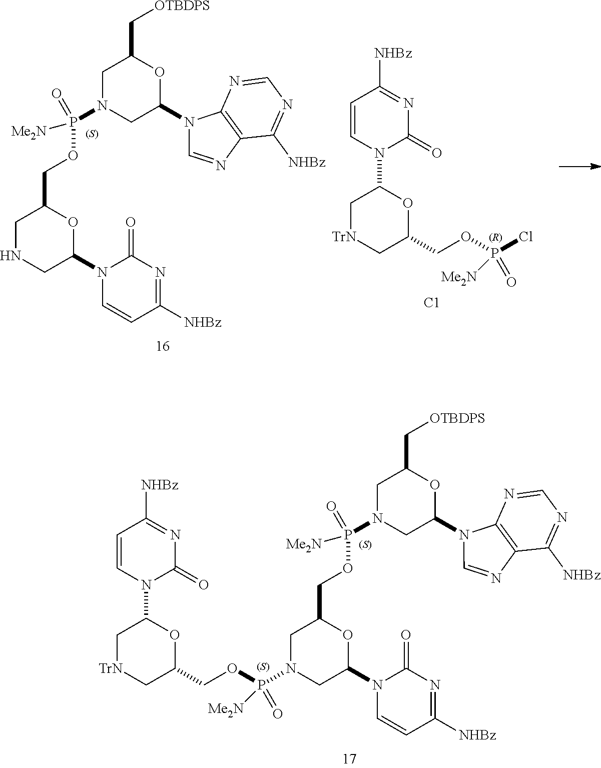

To a solution of starting material 16 (137 mg, 0.135 mmol) in 1,3-dimethyl-2-imidazolidinone (2 mL) was added 1,2,2,6,6-pentamethylpiperidine (73.5 μL, 0.406 mmol), followed by addition of reactant C1 (123 mg, 0.176 mmol) at room temperature. The reaction mixture was stirred at room temperature for 2 h. Into the reaction mixture was added MTBE (20 mL) followed by addition of n-heptane (10 mL). The precipitate was collected by filtration and rinsed with MTBE/n-heptane (9 mL, 2:1 v/v). The precipitate was redissolved in DCM (15 mL) and treated with morpholine (12 μL, 0.14 mmol) at room temperature. The mixture was stirred at room temperature over weekend before it was concentrated and chased with MeCN. The material (17) was directly used for next step without further purification.

MS (ESI) m/z: [M+H]⁺ Calcd for C₈₈H₉₅N₁₆O₁₃P₂Si, 1673.65. Found 1673.45.

3-mer of 3′-PMO: Deprotection

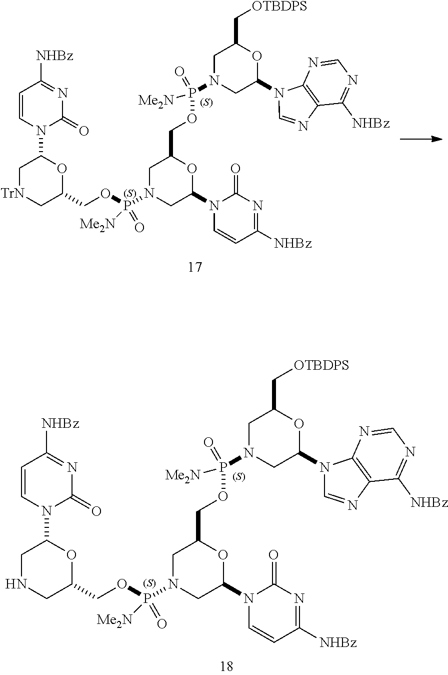

Into a flask charged with starting material 17 (227 mg, 0.136 mmol) was added a solution of TFA (67.9 μL, 0.881 mmol) in DCM (2.3 mL), followed by addition of ethanol (79 μL, 1.4 mmol). The reaction mixture was stirred at room temperature for 40 min before additional TFA (130 μL, 1.68 mmol) in DCM (1.2 mL) was added at room temperature. It was stirred at room temperature for 6 h. Into the mixture was added MTBE (21 mL) and n-heptane (7 mL). The precipitate was collected by filtration and rinsed with MTBE. 238 mg precipitate was obtained. The precipitate was then redissolved in DCM (2 mL), into which was added 1,2,2,6,6-pentamethylpiperidine (198 μL, 1.08 mmol) at room temperature. The mixture was stirred at room temperature for 1 h before MTBE (20 mL) was added, and the resulted suspension was stirred at room temperature overnight. The precipitate was collected by filtration and rinsed with MTBE. 205 mg of target product 18 was obtained.

MS (ESI) m/z: [M+H]⁺ Calcd for C₆₉H₈₁N₁₆O₁₃P₂Si, 1431.54. Found 1431.26.

### 4-Mer of 3′-PMO: Coupling

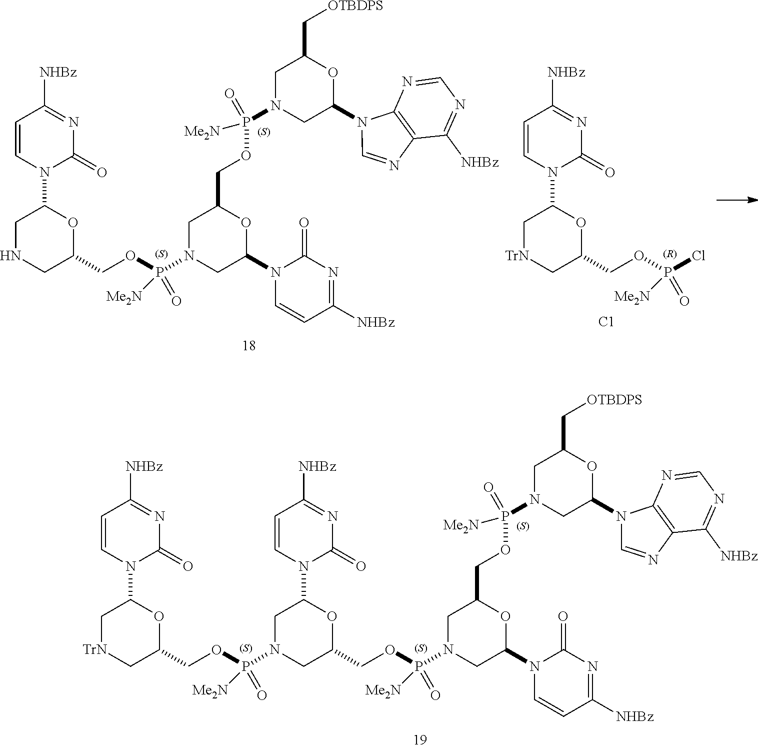

To a solution of starting material 18 (205 mg, 0.143 mmol) in 1,3-dimethyl-2-imidazolidinone (3.0 mL) was added 1,2,2,6,6-pentamethylpiperidine (78 μL, 0.43 mmol), followed by addition of reactant C1 (125 mg, 0.179 mmol) at room temperature. The reaction mixture was stirred at room temperature for 1.5 h before morpholine (12.5 μL, 0.143 mmol) was added. The mixture was stirred at room temperature overnight, into which was then added MTBE until no product in supernatant was detected by LCMS. The precipitate was collected by filtration and rinsed with MTBE. It was then purified by silica gel column chromatography with 12 to 15% MeOH in DCM to give 194 mg of target product 19. MS (ESI) m/z: [M+2H]²⁺ Calcd for C₁₀₆H₁₁₈N₂₁O₁₈P₃Si, 1046.90. Found 1047.16.

4-mer of 3′-PMO: Deprotection

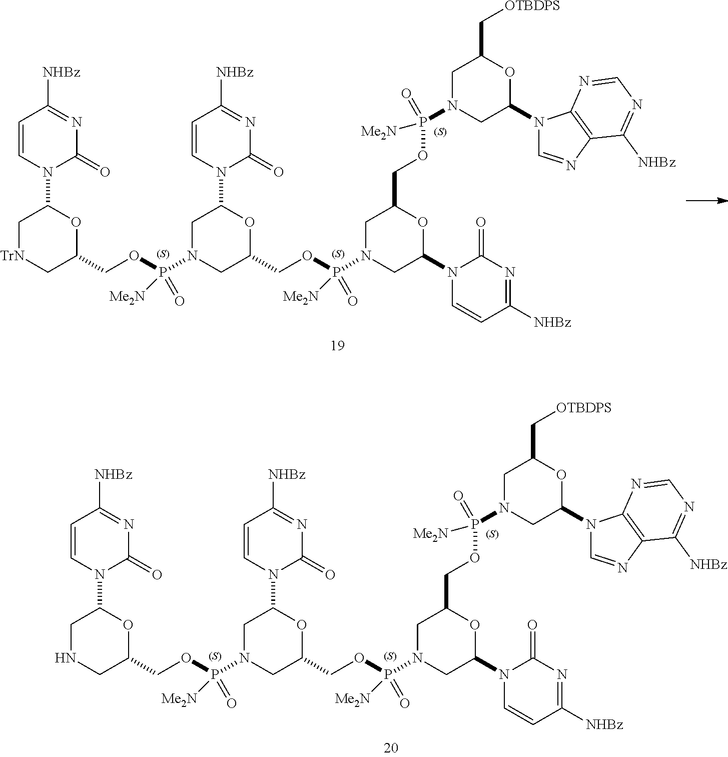

Into a flask charged with starting material 19 (194 mg, 0.093 mmol) was added a solution of TFA (60 μL, 0.78 mmol) in DCM (2.0 mL), followed by addition of ethanol (54.1 μL, 0.93 mmol). The reaction mixture was stirred at room temperature for 5 h before MTBE (20 mL) was added. The precipitate was collected by filtration and rinsed with MTBE. The precipitate was redissolved in DCM (2.0 mL), into which was added 1,2,2,6,6-pentamethylpiperidine (102 μL, 0.556 mmol). The mixture was stirred at room temperature for 20 min before MTBE (20 mL) was added. The precipitate was collected by filtration and rinsed with MTBE. 167 mg of target product 20 was obtained.

MS (ESI) m/z: [M+H]⁺ Calcd for C₈₇H₁₀₃N₂₁O₁₈P₃Si, 1850.68. Found 1850.56.

### 5-Mer of 3′-PMO: Coupling

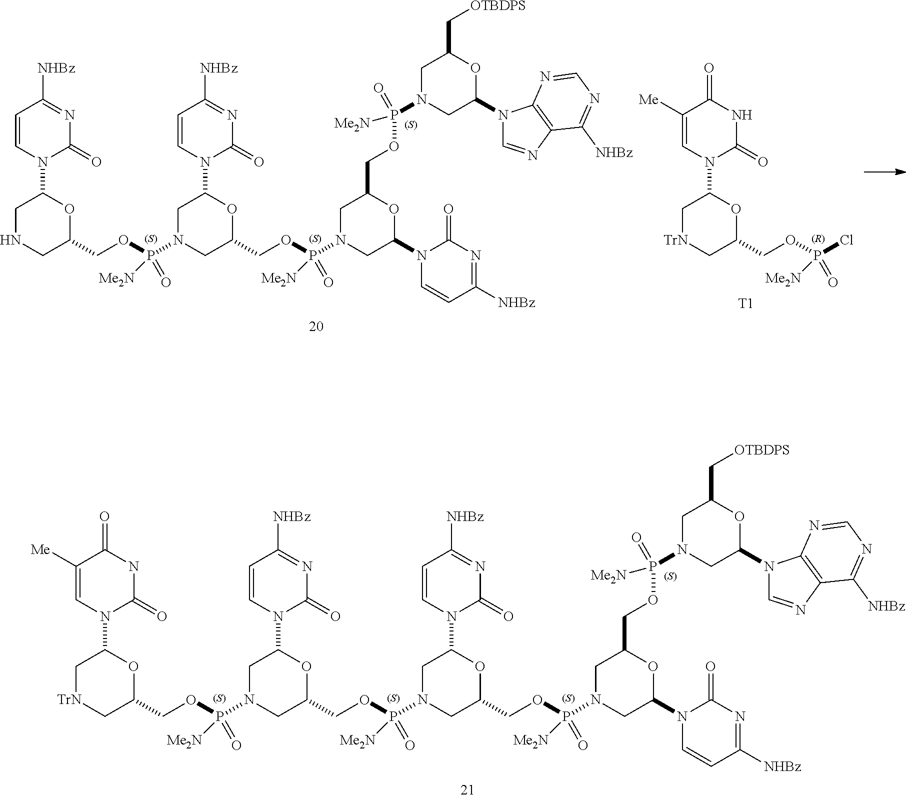

To a solution of starting material 20 (167 mg, 0.09 mmol) in 1,3-dimethyl-2-imidazolidinone (2.0 mL) was added 1,2,2,6,6-pentamethylpiperidine (49.4 μL, 0.271 mmol), followed by addition of reactant T1 (71 mg, 0.12 mmol) at room temperature. The reaction mixture was stirred at room temperature over weekend before MTBE (20 mL) was added. The supernatant was removed by decantation. The residue was purified with silica gel column chromatography. Elution with 10% to 30% MeOH in DCM afforded 202 mg of target product 21.

MS (ESI) m/z: [M+2H]²⁺ Calcd for C₁₁₈H₁₃₇N₂₅O₂₃P₄Si, 1211.95. Found 1212.46.

### 5-Mer of 3′-PMO: Deprotection

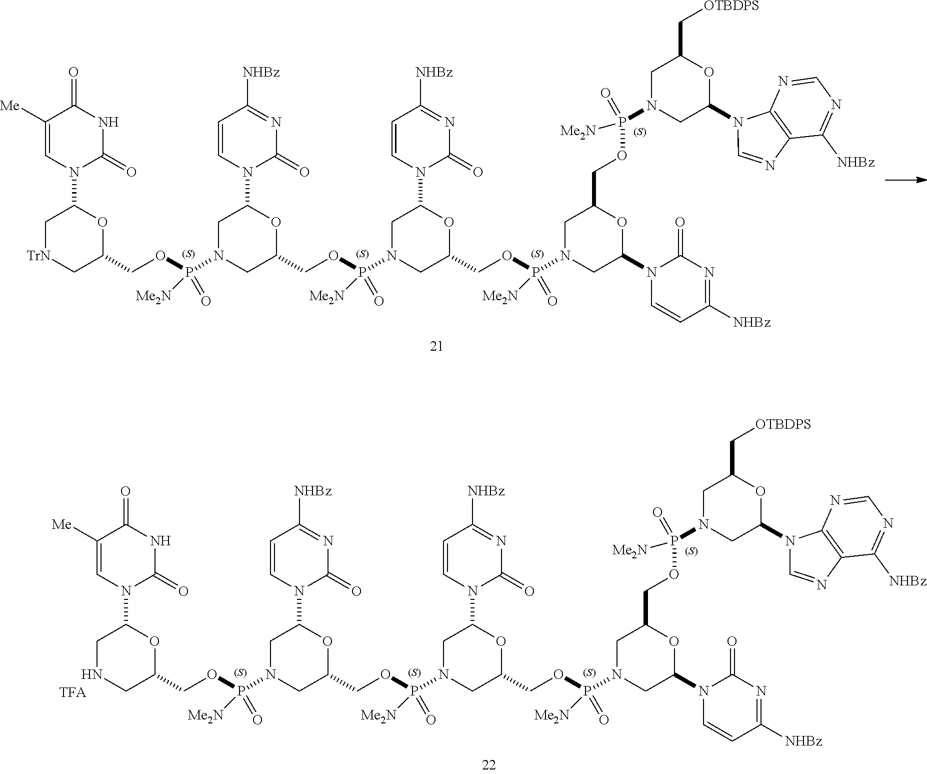

To a solution of starting material 21 (1.65 g, 0.647 mmol) in DCM (15.7 mL) was added ethanol (0.38 mL, 6.5 mmol) and then TFA (0.470 mL, 6.10 mmol). After 1.5 h at room temperature, MTBE (60 mL) was added. The resulting slurry was filtered through a sintered glass filter. The cake was rinsed with a mixture of MTBE/DCM (10 mL/3 mL) and dried in vacuo for 2 h, leading to 1.44 g of target product 22.

MS (ESI) m/z: [M+2H]²⁺ Calcd for C₉₉H₁₂₃N₂₅O₂₃P₄Si, 1090.90. Found 1091.03.

### 5-Mer of 3′-PMO: Deprotection of Bz Groups

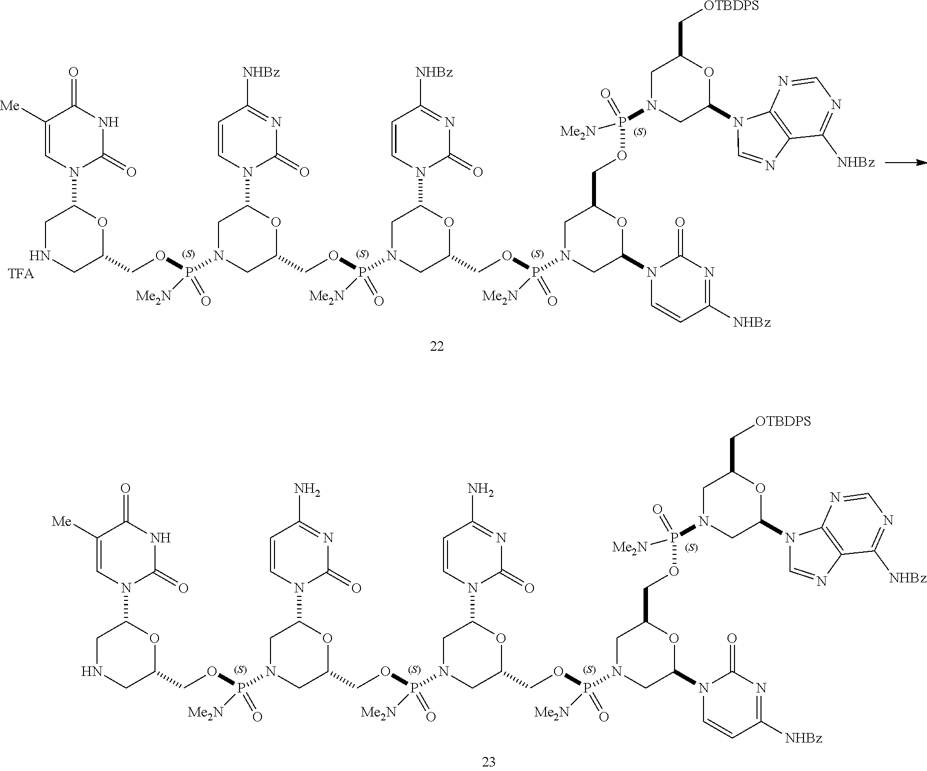

Starting material 22 (0.44 g, 0.19 mmol) was dissolved in a mixture of methanol (6 mL) and 28% ammonium hydroxide (6 mL) at room temperature. The resulting mixture was heated at 50-52° C. for 12 h and cooled to room temperature. Most of solvents were removed by nitrogen purge. The residue was dissolved in DCM/MeOH (6/2 mL) and treated with 40 mL EtOAc. Upon addition of EtOAc, precipitation occurred. The resulting precipitate was collected by filtration and rinsed with a mixture of EtOAc/DCM/MeOH (20 mL/3 mL/1 mL). Drying in vacuo overnight afforded 330 mg of target product 23.

MS (ESI) m/z: [M+H]⁺ Calcd for C₇₁H₁₀₆N₂₅O₁₉P₄Si, 1764.68. Found 1764.99.

### 5-Mer of 3′-PMO: Morpholine Protection

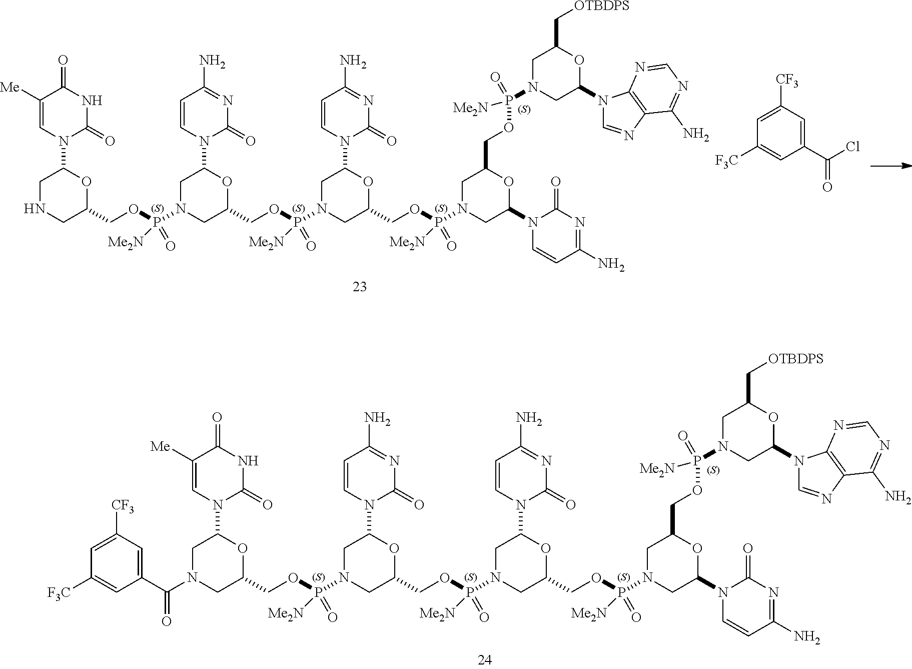

To a solution of starting material 23 (526 mg, 0.298 mmol) in a mixture of THF/Water/MeOH (9 mL/1.6 mL/1 mL) was added 1,2,2,6,6-pentamethylpiperidine (162 mmol) and 3,5-bis(trifluoromethyl)benzoyl chloride (64.8 μL, 0.358 mmol). The resulting mixture was stirred at room temperature while the reaction progress was monitored by LCMS. After 1 h, additional 30 μL of bis(trifluoromethyl) benzoyl chloride was added in two portions. Once the reaction was complete, the reaction mixture was concentrated in vacuo. The resulting residue was dissolved in a mixture of DCM/MeOH (12 mL/3 mL) and then treated with EtOAc (80 mL). Upon addition of EtOAc, precipitation occurred. The resulting precipitate was collected by filtration and rinsed with EtOAc/DCM (4 mL/1 mL) and EtOAc (10 mL). Drying in vacuo for 2 h afforded 547 mg of target product 24. Further precipitation occurred in the resulting filtrate. 24 mg of the 2nd crop was obtained.

MS (ESI) m/z: [M+2H]²⁺ Calcd for C₈₀H₁₀₉F₆N₂₅O₂₀P₄Si, 1002.84. Found 1002.91.

### 5-Mer of 3′-PMO: Deprotection of TBDPS

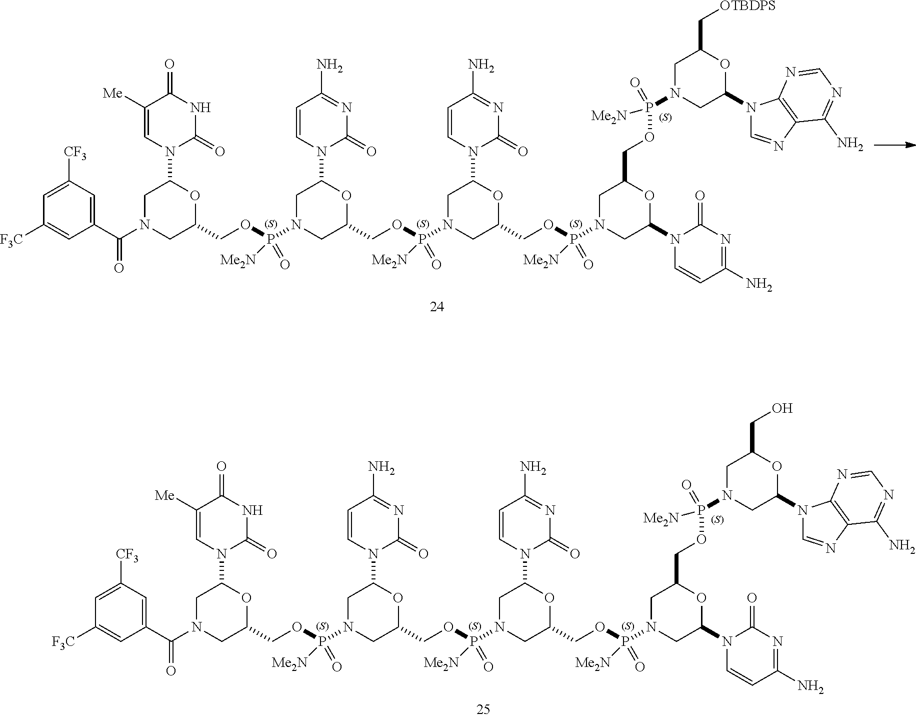

To a solution of starting material 24 (571 mg, 0.285 mmol) in 1,3-dimethyl-2-imidazolidinone (5.7 mL) were added pyridine (8.6 mL) and TEA (8.6 mL) at room temperature. The resulting solution was treated with TEA-3HF (371 μL, 2.278 mmol) and then stirred overnight. Upon completion monitored by LCMS, the reaction mixture was treated with methoxytrimethylsilane (3.4 mL, 25 mmol) and stirred for 1 h at room temperature. MeOH (3 mL) and 1,3-dimethyl-2-imidazolidinone (6 mL) were then added to make a clear solution. The resulting solution was added into EtOAc (60 mL), rinsing with ˜10 mL EtOAc. Upon addition, white precipitation occurred. The slurry was filtered through a sintered glass filter and rinsed with EtOAc (10 mL). The resulting precipitate was dissolved in a mixture of DCM (20 mL)/1,3-dimethyl-2-imidazolidinone (20 mL) and treated with EtOAc (50 mL) at room temperature. Upon addition of EtOAc, precipitation occurred. The resulting precipitate was collected by filtration and rinsed with EtOAc (15 mL). Drying in vacuo with nitrogen purge provided 523 mg of target product 25.

MS (ESI) m/z: [M+H]⁺ Calcd for C₆₄H₉₀F₆N₂₅O₂₀P₄ 1766.56. Found 1766.61.

### Example 4.3: Elongation of DNA

### 6-Mer: Coupling

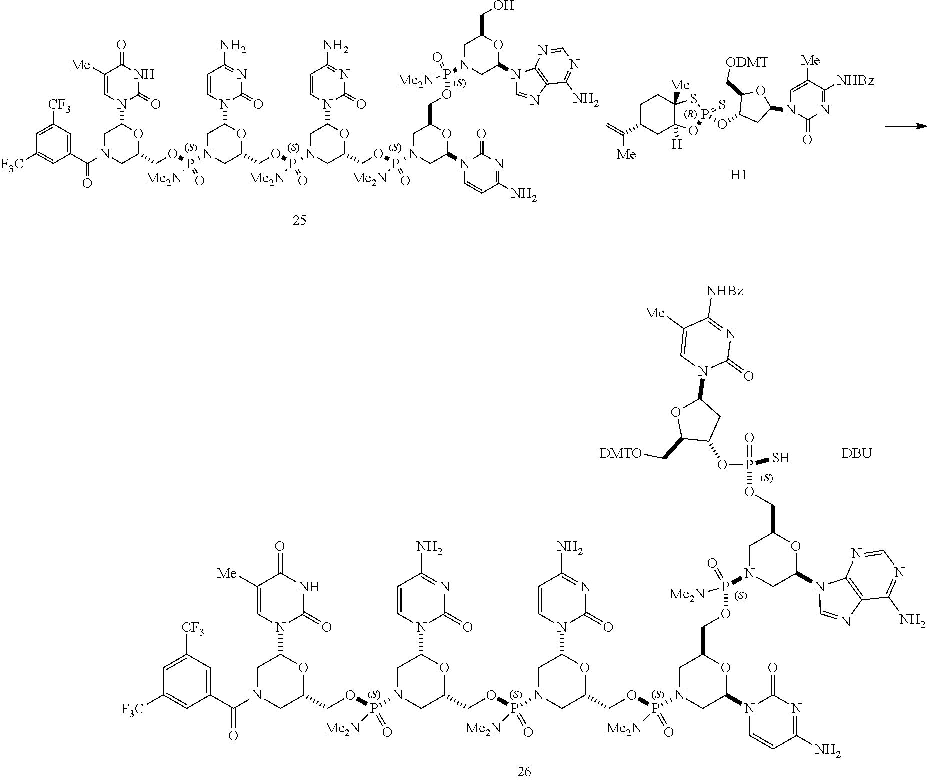

Starting material 25 (125 mg, 0.071 mmol) and reactant 111 (158 mg, 0.177 mmol) were dissolved in 1,3-dimethyl-2-imidazolidinone (3 mL) and the resulting mixture was azeotroped with toluene three times (2 mL each) at 30-32° C. To the resulting solution was added 4 Å molecular sieves (350 mg). The reaction flask was applied to vacuum and filled with nitrogen. The process was repeated two more times. To the resulting mixture was added DBU (0.064 mL, 0.42 mmol) and the reaction mixture was stirred at room temperature overnight (16 h) while the reaction progress was monitored by LCMS. Upon competition, the reaction mixture was filtered through a syringe filter and the filtrate was added into EtOAc (15 mL), rinsing with 1,3-dimethyl-2-imidazolidinone (2 mL). To the resulting slurry was added additional 7.5 mL of EtOAc. The resulting precipitate was collected by filtration and rinsed with a mixture of EtOAc/1,3-dimethyl-2-imidazolidinone (5 mL/1 mL) and EtOAc (10 mL). Drying in vacuo for 40 min provided 228 mg of target product 26.

HRMS (ESI) m/z: [M+H]⁺ Calcd for C₁₀₂H₁₂₆F₆N₂₈O₂₈P₅S, 2492.7643. Found 2492.7361.

### 6-Mer: Deprotection

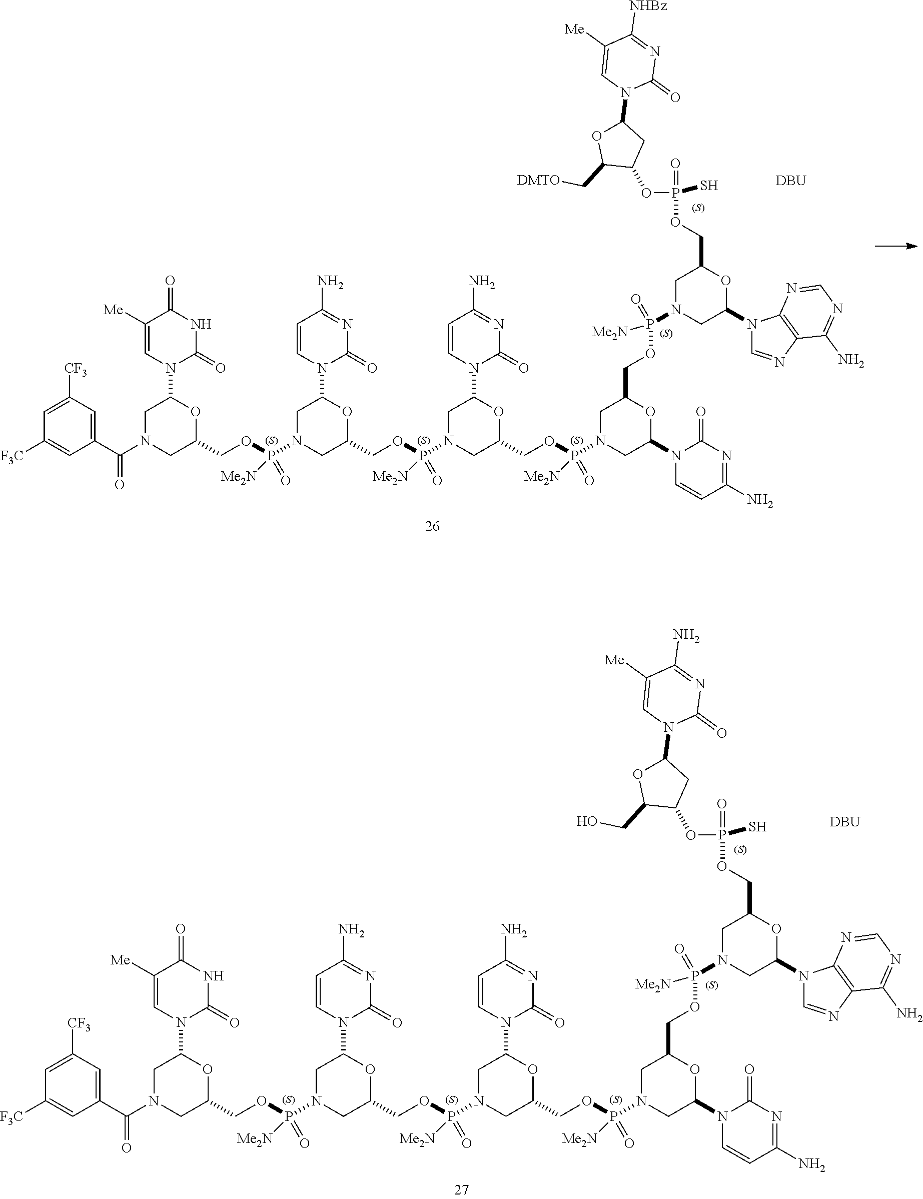

Starting material 26 (228 mg, 0.0710 mmol) was dissolved in a mixture of 1,1,1,3,3,3-hexafluoro-2-propanol (1.5 mL), 2,2,2-trifluoroethanol (0.75 mL), DCM (3.7 mL) and triethylsilane (2.2 mL) and the resulting solution was stirred at room temperature. After 4 h, additional 2 mL of 1,1,1,3,3,3-hexafluoro-2-propanol was added. Once the reaction was complete (monitored by LCMS), 25 mL EtOAc and 33 mL MTBE were added. The resulting precipitate was collected by filtration and rinsed with a mixture of EtOAc/DCM (8 mL/2 mL). Drying in vacuo for overnight provided 150 mg of target product 27.

HRMS (ESI) m/z: [M+H]⁺ Calcd for C₇₄H₁₀₄F₆N₂₈O₂₅P₅S, 2086.6074. Found 2086.5801.

### 7-Mer: Coupling

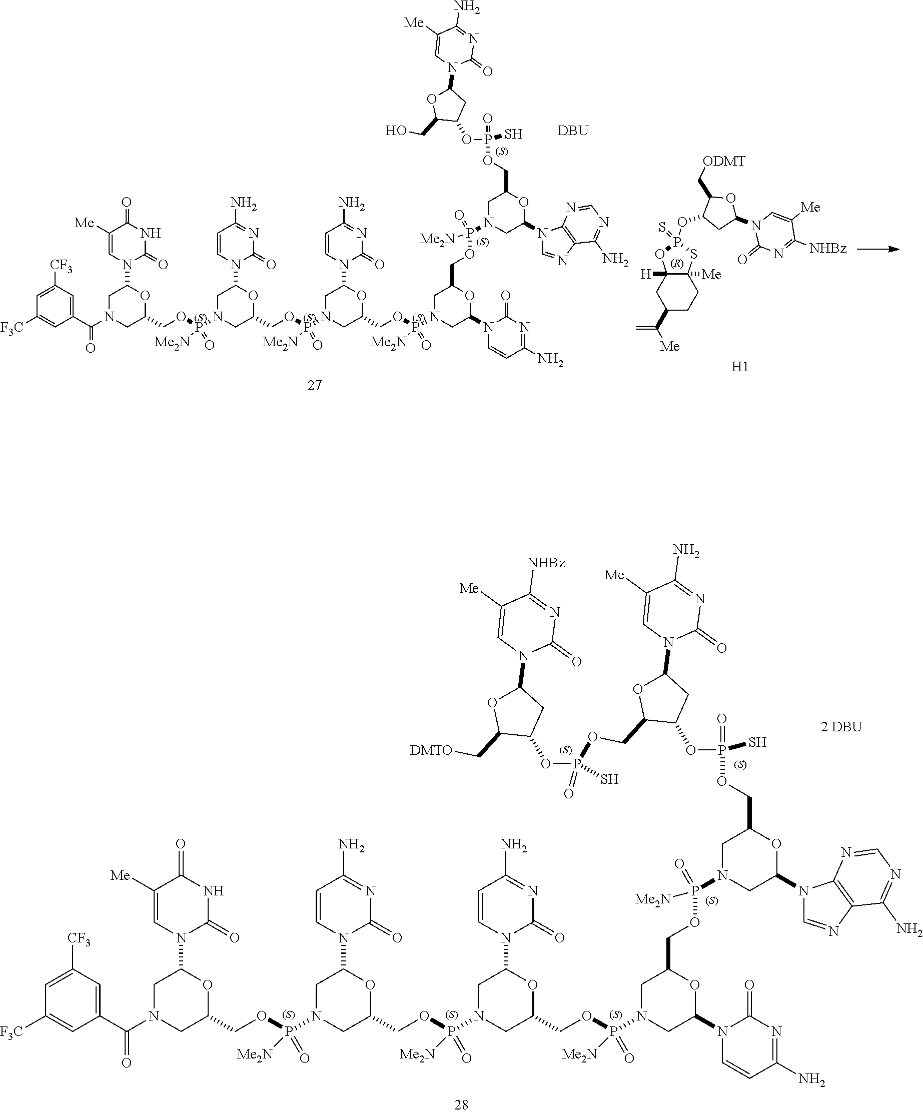

To a mixture of starting material 27 (150 mg, 0.064 mmol) and reactant H1 (172 mg, 0.192 mmol) was added 1,3-dimethyl-2-imidazolidinone (3.6 mL). The resulting mixture was azeotroped with toluene three times (2 mL each time) at 30-33° C. To the resulting solution was added 4 Å molecular sieves (350 mg). The reaction flask was applied to vacuum and filled with nitrogen. The process was repeated two more times. To the resulting mixture was added DBU (0.058 mL, 0.38 mmol) and the reaction mixture was stirred at room temperature overnight (13 h) while the reaction progress was monitored by LCMS. Upon competition, the reaction mixture was filtered through a syringe filter and the filtrate was added into EtOAc (15 mL), rinsing with 2 mL 1,3-dimethyl-2-imidazolidinone. To the resulting slurry was added additional 5 mL of EtOAc. The resulting precipitate was collected by filtration and rinsed with a mixture of EtOAc/1,3-dimethyl-2-imidazolidinone (5 mL/1 mL) and EtOAc (10 mL). Drying in vacuo for 30 min provided 218 mg of target product 28.

HRMS (ESI) m/z: [M-DMT+2H]⁺ Calcd for C₉₁H₁₂₂F₆N₃₁O₃₁P₆S₂ 2509.6728. Found 2509.6360.

### 7-Mer: Deprotection

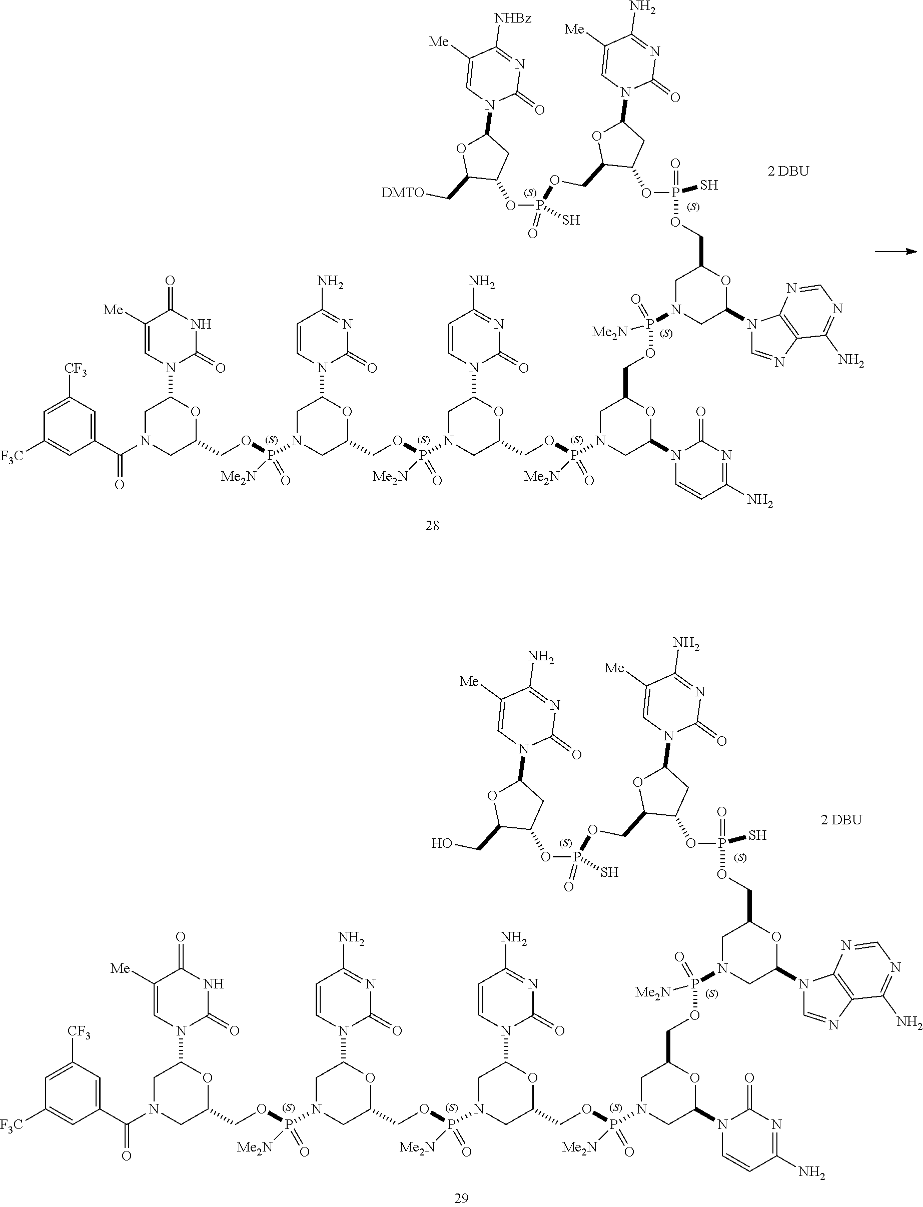

To starting material 28 (218 mg, 0.064 mmol) was added a mixture of 1,1,1,3,3,3-hexafluoro-2-propanol (2 mL), 2,2,2-trifluoroethanol (0.5 mL), triethylsilane (1.5 mL) and DCM (2.5 mL). The resulting solution was stirred at room temperature while the progress was monitored by LCMS. Once the reaction was complete (3 h), 40 mL EtOAc was added. The resulting precipitate was collected by filtration and rinsed with a mixture of EtOAc/DCM (8 mL/2 mL). Drying in vacuo for overnight provided 150 mg of target product 29.

HRMS (ESI) m/z: [M+2H]²⁺ Calcd for C₈₄H₁₁₉F₆N₃₁O₃₀P₆S₂ 1203.3272. Found 1203.3145.

### 8-Mer: Coupling

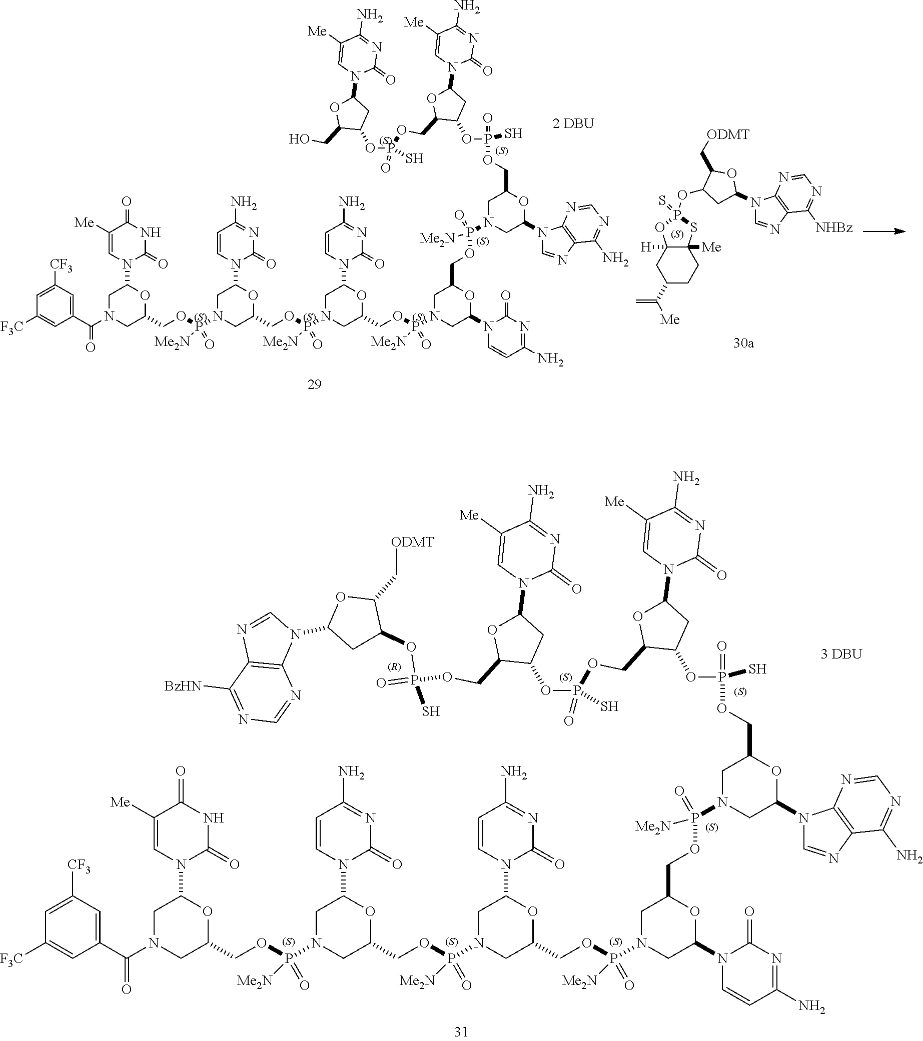

Starting material 29 (150 mg, 0.055 mmol) and reactant 30a (150 mg, 0.166 mmol) were dissolved in 1,3-dimethyl-2-imidazolidinone (5.5 mL). The resulting solution was azeotroped with toluene three times (2 mL each time) at 30-33° C. To the resulting solution was added 4 Å molecular sieves (350 mg). The reaction flask was applied to vacuum and filled with nitrogen. The process was repeated two more times. To the resulting mixture was added DBU (0.067 mL, 0.44 mmol) and the reaction mixture was stirred at room temperature while the reaction progress was monitored by LCMS. Upon competition (2.5 d), the reaction mixture was filtered through a syringe filter and the filtrate was added into EtOAc (24 mL), rinsing with 2.5 mL 1,3-dimethyl-2-imidazolidinone. The resulting precipitate was collected by filtration and rinsed with a mixture of EtOAc/1,3-dimethyl-2-imidazolidinone (8 mL/2 mL) and EtOAc (10 mL). Drying in vacuo overnight at room temperature provided 214 mg of target product 31.

HRMS (ESI) m/z: [M-DMT+2H]⁺ Calcd for C₁₀₁H₁₃₄F₆N₃₆O₃₅P₇S₃ 2838.7075. Found 2838.6948.

### 8-Mer: De-Protection

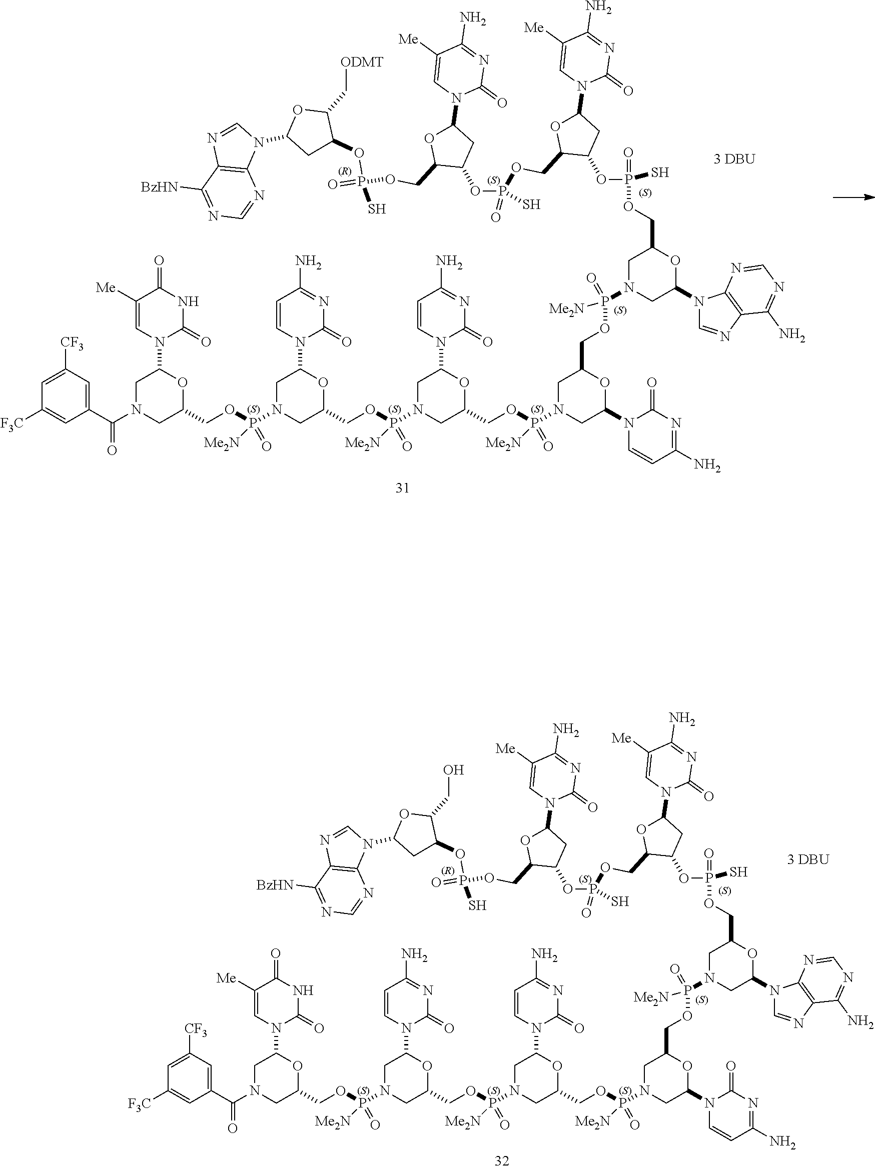

To starting material 31 (214 mg, 0.055 mmol) was added a mixture of 1,1,1,3,3,3-hexafluoro-2-propanol (2 mL), 2,2,2-trifluoroethanol (0.5 mL), triethylsilane (1.5 mL) and DCM (2.5 mL). The resulting solution was stirred at room temperature while the progress was monitored by LCMS. Once the reaction was complete (3 h), 35 mL EtOAc was added. The resulting precipitate was collected by filtration and rinsed with a mixture of EtOAc/DCM (8 mL/2 mL). Drying in vacuo overnight provided 146 mg of target product 32.

MS (ESI) m/z: [M−2H]²⁻ Calcd for C₁₀₁H₁₃₁F₆N₃₆O₃₅P₇S₃ 1418.34. Found 1418.52.

### 9-Mer: Coupling

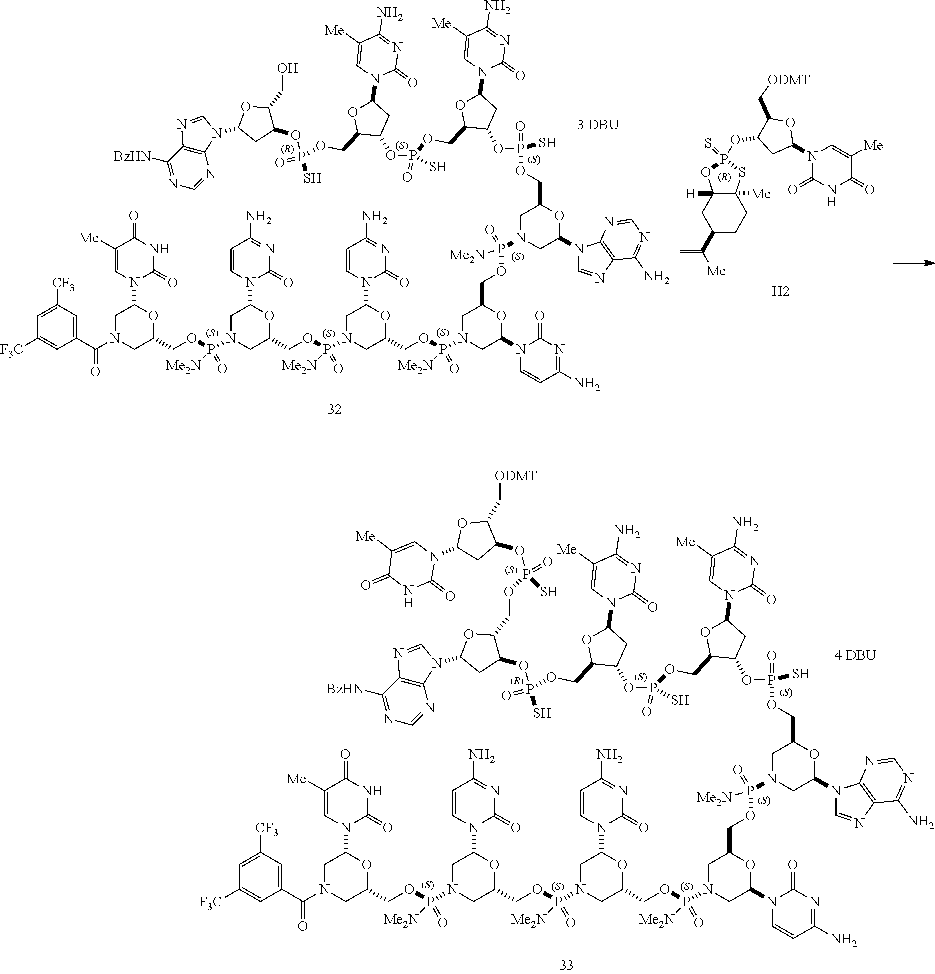

To a solution of starting material 32 (146 mg, 0.044 mmol) in 1,3-dimethyl-2-imidazolidinone (5.0 mL) was added reactant H2 (105 mg, 0.133 mmol). The resulting mixture was azeotroped with toluene three times (2 mL each time) at 30-33° C. To the resulting solution was added 4 Å molecular sieves (400 mg). The reaction flask was applied to vacuum and filled with nitrogen. The process was repeated two more times. To the resulting mixture was added DBU (0.060 mL, 0.40 mmol) and the reaction mixture was stirred at room temperature while the reaction progress was monitored by LCMS. Upon competition (2 d), the reaction mixture was filtered through a syringe filter and the resulting filtrate was added into EtOAc (25 mL), rinsing with 3 mL 1,3-dimethyl-2-imidazolidinone. The resulting precipitate was collected by filtration and rinsed with a mixture of EtOAc/1,3-dimethyl-2-imidazolidinone (6 mL/2 mL) and EtOAc (10 mL). Drying in vacuo for 2 h at room temperature provided 238 mg of target product 33.

MS (ESI) m/z: [M−2H]²⁻ Calcd for C₁₃₂H₁₆₂F₆N₃₈O₄₃P₈S₄ 1729.42. Found 1729.95.

### 9-Mer: Deprotection

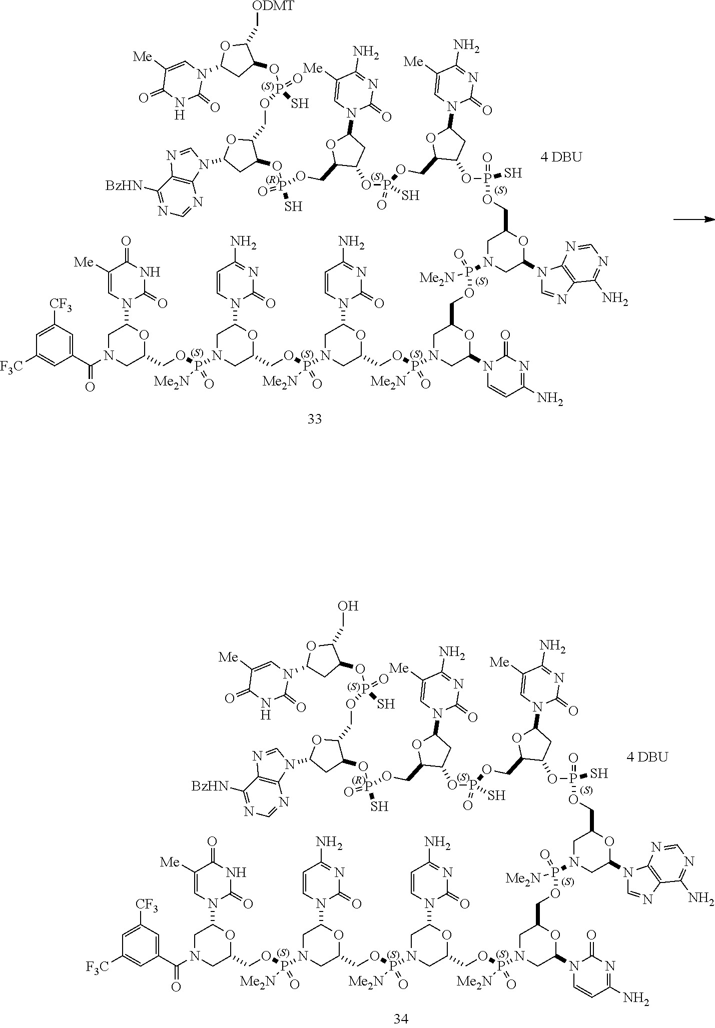

To starting material 33 (238 mg, 0.058 mmol) was added a mixture of 1,1,1,3,3,3-hexafluoro-2-propanol (2 mL), 2,2,2-trifluoroethanol (0.5 mL), triethylsilane (1.5 mL) and DCM (2.5 mL). The resulting solution was stirred at room temperature while the progress was monitored by LCMS. Once the reaction was complete (18 h), 40 mL EtOAc was added. The resulting precipitate was collected by filtration and rinsed with a mixture of EtOAc/DCM (6 mL/2 mL). Drying in vacuo for 3 h provided target product 34 (170 mg in theory).

MS (ESI) m/z: [M−2H]²⁻ Calcd for C₁₁₁H₁₄₄F₆N₃₈O₄₁P₈S₄ 1578.36. Found 1578.94.

### 10-Mer: Coupling

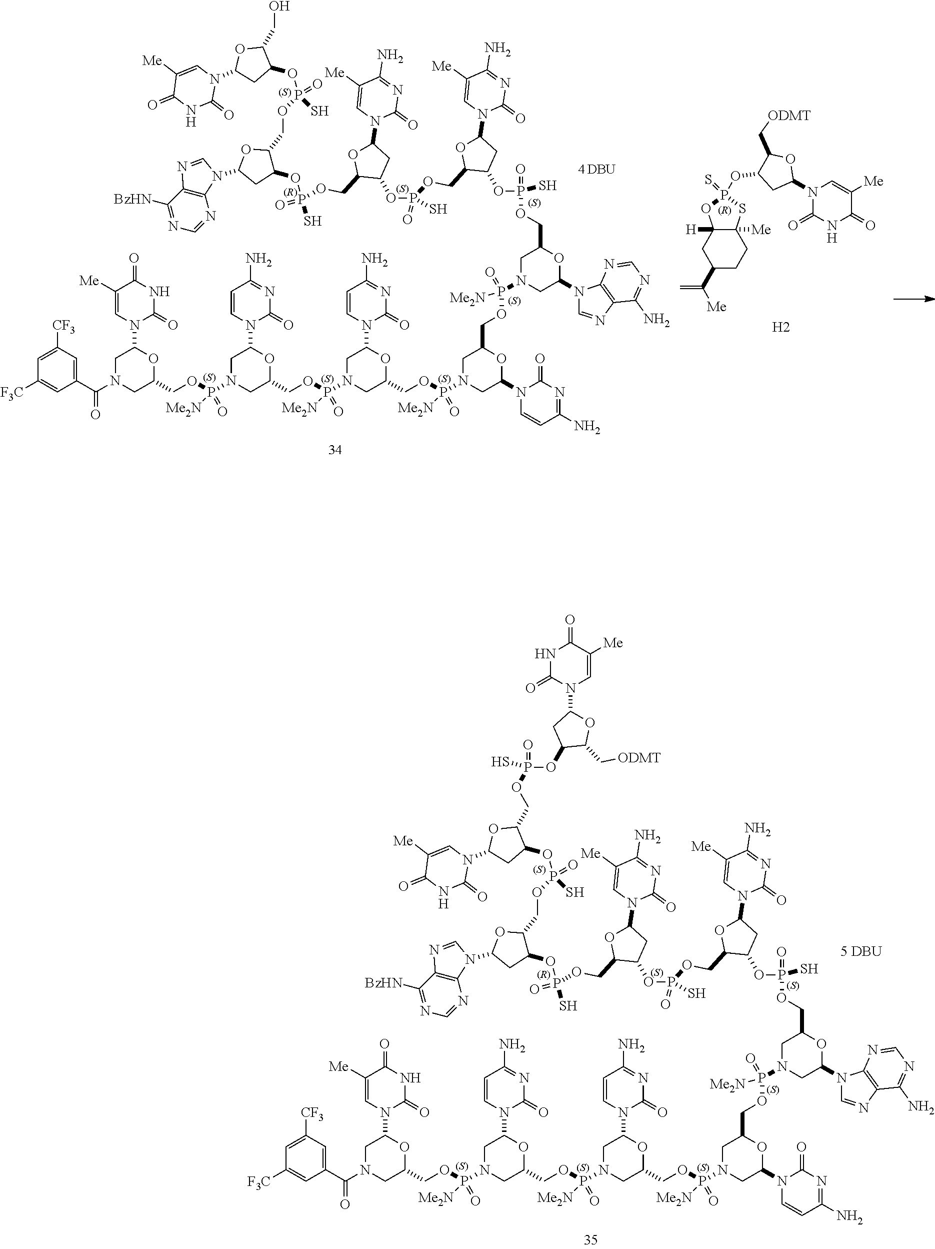

To a solution of starting material 34 (170 mg, 0.045 mmol in theory) in 1,3-dimethyl-2-imidazolidinone (5 mL) was added reactant 112 (107 mg, 0.135 mmol). The resulting mixture was azeotroped with toluene three times (2 mL each time) at 30-33° C. To the resulting solution was added 4 Å molecular sieves (400 mg). The reaction flask was applied to vacuum and filled with nitrogen. The process was repeated two more times. To the resulting mixture was added DBU (0.068 mL, 0.45 mmol) and the reaction mixture was stirred at room temperature while the reaction progress was monitored by LCMS. Upon competition (3 d), the reaction mixture was filtered through a syringe filter and the resulting filtrate was added into EtOAc (24 mL), rinsing with 1,3-dimethyl-2-imidazolidinone (4 mL). The resulting precipitate was collected by filtration and rinsed with a mixture of EtOAc/1,3-dimethyl-2-imidazolidinone (6 mL/2 mL) and EtOAc (10 mL). Drying in vacuo at room temperature provided target product 35 (205 mg in theory).

MS (ESI) m/z: [M−2H]²⁻ Calcd for C₁₄₂H₁₇₅F₆N₄₀O₄₉P₉S₅ 1890.43. Found 1890.37.

### 10-Mer: Deprotection

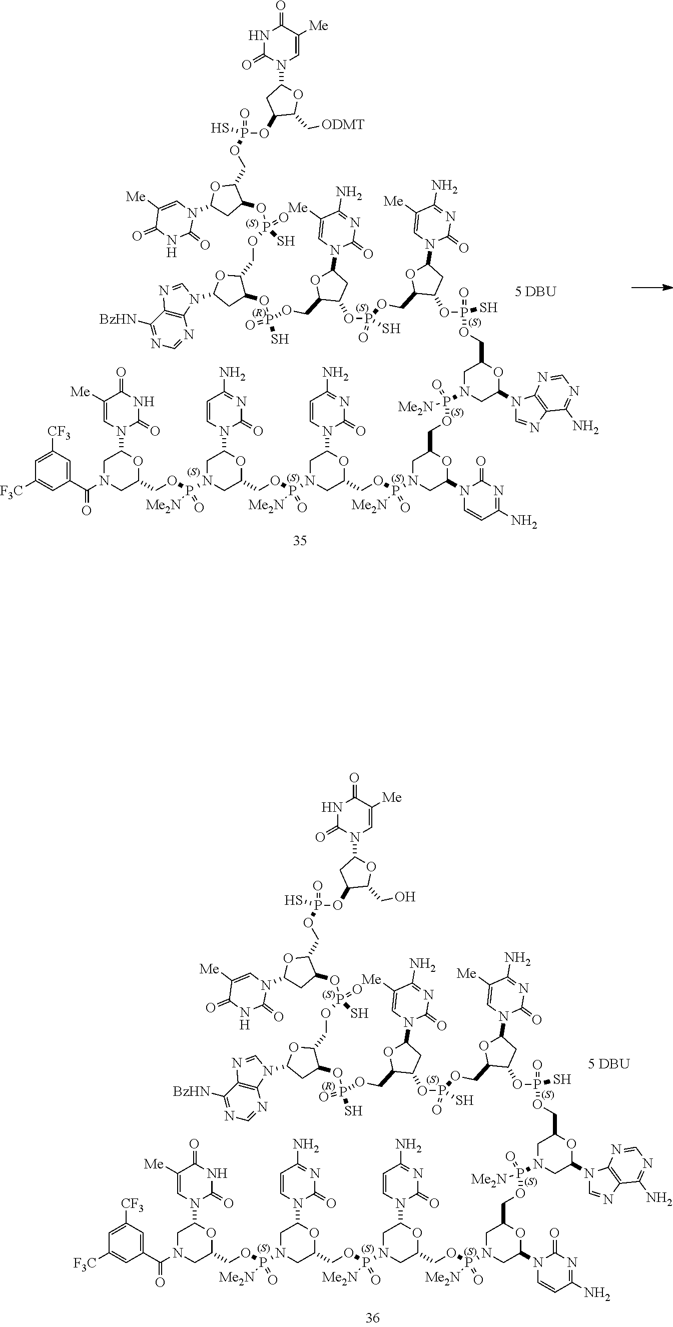

To starting material 35 (205 mg, 0.045 mmol in theory) was added a mixture of 1,1,1,3,3,3-hexafluoro-2-propanol (3 mL), 2,2,2-trifluoroethanol (0.75 mL), triethylsilane (2.25 mL) and DCM (3.75 mL) and the resulting solution was stirred at room temperature while the progress was monitored by LCMS. Once the reaction was complete (5.5 h), 45 mL EtOAc was added. The resulting precipitate was collected by filtration and rinsed with a mixture of EtOAc/DCM (6 mL/2 mL). Drying in vacuo for 3 h provided 165 mg of target product 36.

MS (ESI) m/z: [M−2H]²⁻ Calcd for C₁₂₁H₁₅₇F₆N₄₀O₄₇P₉S₅ 1737.86. Found 1738.55.

### 11-Mer: Coupling

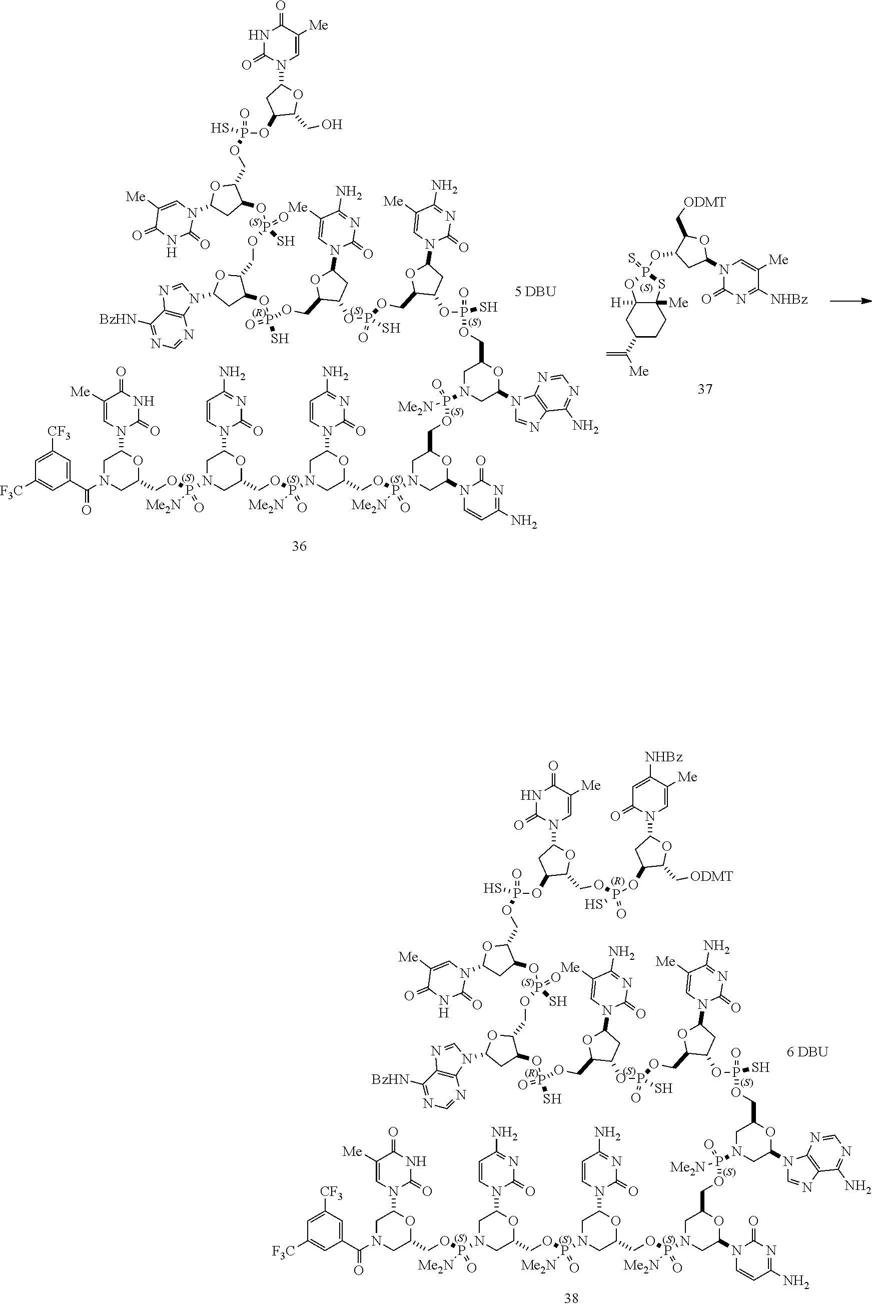

To a solution of starting material 36 (165 mg, 0.039 mmol) in 1,3-dimethyl-2-imidazolidinone (5 mL) was added reactant 37 (104 mg, 0.117 mmol). The resulting mixture was azeotroped with toluene three times (2 mL each time) at 30-33° C. To the resulting solution was added 4 Å molecular sieves (400 mg). The reaction flask was applied to vacuum and filled with nitrogen. The process was repeated two more times. To the resulting mixture was added DBU (0.070 mL, 0.47 mmol) and the reaction mixture was stirred at room temperature while the reaction progress was monitored by LCMS. Upon competition (2 d), the reaction mixture was filtered through a syringe filter and the resulting filtrate was added into EtOAc (30 mL), rinsing with 1,3-dimethyl-2-imidazolidinone (5 mL). The resulting slurry mixture was centrifuged (2000 rpm, 15 min). The resulting pallet was collected by filtration and rinsed with a mixture of EtOAc/1,3-dimethyl-2-imidazolidinone (6 mL/2 mL) and EtOAc (10 mL). Drying in vacuo at room temperature provided target product 38 (199 mg in theory).

MS (ESI) m/z: [M-DMT-2H]³⁻ Calcd for C₁₃₈H₁₇₄F₆N₄₃O₅₃P₁₀S₆ 1299.26. Found 1299.95.

### 11-Mer: Deprotection

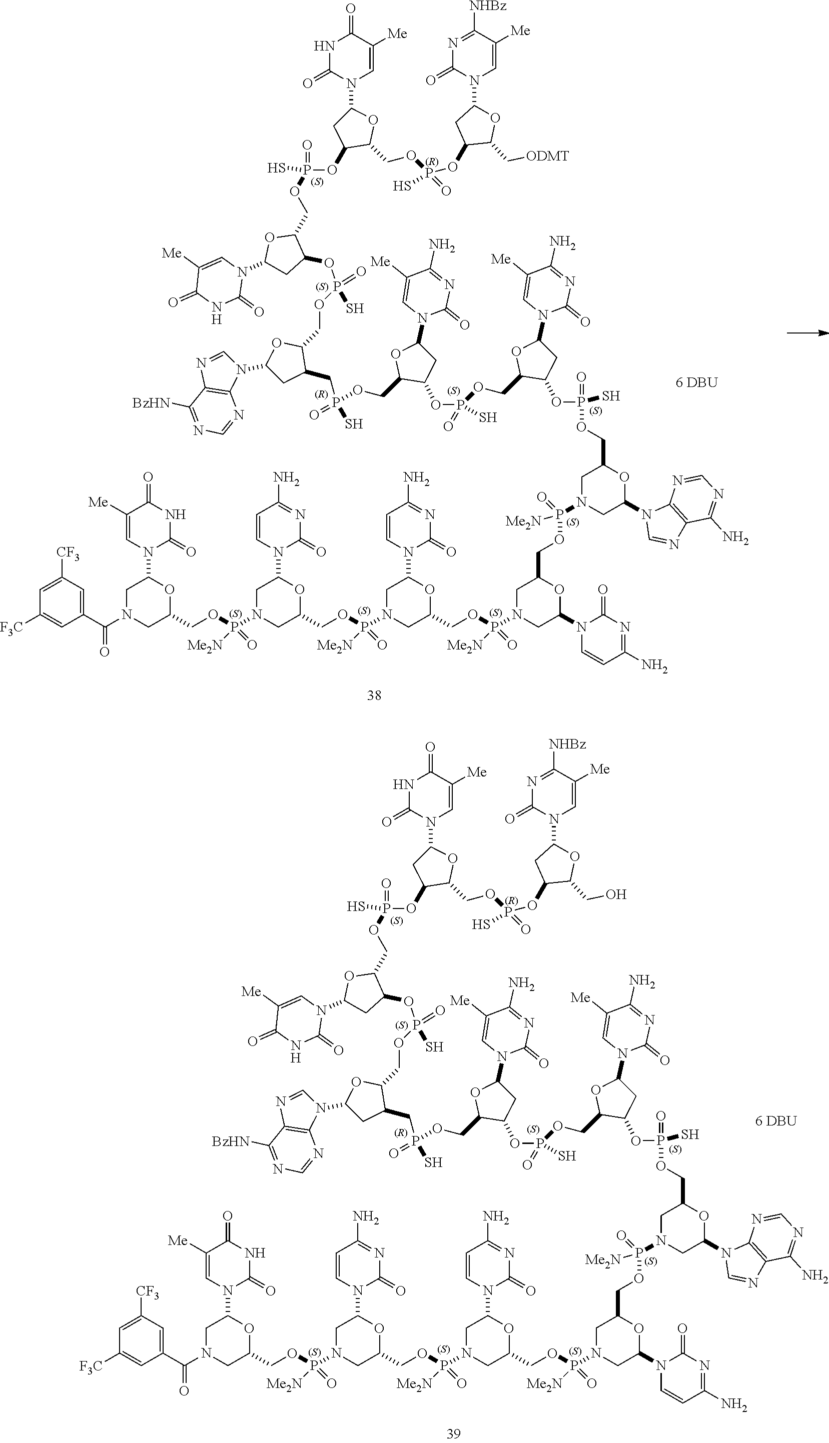

To starting material 38 (199 mg, 0.039 mmol in theory) was added a mixture of 1,1,1,3,3,3-hexafluoro-2-propanol (3 mL), 2,2,2-trifluoroethanol (0.75 mL), triethylsilane (2.25 mL) and DCM (3.75 mL). The resulting solution was stirred at room temperature overnight and then treated with 40 mL EtOAc. The resulting precipitate was collected by filtration and rinsed with a mixture of EtOAc/DCM (6 mL/2 mL). Drying in vacuo for 2 h provided 170 mg of target product 39.

MS (ESI) m/z: [M−3H]³⁻ Calcd for C₁₃₈H₁₇₄F₆N₄₃O₅₃P₁₀S₆ 1299.26. Found 1300.75.

### 12-Mer: Coupling

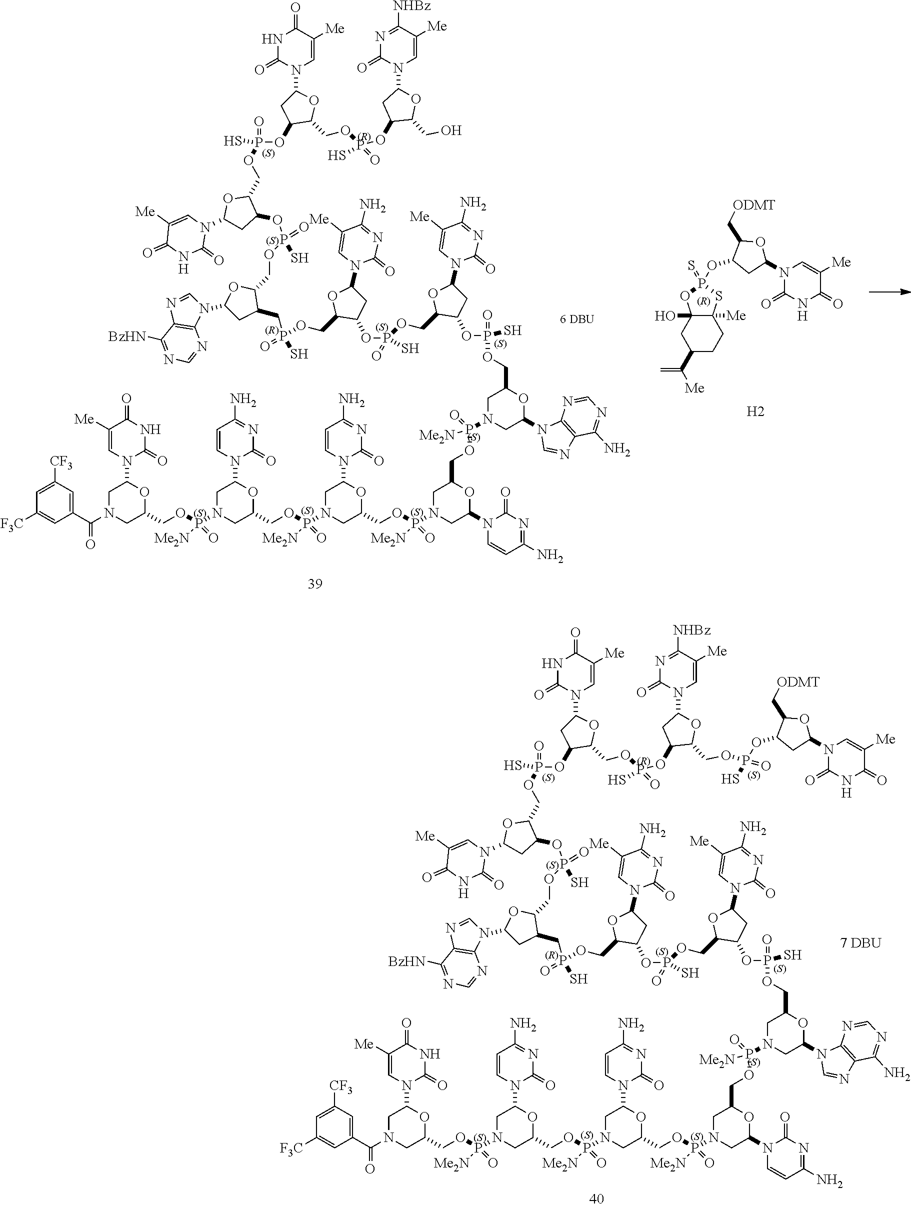

To a solution of starting material 39 (171 mg, 0.036 mmol) in 1,3-dimethyl-2-imidazolidinone (5 mL) was added reactant 112 (84 mg, 0.107 mmol). The resulting mixture was azeotroped with toluene three times (2 mL each time) at 30-33° C. To the resulting solution was added 4 Å molecular sieves (500 mg). The reaction flask was applied to vacuum and filled with nitrogen. The process was repeated two more times. To the resulting mixture was added DBU (0.070 mL, 0.47 mmol) and the reaction mixture was stirred at room temperature while the reaction progress was monitored by LCMS. Upon competition (3 d), the reaction mixture was filtered through a syringe filter and the resulting filtrate was added into EtOAc (30 mL), rinsing with 5 mL 1,3-dimethyl-2-imidazolidinone. The resulting slurry mixture was centrifuged (2000 rpm, 15 min). The resulting pallet was collected by filtration and rinsed with a mixture of EtOAc/1,3-dimethyl-2-imidazolidinone (6 mL/2 mL) and EtOAc (10 mL). Drying in vacuo at room temperature provided target product 40 (199 mg in theory; MS was not observed at LCMS conditions but this product yielded other tested products).

### 12-Mer: Deprotection

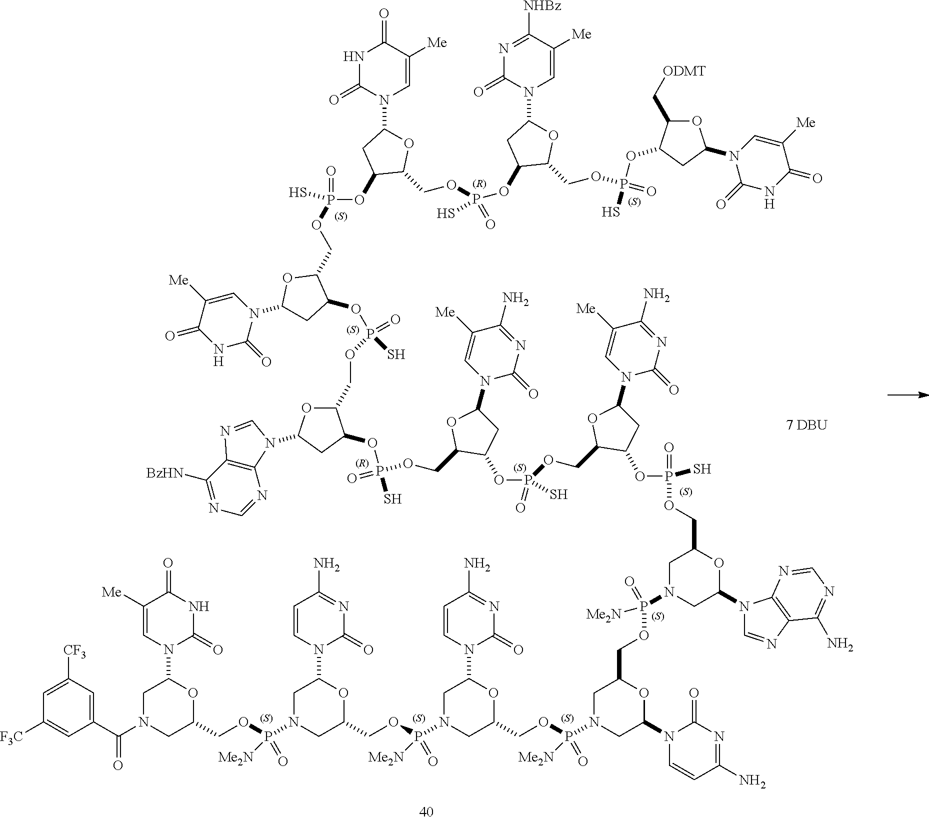

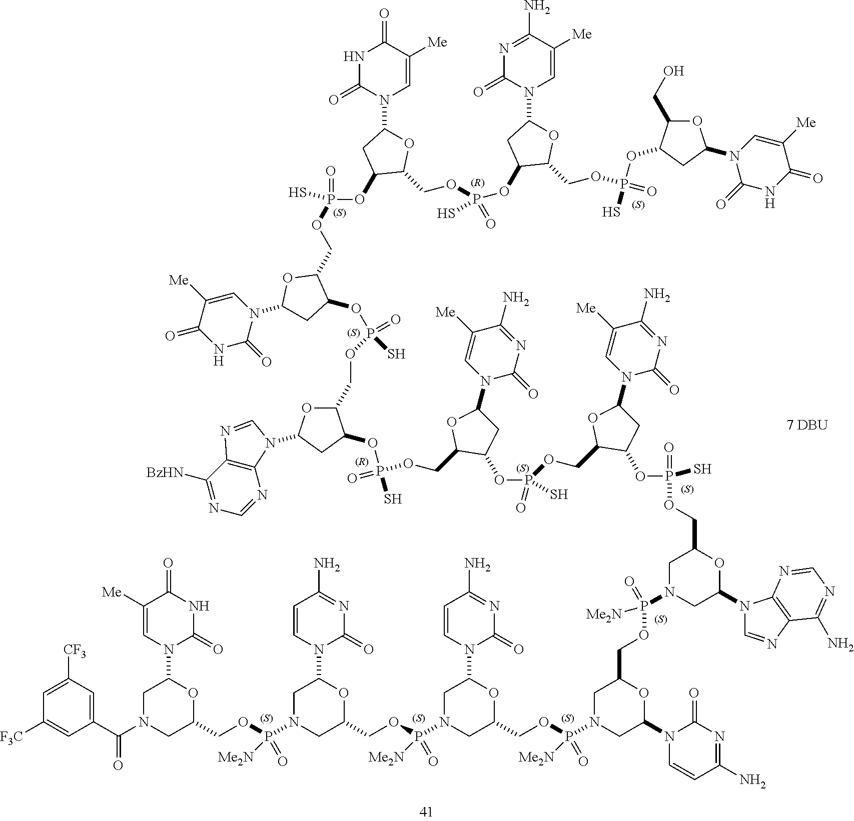

To starting material 40 (199 mg, 0.036 mmol) was dissolved in a mixture of 1,1,1,3,3,3-hexafluoro-2-propanol (4 mL), 2,2,2-trifluoroethanol (1 mL), triethylsilane (3 mL) and DCM (5 mL). The resulting solution was stirred at room temperature while the progress was monitored by LCMS. Once the reaction was complete (15 h), the reaction mixture was treated with 40 mL EtOAc and 15 mL MTBE. The resulting precipitate was collected by filtration and rinsed with a mixture of EtOAc/DCM (6 mL/2 mL). Drying in vacuo overnight provided 174 mg of target product 41.

MS (ESI) m/z: [M−3H]³⁻ Calcd for C₁₄₁H₁₈₃F₆N₄₅O₅₈P₁₁S₇ 1371.26. Found 1371.87.

### Example 4.4: 12+6 Coupling

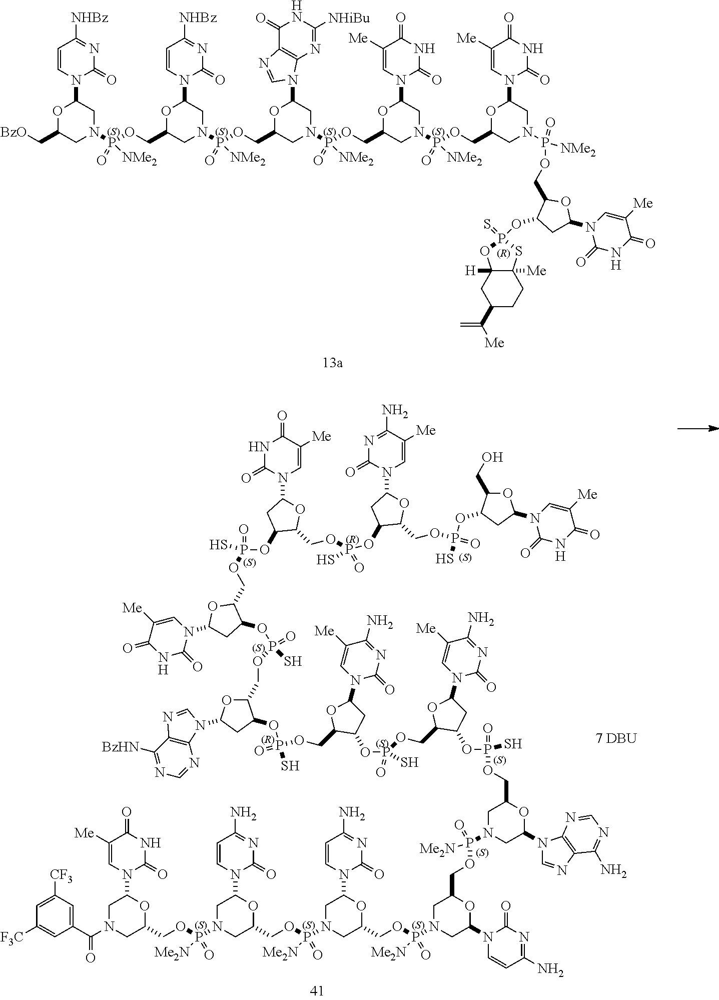

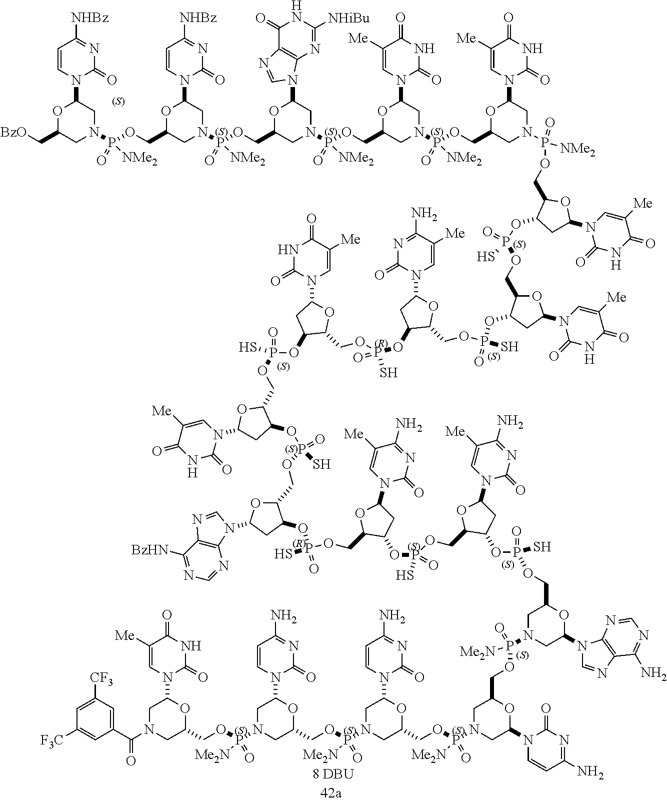

To a mixture of starting material 41 (163 mg, 0.0310 mmol) and reactant 13a (170 mg, 0.068 mmol) was added 1,3-dimethyl-2-imidazolidinone (6 mL). The resulting mixture was azeotroped with toluene (2.5 mL each time) four times at 30-33° C. To the resulting solution was added 4 Å molecular sieves (450 mg). The reaction flask was applied to vacuum and filled with nitrogen. The process was repeated two more times. To the resulting mixture was added DBU (0.071 mL, 0.47 mmol) and the reaction mixture was stirred at room temperature while the reaction progress was monitored by LCMS. Upon competition (24 h), the reaction mixture was filtered through a syringe filter and the filtrate was added into EtOAc (12 mL), rinsing with 3 mL 1,3-dimethyl-2-imidazolidinone. The resulting slurry mixture was centrifuged (3000 rpm, 30 min). The resulting pellet was collected by decantation and dissolved in a mixture of DCM/EtOH (14 mL/7 mL). To the resulting solution was added EtOAc (20 mL). The resulting precipitate was collected by filtration and rinsed with a mixture of EtOAc/DCM/EtOH (3 mL/2 mL/1 mL). Drying in vacuo at room temperature for 1 h provided 0.20 g of target product 42a. The material was used in next step without further purification.

MS (ESI) m/z: [M+5H]⁵⁺ Calcd for C₂₃₄H₃₁₄F₆N₇₂O₉₀P₁₇S₈ 1294.11. Found 1294.25.

Alternative 12+6 Coupling with 13b

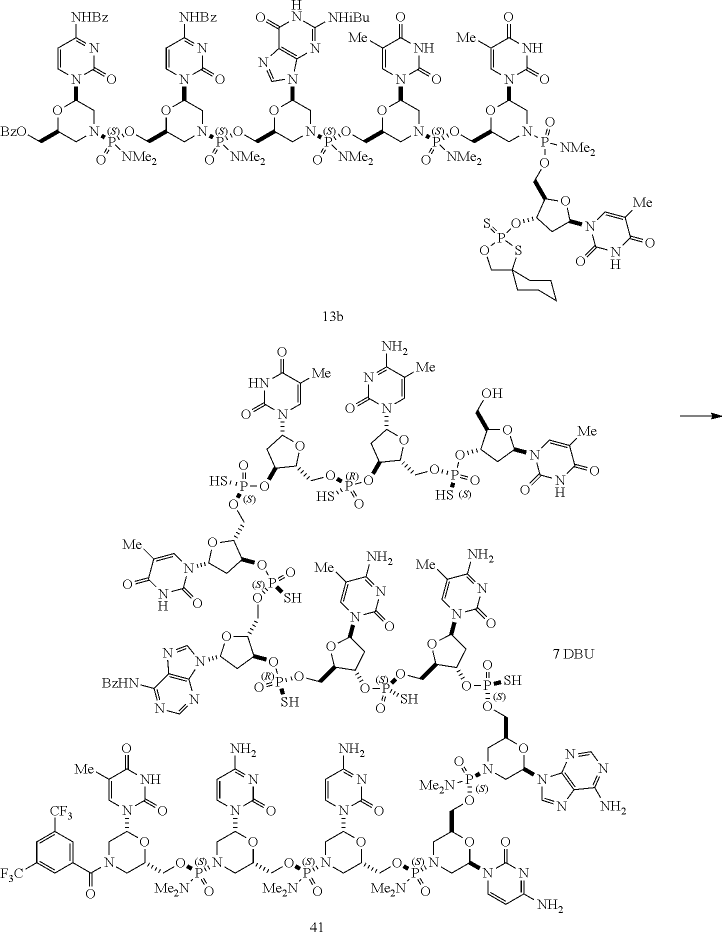

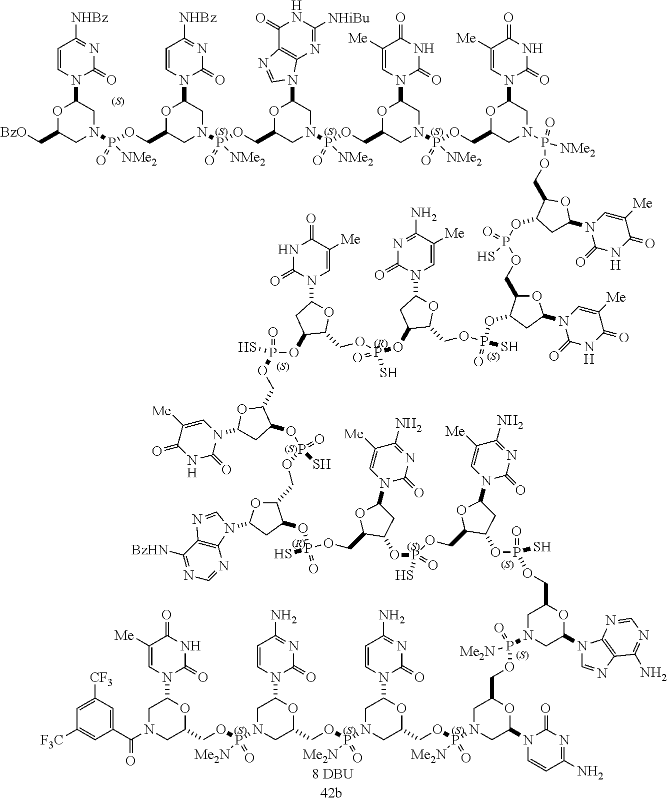

A mixture of starting material 41 (0.53 g, 0.10 mmol) and reactant 13b (0.74 g, 0.30 mmol) is dissolved in 1,3-dimethyl-2-imidazolidinone and azeotroped with toluene three times. To the resulting solution are added 4 A MS and 1,4-diazabicyclo[5.4.0]undec-7-ene (DBU, 1.5 mmol). The resulting solution is stirred at room temperature overnight, filtered, and added into EtOAc. The resulting precipitate is collected by filtration and is rinsed with a mixture of EtOAc/DCM/EtOH (3/2/1). Drying in vacuo provides target product 42b.

### Example 4.5: Final Deprotection

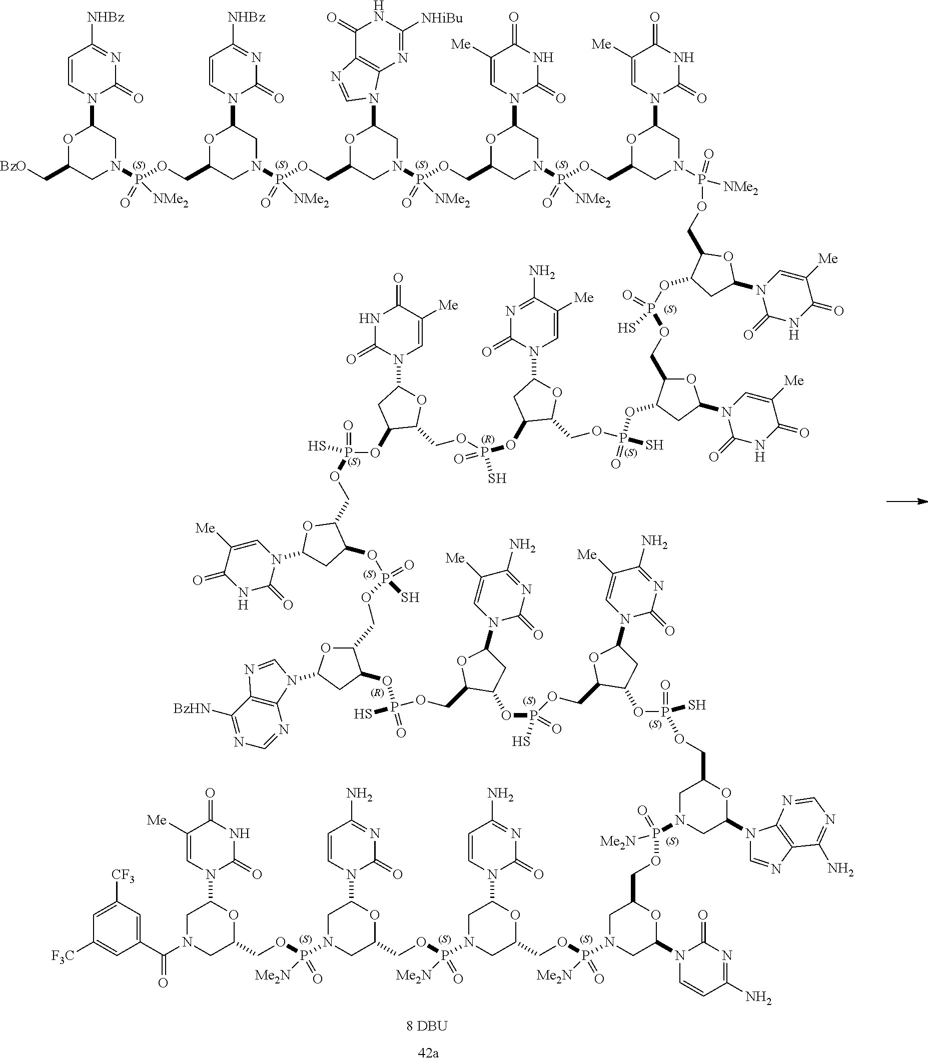

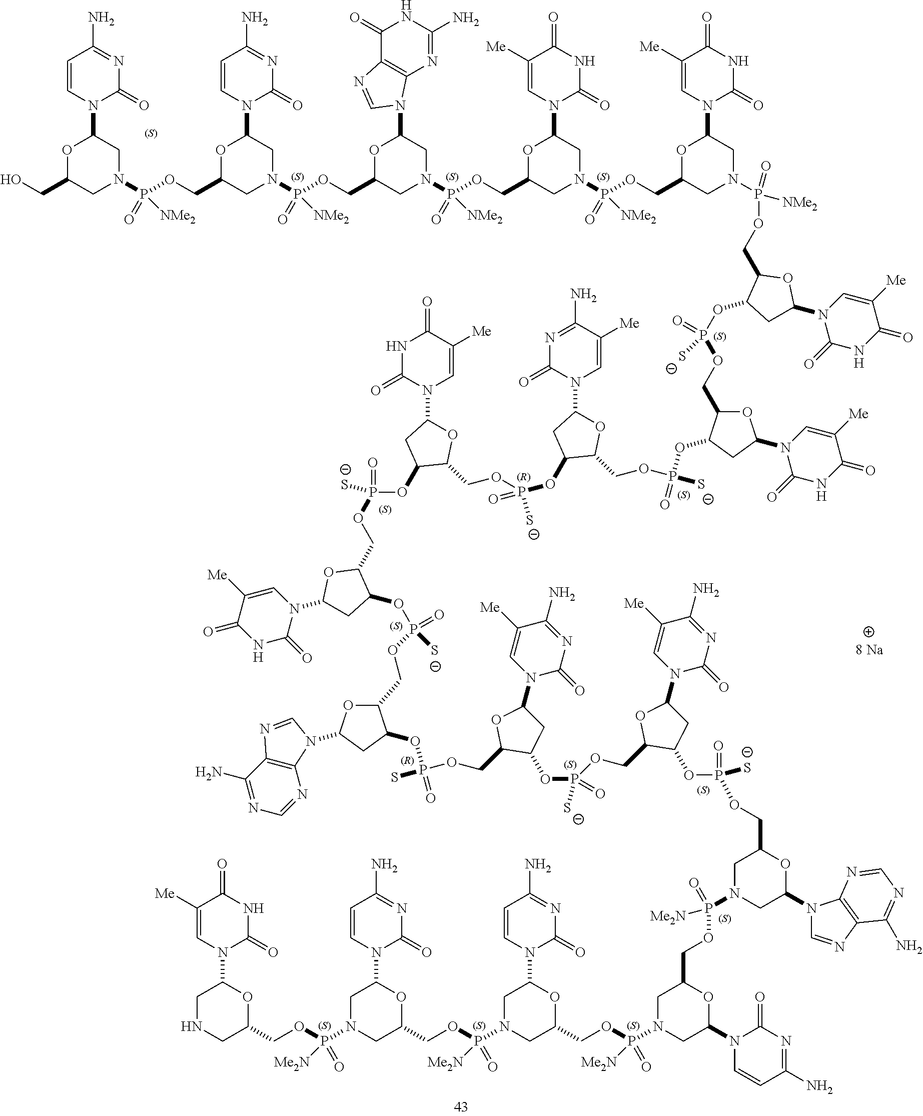

To a solution of starting material 42a (0.20 g) in a mixture of methanol (5 mL) and 28% ammonium hydroxide (5 mL) was added DL-dithiothreitol (0.026 g, 0.17 mmol). The resulting mixture was stirred at 53-55° C. for 20 h and cooled to room temperature. Additional MeOH (2 mL) and 28% ammonium hydroxide (2 mL) were added. The resulting mixture was stirred for additional 10 h at 50-55° C. and for 2 days at room temperature. A mixture of MeCN/EtOAc (60 mL/20 mL) was added and the resulting slurry was subjected to centrifuge (4000 rpm, 30 min). The resulting pellet was isolated and dissolved in water (˜20 mL). The aqueous solution was subjected to ultrafiltration (Amicon Ultra-15, ultracel 3K, 3500 rpm, 45 min) four times. The resulting solution was diluted with 5 mL water and purified by IEX-HPLC under the following conditions depicted in Table 5.

TABLE 5
IEX-HPLC conditions

| Column | TSKgel SuperQ-5PW, 7.5 mm ID × 7.5 cm, | |
|---|---|---|
| | 10 μm, TOSOH cat no. K0080-103KNM | |
| | (Three columns connected in serial for purification) | |
| Instrument | Agilent 1200 | |
| Mobile phase A | 10 mM NaOH pH = 12 | |
| Mobile phase B | 10 mM NaOH pH = 12 w/1M NaCl | |
| Column | 45 | |
| Temperature (° C.) | | |
| Gradient Time (min) | A % | B % |
| 0 | 60 | 40 |
| 20 | 25 | 75 |
| 22 | 5 | 95 |
| 25 | 5 | 95 |
| 25.1 | 60 | 40 |
| 29 | 60 | 40 |

| | |
|---|---|
| Flow Rate (mL/min) | 3.0 |
| Wavelength (nm) | 260 |

Desalting of the purified product was conducted with Amicon Ultra-15, Ultracel-3K (3500 rpm, 45 min). Freeze-drying of the resulting solution (10 mL) for 3 days provided 20 mg of target product 43.

MS (ESI) m/z: [M+5H]⁵⁺ Calcd for C₁₉₃H₂₉₀N₇₂O₈₄P₁₇S₈ 1148.9. Found 1149.2.

### Example 5: Solution-Phase Synthesis of Stereodefined 4-10-4 PMO-Gapmers

Examples 5.1 through 5.5 report the preparation of a stereospecific 4-10-4 gapmer having SEQ ID NO: 12.

| Compound No. | |
|---|---|
| (SEQ ID NO:) | Sequence (5′-3′) |
| 132 | AGCAGATGA^{m}C^{m}C^{m}C TTAGAC |
| (SEQ ID NO: 12) | ^{m}C: 5-MethylCytosine |

The synthesized gapmer has a chirality represented herein as:

SSSSSSSRSSSSSSSSS (compound 132m), SSSRSSSRSSSSSSSSS (compound 132n) or SSSMSSSRSSSSSSSSS (compound 132f)

“M” means a mixture of R configuration and S configuration.

With the benefit of this specification, including the other examples presented herein, a person of skill in the art would recognize that gapmers with the same sequence but different chirality could be prepared with reference to the chirality of the added reagents in the coupling steps.

### Example 5.1 Preparation of 5′-PMO Wing

### 2-Mer of 5′-PMO: Coupling

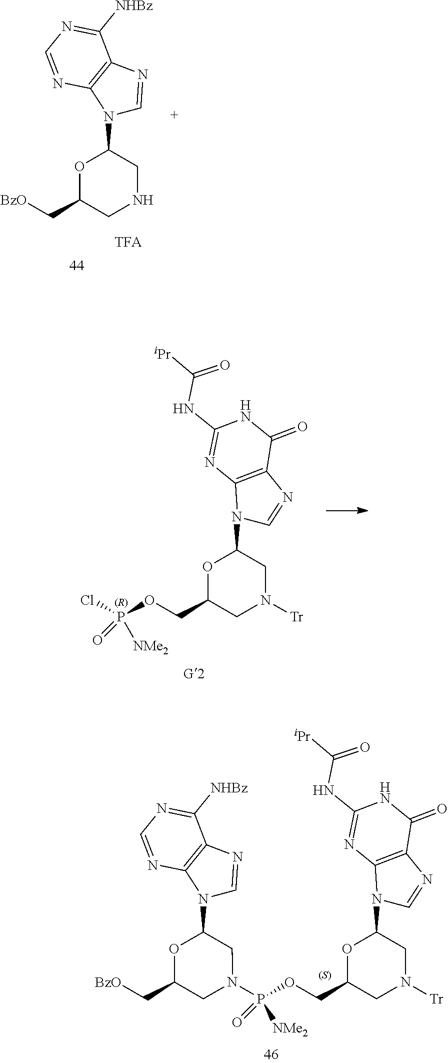

To a solution of starting material 44 (1.00 g, 1.42 mmol) in 1,3-Dimethyl-2-imidazolidinone (10 mL) was added reactant G′2 (0.854 g, 1.491 mmol) and 1,2,2,6,6-pentamethylpiperidine (1.03 mL, 5.68 mmol) at ambient temperature. The reaction solution was stirred overnight and treated with THF (10 mL) followed by MTBE (100 mL) and n-heptane (100 mL). The supernatant was decanted/filtered and the sticky stuff was rinsed with a mixture of THF/MTBE/n-heptane (20 mL/100 mL/100 mL). The leftover material was dissolved in CH₂Cl₂ and purified on silica gel column chromatography with a gradient of 0% to 20% MeOH in EtOAc to afford target compound 46 (1.33 g).

MS (ESI) m/z: [M+H]⁺ Calcd for C₅₉H₆₁N₁₃O₉P, 1126.44. Found 1126.29.

### 2-Mer of 5′-PMO: Deprotection

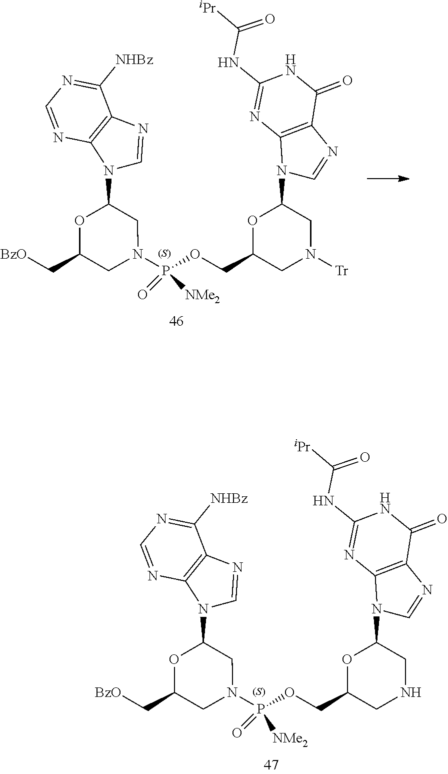

To a flask charged with starting material 46 (1.33 g, 1.18 mmol) was added ethanol (0.690 mL, 11.8 mmol) followed by a solution of TFA (0.364 mL, 4.72 mmol) in CH₂Cl₂ (20 mL) at ambient temperature. The reaction solution was stirred for 25 min and treated with EtOAc (7.5 mL) followed by n-heptane (40 mL). The slurry was filtered and the cake was rinsed with a mixture of CH₂Cl₂ (15 mL), EtOAc (7.5 mL) and n-heptane (40 mL). The TFA salt was then redissolved in CH₂Cl₂ (20 mL) at ambient temperature, and 1,2,2,6,6-pentamethylpiperidine (2.14 mL, 11.8 mmol) was added. The reaction mixture was stirred for 5-10 min before n-heptane (100 mL) was added. The slurry was sonicated to break down any aggregated pieces, and then filtered. The cake was rinsed with a mixture of CH₂Cl₂ (20 mL) and n-heptane (100 mL) to afford target compound 47 (0.93 g).

MS (ESI) m/z: [M+H]⁺ Calcd for C₄₀H₄₇N₁₃O₉P, 884.34. Found 884.26.

### 3-Mer of 5′-PMO: Coupling

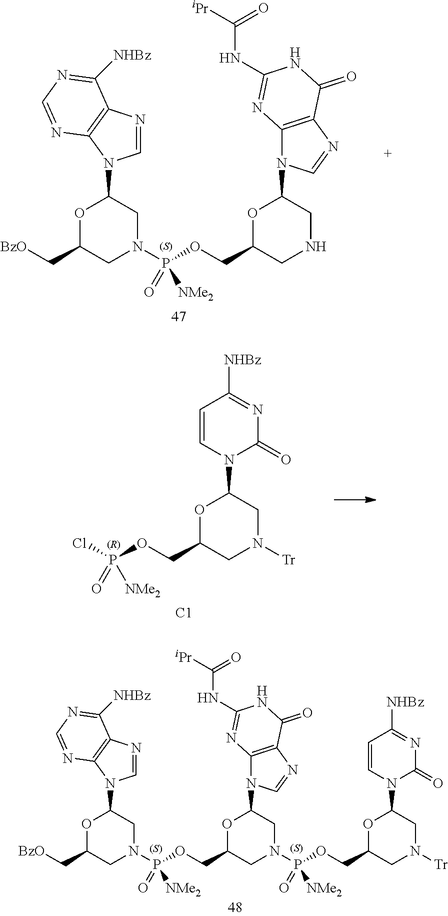

To a solution of starting material 47 (0.930 g, 1.05 mmol) in 1,3-dimethyl-2-imidazolidinone (9.24 mL) was added 1,2,2,6,6-pentamethylpiperidine (0.571 mL, 3.16 mmol) followed by reactant C1 (0.918 g, 1.32 mmol) at ambient temperature. The reaction solution was stirred overnight and treated with EtOAc (10 mL) followed by MTBE (150 mL) and n-heptane (50 mL). The slurry was filtered and the cake was rinsed with a mixture of EtOAc (10 mL), MTBE (75 mL) and n-heptane (25 mL) to afford target compound 48 (1.70 g).

MS (ESI) m/z: [M+H]⁺ Calcd for C₇₇H₈₃N₁₈O₁₄P₂ 1545.58. Found 1545.58.

### 3-Mer of 5′-PMO: Deprotection

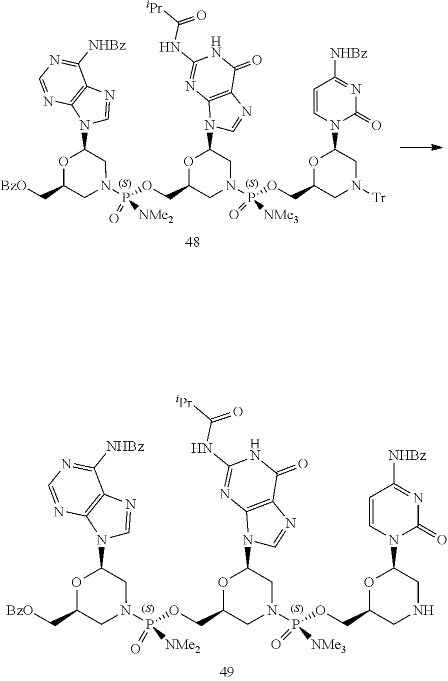

To a flask charged with a solution of starting material 48 (1.70 g, 1.10 mmol) was added ethanol (0.642 mL, 11.0 mmol) followed by a solution of TFA (0.339 mL, 4.40 mmol) in CH₂Cl₂ (25.5 mL) at ambient temperature. The reaction solution was stirred for 1 h and treated with EtOAc (12.5 mL) followed by n-heptane (45 mL). The slurry was filtered and the cake was rinsed with a mixture of CH₂Cl₂ (25 mL), EtOAc (12.5 mL) and n-heptane (40 mL). The TFA salt was then dissolved in CH₂Cl₂ (25.5 mL) at ambient temperature, and 1,2,2,6,6-pentamethylpiperidine (1.99 mL, 11.0 mmol) was added. The reaction solution was stirred for ca. 10 min and treated with EtOAc (12.5 mL) followed by MTBE (70 mL). The slurry was then filtered and the cake was rinsed with a mixture of CH₂Cl₂ (25.5 mL), EtOAc (12.5 mL) and MTBE (70 mL) to afford target compound 49 (1.19 g).

MS (ESI) m/z: [M+H]⁺ Calcd for C₅₈H₆₉N₁₈O₁₄P₂ 1303.47. Found 1303.45.

### 4-Mer of 5′-PMO: Coupling

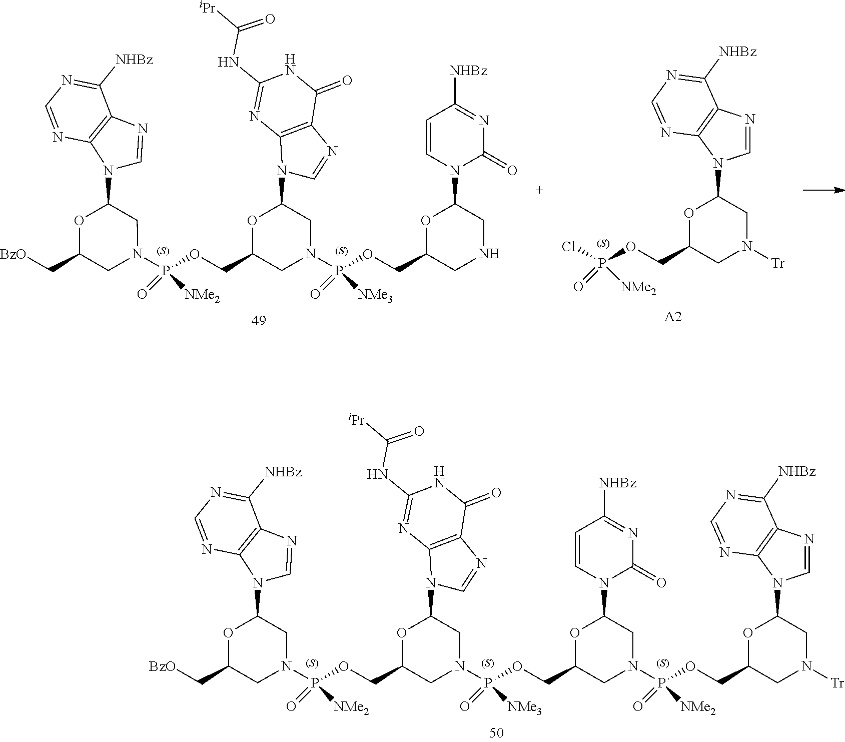

To a solution of staring material 49 (1.19 g, 0.913 mmol) in 1,3-dimethyl-2-imidazolidinone (8.0 mL) was added 1,2,2,6,6-pentamethylpiperidine (0.496 mL, 2.74 mmol) followed by reactant A2 (0.824 g, 1.14 mmol) at ambient temperature. The reaction solution was stirred overnight and treated with EtOAc (8 mL) followed by MTBE (100 mL). The slurry was filtered and rinsed with a mixture of EtOAc (16 mL) and MTBE (100 mL) to afford target compound 50 (2.04 g).

MS (ESI) m/z: [M+H]⁺ Calcd for C₉₆H₁₀₅N₂₅O₁₈P₃ 1988.73. Found 1988.67.

### 4-Mer of 5′-PMO: Deprotection

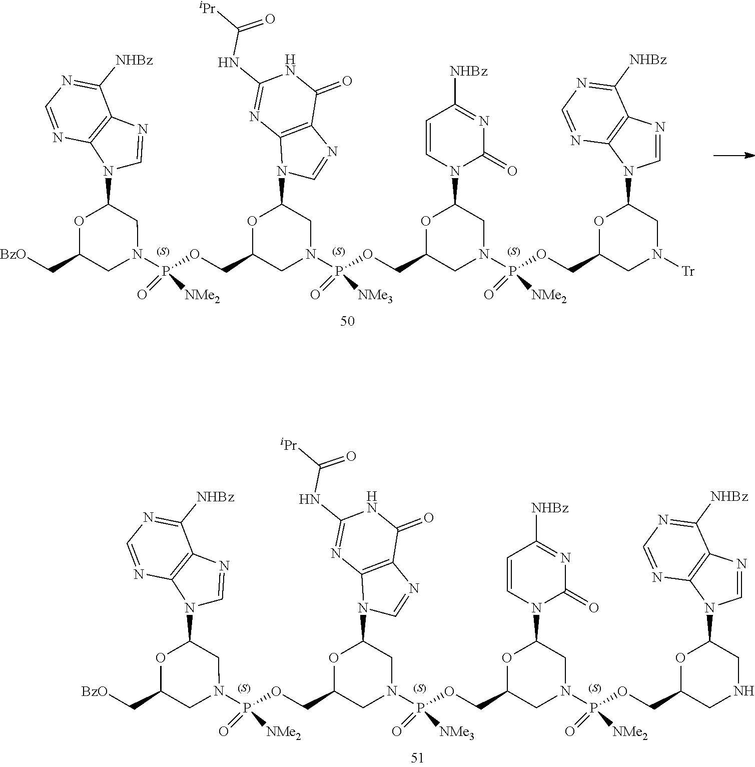

To a flask charged with starting material 50 (2.04 g, 1.03 mmol) was added ethanol (0.599 mL, 10.3 mmol) followed by a solution of TFA (0.474 mL, 6.15 mmol) in CH₂Cl₂ (24 mL) at ambient temperature. The reaction solution was stirred for 1.5 h and treated with EtOAc (12 mL) followed by n-heptane (40 mL). The slurry was filtered and the cake was rinsed with a mixture of CH₂Cl₂ (24 mL), EtOAc (12 mL) and n-heptane (40 mL). The TFA salt was then dissolved in CH₂Cl₂ (23.8 mL), and treated with 1,2,2,6,6-pentamethylpiperidine (1.856 mL, 10.26 mmol) for ca. 10 min before EtOAc (48 mL) was added followed by addition of MTBE (48 mL). The slurry was filtered and rinsed with a mixture of CH₂Cl₂ (24 mL), EtOAc (48 mL) and MTBE (48 mL) to afford target compound 51 (1.50 g).

MS (ESI) m/z: [M+H]⁺ Calcd for C₇₇H₉₁N₂₅O₁₈P₃ 1746.62. Found 1746.51.

### 5-Mer of 5′ PMO: Coupling

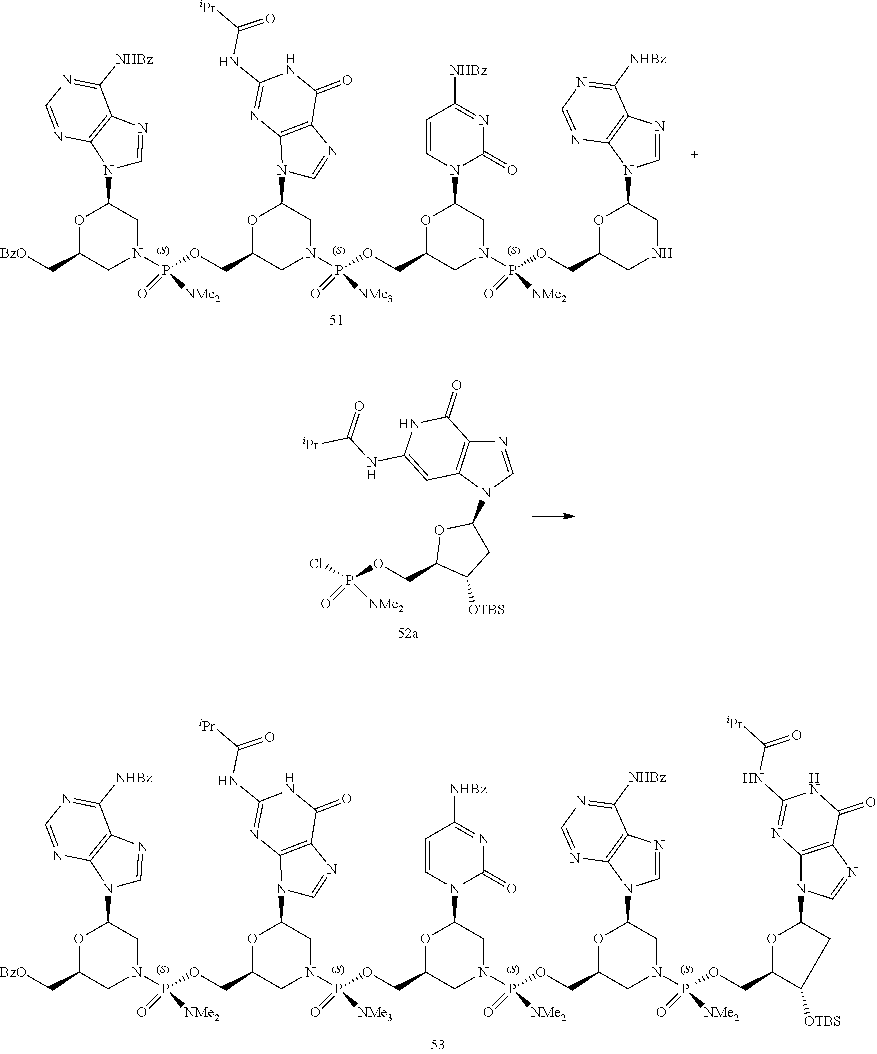

To a solution of starting material 51 (500 mg, 0.286 mmol) in 1,3-dimethyl-2-imidazolidinone (7.5 mL) was added 1,2,2,6,6-pentamethylpiperidine (0.16 mL, 0.86 mmol) followed by reactant 52a (206 mg, 0.358 mmol) (synthesized according to the process reported below) at ambient temperature. The reaction solution was stirred overnight and treated with EtOAc (7.5 mL) followed by MTBE (100 mL). The slurry was filtered and rinsed with a mixture of EtOAc (15 mL) and MTBE (100 mL) to give target compound 53 (710 mg).

³¹P NMR (162 MHz, METHANOL-d4) δ ppm 17.42 (s, 1P), 17.07 (s, 1P), 17.02 (s, 1P), 16.82 (s, 1P).

MS (ESI) m/z: [M+2H]²⁺ Calcd for C₉₉H₁₂₉N₃₁O₂₄P₄Si, 1143.93. Found 1144.03.

### 5-Mer of 5′ PMO: Deprotection

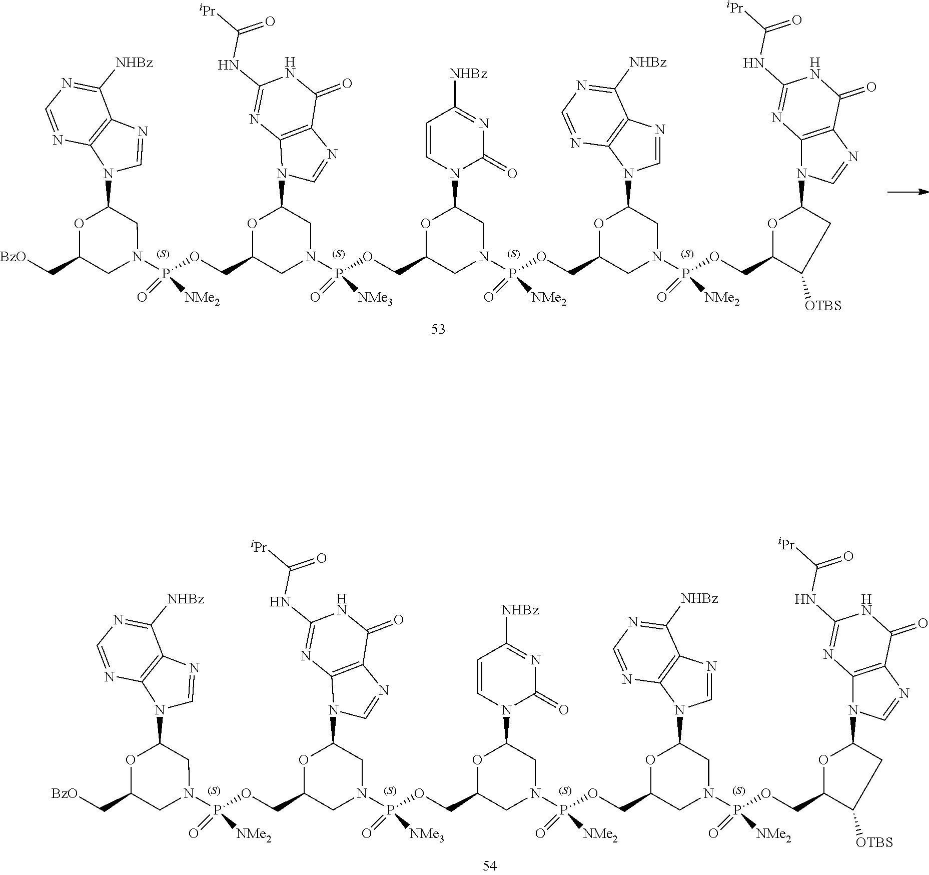

To a flask charged with starting material 53 (710 mg, 0.31 mmol) at ambient temperature was added pyridine (5.90 mL, 73.0 mmol), triethylamine (5.93 mL, 42.5 mmol) and CH₂Cl₂ (5.9 mL). The solution was then treated with triethylamine trihydrofluoride (759 μL, 4.66 mmol). The reaction solution was stirred overnight, cooled in an ice bath, and then treated with methoxytrimethylsilane (2.95 ml, 21.4 mmol). The mixture was stirred in the ice bath for 1 h and treated with 1,3-dimethyl-2-imidazolidinone (5.9 mL) followed by EtOAc (100 mL) and MTBE (50 mL). The slurry was filtered and rinsed with a mixture of CH₂Cl₂ (5.9 mL), EtOAc (118 mL) and MTBE (50 mL) to afford target compound 54 (627 mg). ³¹P NMR (162 MHz, CHLOROFORM-d) δ ppm 17.37 (s, 1P), 17.08 (s, 1P), 17.03 (s, 1P), 16.82 (s, 1P).

MS (ESI) m/z: [M+2H]²⁺ Calcd for C₉₃H₁₁₅N₃₁O₂₄P₄ 1087.39. Found 1087.17.

5-Mer of 5′ PMO: Activation with (−)-PSI

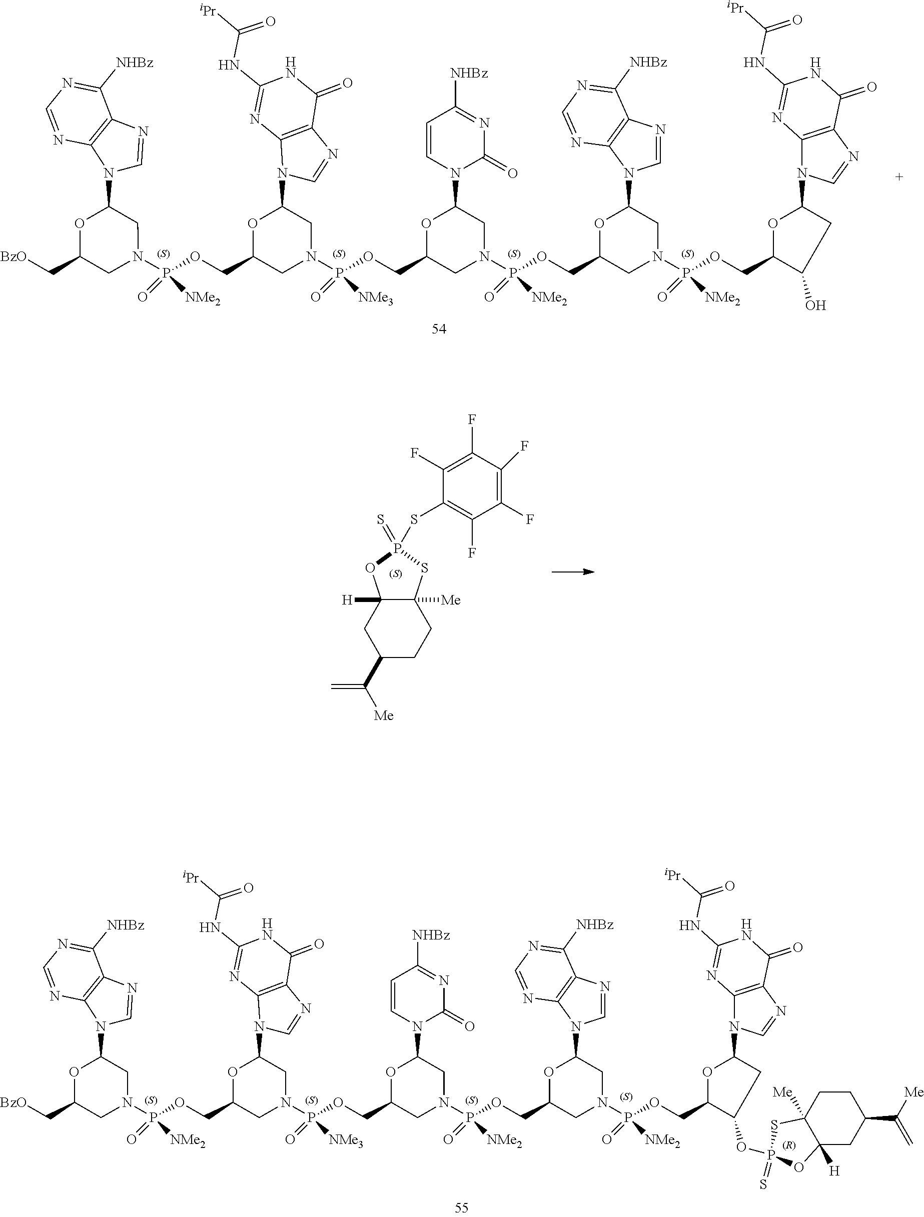

To a solution of starting material 54 (510 mg, 0.235 mmol) in a mixture of CH₂Cl₂ (21.9 mL), THF (7.1 mL) and 1,3-dimethyl-2-imidazolidinone (1.7 mL) was added (−)-PSI (Aldrich, CAS: 2245335-70-8, 194 mg, 0.434 mmol) at ambient temperature followed by activated 4 Å molecular sieves (2.5 g). The mixture was stirred for 50 min and treated dropwise with a solution of DBU (49.5 μL, 0.329 mmol) in CH₂Cl₂ (0.872 mL). The reaction mixture was then stirred for 30 min. The precipitate was filtered and the cake was rinsed with a mixture of CH₂Cl₂ (43.6 mL), THF (14.2 mL) and 1,3-dimethyl-2-imidazolidinone (3.5 mL). The filtrate was treated with MTBE (218 mL), the resulting precipitate was filtered, and the cake was rinsed with a mixture of CH₂Cl₂ (31.8 mL), THF (10.6 mL) and MTBE (100 mL) to afford target product 55 (548 mg).

³¹P NMR (162 MHz, CD₂Cl₂) δ ppm 101.46 (s, 1P), 16.74 (s, 1P), 16.46 (s, 1P), 16.32 (s, 1P), 16.13 (s, 1P).

MS (ESI) m/z: [M+2H]²⁺ Calcd for C₁₀₃H₁₃₀N₃₁O₂₅P₅S₂ 1210.40. Found 1210.09.

### Synthesis of Compounds 52a and 52b

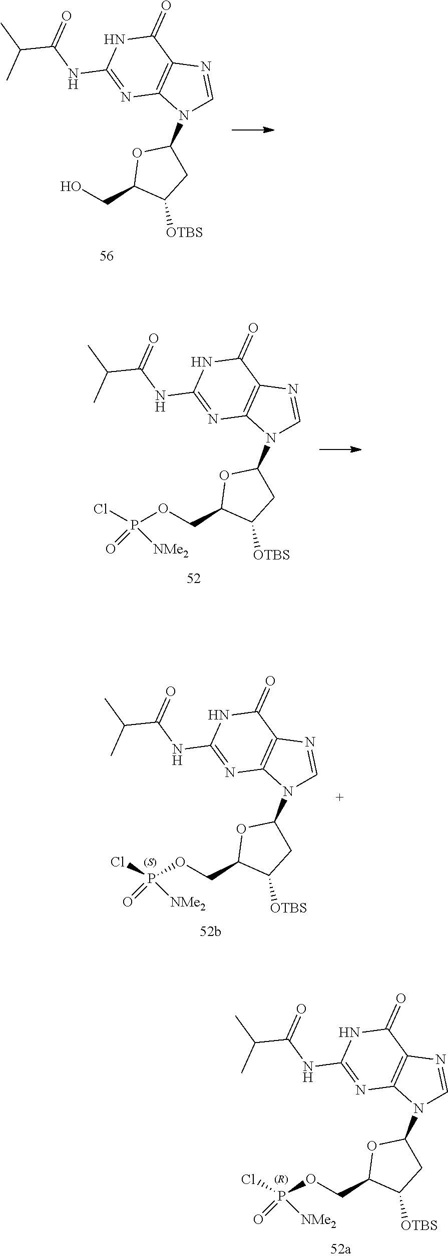

To a solution of N-(9-((2R,4S,5R)-4-((tert-butyldimethylsilyl)oxy)-5-(hydroxymethyl)tetrahydrofuran-2-yl)-6-oxo-6,9-dihydro-1H-purin-2-yl)isobutyramide 56 (2.76 g, 6.11 mmol) in acetonitrile (40 mL) and CH₂Cl₂ (40 mL) were added DBU (3.04 mL, mmol) and LiBr (1.75 g, 20.2 mmol) followed by dimethylphosphoramidic dichloride (1.16 mL, 9.78 mmol) at 0° C. The reaction solution was stirred at 0° C. for 1 h and then quenched with 10% aqueous citric acid (77 mL). The mixture was extracted two times with CH₂Cl₂ (200 mL each time). The combined organic layers were subsequently washed twice with water and 15 we % NaCl aqueous solution, dried over Na₂SO₄, and concentrated in vacuo. Biotage purification with a gradient of 90% to 100% EtOAc in n-heptane afforded target product 52 (1.91 g) as a mixture of two diastereomers 52a and 52b. The mixture of two diastereomers was subjected to prep. HPLC separation to afford 52b (444 mg) and 52a (304 mg).

### HPLC Conditions for Separation

Column: Chiralpak IA, 21×250 mm, 5μ
Flowrate: 20 mL/min
Mobile Phase: 100% EtOAc
Gradient: Isocratic
Runtime 20 mins
Injection Volume: 500 uL 150 mg/ml concentration
Detection: 254 nm

### Peak1 (Rt 9.3 Min)

((2R,3S,5R)-3-((tert-butyldimethyl silyl)oxy)-5-(2-isobutyramido-6-oxo-1,6-dihydro-9H-purin-9-yl)tetrahydrofuran-2-yl)methyl (S)-dimethylphosphoramidochloridate (52b):

¹H NMR (400 MHz, CHLOROFORM-d) δ=12.19 (br s, 1H), 9.93 (br s, 1H), 7.76 (br s, 1H), 6.25 (br t, J=7.3 Hz, 1H), 4.98-4.90 (m, 1H), 4.67 (br d, J=4.3 Hz, 1H), 4.39-4.26 (m, 2H), 3.08-2.99 (m, 1H), 2.82-2.73 (m, 1H), 2.73 (s, 3H), 2.69 (s, 3H), 2.28 (br dd, J=13.5 Hz, 1H), 1.26 (d, J=6.9 Hz, 3H), 1.22 (d, J=6.8 Hz, 3H), 0.93 (s, 9H), 0.14 (s, 3H), 0.14 (s, 3H).

³¹P NMR (162 MHz, CHLOROFORM-d) δ ppm 20.39 (s, 1P).

MS (ESI) m/z: [M+H]⁺ Calcd for C₂₂H₃₉ClN₆O₆PSi 577.21. Found 577.07.

### Peak2 (Rt 15.3 Min)

((2R,3S,5R)-3-((tert-butyldimethylsilyl)oxy)-5-(2-isobutyramido-6-oxo-1,6-dihydro-9H-purin-9-yl)tetrahydrofuran-2-yl)methyl (R)-dimethylphosphoramidochloridate (52a).

¹H NMR (400 MHz, CHLOROFORM-d) δ=12.24 (br s, 1H), 10.34 (br s, 1H), 7.88 (br s, 1H), 6.27 (br t, J=6.8 Hz, 1H), 5.27-5.13 (m, 1H), 4.91-4.85 (m, 1H), 4.37-4.26 (m, 1H), 4.15-4.07 (m, 1H), 3.24-3.16 (m, 1H), 2.80 (s, 3H), 2.76 (s, 3H), 2.75-2.71 (m, 1H), 2.37 (br dd, J=6.9, 12.1 Hz, 1H), 1.25 (d, J=6.8 Hz, 3H), 1.24 (d, J=6.8 Hz, 3H), 0.92 (s, 9H), (s, 3H), 0.12 (s, 3H)

³¹P NMR (162 MHz, CHLOROFORM-d) δ ppm 19.67 (s, 1P).

MS (ESI) m/z: [M+H]⁺ Calcd for C₂₂H₃₉ClN₆O₆PSi 577.21. Found 577.07.

### Example 5.2: Preparation of 3′-PMO Wing

### 2-Mer of 3′-PMO: Coupling

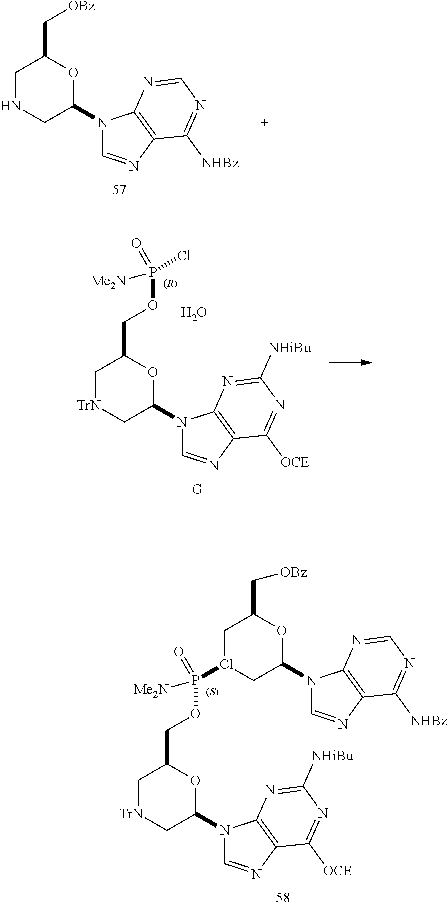

To a solution of starting material 57 (1.33 g, 2.32 mmol) in THF (16 mL) was added 1,2,2,6,6-pentamethylpiperidine (1.15 mL, 6.34 mmol). The resulting solution was cooled to ° C. and treated with reactant G2 (1.60 g, 2.11 mmol). The reaction mixture was warmed to ambient temperature and stirred overnight. A saturated NaHCO₃ solution (25 mL) and water (10 mL) were added, and the resulting mixture was extracted with CH₂Cl₂ (40 mL each) three times. The combined organic layers were washed with 30 wt % NaCl aqueous solution (20 mL), dried over MgSO₄, filtered, and concentrated in vacuo. The residue was purified by silica gel column chromatography. Elution with 3-15% MeOH in EtOAc afforded 2.316 g of target product 58.

MS (ESI) m/z: [M+H]⁺ Calcd for C₆₂H₆₄N₁₄O₉P, 1179.47. Found 1179.41.

### 2-Mer of 3′-PMO: Deprotection

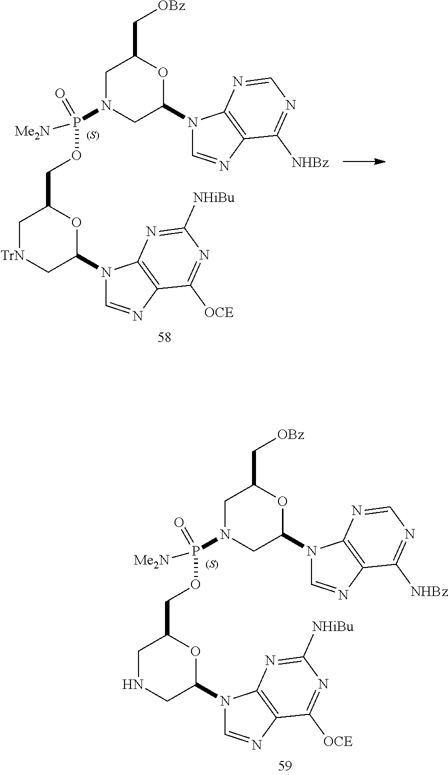

To a solution of starting material 58 (2.316 g, 1.964 mmol) in CH₂Cl₂ (35 mL) at ambient temperature was added ethanol (1.2 mL, 20 mmol) followed by TFA (0.91 mL, 12 mmol). The reaction mixture was stirred at ambient temperature for 1 h, and then treated with 1,2,2,6,6-pentamethylpiperidine (2.7 mL, 15 mmol). The resulting mixture was concentrated in vacuo. The residue was treated with EtOAc (25 mL) followed by MTBE (50 mL). The resulting slurry was filtered through a glass filter and rinsed with a mixture of MTBE and EtOAc (15 mL/5 mL). The filter cake was dried in vacuo for 2 h to provide 1.75 g of target product 59.

MS (ESI) m/z: [M+H]⁺ Calcd for C₄₃H₅₀N₁₄O₉P, 937.36. Found 937.10.

### 3-Mer of 3′-PMO: Coupling

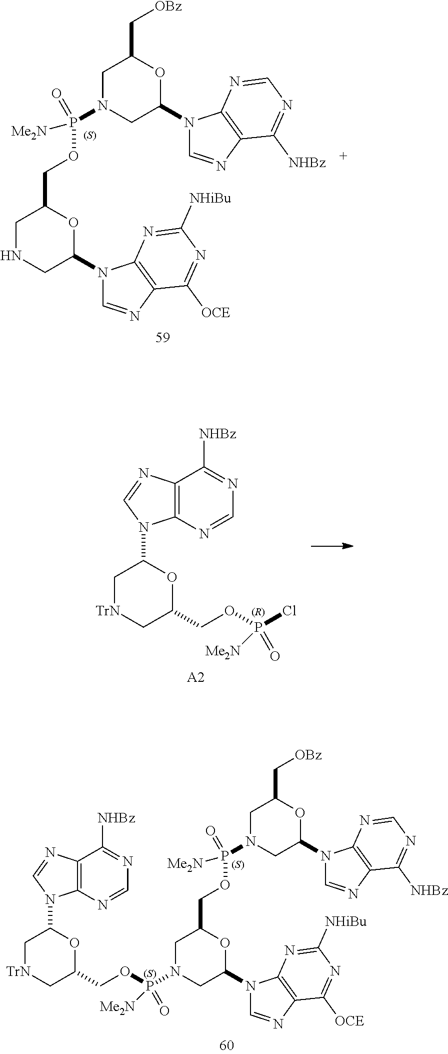

To a solution of starting material 59 (1.75 g, 1.87 mmol) in 1,3-dimethyl-2-imidazolidinone (20 mL) at 0° C. was added 1,2,2,6,6-pentamethylpiperidine (0.68 mL, 3.7 mmol) followed by reactant A2 (1.42 g, 1.96 mmol). The reaction mixture was warmed to ambient temperature and stirred overnight. To the reaction mixture was added EtOAc (20 mL) followed by MTBE (60 mL) and n-heptane (80 mL). The precipitate was collected by decantation. The isolated product (60) was directly used for the next step without further purification.

MS (ESI) m/z: [M+H]⁺ Calcd for C₈₁H₈₆N₂₁O₁₃P₂ 1622.62. Found 1622.59.

### 3-Mer of 3′-PMO: Deprotection

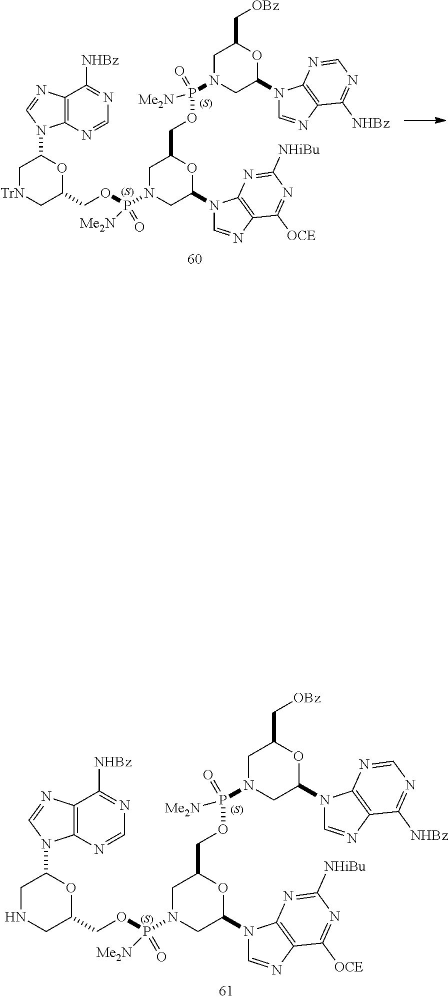

To a solution of starting material 60 (3.03 g, 1.87 mmol in theory) in CH₂Cl₂ (24 mL) at ambient temperature were added ethanol (1.1 mL, 19 mmol) and TFA (0.86 mL, 11.2 mmol). The reaction mixture was stirred for 30 min before additional TFA (0.43 mL, 5.6 mmol) was added. After being stirred for 2 h, the reaction mixture was treated with EtOAc (75 mL) followed by MTBE (50 mL). The precipitate was collected by filtration and rinsed with EtOAc/MTBE (10 mL/10 mL). The resulting solid was dissolved in CH₂Cl₂ (25 mL) and treated with 1,2,2,6,6-pentamethylpiperidine (1.02 mL, 5.60 mmol) at ambient temperature. The mixture was stirred for 10 min before EtOAc (75 mL) and MTBE (50 mL) were added. The resulting precipitate was collected by filtration and rinsed with EtOAc/MTBE (15 mL/15 mL). Drying the filter cake in vacuo provided 2.25 g of target product 61.

MS (ESI) m/z: [M+H]⁺ Calcd for C₆₂H₇₂N₂₁O₁₃P₂ 1380.51. Found 1380.31.

### 4-Mer of 3′-PMO: Coupling

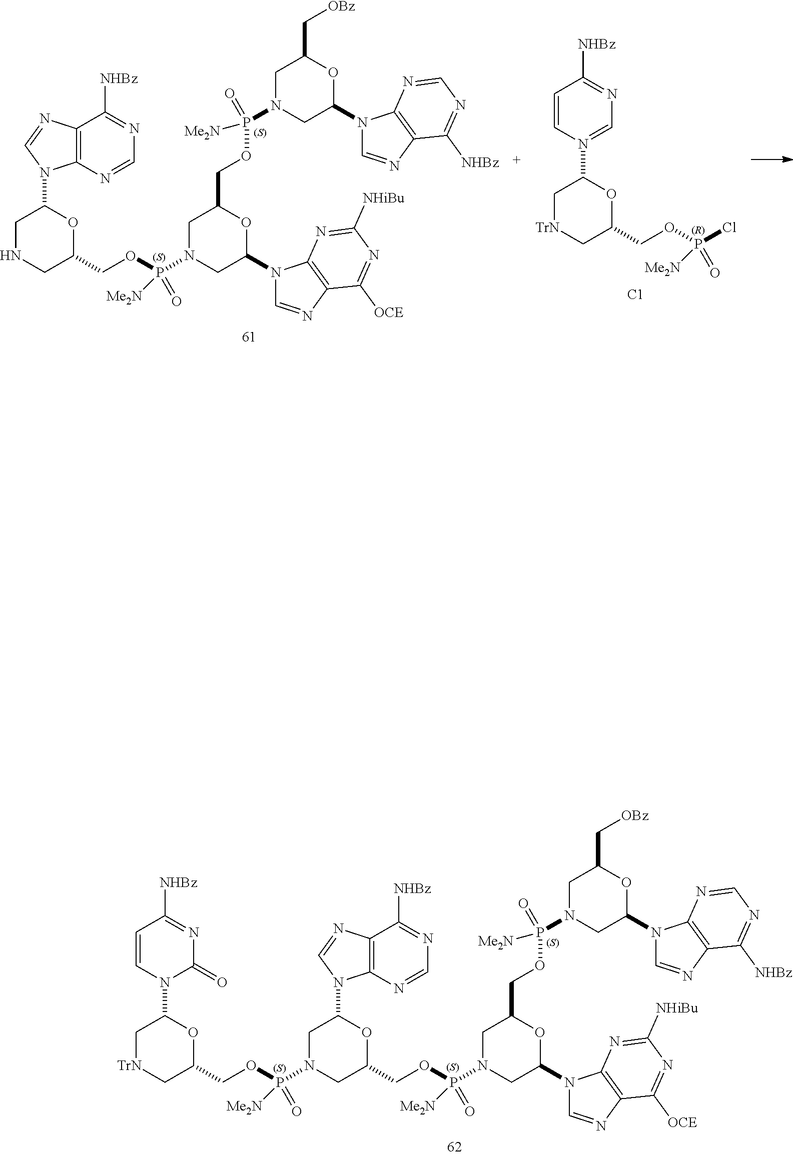

To a solution of starting material 61 (2.20 g, 1.59 mmol) in 1,3-dimethyl-2-imidazolidinone (20 mL) at ambient temperature was added 1,2,2,6,6-pentamethylpiperidine (0.73 mL, 4.0 mmol) followed by reactant C1 (1.22 g, 1.75 mmol). The reaction mixture was stirred overnight before additional C1 (0.20 g, 0.29 mmol) was added. After being stirred for additional 4 h, the reaction mixture was treated with morpholine (42 μL, 0.48 mmol). After min, EtOAc (20 mL) and MTBE (150 mL) were added. The resulting precipitate was collected by filtration, rinsed with a mixture of EtOAc/MTBE (10 mL/20 mL) and dried in vacuo overnight. The resulting solid (3.74 g) was dissolved in CH₂Cl₂ (25 mL). To the solution was added EtOAc (25 mL) followed by MTBE (100 mL). The resulting precipitate was collected by filtration, rinsed with a mixture of EtOAc/MTBE (10 mL/30 mL), and dried in vacuo overnight. 3.20 g of target product 62 was obtained.

MS (ESI) m/z: [M-Tr+2H]⁺ Calcd for C₈₀H₉₄N₂₆O₁₈P₃1800.65. Found 1800.05.

4-mer of 3′-PMO: Deprotection

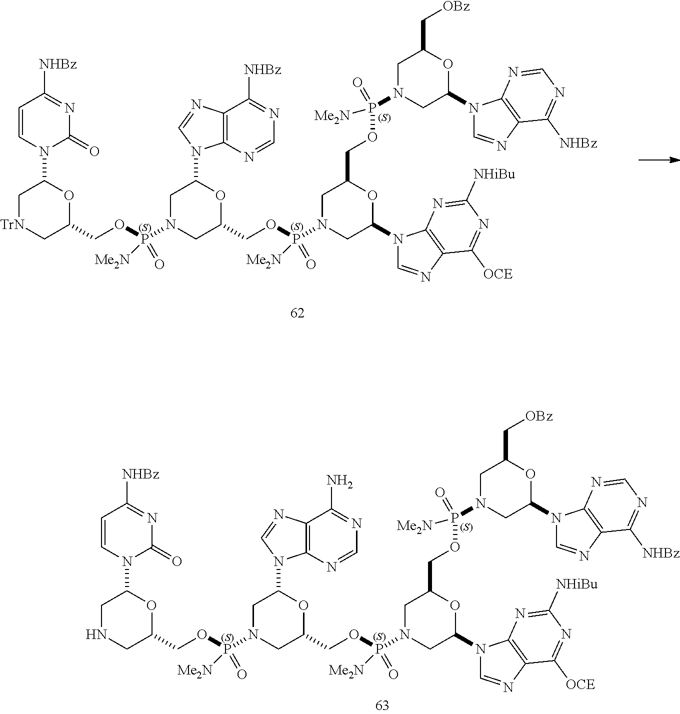

To a solution of starting material 62 (194 mg, 1.57 mmol) in CH₂Cl₂ (42 mL) at ambient temperature were added EtOH (0.92 mL) and TFA (0.96 mL, 12 mmol). The reaction mixture was stirred for 2 h and treated with EtOAc (4 mL) followed by MTBE (80 mL). The resulting precipitate was collected by filtration and rinsed with a mixture of EtOAc/MTBE (10 mL/20 mL). The resulting solid was dissolved in CH₂Cl₂ (42 mL) and treated with 1,2,2,6,6-pentamethylpiperidine (0.85 mL, 4.7 mmol). The resulting solution was stirred at ambient temperature for 10 min before EtOAc (40 ml) and MTBE (100 mL) were added. The precipitate was collected by filtration, rinsed with a mixture of EtOAc/MTBE (20 mL/40 mL), and dried in vacuo for 2 h. The solid was dissolved in CH₂Cl₂ (40 mL). To the solution was added EtOAc (40 mL) followed by MTBE (60 mL). The resulting precipitate was collected by filtration and rinsed with a mixture of EtOAc/MTBE (20 mL/20 mL). The solid was dissolved in CH₂Cl₂ (40 mL) and treated with EtOAc (80 mL). The resulting precipitate was collected by filtration and rinsed with EtOAc (˜30 mL). Drying the filter cake in vacuo provided 2.05 g of target product 63.

MS (ESI) m/z: [M+H]⁺ Calcd for C₈₀H₉₄N₂₆O₁₈P₃ 1800.65. Found 1800.68.

### 4-Mer of 3′-PMO: Global Deprotection

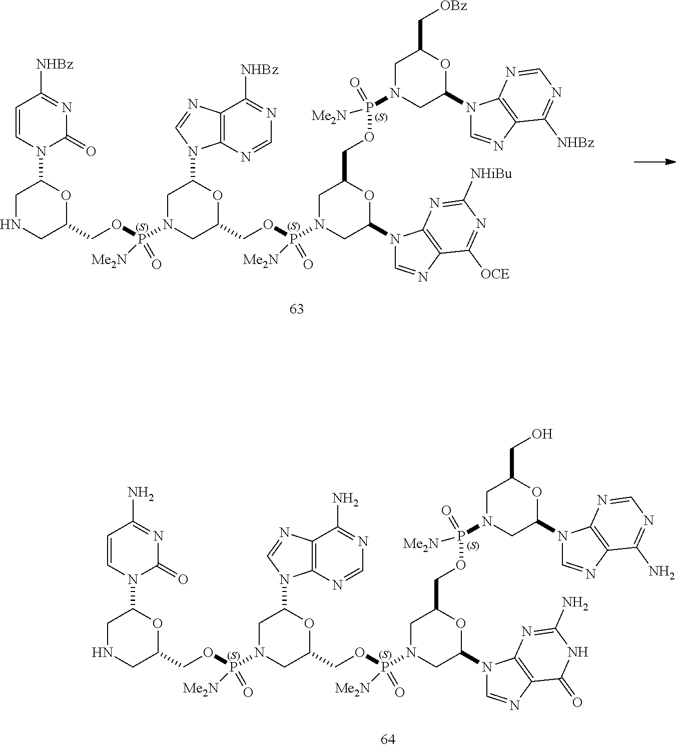

Starting material 63 (1.25 g, 0.695 mmol) was dissolved in a mixture of methanol (20 mL) and 28% ammonium hydroxide (20 mL) at ambient temperature. To the solution was added morpholine (0.73 mL, 8.3 mmol). The resulting mixture was heated at 50-52° C. for 15 h and cooled to ambient temperature. After concentration in vacuo, the residue was dissolved in CH₂Cl₂/MeOH (12.5 mL/5 mL) and treated with EtOAc (60 mL). The resulting precipitate was collected by filtration and rinsed with a mixture of EtOAc/CH₂Cl₂/MeOH (20 mL/2.5 mL/1 mL). Drying the filter cake in vacuo overnight afforded 928 mg of target product 64. MS (ESI) m/z: [M+H]⁺ Calcd for C₄₅H₆₉N₂₅O₁₃P₃ 1260.47. Found 1260.98.

### 4-Mer of 3′-PMO: Morpholine Protection

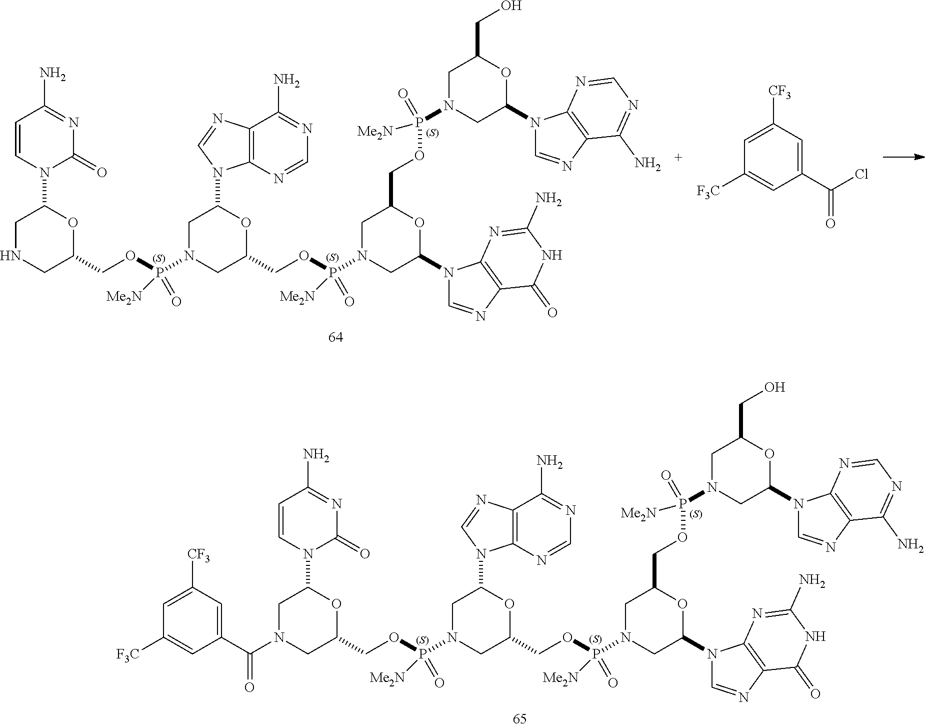

To a solution of starting material 64 (928 mg, 0.405 mmol in theory) in a mixture of THF/Water/MeOH (15 mL/2.5 mL/4.5 mL) were added 1,2,2,6,6-pentamethylpiperidine (0.367 mL, 2.02 mmol) and 3,5-bis(trifluoromethyl)benzoyl chloride (0.11 mL, 0.61 mmol). After 3 h, additional 0.025 mL of bis(trifluoromethyl)benzoyl chloride was added. After being stirred overnight, the reaction mixture was treated with EtOAc (60 mL). The resulting gummy solid was isolated by decantation and dissolved in a mixture of MeOH/CH₂Cl₂ (2 mL/8 mL). To the solution was added EtOAc (50 mL). The resulting precipitate was isolated by filtration, rinsed with EtOAc, and dried in vacuo for 20 min. The resulting solid was treated with a mixture of MeCN/EtOAc (7.5 mL/7.5 mL). The slurry was filtered through a glass filter and rinsed with a mixture of MeCN/EtOAc (2.5 mL/2.5 mL). Drying the filter cake in vacuo for 1 h afforded 550 mg of target product 65.

³¹P NMR (162 MHz, METHANOL-d4) δ=17.16 (s, 1P), 17.11 (s, 1P), 16.97 (s, 1P) MS (ESI) m/z: [M+H]⁺ Calcd for C₅₄H₇₁F₆N₂₅O₁₄P₃ 1500.47. Found. 1500.22.

### Example 5.3: Elongation of DNA

### 5-Mer: Coupling

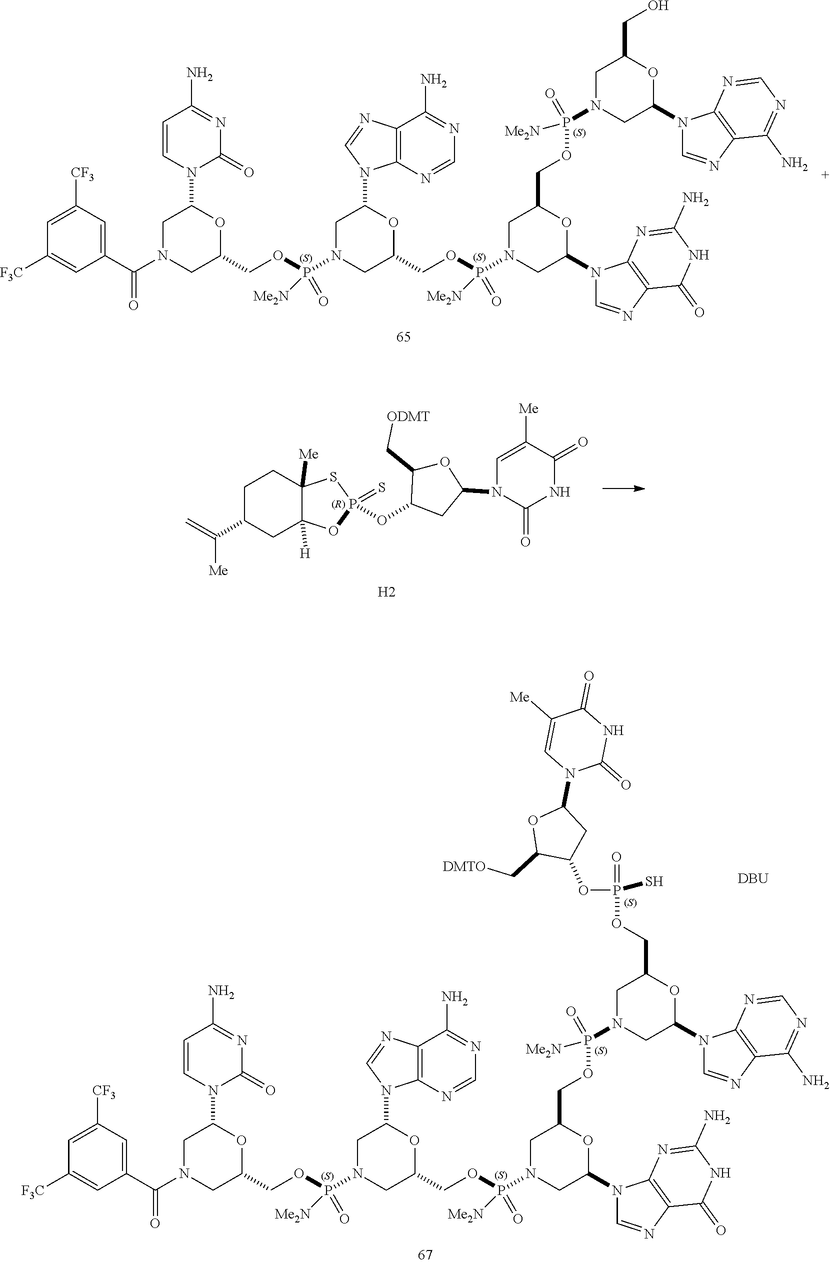

Starting material 65 (550 mg, 0.367 mmol) and reactant 112 (783 mg, 0.99 mmol) were dissolved in 1,3-dimethyl-2-imidazolidinone (19 mL). To the resulting solution was added 4 Å molecular sieves (1.7 g). The reaction flask was applied to vacuum and filled with nitrogen. The process was repeated two more times. After being stirred for 30 min, the resulting mixture was treated with DBU (0.22 mL, 1.47 mmol). The reaction mixture was stirred for 1 hr at ambient temperature and then filtered through a syringe filter. The filtrate was added into EtOAc (30 mL), rinsing with 1,3-dimethyl-2-imidazolidinone (6 mL). To the resulting slurry was added additional EtOAc (50 mL). The resulting precipitate was collected by filtration and rinsed with a mixture of EtOAc/MeCN (10 mL/10 mL). The filter cake was treated with MeCN (20 mL) followed by EtOAc (20 mL). After 10 min, the resulting slurry was filtered through a glass filter and rinsed with EtOAc/MeCN (5 mL/5 mL). Drying the filter cake in vacuo for 3 days afforded 790 mg of target product 67.

³¹P NMR (162 MHz, METHANOL-d4) δ=57.76 (s, 1P), 17.10 (s, 1P), 17.02 (s, 1P), 16.90 (s, 1P).

MS (ESI) m/z: [M-DMT+2H]⁺ Calcd for C₆₄E₁₈₄F₆N₂₇O₂₀P₄S, 1820.50. Found 1820.18.

### 5-Mer: Deprotection

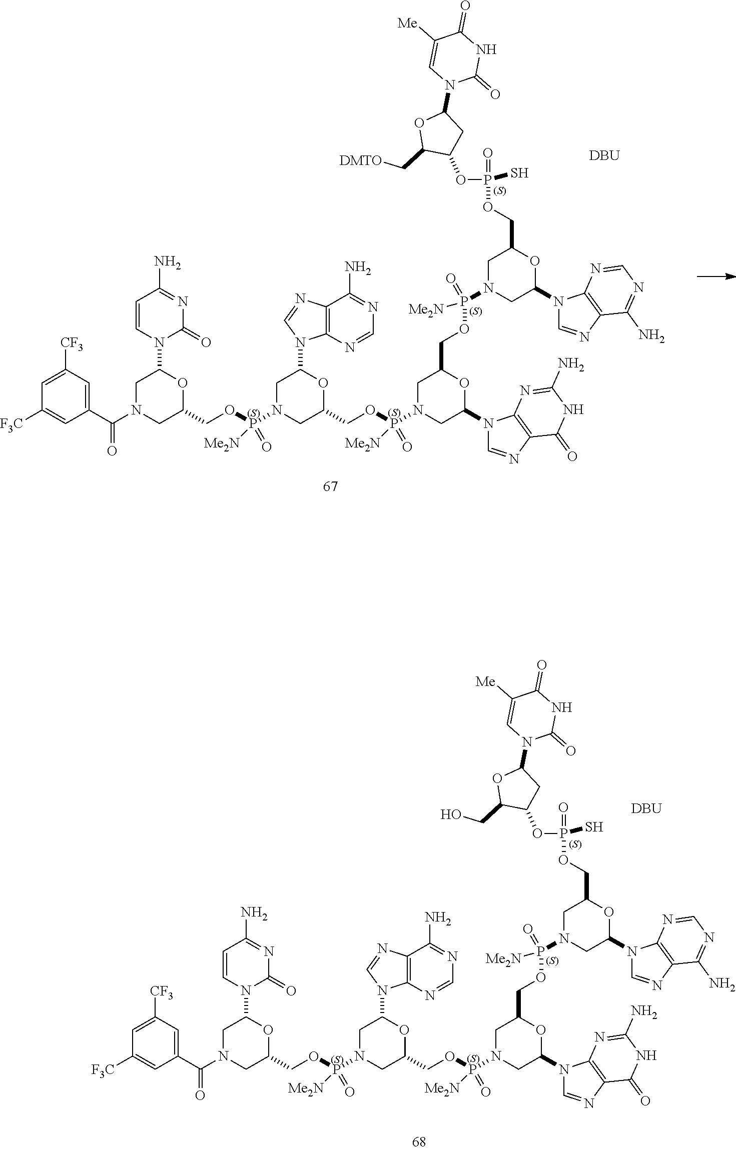

Starting material 67 (0.790 g, 0.347 mmol) was dissolved in a mixture of 1,1,1,3,3,3-hexafluoro-2-propanol (8 mL), 2,2,2-trifluoroethanol (2 mL), CH₂Cl₂ (10 mL) and triethylsilane (6 mL). The reaction mixture was stirred for 3 h at ambient temperature, and an additional mixture of 1,1,1,3,3,3-hexafluoro-2-propanol (2 mL), 2,2,2-trifluoroethanol (0.5 mL), CH₂Cl₂ (2.5 mL) and triethylsilane (1.5 mL) was added. After additional 1 h stirring, the reaction mixture was treated with EtOAc (150 mL) followed by MTBE (75 mL). The resulting precipitate was collected by centrifuge (3500 rpm, 35 min) and rinsed with a mixture of EtOAc/MeCN (10 mL/10 mL). The pellet was treated with MeCN (25 mL) to make a slurry. After 5 min stirring, EtOAc (25 mL) was added. The resulting slurry was filtered through a glass filter and rinsed with MeCN/EtOAc (10 mL/10 mL). Drying the filter cake in vacuo overnight provided 646 mg of target product 68.

MS (ESI) m/z: Calcd for C₆₄H₈₂F₆N₂₇O₂₀P₄S, 1818.48. Found 1818.37.

### 6-Mer: Coupling

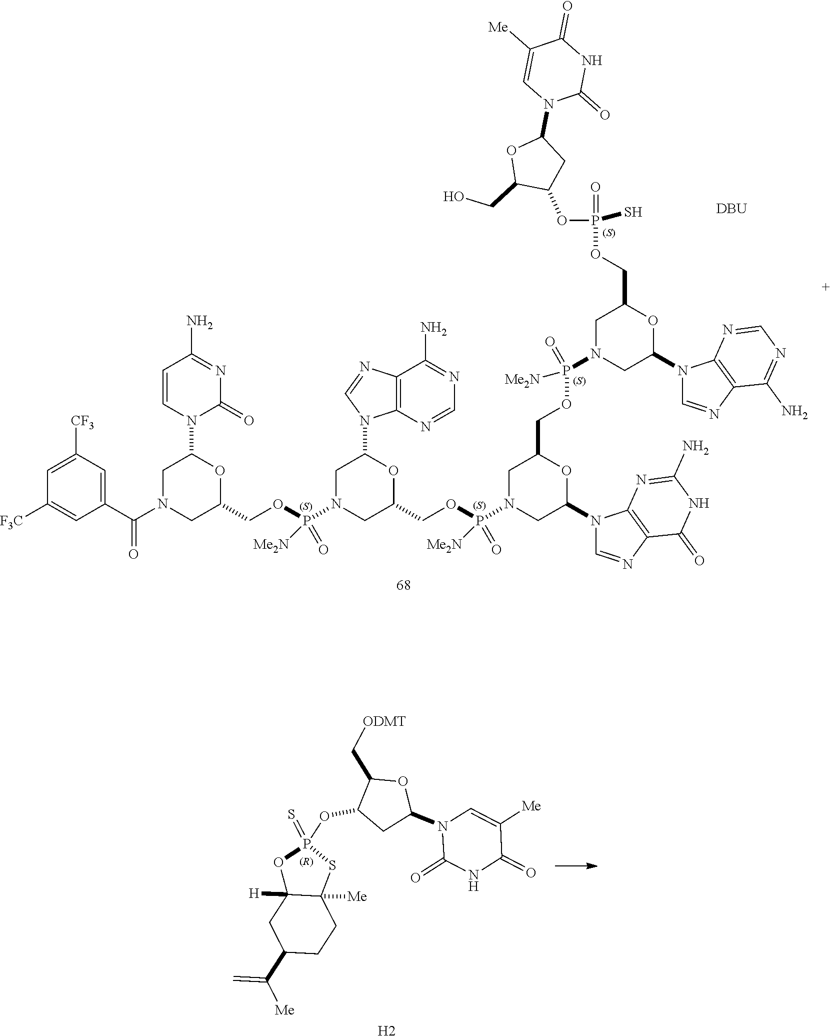

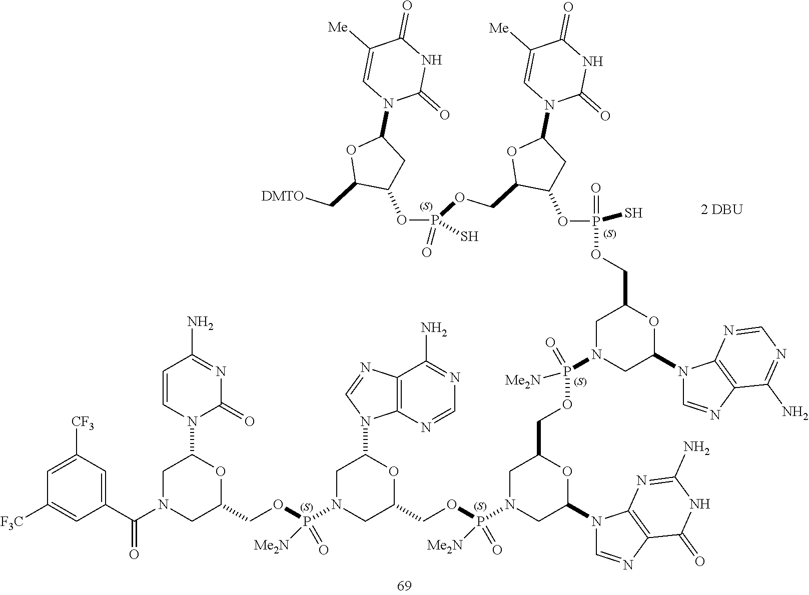

Starting material 68 (646 mg, 0.327 mmol) and reactant 112 (777 mg, 0.982 mmol) were dissolved in 1,3-dimethyl-2-imidazolidinone (16 mL). To the resulting solution was added 4 Å molecular sieves (2 g). The reaction flask was applied to vacuum and filled with nitrogen. The process was repeated two more times. After being stirred for 30 min, the resulting mixture was treated with DBU (0.25 mL, 1.64 mmol). The reaction mixture was stirred for 2 h at ambient temperature and then filtered through a syringe filter. The filtrate was added into EtOAc (35 mL), rinsing with 1,3-dimethyl-2-imidazolidinone (4 mL). To the resulting slurry was added additional EtOAc (40 mL). The precipitate was isolated by filtration and rinsed with MeCN/EtOAc (5 mL/5 mL). The resulting solid was treated with MeCN (20 mL) followed by EtOAc (20 mL). The resulting slurry was filtered through a glass filter and rinsed with EtOAc/MeCN (5 mL/5 mL). Drying the filter cake in vacuo overnight provided 0.90 g of target product 69.

MS (ESI) m/z: [M−2H]²⁻ Calcd for C₉₅H₁₁₃F₆N₂₉O₂₈P₅S₂ 1220.32. Found 1220.47.

### 6-Mer: Deprotection

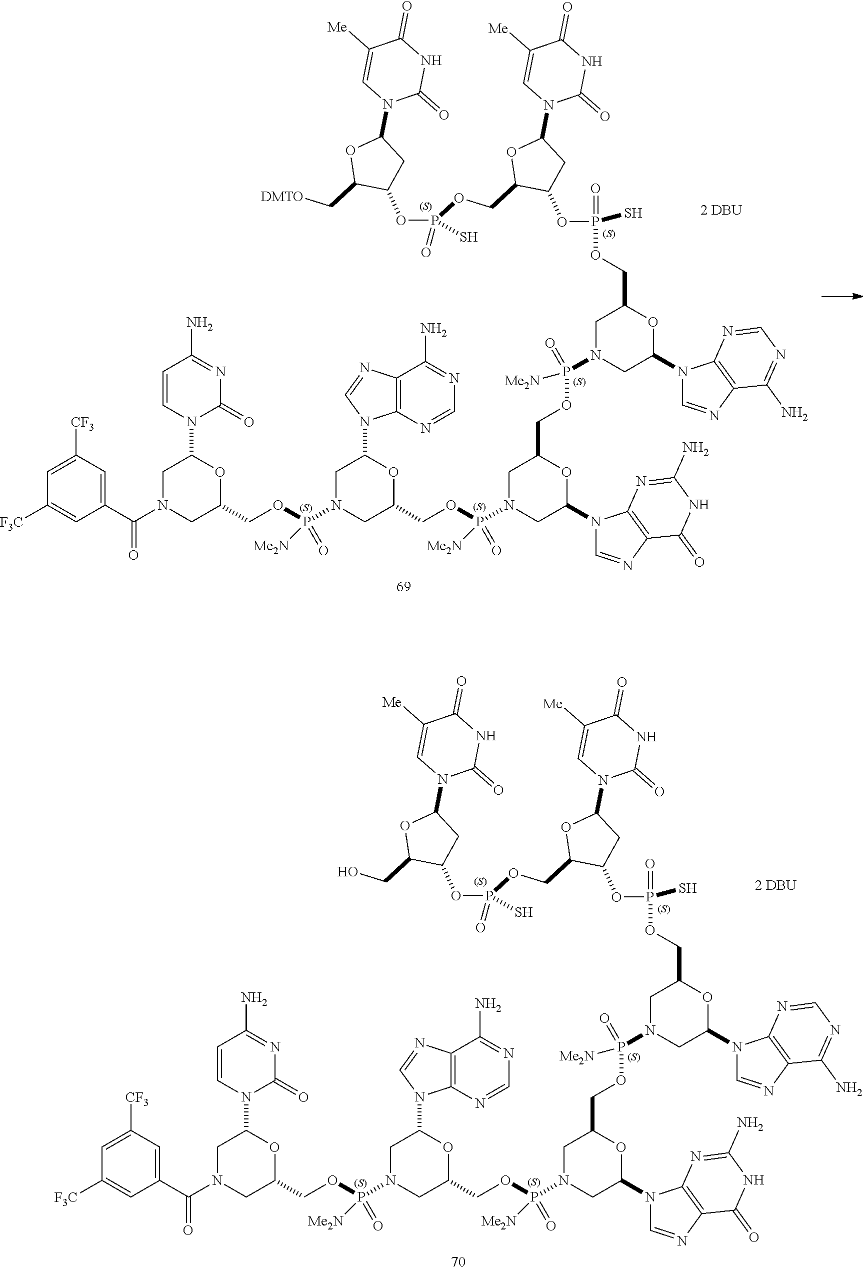

To starting material 69 (0.90 g, 0.328 mmol) was added a mixture of 1,1,1,3,3,3-hexafluoro-2-propanol (10.8 mL), 2,2,2-trifluoroethanol (2.7 mL), triethylsilane (8.1 mL) and CH₂Cl₂ (13.5 mL). After being stirred at ambient temperature overnight, the reaction mixture was treated with EtOAc (150 mL) followed by MTBE (100 mL). The resulting precipitate was isolated by filtration and rinsed with a mixture of EtOAc/MeCN (10 mL/10 mL). The filter cake was treated with MeCN (25 mL) to make a slurry. After 5 min stirring, EtOAc (25 mL) was added. The resulting slurry was filtered through a glass filter and rinsed with MeCN/EtOAc (10 mL/10 mL). Drying the filter cake in vacuo for 1 h provided 800 mg of target product 70.

MS (ESI) m/z: [M+2H]²⁺ Calcd for C₇₄H₉₈F₆N₂₉O₂₆P₅S₂ 1070.76. Found 1070.66.

### 7-Mer: Coupling

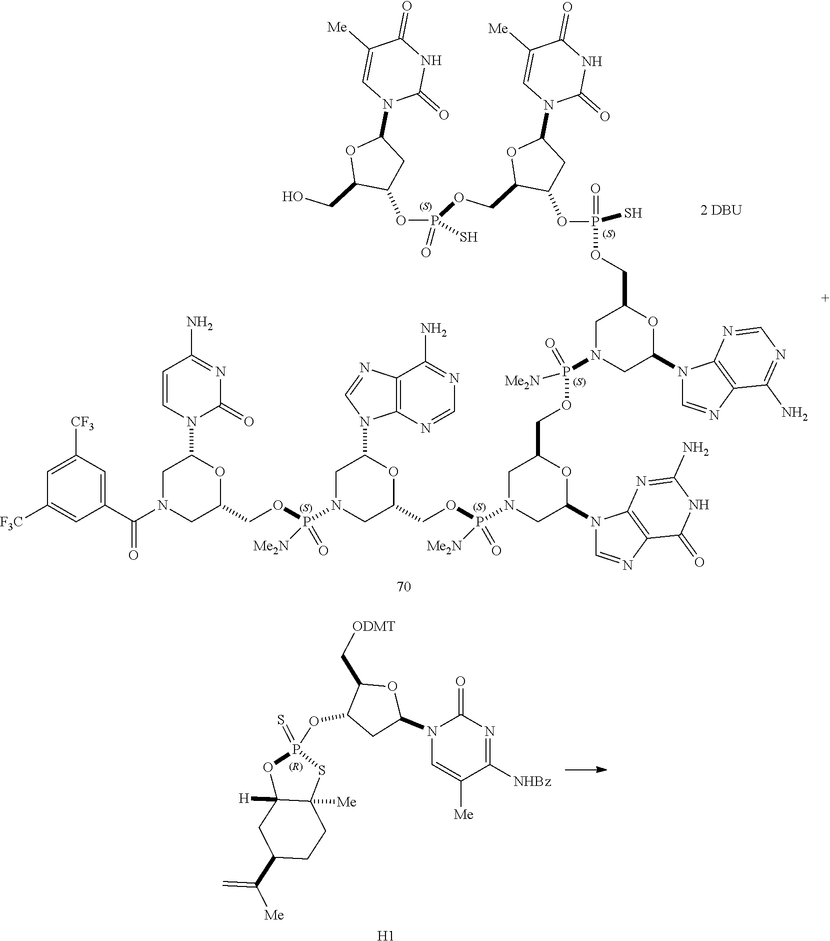

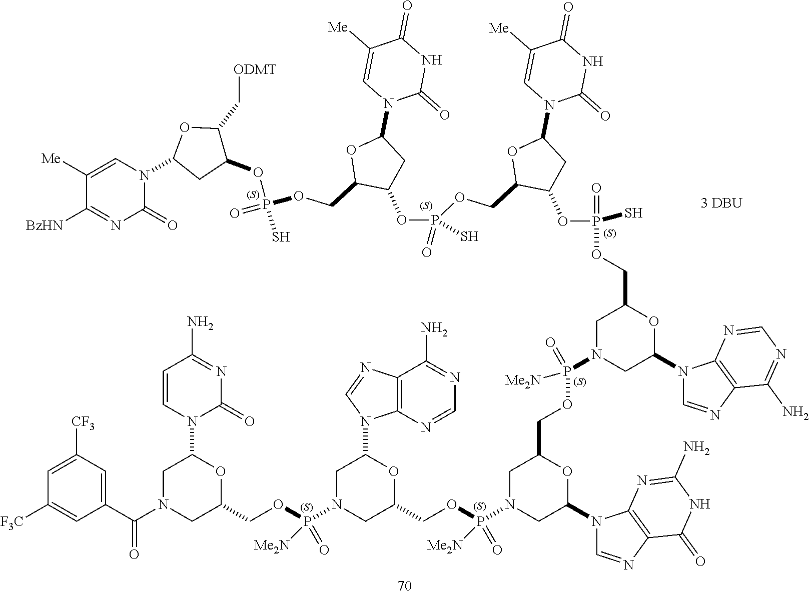

Starting material 70 (950 mg, 0.389 mmol) and reactant H1 (1042 mg, 1.17 mmol) were dissolved in 1,3-dimethyl-2-imidazolidinone (23.8 mL). To the resulting solution was added 4 Å molecular sieves (1 g). The reaction flask was applied to vacuum and filled with nitrogen. The process was repeated two more times. After being stirred for 30 min, the resulting mixture was treated with DBU (0.35 mL, 2.33 mmol). The reaction mixture was stirred for 16 h at ambient temperature and then filtered through a syringe filter. The filtrate was added into EtOAc (40 mL), rinsing with 1,3-dimethyl-2-imidazolidinone (5 mL). To the resulting slurry was added additional EtOAc (35 mL). The precipitate was isolated by filtration and rinsed with MeCN/EtOAc (10 mL/10 mL). The resulting solid was treated with MeCN (20 mL) followed by EtOAc (20 mL). The resulting slurry was filtered through a glass filter and rinsed with EtOAc/MeCN (7.5 mL/7.5 mL). Drying the filter cake in vacuo for 4 h provided 1.20 g of target product 71.

³¹P NMR (162 MHz, METHANOL-d4) δ=57.13 (s, 1P), 56.94 (s, 2P), 17.05 (s, 1P), 16.98 (s, 1P), 16.79 (s, 1P).

MS (ESI) m/z: [M−2H]²⁻ Calcd for C₁₁₂H₁₃₀F₆N₃₂O₃₄P₆S₃ 1431.35. Found 1431.26.

### 7-Mer: Deprotection

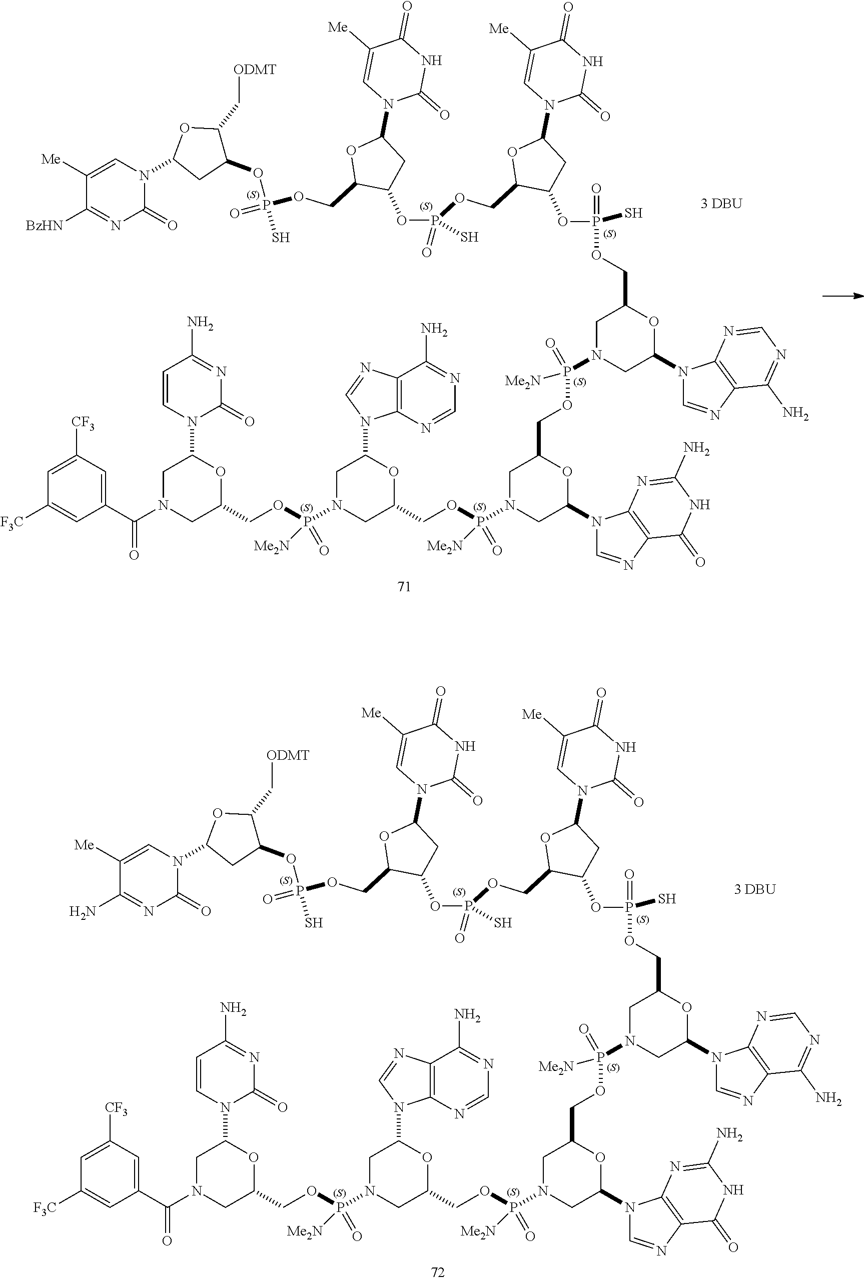

To starting material 71 (1.20 g, 0.361 mmol) was added a mixture of 1,1,1,3,3,3-hexafluoro-2-propanol (14.4 mL), 2,2,2-trifluoroethanol (3.6 mL), triethylsilane (10.8 mL) and CH₂Cl₂ (18 mL). After being stirred at ambient temperature overnight, the resulting solution was treated with EtOAc (100 mL) followed by MTBE (50 mL). The resulting precipitate was collected by filtration and rinsed with a mixture of EtOAc/MeCN (10 mL/10 mL). The filter cake was treated with MeCN (25 mL) followed by EtOAc (25 mL). The resulting slurry was filtered through a glass filter and rinsed with MeCN/EtOAc (10 mL/10 mL). Drying the filter cake in vacuo for 3 h provided 1.0 g of target product 72. MS (ESI) m/z: [M−2H]²⁻ Calcd for C₈₄H₁₀₈F₆N₃₂O₃₁P₆S₃ 1228.27. Found 1228.50.

### 8-Mer: Coupling

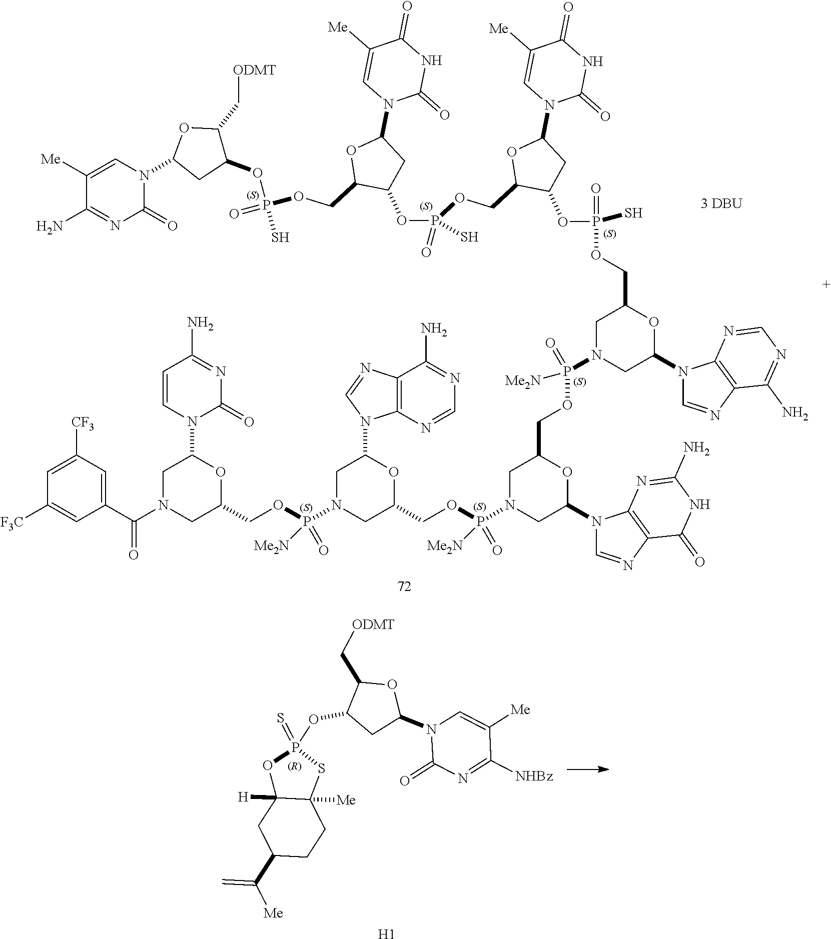

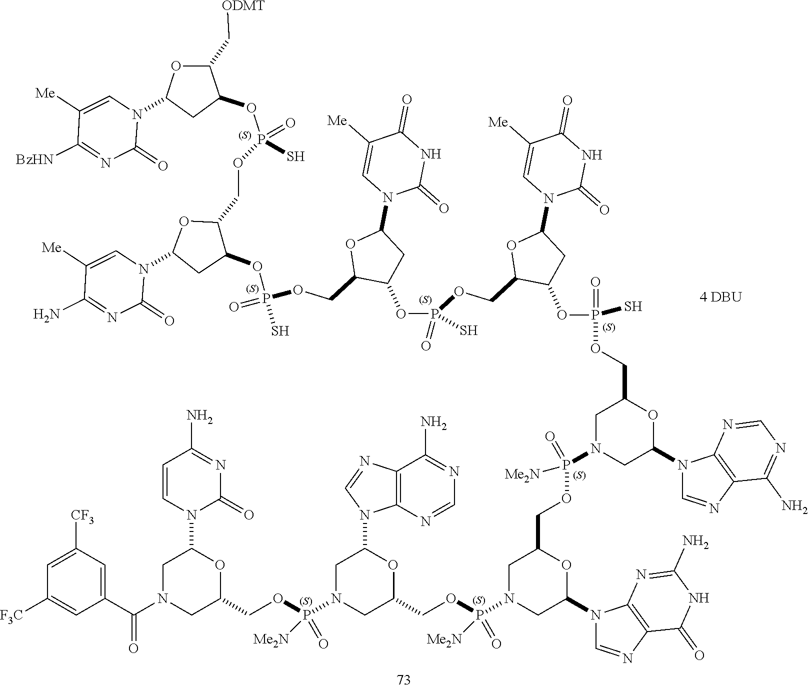

To a solution of starting material 72 (300 mg, 0.103 mmol) in 1,3-dimethyl-2-imidazolidinone (9.0 mL) was added reactant H1 (276 mg, 0.309 mmol). To the resulting solution was added 4 Å molecular sieves (1.0 g). The reaction flask was applied to vacuum and filled with nitrogen and the process was repeated two more times. After being stirred for 30 min, the resulting mixture was treated with DBU (0.11 mL, 0.72 mmol). The reaction mixture was stirred for 4 h at ambient temperature and then filtered through a syringe filter. The filtrate was added into EtOAc (25 mL), rinsing with 1,3-dimethyl-2-imidazolidinone (4.5 mL). To the resulting slurry was added additional EtOAc (20 mL). The precipitate was isolated by filtration and rinsed with MeCN/EtOAc (7.5 mL/7.5 mL). The resulting solid was treated with MeCN (10 mL) followed by EtOAc (10 mL). The resulting slurry was filtered through a glass filter and rinsed with EtOAc/MeCN (5 mL/5 mL). Drying the filter cake in vacuo overnight provided 0.36 g of target product 73.

³¹P NMR (162 MHz, METHANOL-d4) δ=57.36 (s, 1P), 57.31 (s, 1P), 56.90 (s, 1P), 56.27 (s, 1P) 16.96 (s, 1P), 16.94 (s, 1P), 16.67 (s, 1P).

MS (ESI) m/z: [M−2H]²⁻ Calcd for C₁₂₂H₁₄₄F₆N₃₅O₃₉P₇S₄1591.37. Found 1591.35.

### 8-Mer: Deprotection

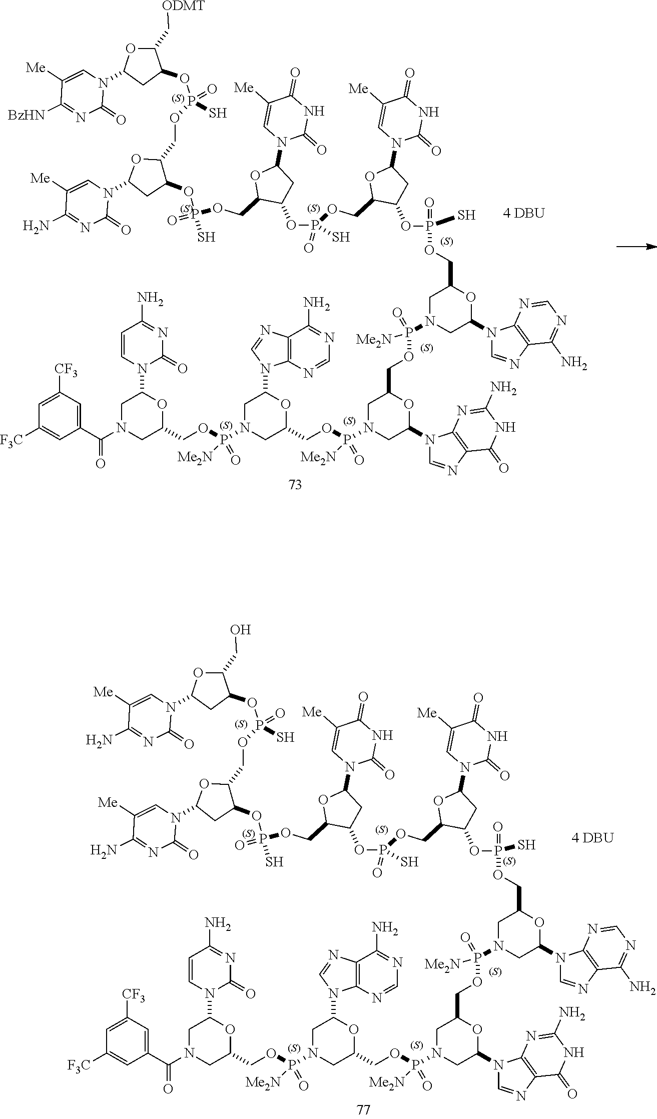

To starting material 73 (360 mg, 0.095 mmol) was added a mixture of 1,1,1,3,3,3-hexafluoro-2-propanol (4.3 mL), 2,2,2-trifluoroethanol (1.1 mL), triethylsilane (3.2 mL) and CH₂Cl₂ (5.4 mL). The resulting solution was stirred at ambient temperature for 17 h and treated with EtOAc (75 mL) followed by MTBE (15 mL). The resulting precipitate was collected by filtration and rinsed with a mixture of EtOAc/MeCN (5 mL/5 mL). The filter cake was treated with MeCN (15 mL) followed by EtOAc (15 mL). The resulting slurry was filtered through a glass filter and rinsed with MeCN/EtOAc (5 mL/5 mL). Drying the filter cake in vacuo for 2 h provided 0.305 g of target product 74.

MS (ESI) m/z: [M−2H]²⁻ Calcd for C₉₄H₁₂₂F₆N₃₅O₃₆P₇S₄ 1388.29. Found 1388.26.

### 9-Mer: Coupling

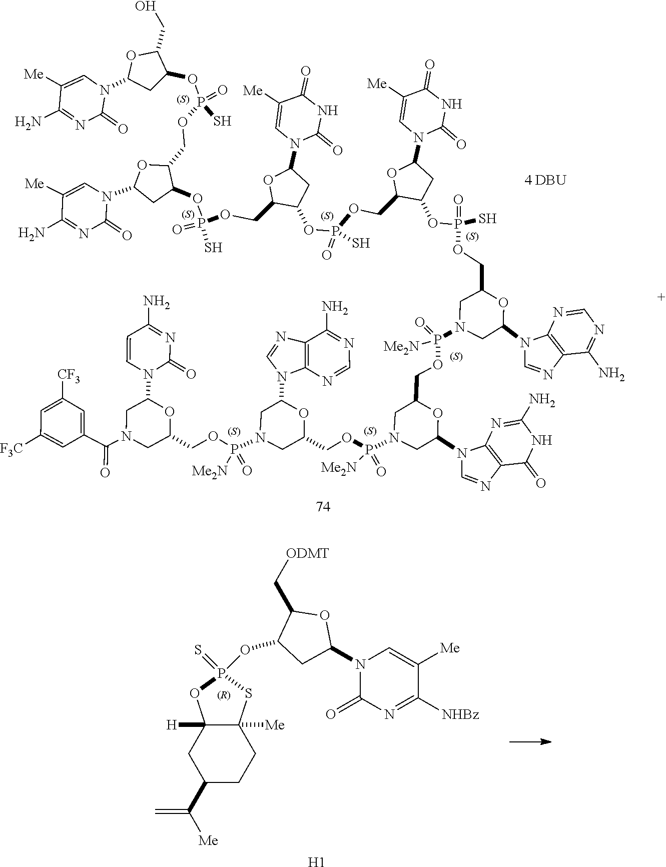

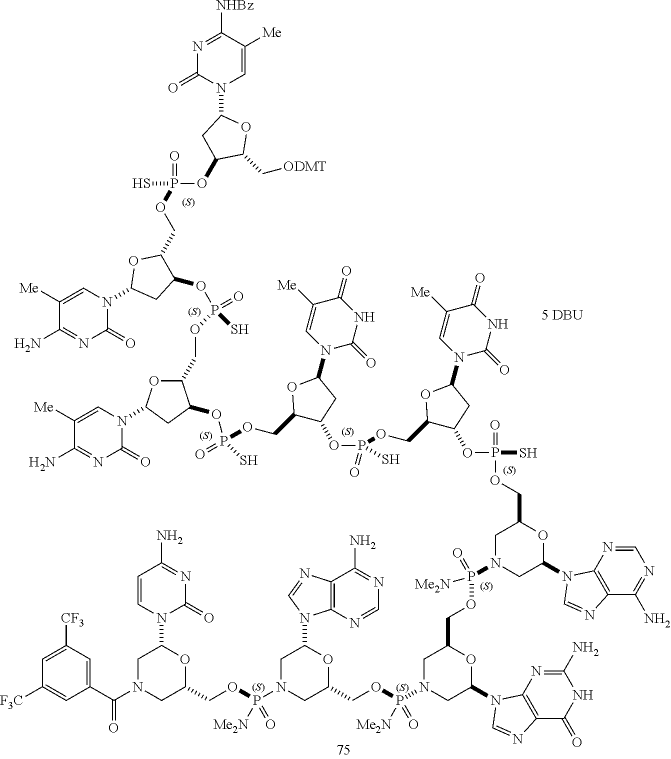

To a solution of starting material 74 (305 mg, 0.090 mmol) in 1,3-dimethyl-2-imidazolidinone (12 mL) was added reactant H1 (241 mg, 0.270 mmol). To the resulting solution was added 4 Å molecular sieves (1 g). The reaction flask was applied to vacuum and filled with nitrogen. The process was repeated two more times. After being stirred for 30 min, the resulting mixture was treated with DBU (0.11 mL, 0.72 mmol). The reaction mixture was stirred for 2.5 days at ambient temperature and then filtered through a syringe filter. The filtrate was added into EtOAc (20 mL), rinsing with 1,3-dimethyl-2-imidazolidinone (4 mL). To the resulting slurry was added additional EtOAc (20 mL). The resulting precipitate was collected by centrifuge (3500 rpm, 30 min). The resulting pellet was rinsed with a mixture of MeCN/EtOAc (5 mL/5 mL), and treated with MeCN (15 mL) followed by EtOAc (15 mL). The resulting slurry was subjected to centrifuge (3500 rpm, 10 min). The pellet was rinsed with a mixture of MeCN/EtOAc (5 mL/5 mL), and dried in vacuo for 1 h. 385 mg of target product 75 was obtained.

³¹P NMR (162 MHz, METHANOL-d4) δ=57.44 (s, 1P), 57.35 (s, 1P), 56.88 (s, 2P), 56.17 (s, 1P) 16.95 (s, 1P), 16.92 (s, 1P), 16.74 (s, 1P).

MS (ESI) m/z: [M−2H]²⁻ Calcd for C₁₃₂H₁₅₈F₆N₃₈O₄₄P₈S₅ 1751.89. Found 1751.73.

### 9-Mer: Deprotection

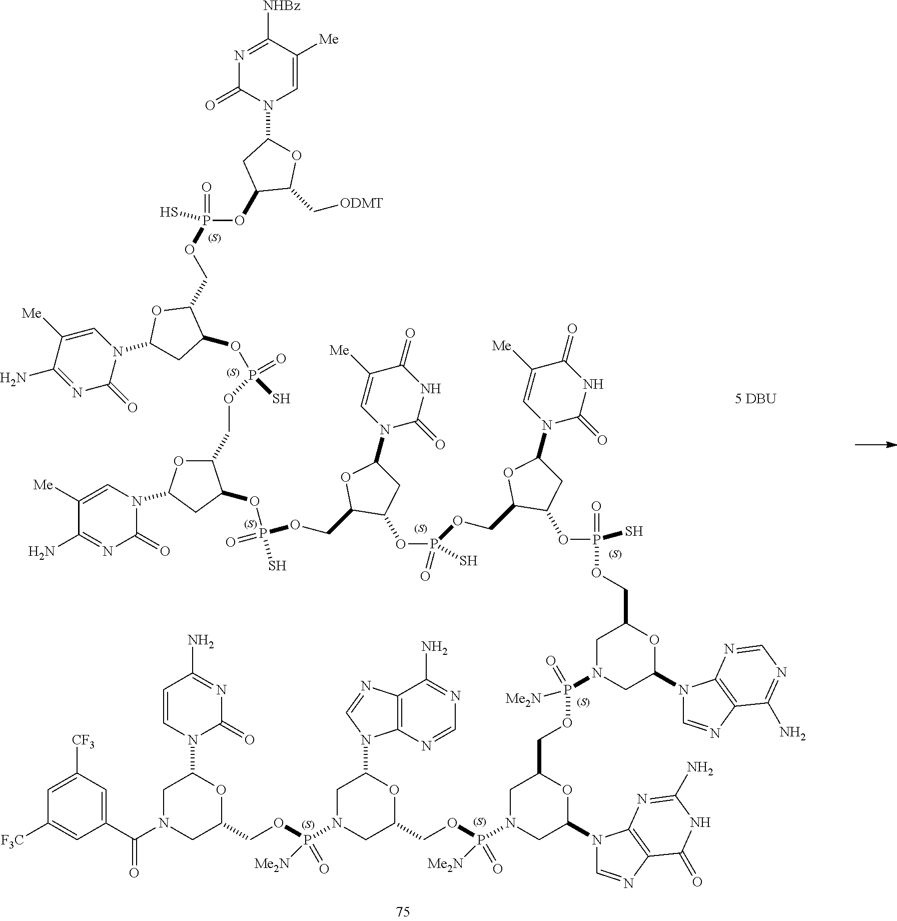

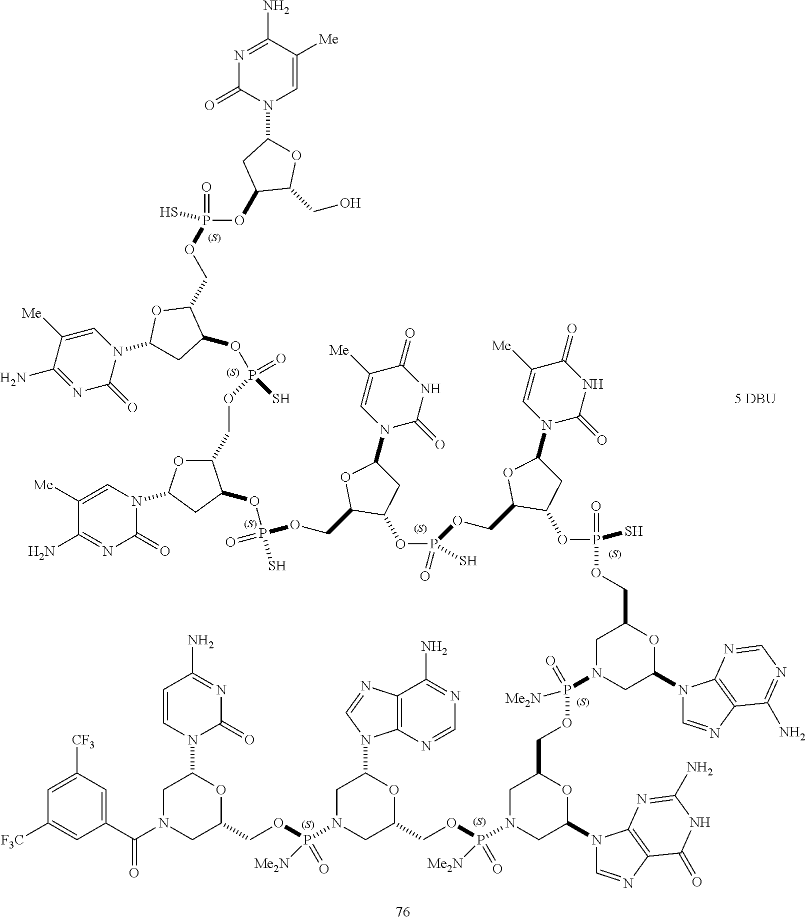

To starting material 75 (385 mg, 0.090 mmol) was added a mixture of 1,1,1,3,3,3-hexafluoro-2-propanol (4.6 mL), 2,2,2-trifluoroethanol (1.2 mL), triethylsilane (3.5 mL) and CH₂Cl₂ (5.8 mL). The resulting solution was stirred at ambient temperature overnight, and treat with EtOAc (90 mL). The resulting precipitate was collected by filtration and rinsed with a mixture of EtOAc/MeCN (10 mL/10 mL). Drying the filter cake in vacuo for 5 h provided 320 mg of target product 76.

MS (ESI) m/z: [M−2H]²⁻ Calcd for C₁₀₄H₁₃₆F₆N₃₈O₄₁P₈S₅ 1547.81. Found 1547.81.

### 10-Mer: Coupling

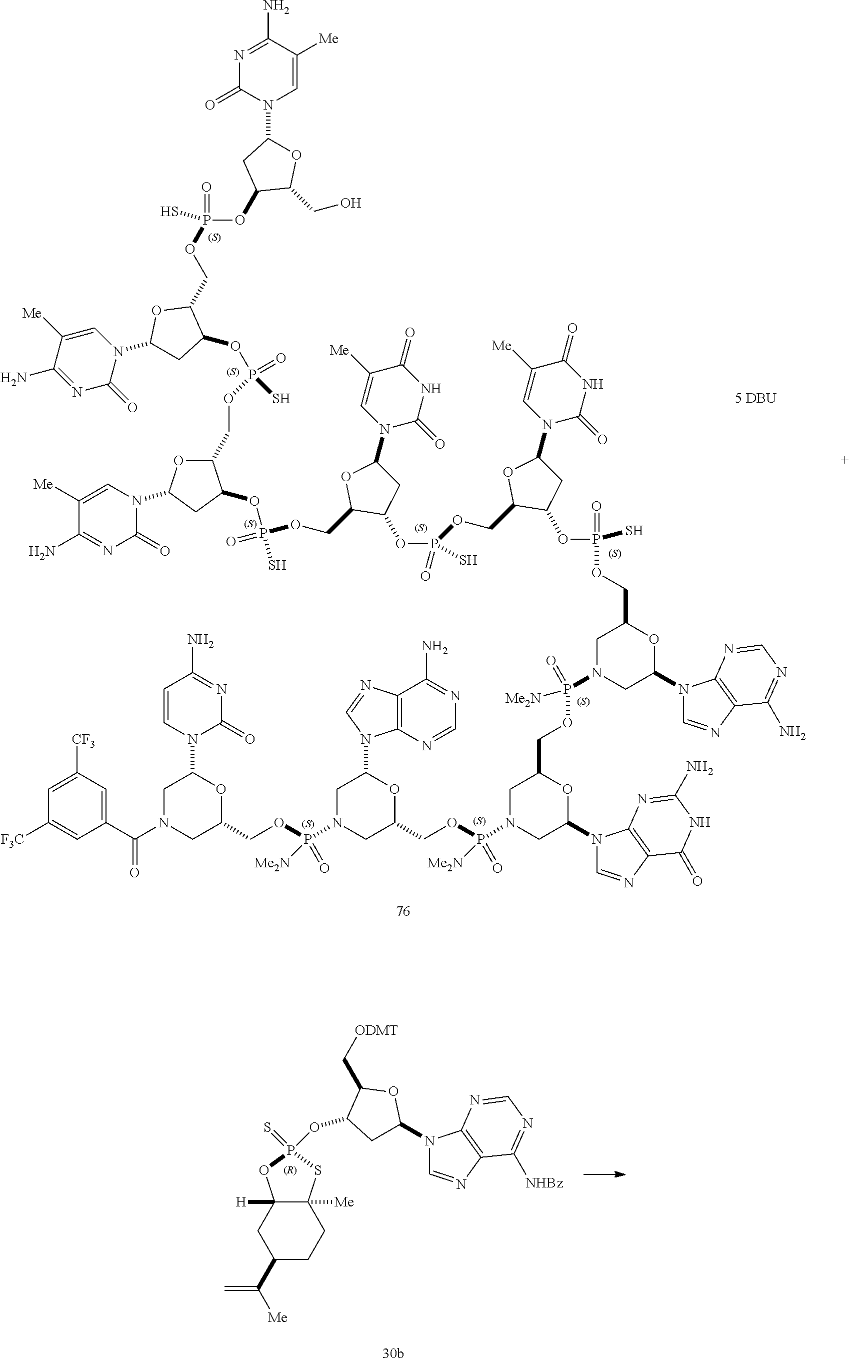

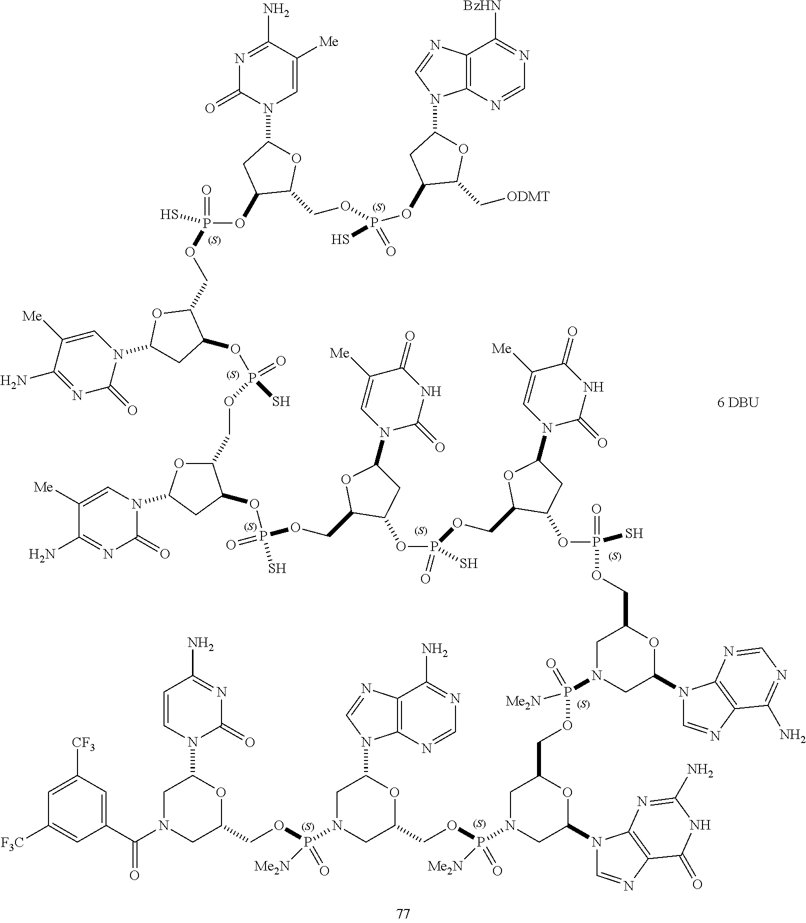

To a solution of starting material 76 (320 mg, 0.083 mmol) in 1,3-dimethyl-2-imidazolidinone (13 mL) was added reactant 30b (225 mg, 0.249 mmol). To the resulting solution was added 4 Å molecular sieves (1.0 g). The reaction flask was applied to vacuum and filled with nitrogen. The process was repeated two more times. After being stirred for 30 min, the resulting mixture was treated with DBU (0.112 mL, 0.746 mmol). The reaction mixture was stirred for 17 hr at ambient temperature and then filtered through a syringe filter. The filtrate was added into EtOAc (20 mL), rinsing with 1,3-dimethyl-2-imidazolidinone (5 mL). To the resulting slurry was added additional EtOAc (20 mL). The resulting slurry was centrifuged (3500 rpm, 30 min). To the pellet was added MeCN (20 mL) followed by EtOAc (20 mL). The resulting slurry was centrifuged (3500 rpm, 20 min). The pellet was rinsed with EtOAc/MeCN (5 mL/5 mL) and dried in vacuo for 1 h. 420 mg of target product 77 was obtained and used in next step without further purification.

³¹P NMR (162 MHz, METHANOL-d4) δ=57.29 (s, 1P), 56.99 (s, 1P), 56.95 (s, 1P), 56.78 (s, 2P), 56.23 (s, 1P), 16.95 (s, 2P), 16.72 (s, 1P).

MS (ESI) m/z: [M−2H]²⁻ Calcd for C₁₄₂H₁₇₀F₆N₄₃O₄₈P₉S₆ 1915.41. Found 1915.21.

### 10-Mer: Deprotection

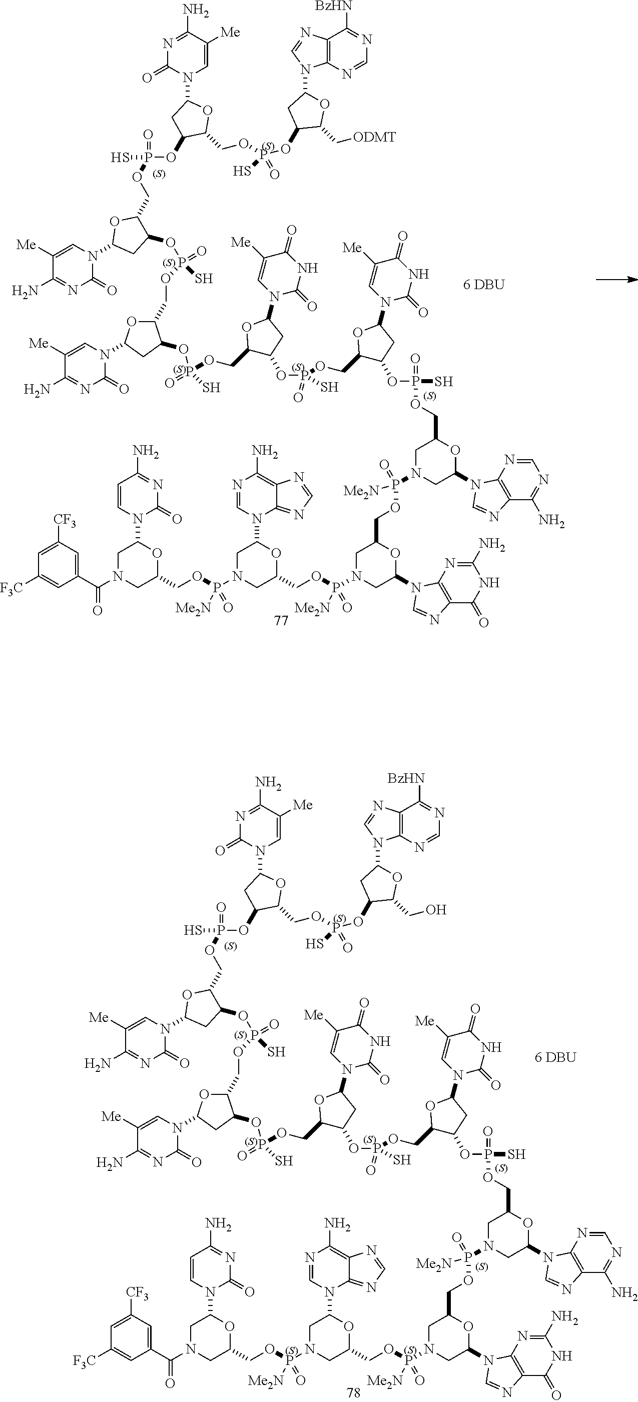

To starting material 77 (430 mg, 0.84 mmol in theory) was added a mixture of 1,1,1,3,3,3-hexafluoro-2-propanol (4.8 mL), 2,2,2-trifluoroethanol (1.2 mL), triethylsilane (3.6 mL) and CH₂Cl₂ (6.0 mL). The resulting solution was stirred at ambient temperature for 30 min and treated with EtOAc (90 mL). The resulting precipitate was collected by filtration and rinsed with a mixture of EtOAc/MeCN (10 mL/10 mL). The filter cake was treated with MeCN (20 mL) followed by EtOAc (20 mL). The resulting slurry was filtered through a glass filter and rinsed with a mixture of EtOAc/MeCN (10 mL/10 mL). Drying the filter cake in vacuo overnight provided 316 mg of target product 78.

MS (ESI) m/z: [M−2H]²⁻ Calcd for C₁₂₁H₁₅₂F₆N₄₃O₄₆P₉S₆ 1764.34. Found 1764.19.

### 11-Mer: Coupling

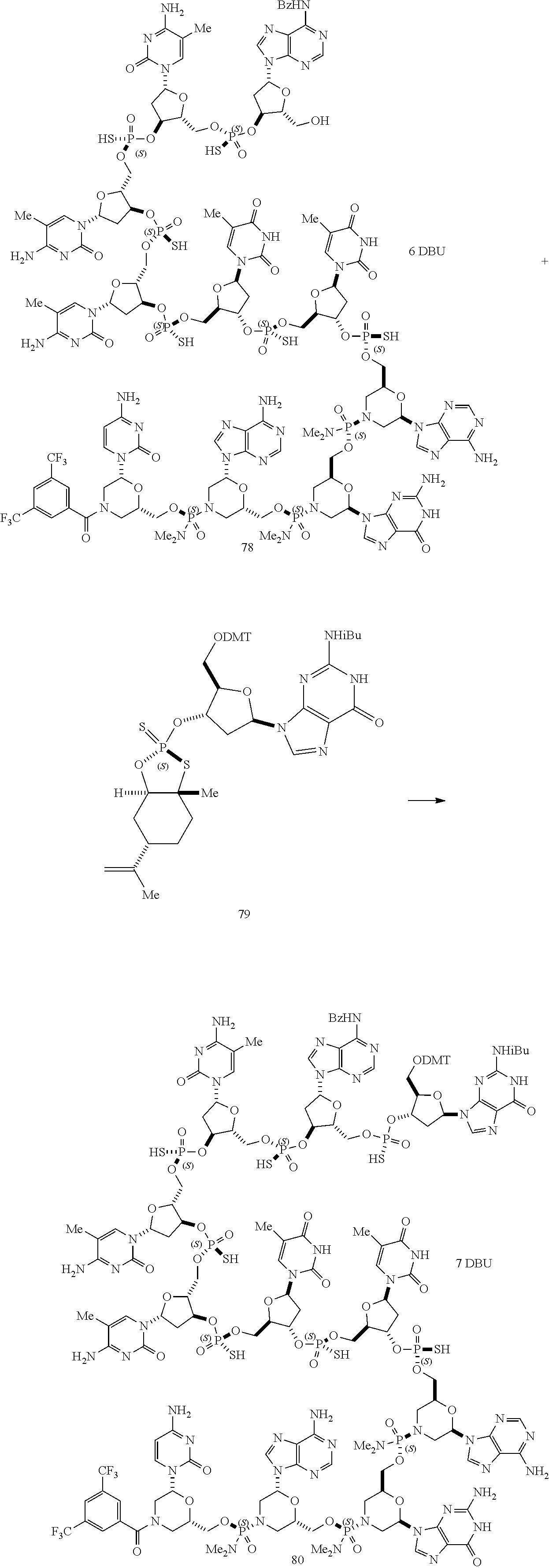

To a solution of starting material 78 (316 mg, 0.071 mmol) in 1,3-dimethyl-2-imidazolidinone (12.6 mL) was added reactant 79 (189 mg, 0.213 mmol). To the resulting solution was added 4 Å molecular sieves (1.4 g). The reaction flask was applied to vacuum and filled with nitrogen. The process was repeated two more times. After being stirred for 30 min, the resulting mixture was treated with DBU (0.11 mL, 0.71 mmol). The reaction mixture was stirred at ambient temperature overnight and additional reactant 79 (92 mg) was added. After being stirred for 2 days, the reaction mixture was filtered through a syringe filter and the resulting filtrate was added into EtOAc (20 mL), rinsing with 1,3-dimethyl-2-imidazolidinone (3 mL). The resulting slurry mixture was centrifuged (3500 rpm, 30 min). The resulting pellet was treated with MeCN (20 mL) followed by EtOAc (20 mL). The resulting slurry was filtered through a glass filter and rinsed with MeCN/EtOAc (5 mL/5 mL). Drying the filter cake in vacuo at ambient temperature for 4 h provided 375 mg of target product 80.

³¹P NMR (162 MHz, METHANOL-d4) δ=57.27 (s, 1P), 56.95 (s, 1P), 56.91 (s, 1P), 56.83 (s, 1P), 56.81 (s, 1P), 56.75 (s, 1P), 56.24 (s, 1P), 16.95 (s, 2P), 16.71 (s, 1P).

MS (ESI) m/z: [M−3H]³⁻ Calcd for C₁₅₆H₁₈₇F₆N₄₈O₅₄P₁₀S₇ 1414.96. Found 1414.94.

### 11-Mer: Deprotection

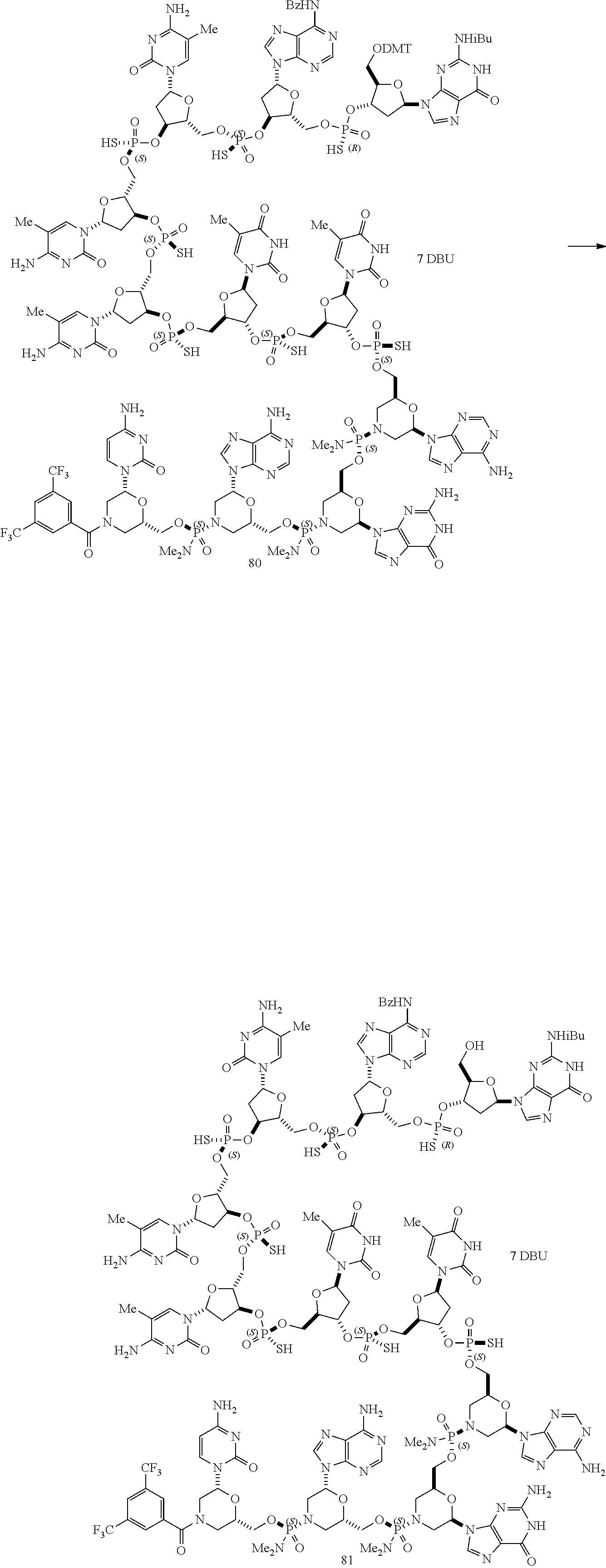

Starting material 80 (375 mg, 0.071 mmol) was dissolved in a mixture of 1,1,1,3,3,3-hexafluoro-2-propanol (4.5 mL), 2,2,2-trifluoroethanol (1.1 mL), triethylsilane (3.4 mL) and CH₂Cl₂ (5.6 mL). The resulting solution was stirred at ambient temperature for 40 min and treated with EtOAc (75 mL) followed MTBE (25 mL). The resulting precipitate was collected by filtration and rinsed with a mixture of EtOAc/MeCN (10 mL/10 mL). The filter cake was treated with MeCN (20 mL) followed by EtOAc (20 mL). The resulting slurry was filtered through a filter and rinsed with MeCN/EtOAc (5 mL/5 mL). Drying the filter cake in vacuo overnight provided 343 mg of target product 81.

MS (ESI) m/z: [M−2H]²⁻ Calcd for C₁₃₅H₁₇₀F₆N₄₈O₅₂P₁₀S₇ 1971.88. Found 1971.73.

### 12-Mer: Coupling

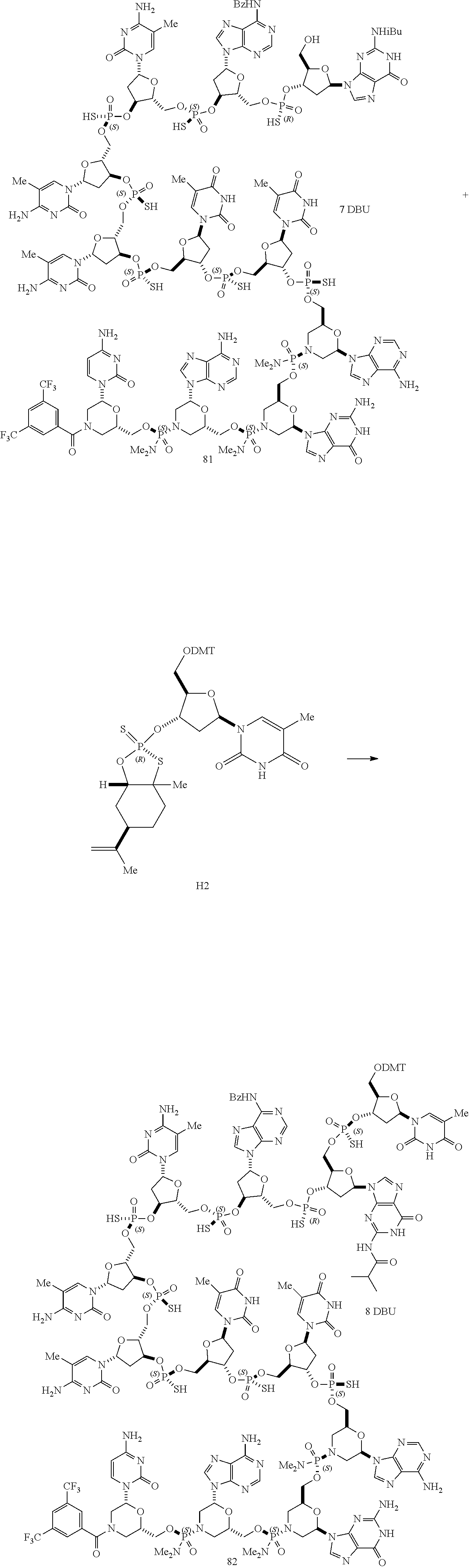

To a solution of starting material 81 (343 mg, 0.068 mmol) in 1,3-dimethyl-2-imidazolidinone (12 mL) was added reactant 112 (189 mg, 0.239 mmol). To the resulting solution was added 4 Å molecular sieves (1.5 g). The reaction flask was applied to vacuum and filled with nitrogen. The process was repeated two more times. After being stirred for 30 min, the resulting mixture was treated with DBU (0.113 mL, 0.753 mmol). The reaction mixture was stirred for 23 h at ambient temperature and then filtered through a syringe filter. The filtrate was added into EtOAc (20 mL), rinsing with 1,3-dimethyl-2-imidazolidinone (5 mL). Additional EtOAc (20 mL) was added. The resulting slurry was centrifuged (3500 rpm, 30 min). The resulting pellet was treated with MeCN (20 mL) followed by EtOAc (20 mL). The resulting slurry was filtered through a glass filter and rinsed with MeCN/EtOAc (5 mL/5 mL). Drying the filter cake in vacuo at ambient temperature for 3 h provided target product 82.

³¹P NMR (162 MHz, METHANOL-d4) δ=57.28 (s, 1P), 57.24 (s, 1P), 56.94 (s, 1P), 56.81 (s, 2P), 56.74 (s, 2P), 56.22 (s, 1P), 16.95 (s, 2P), 16.70 (s, 1P)

MS (ESI) m/z: Calcd for C₁₆₆H₂₀₀F₆N₅₀O₆₀P₁₁S₈ 1521.63. Found 1521.41.

### 12-Mer: Deprotection

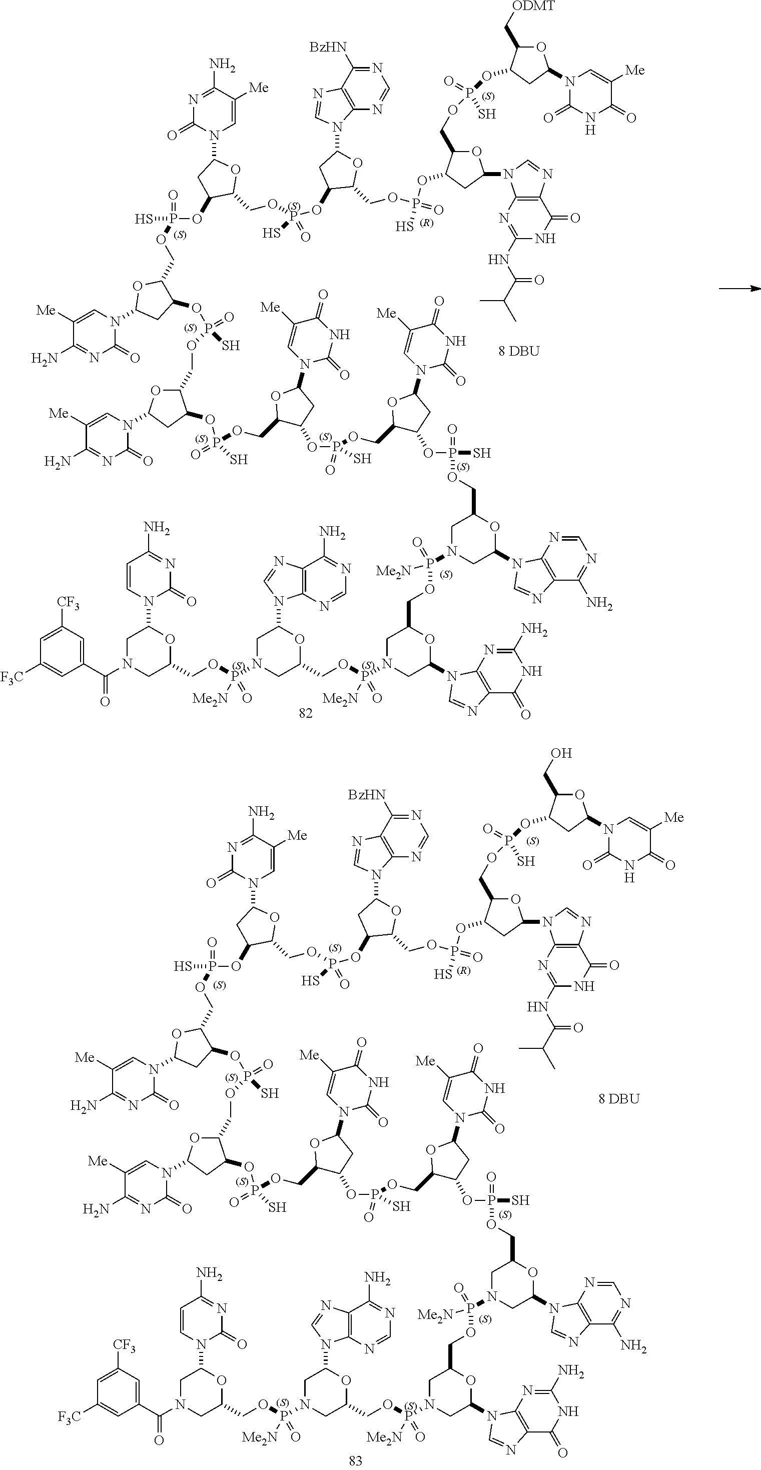

Starting material 82 (396 mg, 0.068 mmol in theory) was dissolved in a mixture of 1,1,1,3,3,3-hexafluoro-2-propanol (4.8 mL), 2,2,2-trifluoroethanol (1.2 mL), triethylsilane (3.6 mL) and CH₂Cl₂ (6.0 mL). The resulting solution was stirred at ambient temperature for 16 h and treated with EtOAc (100 mL). The resulting precipitate was collected by filtration and rinsed with a mixture of EtOAc/MeCN (5 mL/5 mL). The filter cake was treated with MeCN (20 mL) followed by EtOAc (20 mL). The resulting slurry was filtered through a glass filter and rinsed with MeCN/EtOAc (5 mL/5 mL). Drying the filter cake in vacuo for 1 h provided 310 mg of target product 83.

MS (ESI) m/z: [M−3H]₃₋ Calcd for C₁₄₅H₁₈₂F₆N₅₀O₅₈P₁₁S₈ 1421.26. Found 1421.32.

### 13-Mer: Coupling

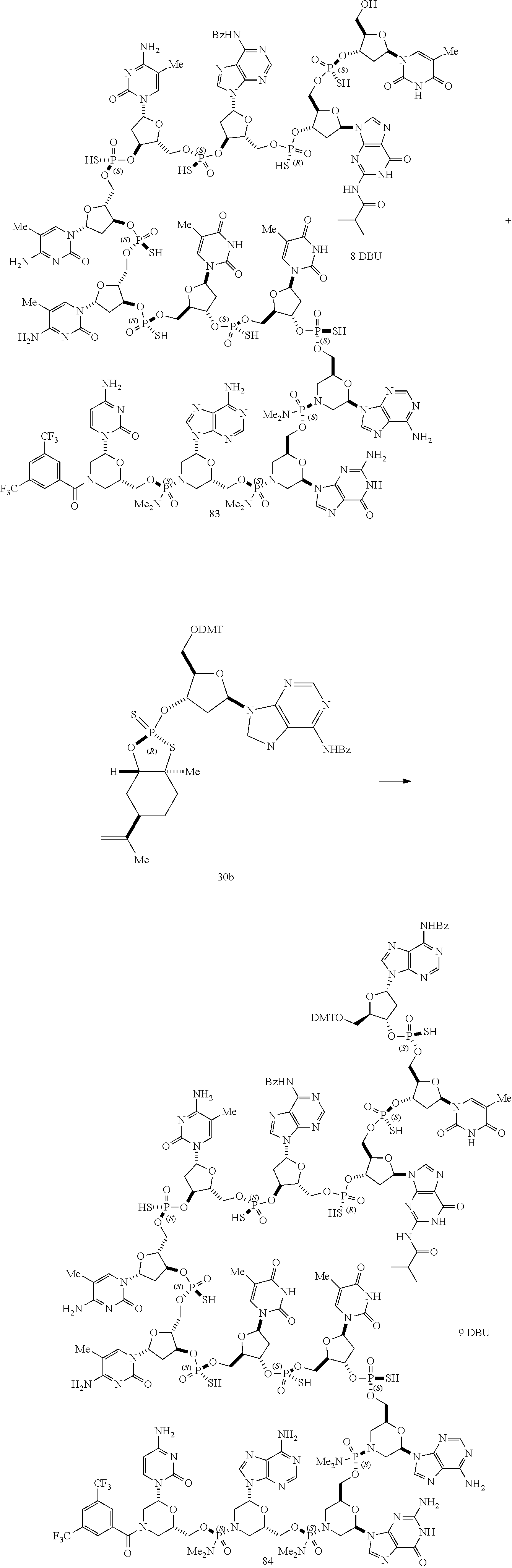

To a solution of starting material 83 (310 mg, 0.057 mmol) in 1,3-dimethyl-2-imidazolidinone (11 mL) was added reactant 30b (179 mg, 0.198 mmol). To the resulting solution was added 4 Å molecular sieves (1.2 g). The reaction flask was applied to vacuum and filled with nitrogen. The process was repeated two more times. After being stirred for 30 min, the resulting mixture was treated with DBU (0.102 mL, 0.678 mmol). The reaction mixture was stirred overnight at ambient temperature and then filtered through a syringe filter. The filtrate was added into EtOAc (20 mL), rinsing with 1,3-dimethyl-2-imidazolidinone (5 mL). Additional EtOAc (20 mL) was added. The resulting slurry was centrifuged (3500 rpm, 30 min). The resulting pellet was treated with MeCN (20 mL) followed by EtOAc (20 mL). The resulting slurry was filtered through a glass filter and rinsed with MeCN/EtOAc (5 mL/5 mL). The filter cake was dried in vacuo at ambient temperature for 3 days, and then treated with 25 mL MeCN to make a slurry. After being stirred for 30 min, the resulting slurry was filtered through a glass filter and rinsed with MeCN/EtOAc (5 mL/5 mL). Drying the filter cake in vacuo for 1 h provided 365 mg of target product 84.

³¹P NMR (162 MHz, METHANOL-d4) δ=57.22 (s, 1P), 56.96 (s, 2P), 56.89 (s, 1P), 56.78 (s, 2P), 56.74 (s, 2P), 56.27 (s, 1P), 16.96 (s, 2P), 16.72 (s, 1P).

MS (ESI) m/z: [M−3H]³⁻ Calcd for C₁₈₃H₂₁₆F₆N₅₅O₆₅P₁₂S₉ 1666.32. Found 1666.24.

### 13-Mer: Deprotection

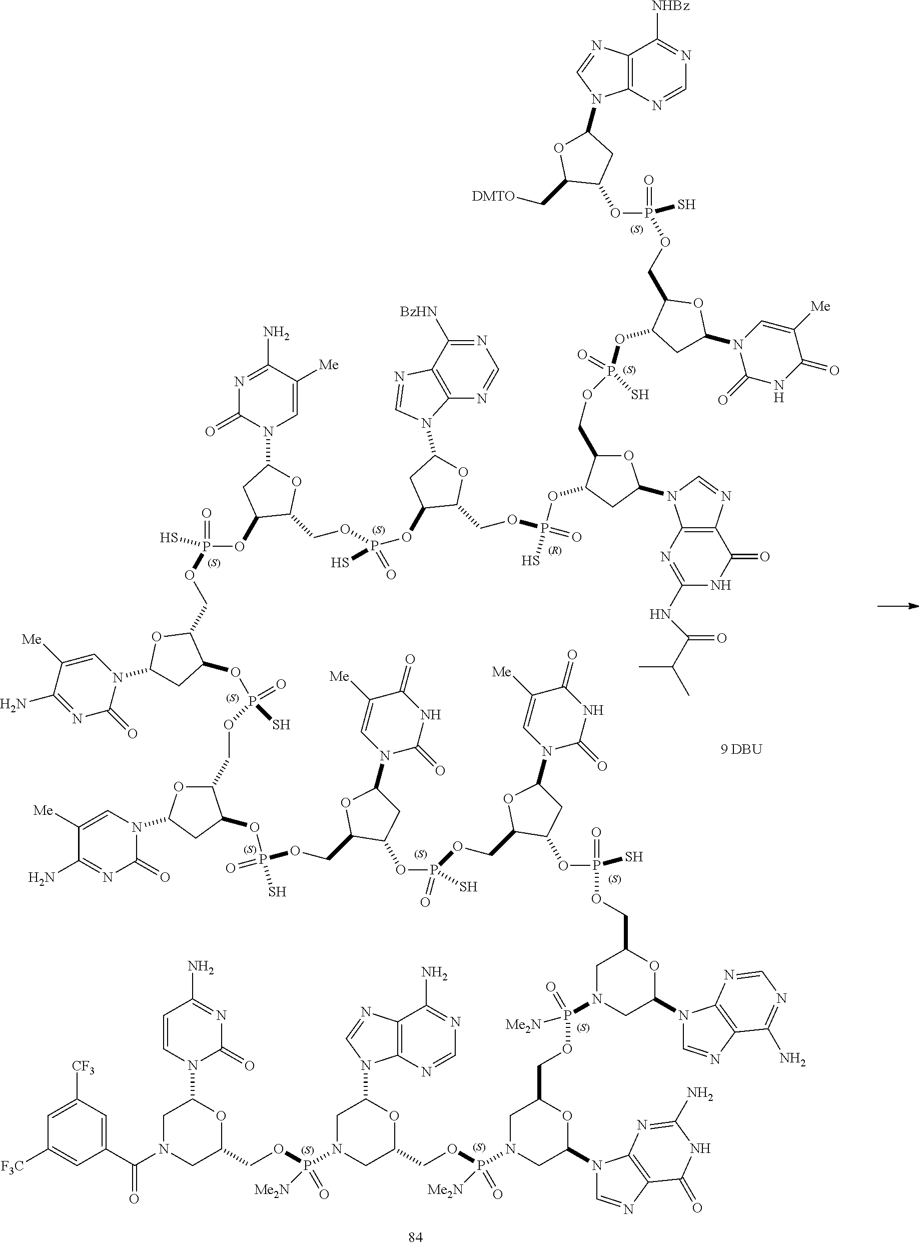

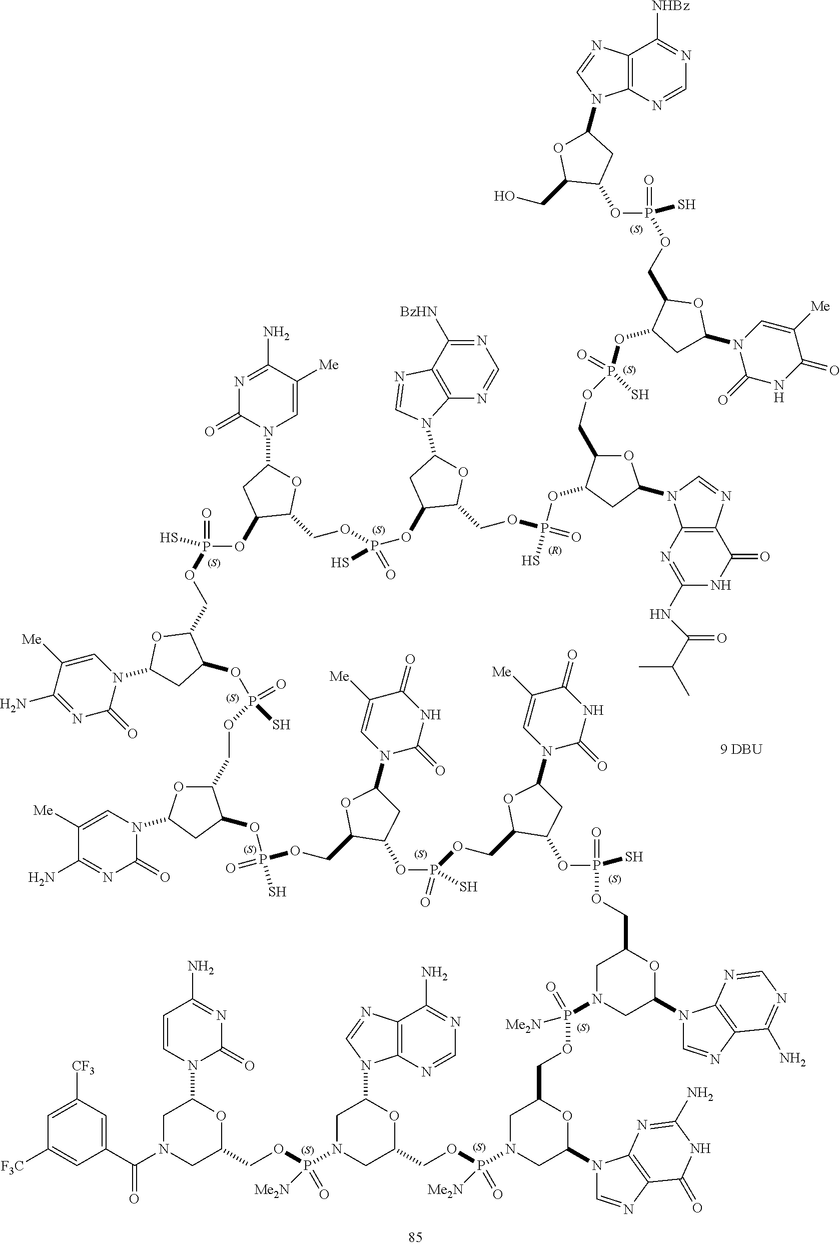

Starting material 84 (365 mg, 0.057 mmol) was dissolved in a mixture of 1,1,1,3,3,3-hexafluoro-2-propanol (4.4 mL), 2,2,2-trifluoroethanol (1.1 mL), triethylsilane (3.3 mL) and CH₂Cl₂ (5.5 mL). The resulting solution was stirred at ambient temperature for 20 min and treated with 125 mL EtOAc. The resulting precipitate was collected by filtration and rinsed with a mixture of EtOAc/MeCN (10 mL/10 mL). The filter cake was treated with MeCN (20 mL) followed by EtOAc (10 mL). The resulting slurry was centrifuged (4000 rpm, 60 min). The resulting pellet was isolated by decantation and rinsed with MeCN/EtOAc (5 mL/5 mL). Drying in vacuo overnight provided 328 mg of target product 85.

MS (ESI) m/z: [M−3H]³⁻ Calcd for C₁₆₂H₁₉₈F₆N₅₅O₆₃P₁₂S₉ 1565.61. Found 1565.65.

### Example 5.4: 13+5 Coupling

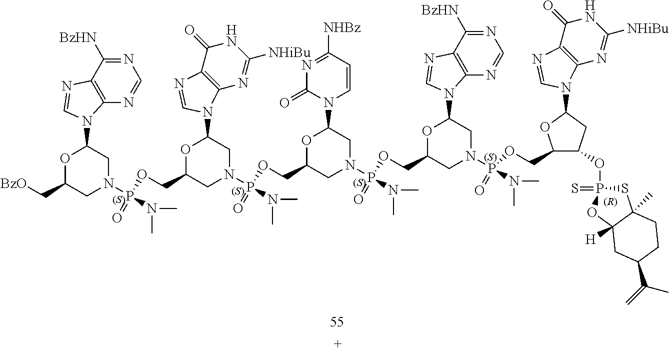

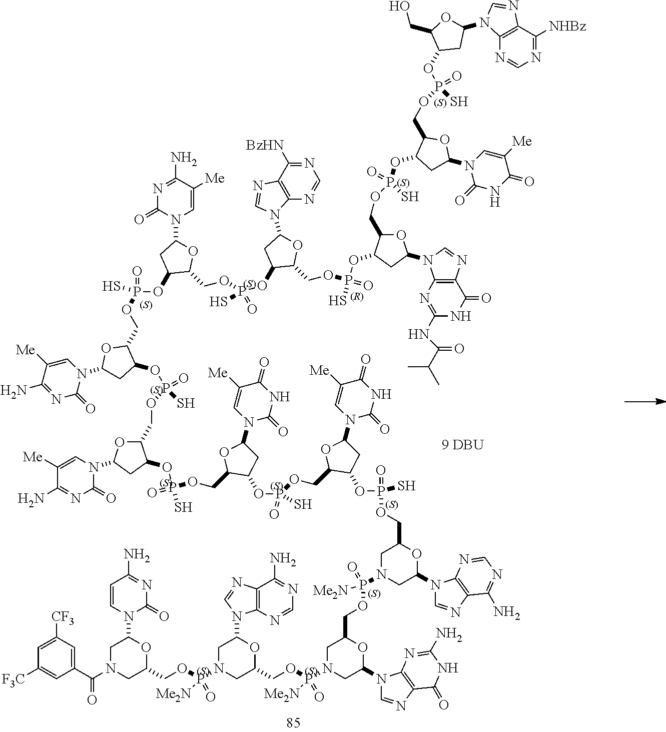

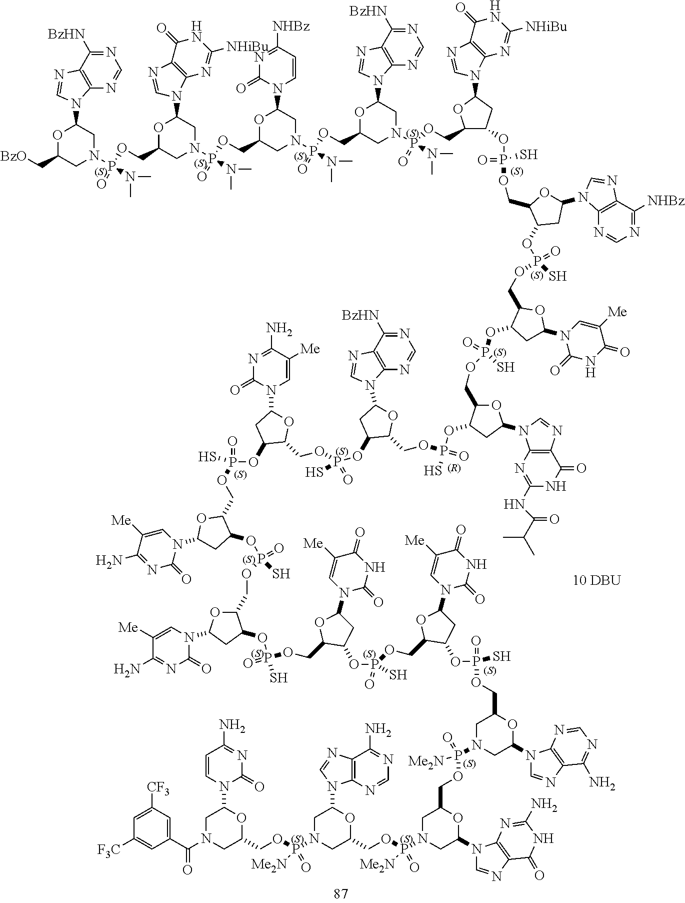

To a mixture of starting material 85 (100 mg, 0.016 mmol) and reactant 55 (139 mg, 0.058 mmol) was added 1,3-dimethyl-2-imidazolidinone (3.5 mL). The resulting mixture was azeotroped with toluene (2 mL each time) three times at 30-33° C. To the resulting solution was added 4 Å molecular sieves (0.40 g). The reaction flask was applied to vacuum and filled with nitrogen. The process was repeated two more times. After being stirred for 30 min, the resulting mixture was treated with DBU (0.032 mL, 0.21 mmol). The reaction mixture was stirred for 3 days at ambient temperature and then filtered through a syringe filter. The filtrate was added into EtOAc (15 mL), rinsing with 1,3-dimethyl-2-imidazolidinone (2.5 mL). The resulting slurry was centrifuged (3500 rpm, 20 min). The pellet was dissolved in EtOH (3 mL) and CH₂Cl₂ (6 mL). To the resulting solution was added EtOAc (20 mL). The resulting slurry was filtered through a glass filter and rinsed with MeCN (10 mL). Drying the filter cake in vacuo at ambient temperature for 0.5 h provided 0.13 g of target product 87.

³¹P NMR (162 MHz, METHANOL-d4) δ=57.30 (s, 1P), 57.19 (s, 1P), 56.91 (s, 2P), 56.80 (s, 2P), 56.73 (s, 2P), 56.62 (s, 1P), 56.18 (s, 1P), 17.07 (s, 2P), 16.94 (s, 2P), 16.91 (s, 1P), 16.85 (s, 1P), 16.67 (s, 1P)

MS (ESI) m/z: [M−4H]⁴⁻ Calcd for C₂₅₅H₃₀₉F₆N₈₆O₈₈P₁₇S₁₀ 1736.38. Found 1736.31.

### Example 5.5: Final Deprotection

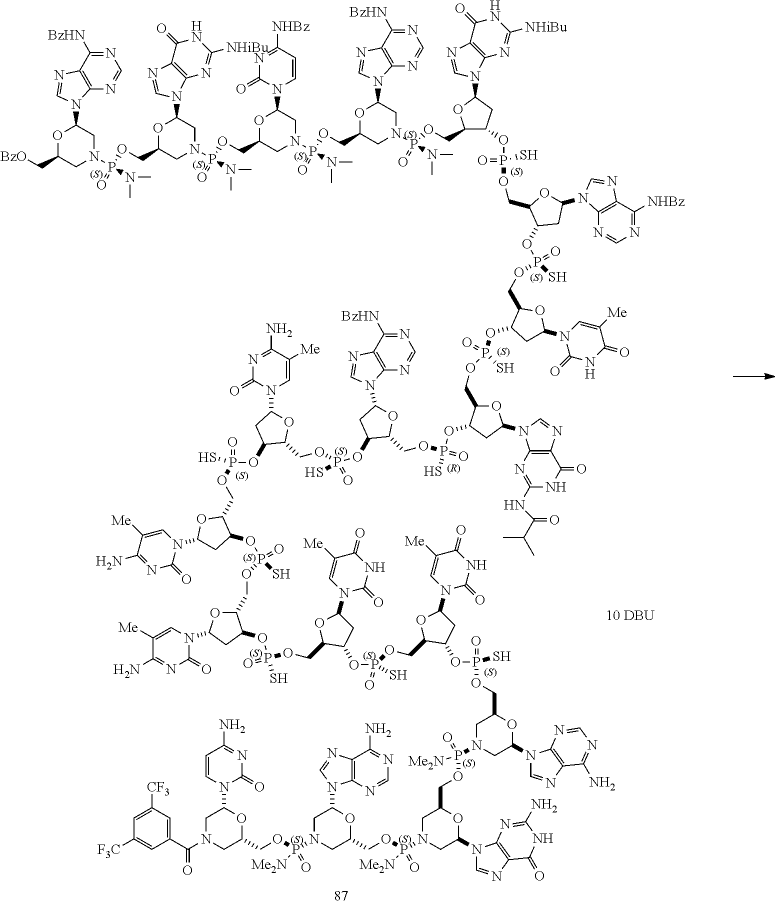

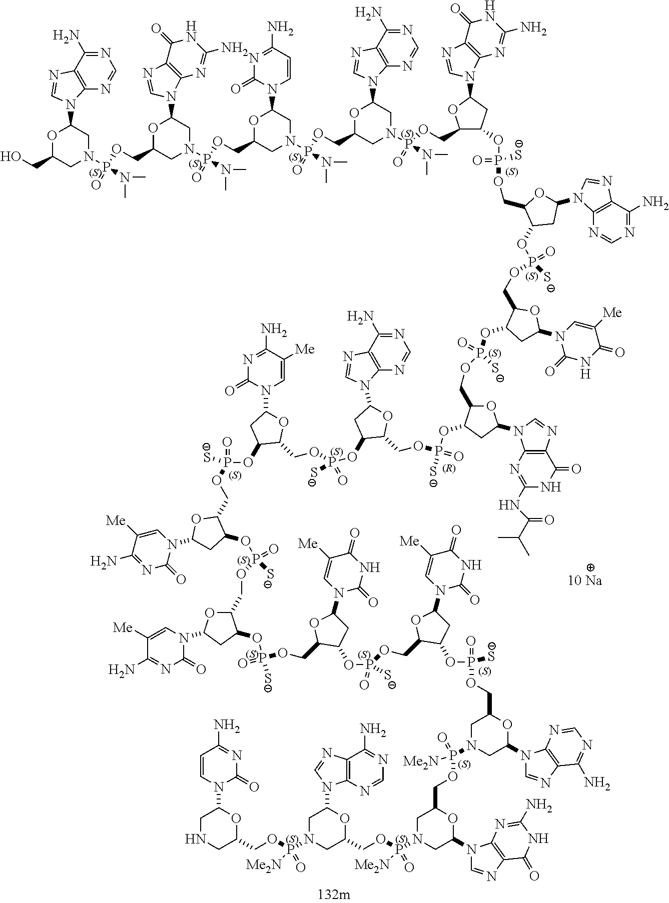

To a solution of starting material 87 (0.130 mg, 0.015 mmol) in a mixture of methanol (4.6 mL) and 28% ammonium hydroxide (4.6 mL) was added DL-dithiothreitol (0.024 g, 0.15 mmol). The resulting mixture was stirred at 53-55° C. for 23 h and cooled to ambient temperature. A mixture of MeCN/EtOAc (20 mL/20 mL) was added and the resulting slurry was subjected to centrifuge (4000 rpm, 90 min). The resulting pellet was isolated and dissolved in water (30 mL). The aqueous solution was subjected to ultrafiltration (Amicon Ultra-15, ultracel 3K, 3500 rpm, 35 min). The remaining solution was diluted with water (30 mL) and subjected to ultrafiltration (Amicon Ultra-15, ultracel 3K, 3500 rpm, 35 min). The remaining solution was filtered through a syringe filter and rinsed with water. The filtrate (ca. 5 mL) was subjected to centrifuge (4000 rpm, 30 min) and the supernatant was purified by prep-HPLC using the conditions in Table 6 and then the conditions in Table 7.

TABLE 6
RP-HPLC conditions

| Column | Waters, XBridge Prep C18 5 μm OBD, 19 × 100 mm (Part Number: 186002978) | | | | |
|---|---|---|---|---|---|
| Instrument | Waters 2545 Binary Gradient Module, Waters 3100 Mass Detector | | | | |
| Mobile phase A | 100 mM HFIP (Hexafluoroisopropanol) + 8.6 mM TEA (Triethylamine) in water | | | | |
| Mobile phase B | Methanol 100% | | | | |
| Column Temperature (° C.) | 60 | | | | |
| | | | | Flow rate | |
| | TIME (min) | A % | B % | (mL/min) | comments |
| Gradient | 0 | 90 | 10 | 25 | Initial |
| | 2.2 | 90 | 10 | 25 | |
| | 4.4 | 80 | 20 | 30 | Elution Gradient |
| | 11.1 | 50 | 50 | 30 | |
| | 11.2 | 0 | 100 | 30 | Wash |
| | 17.9 | 0 | 100 | 30 | |
| | 18.0 | 90 | 10 | 30 | Reset Conditions |
| | 20.2 | 90 | 10 | 30 | |

| | |
|---|---|
| Flow Rate (mL/min) | See the table |
| Wavelength (nm) | 260 |

TABLE 7
IEX-HPLC conditions

| Column | TOSOH Bioscience, TSKgel SuperQ-5PW, | | | |
|---|---|---|---|---|
| | 7.5 mm ID × 7.5 cm, 10 μm (Part No: 0018257) | | | |
| Instrument | Agilent 1200 | | | |
| Mobile phase A | 10 mM NaOH in water | | | |
| Mobile phase B | 10 mM NaOH + 1M NaCl in water | | | |
| Column | 45 | | | |
| Temperature (° C.) | | | | |
| | TIME (min) | A % | B % | comments |
| Gradient: | 0 | 50 | 50 | Initial |
| | 1.7 | 30 | 70 | Elution Gradient |
| | 11.6 | 0 | 100 | Wash |
| | 13.3 | 0 | 100 | |
| | 13.4 | 50 | 50 | Reset Conditions |
| | 15.1 | 50 | 50 | |

| | |
|---|---|
| Flow Rate | 2.0 |
| (mL/min) | |
| Wavelength (nm) | 260 |

Desalting of the purified product was conducted 4 times with Amicon Ultra-15, Ultracel-3K (3500 rpm, 45 min). Freeze-drying of the resulting solution (12.5 mL) for 2 days provided 18 mg of target product 132m.

HRMS (ESI) m/z: [M−3H]³⁻ Calcd for C₁₉₂H₂₆₆N₈₆O₇₈P₁₇S₁₀ 1957.7415. Found 1957.7418.

### Example 5.6: Preparation of Compound 132n

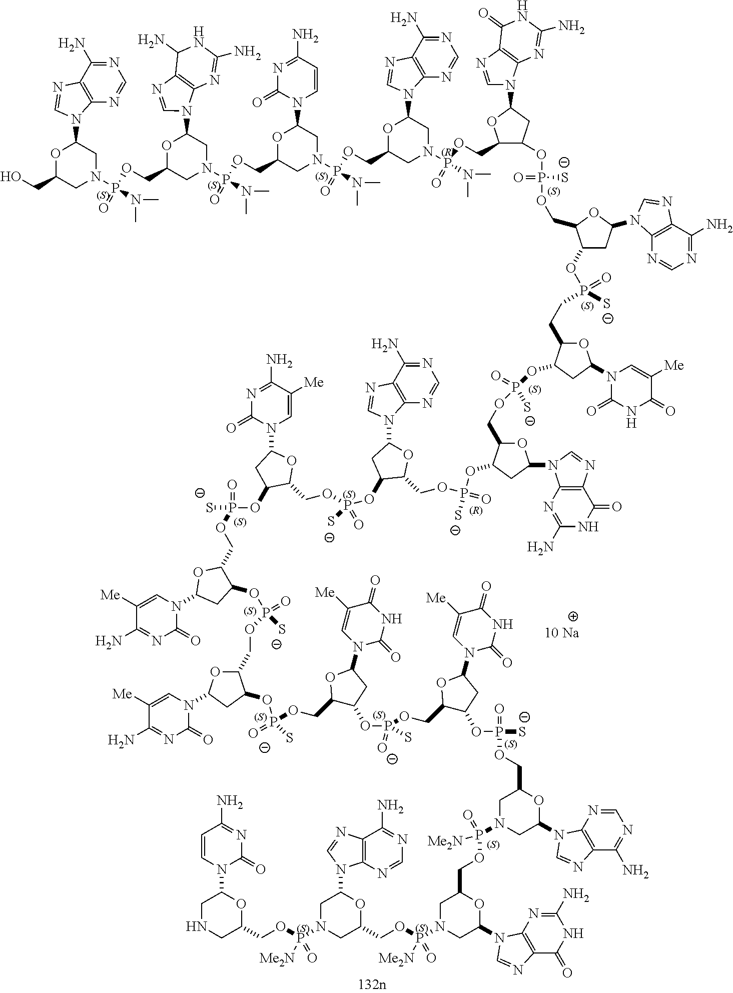

With compound 52b instead of compound 52a in the preparation of the 5′ wing 5-mer (compound 53), Compound 132n was prepared via the same reaction sequences as described for Compound 132f.

HRMS (ESI) m/z: [M−3H]³⁻ Calcd for C₁₉₂H₂₆₆N₈₆O₇₈P₁₇S₁₀ 1957.7415. Found 1957.7422.

### Example 5.7: Preparation of Compound 132f

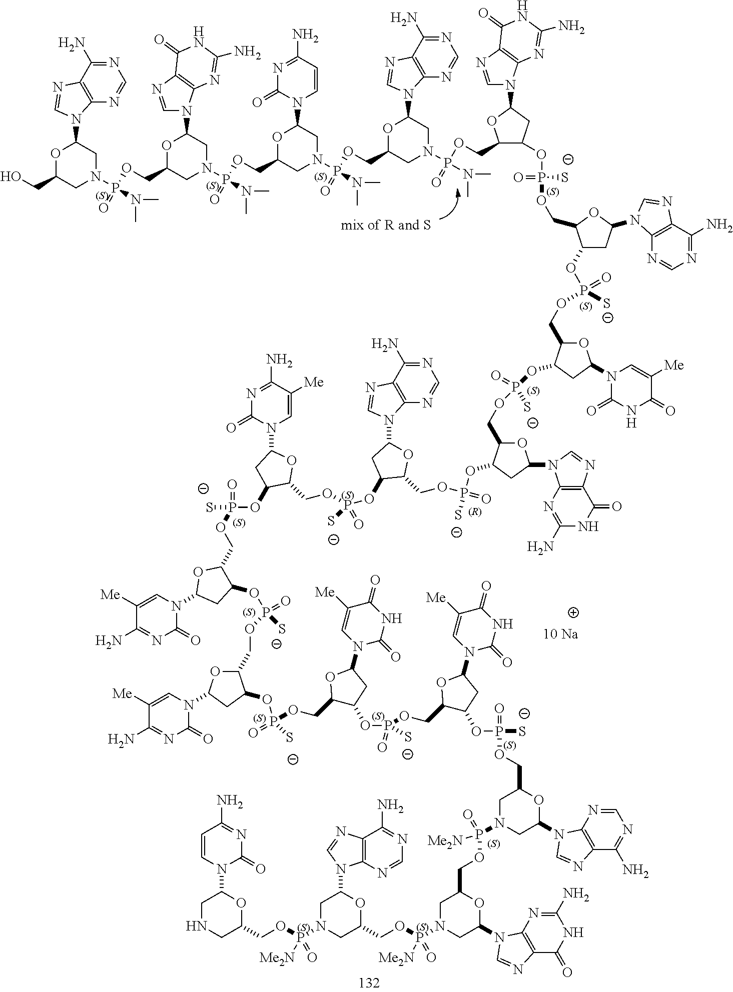

With ((2R,3S,5R)-3-(bis(4-methoxyphenyl)(phenyl)methoxy)-5-(2-isobutyramido-6-oxo-1,6-dihydro-9H-purin-9-yl)tetrahydrofuran-2-yl)methyl dimethylphosphoramidochloridate (52) instead of compound 52a in the preparation of the 5′ wing 5-mer (compound 53), Compound 132f was prepared via the same reaction sequences as described for Compound 132m.

HRMS (ESI) m/z: [M−3H]³⁻ Calcd for C₁₉₂H₂₆₆N₈₆O₇₈P₁₇S₁₀ 1957.7415. Found 1957.7439.

### Example 6: Preparation of PMO-Gapmer Conjugated with Lipid

Preparation of PMO-Gapmer with 3′ Lipid—Installation of PEG Linker

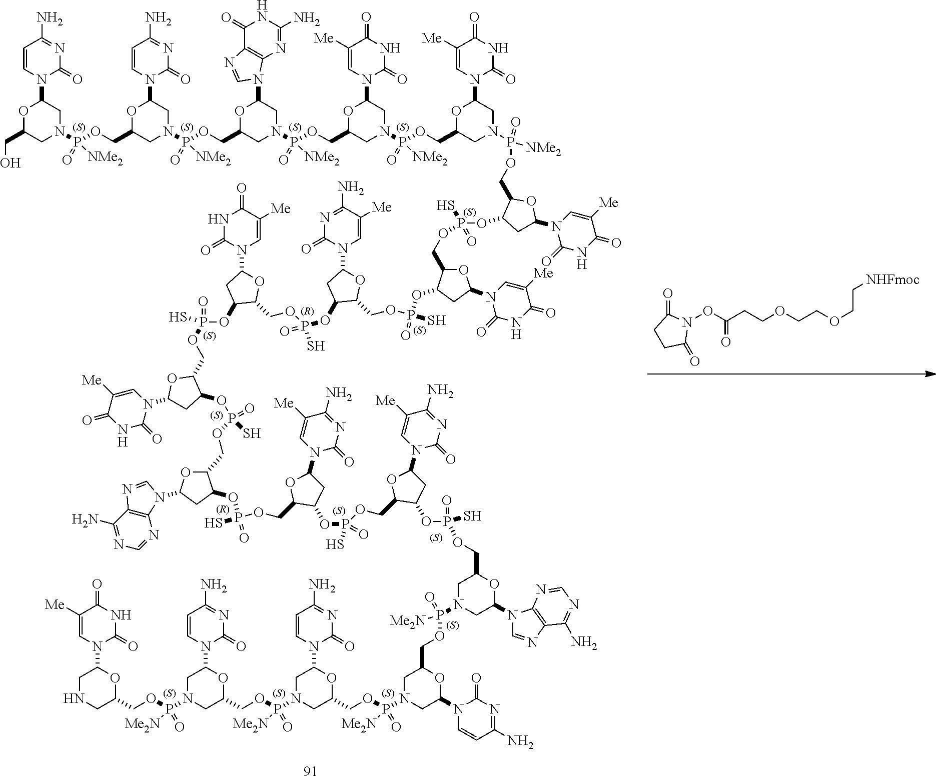

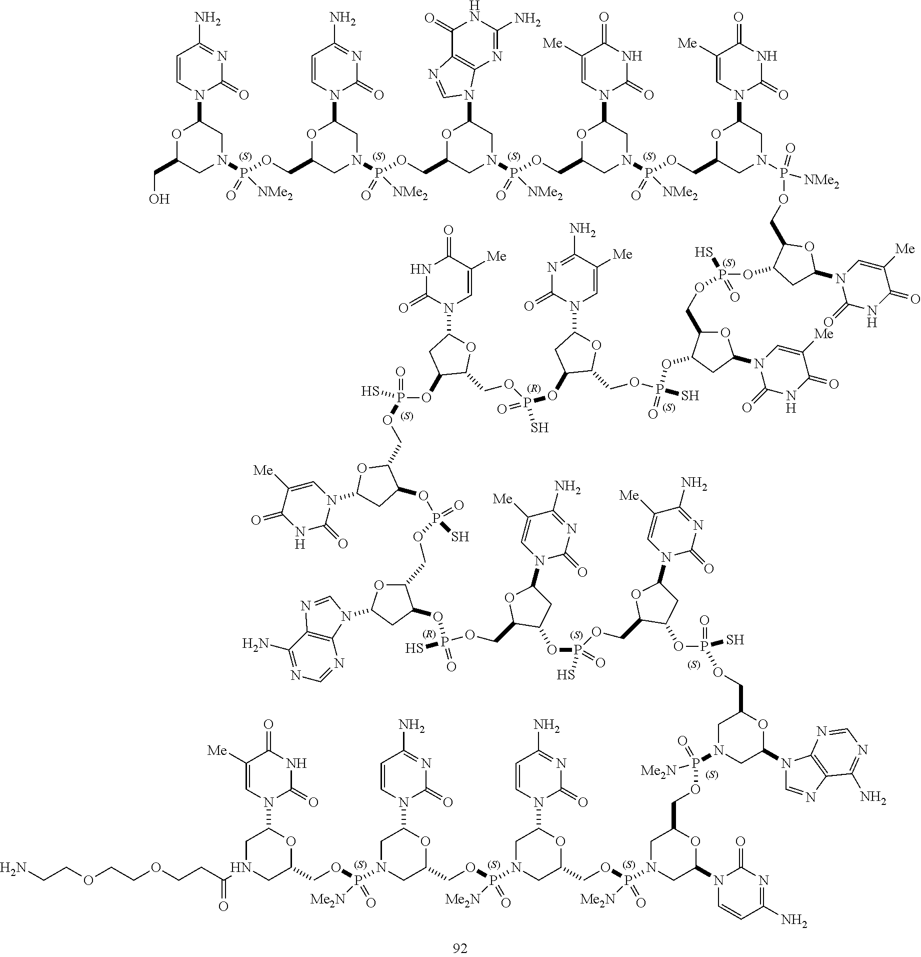

To starting material 91 (9 mg, 1.523 μmol) in a 4 mL vial was added 1,3-dimethyl-2-imidazolidinone (1.5 mL). After sonicated for ca. 1 min, the resulting mixture was treated with a saturated aqueous NaHCO₃ (8%, 0.5 mL) and water (0.25 mL). To the resulting slurry was added 2,5-dioxopyrrolidin-1-yl 1-(9H-fluoren-9-yl)-3-oxo-2,7,10-trioxa-4-azatridecan-13-oate (9.1 mg, 0.018 mmol). The reaction mixture was stirred at 35° C. overnight (ca. 18 h), diluted with water (20 mL), and subjected to ultrafiltration (Amicon Ultra-15, ultracel 3K, 3500 rpm, 45 min) three times. The crude product (a mixture of −30% product and ˜70% staring material) in water (˜3 mL) was re-subjected to the above reaction conditions four more time until >90% conversion was achieved.

The coupling product in water (˜3 mL) was treated with 1.0 M aqueous NaOH (0.7 mL) and stirred at room temperature overnight. The reaction mixture was filtered through a syringe filter, diluted with water (30 mL), and subjected to ultrafiltration (Amicon Ultra-15, ultracel 3K, 3500 rpm, 45 min) twice. The resulting product (92) in water (2.5 mL) was used in next step without further purification.

MS (ESI) m/z: [M+5H]⁵⁺ Calcd for C₂₀₀H₃₀₃N₇₃O₈₇P₁₇S₈ 1180.3. Found 1180.9.

Conjugation with Palmitoyl Lipid

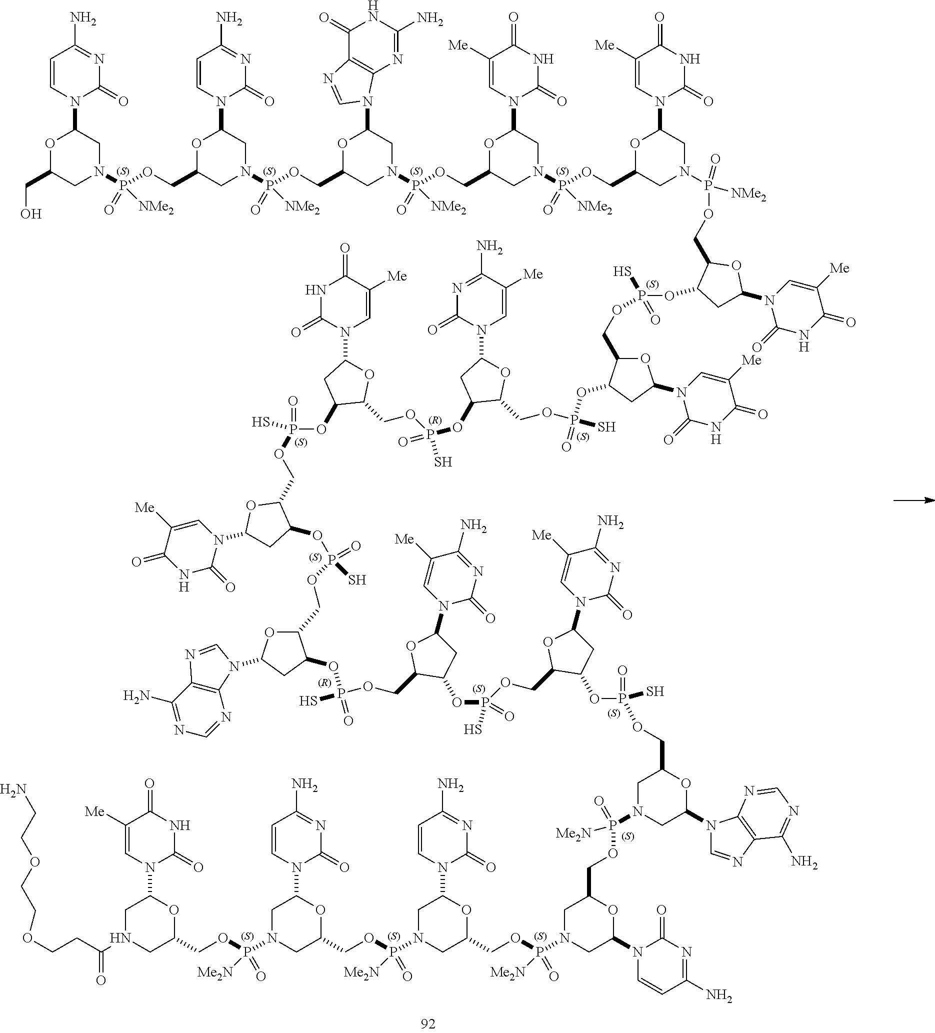

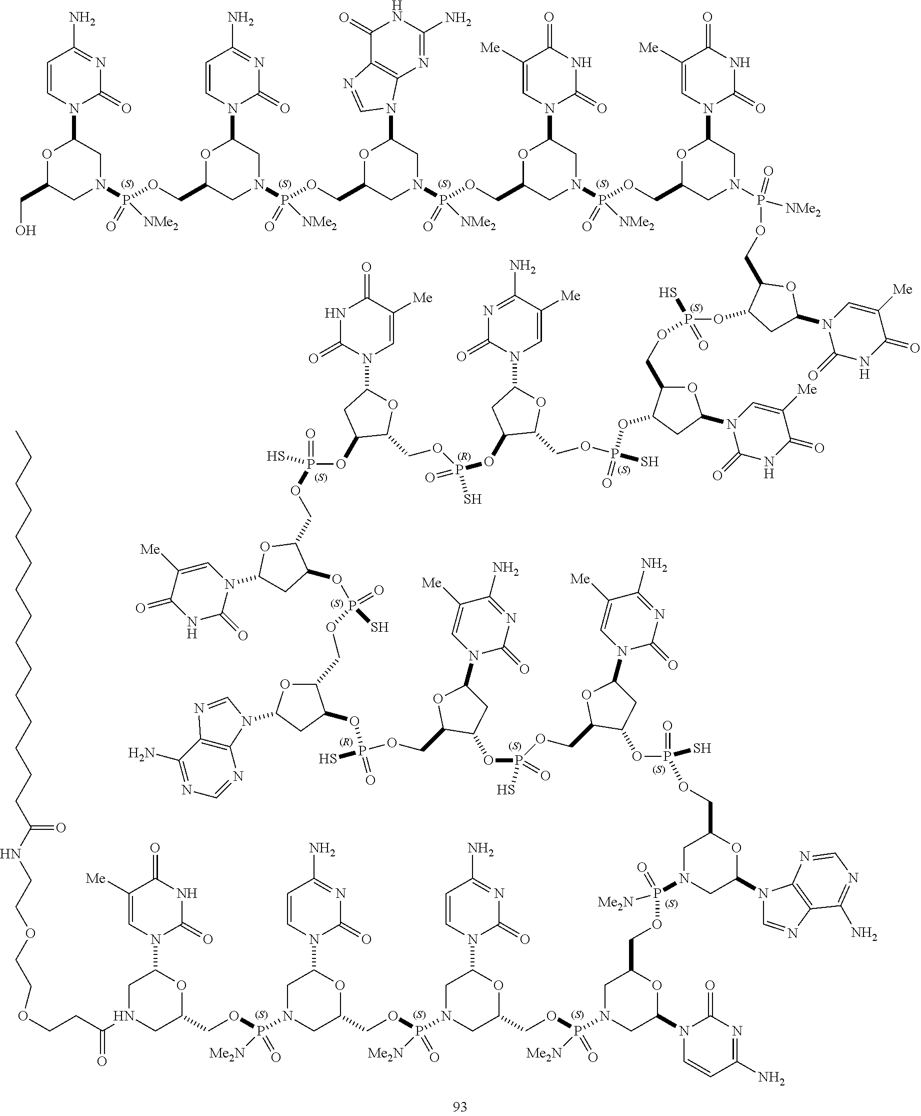

To a solution of starting material 92 (9.24 mg, 1.521 μmol) in water (2.5 mL) was added a saturated aqueous NaHCO₃ (8%) (0.5 mL), DMSO (1.5 mL), acetonitrile (1.5 mL), TEA (0.050 mL, 0.36 mmol), and then Perfluorophenyl palmitate (32.1 mg, 0.076 mmol). The resulting mixture was stirred at 35° C. for 2 days, diluted with 8 mL water, filtered through a syringe filter, and subjected to ultrafiltration (Amicon Ultra-15, ultracel 3K, 3500 rpm, 45 min) twice. The resulting solution (˜4 mL) was re-subjected to the above coupling conditions one more times. The crude product was purified with Sep-Pak Vac C18 6 cc/1 g, eluting with MeCN in Water (from 0% to 40%). The fractions containing the desired product were combined, concentrated, dissolved in water (˜3 mL), and subjected to freeze-drying over 2 day. 2.2 mg of product 93.

MS (ESI) m/z: [M+5H]⁵⁺ Calcd for C₂₁₆H₃₃₃N₇₃O₈₈P₁₇S₈ 1227.7. Found 1227.9.

### Example 7: Preparation of PMO-Gapmer with 5′ Lipid

### Deprotection of TBDPS

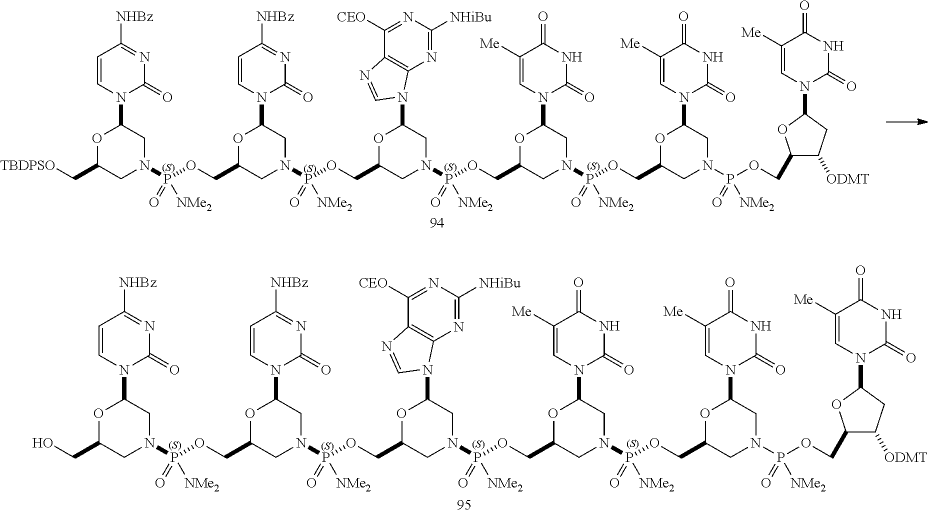

To a solution of starting material 94 (290 mg, 0.105 mmol) in pyridine (2 mL) and TEA (2 mL) at room temperature was added TEA-3HF (0.257 mL, 1.576 mmol). The resulting solution was stirred overnight, and treated with methoxytrimethylsilane (1 mL, 7.254 mmol). After 1 h stirring at room temperature, 1,3-dimethyl-2-imidazolidinone (2 mL) was added to make a clear solution. The resulting solution was added into EtOAc (12 mL) and MTBE (36 mL) was added slowly. After 30 min, the slurry was filtered through a sintered glass filter, rinsing with MTBE/EtOAc (3/1, 10 mL). Drying of the cake in vacuo provided 245 mg of target product 95. MS (ESI) m/z: [M+2H]²⁺ Calcd for C₁₁₀H₁₄₃N₂₈O₃₂P₅ 1261.75. Found 1261.45.

### Installation of Hexylamino Linker

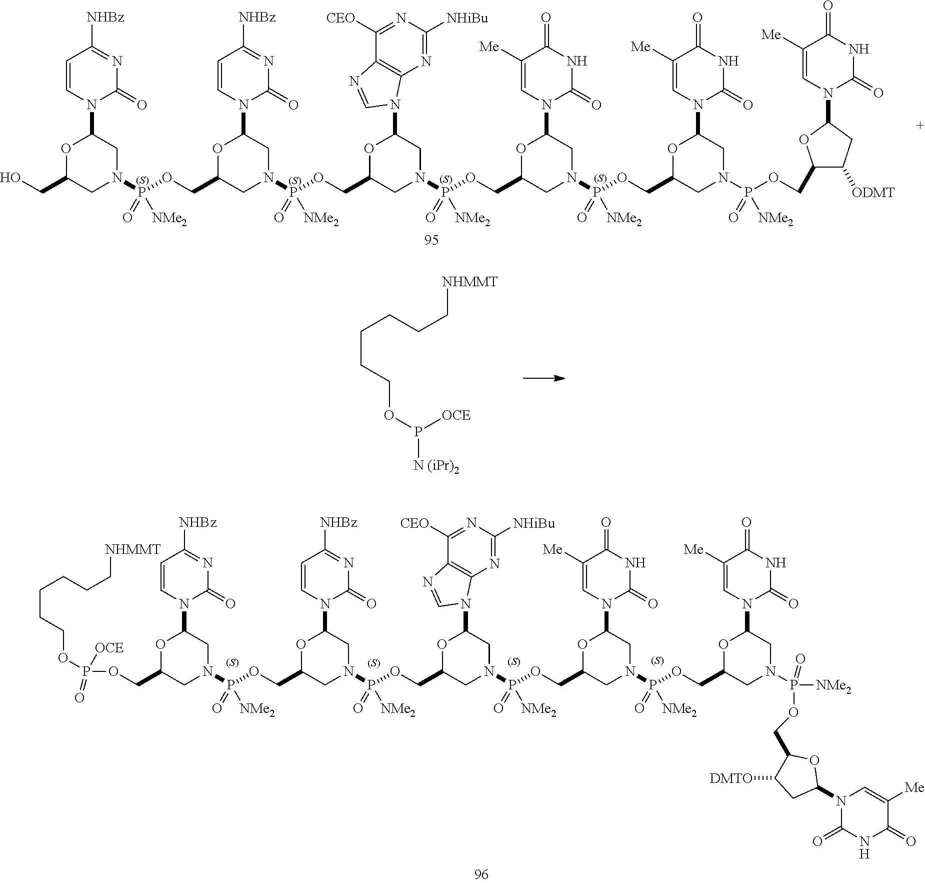

Compound 95 (225 mg, 0.089 mmol) was dissolved in MeCN (5.6 mL) and 6 mL DCM, and concentrated in vacuo. This process was repeated two more times. The resulting residue was dissolved in DCM (9.0 mL) and MeCN (5.6 mL). To the resulting solution was added MMT-hexylaminolinker phosphoramidite (158 mg, 0.268 mmol) and 4,5-dicyanoimidazole (42.1 mg, 0.357 mmol). After 1 h, additional MMT-hexylaminolinker phosphoramidite (50 mg) and 4,5-dicyanoimidazole (10 mg) were added. After 30 min, a solution of tert-butyl hydro peroxide in decane (5.5 M, 0.081 mL, 0.446 mmol) was added. After stirred at room temperature overnight, the reaction mixture was added into 35 mL MTBE, rinsing with 4 mL DCM. Additional 7 mL MTBE was then added and the resulting solid was collected by filtration and rinsed with a mixture of MTBE/DCM (4/1, 15 mL). Drying of the cake in vacuo overnight gave 270 mg of compound 96.

MS (ESI) m/z: [M+2H]²⁺ Calcd for C₁₃₉H₁₇₆N₃₀O₃₆P₆ 1513.56. Found 1513.88.

### Deprotection of MMT and DMT Groups

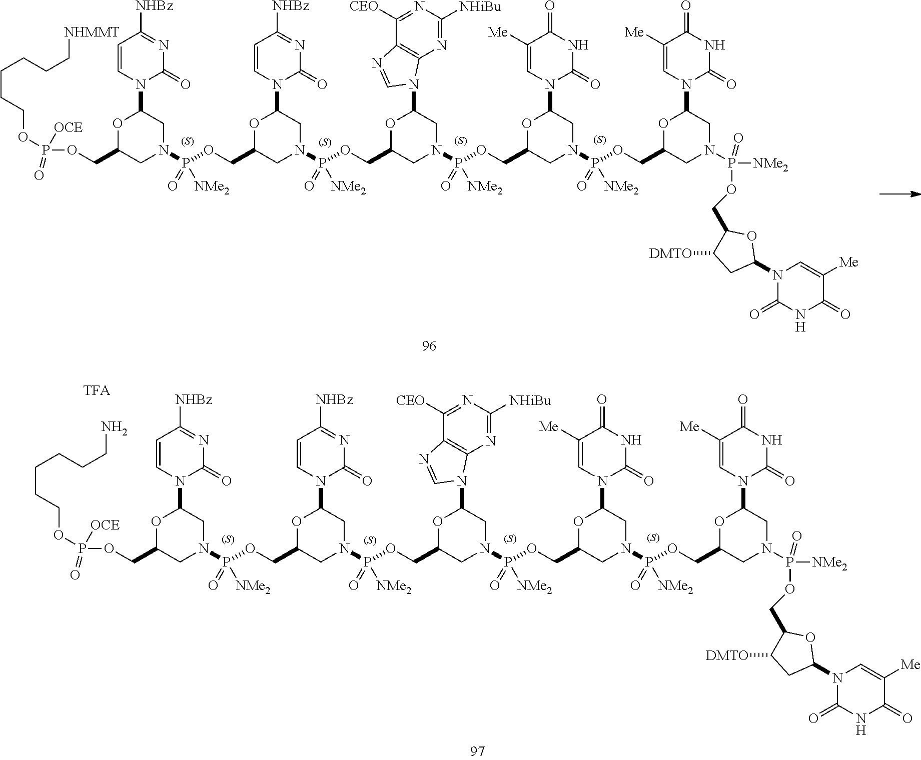

To a solution of compound 96 (270 mg, 0.089 mmol) in dichloromethane (10 mL) was added ethanol (0.5 mL, 8.563 mmol) and TFA (0.5 mL, 6.49 mmol). After 1 h at room temperature, the reaction mixture was added into EtOAc (30 mL) and 30 mL MTBE was added. After 30 min, the solid was collected by filtration and rinsed with MTBE/EtOAc (1/1, mL). Drying of the cake in vacuo for 2 h provided 210 mg of the target product (97). MS (ESI) m/z: [M+2H]²⁺ Calcd for C₉₈H₁₄₂N₃₀O₃₃P₆ 1226.44. Found 1226.68.

### Installation of Palmitoyl Lipid

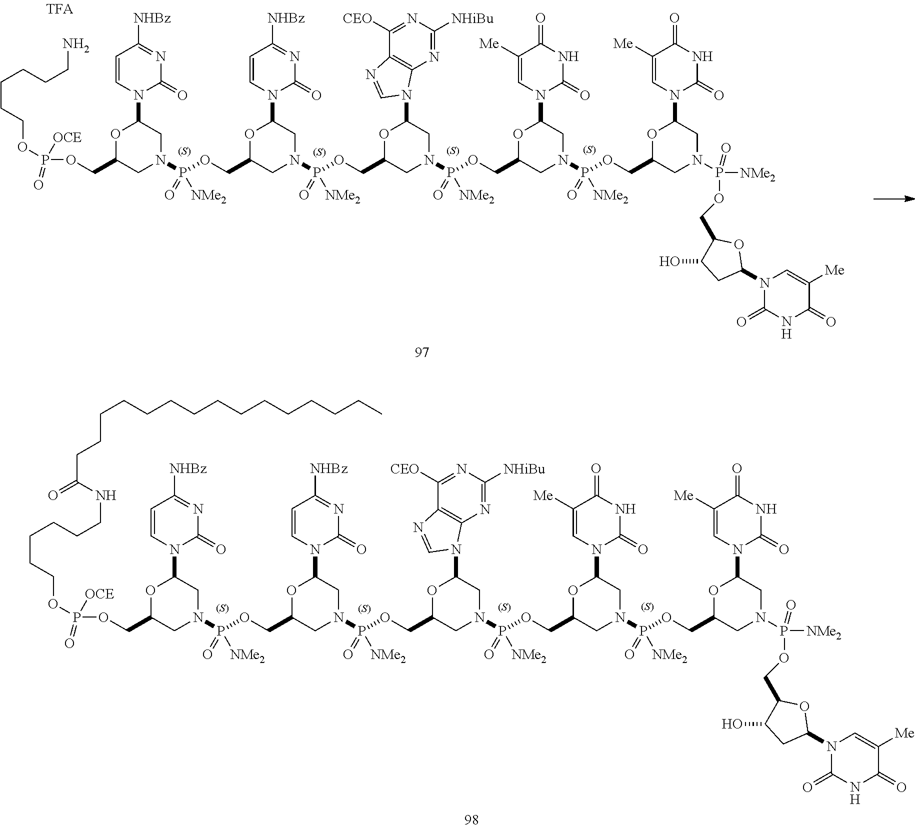

To a solution of starting material 97 (210 mg, 0.082 mmol) in MeCN (10.5 mL) and methanol (3.4 mL) was added TEA (0.103 mL, 0.736 mmol) and perfluorophenyl palmitate (114 mg, 0.27 mmol). After 1 h at room temperature, the reaction mixture was treated with 120 mL MTBE portionwise. The resulting solid was collected by filtration and rinsed with MTBE. Drying of the cake in vacuo at room temperature for 2 days gave 169 mg of the target product (98).

MS (ESI) m/z: [M+2H]²⁺ Calcd for C₁₁₄H₁₇₂N₃₀O₃₄P₆ 1345.55. Found 1345.53.

Activation with (−)-PSI

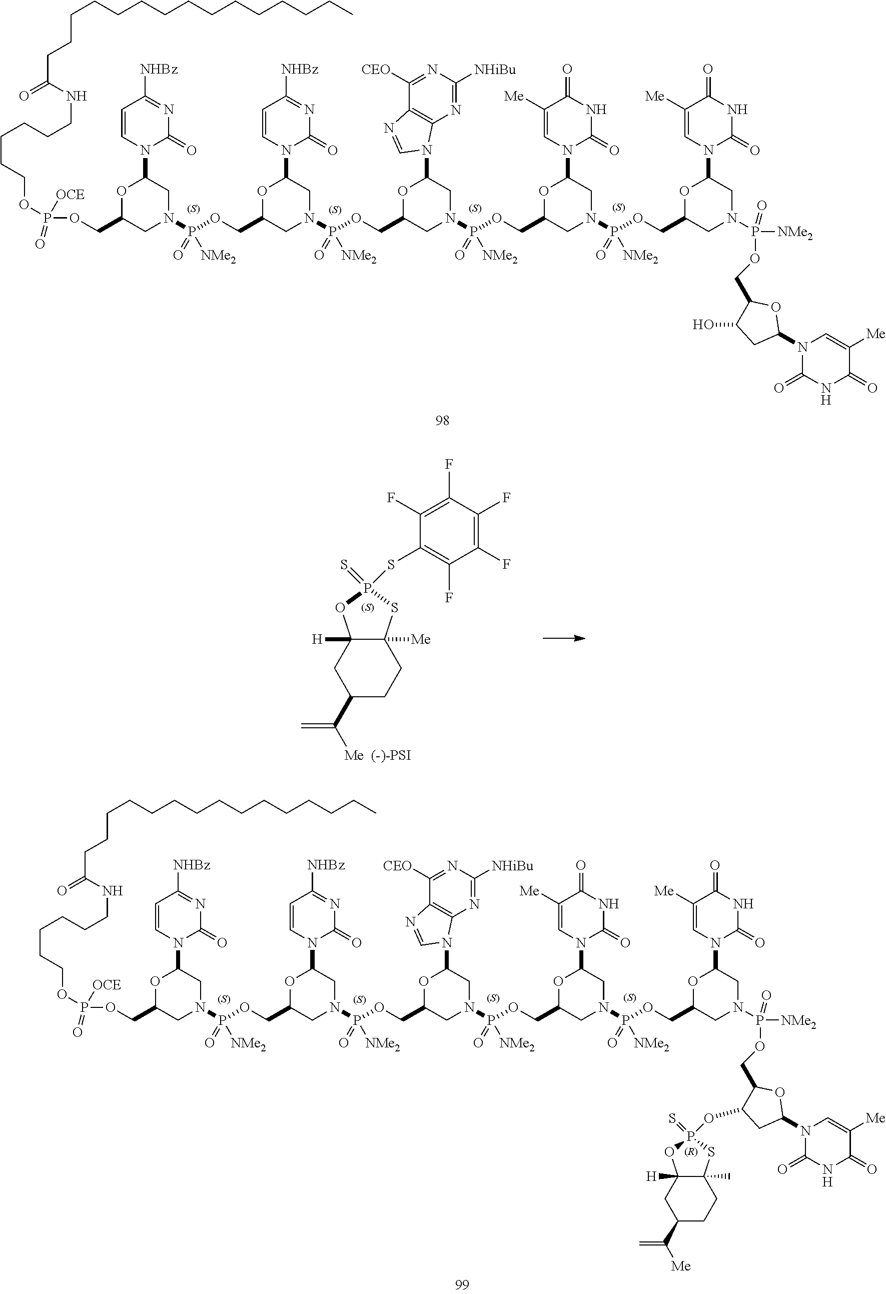

Starting material 98 (169 mg, 0.063 mmol) and (−)-PSI reagent (Aldrich, CAS: 2245335-70-8, 56.1 mg, 0.126 mmol) were dissolved in THF (3 mL) and concentrated in vacuo. The process was repeated two more times. The resulting reside was dissolved in THF (4 mL) and treated with DBU (0.014 mL, 0.094 mmol) at room temperature. The reaction mixture was stirred for 1 h and treated with MTBE (20 mL). The resulting slurry was filtered, rinsing with MTBE (2×3 mL). Drying of the cake in vacuo at room temperature overnight gave 187 mg of target product 99.

MS (ESI) m/z: [M+2H]²⁺ Calcd for C₁₂₄H₁₈₇N₃₀O₃₅P₇S₂ 1468.57. Found 1468.93.

### 12+6 Coupling

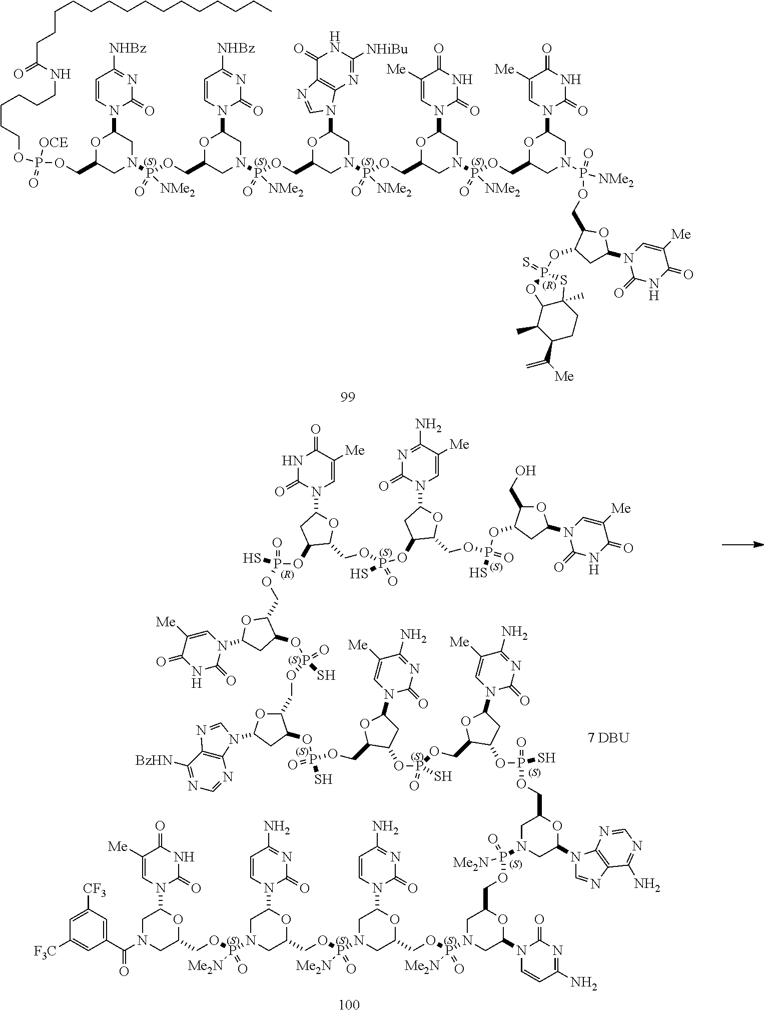

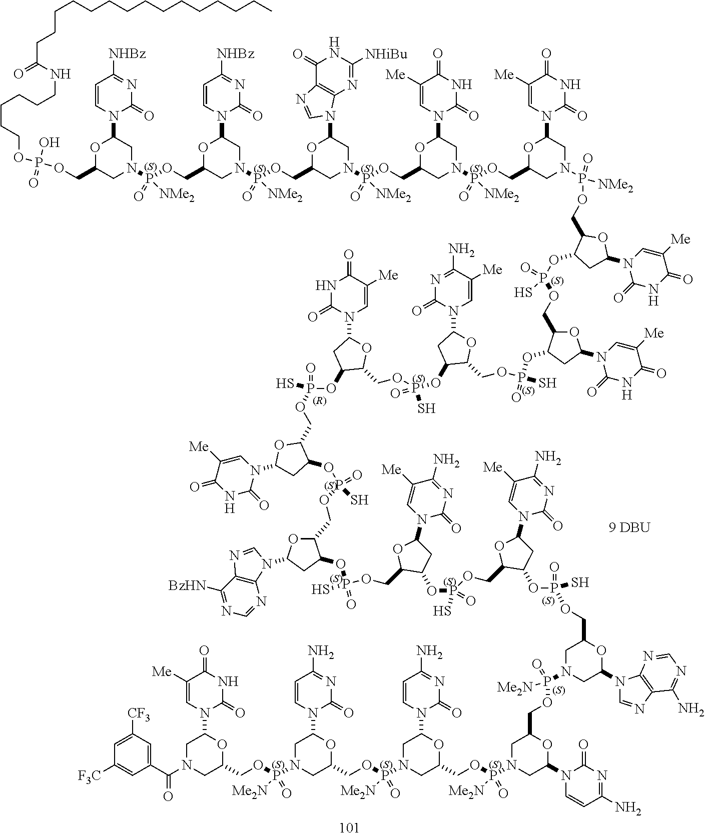

To a mixture of starting material 99 (100 mg, 0.019 mmol) and reactant 100 (187 mg, 0.064 mmol) was added 1,3-dimethyl-2-imidazolidinone (4 mL). The resulting mixture was azeotroped with toluene (2.5 mL each time) four times at 30-33° C. To the resulting solution was added 4 Å molecular sieves (250 mg). The reaction flask was applied to vacuum and filled with nitrogen. The process was repeated two more times. To the resulting mixture was added morpholine (0.034 mL, 0.386 mmol) and then DBU (0.041 mL, 0.27 mmol). After being stirred for 24 h at room temperature, the reaction mixture was filtered through a syringe filter and the filtrate was added into EtOAc (15 mL), rinsing with 4 mL 1,3-dimethyl-2-imidazolidinone. The resulting slurry mixture was centrifuged (3000 rpm, 20 min). The resulting pallet was collected by decantation, dissolved in a mixture of DCM/EtOH (10 mL/5 mL), and treated with EtOAc (20 mL). The resulting solid was collected by filtration and rinsed with a mixture of EtOAc/DCM (4 mL/2 mL). Drying of the cake in vacuo at room temperature for 1 h provided 123 mg of target product 101 contaminated with the remaining starting material (100). The material was used in next step without further purification.

MS (ESI) m/z: [M−4H]⁴⁻ Calcd for C₂₄₉H₃₄₅F₆N₇₃O₉₃P₁₈S₈ 1693.19. Found 1693.6.

### Final Deprotection/Purification

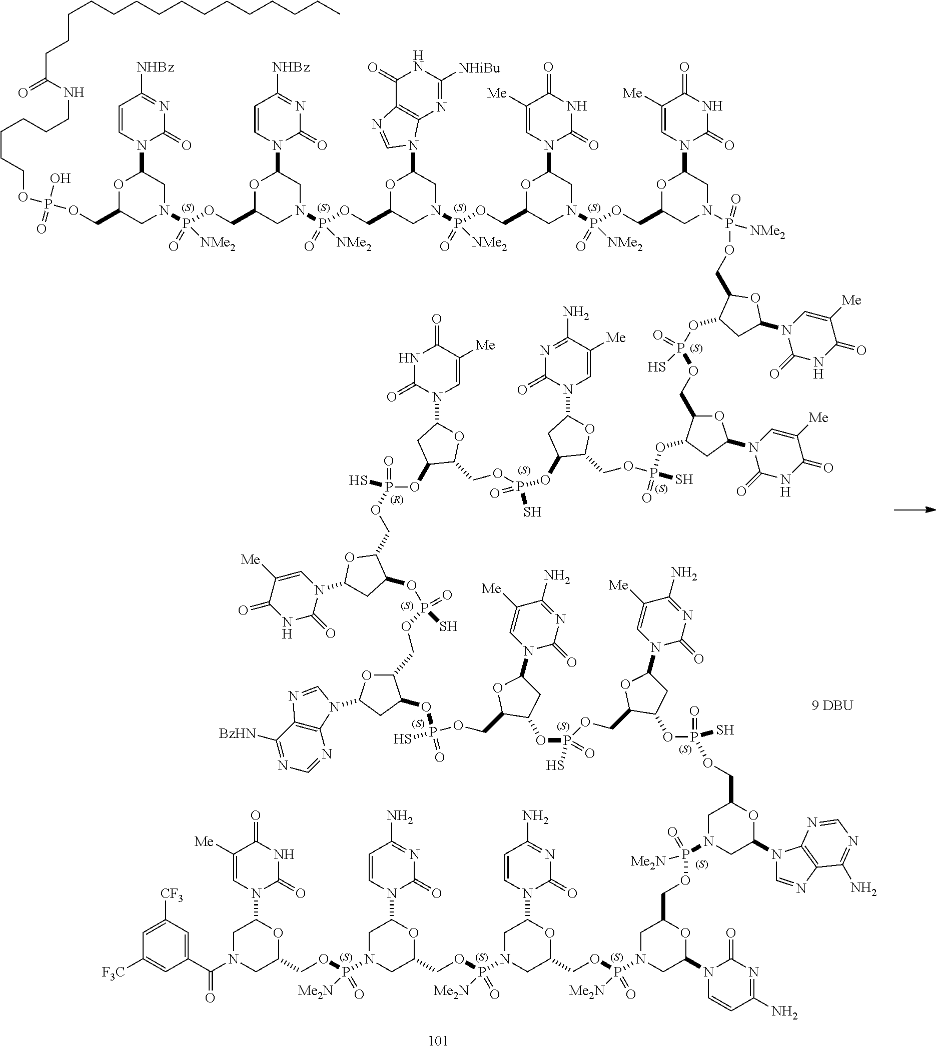

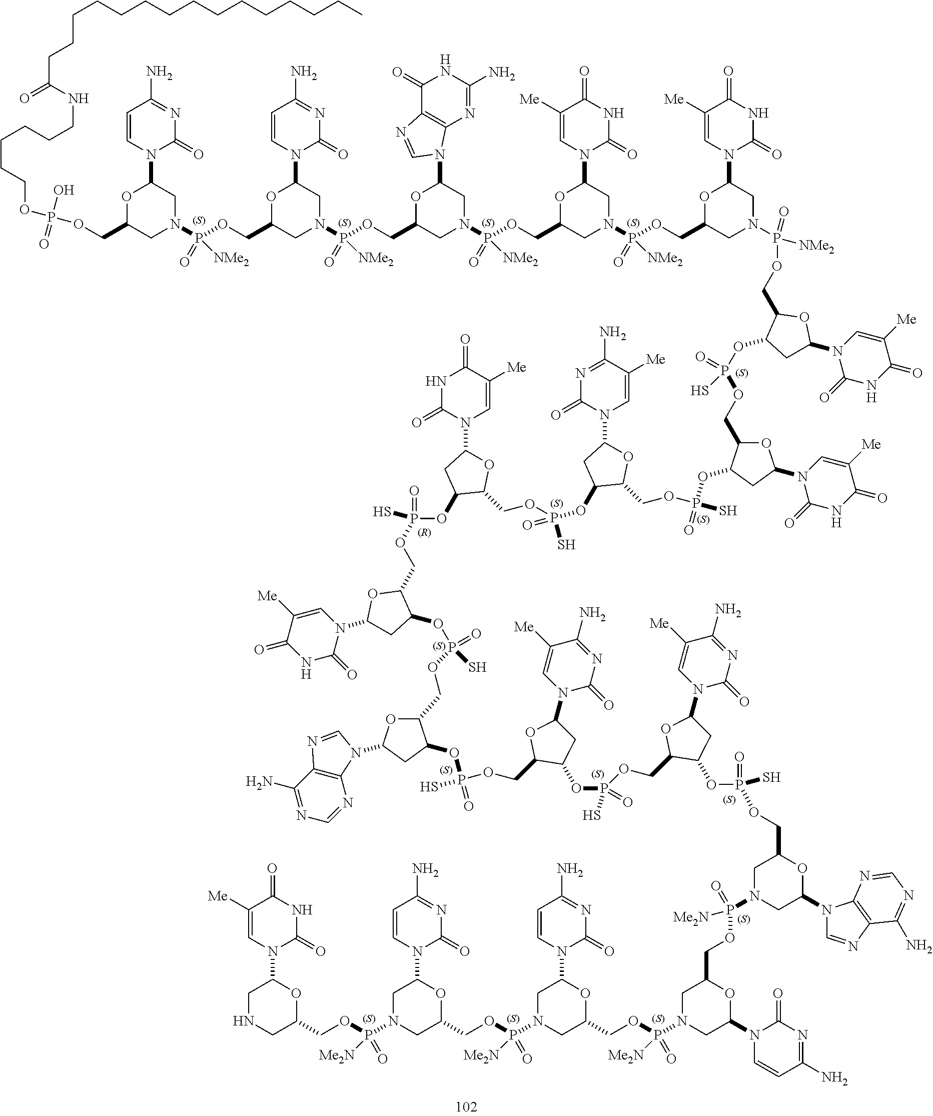

To a solution of starting material 101 (0.123 g) in methanol (5 mL) was added 28% ammonium hydroxide (5 mL) and DL-dithiothreitol (0.024 g, 0.15 mmol). The resulting mixture was stirred at 53-55° C. for 24 h and cooled to room temperature. A mixture of MeCN/EtOAc (60 mL/20 mL) was added and the resulting slurry was centrifuged (3500 ppm, 20 min). The resulting pallet was isolated and dissolved in water (˜10 mL). The aqueous solution was subjected to ultrafiltration (Amicon Ultra-15, ultracel 3K, 3500 rpm, 45 min) five times. The resulting solution was diluted with 4 mL water and purified by IEX-HPLC under the following conditions depicted in Table 8.

TABLE 8
IEX-HPLC conditions

| Column | TSKgel SuperQ-5PW, 7.5 mm ID × 7.5 cm, | |
|---|---|---|
| | 10 μm, TOSOH cat no. K0080-103KNM | |
| | (Three columns connected in serial for | |
| | purification) | |
| Instrument | Agilent 1200 | |
| Mobile phase A | 10 mM NaOH pH = 12 | |
| Mobile phase B | 10 mM NaOH pH = 12 w/1M NaCl | |
| Column Temperature (° C.) | 45 | |
| Gradient Time (min) | A % | B % |
| 0 | 60 | 40 |
| 10 | 25 | 75 |
| 15 | 5 | 95 |
| 20 | 5 | 95 |
| 25 | 60 | 40 |
| 29 | 60 | 40 |

| | |
|---|---|
| Flow Rate (mL/min) | 3.0 |
| Wavelength (nm) | 260 |

Desalting of the purified product was conducted with Amicon Ultra-15, Ultracel-3K (3500 rpm, 45 min)×5 times. Freeze-drying of the resulting solution (5 mL) for 2 days provided 4.2 mg of target product 102.

MS (ESI) m/z: [M+5H]⁵⁺ Calcd for C₂₁₅H₃₃₄N₇₃O₈₈P₁₈S₈ 1231.93. Found 1232.4.

### Example 8: In Vitro Activity of PMO-Gapmers Targeting the MAPT Gene Transcripts

The ability of the disclosed PMO-gapmers to reduce gene translation was evaluated by measuring their ability to reduce the expression of MAPT gene transcripts, transcripts which have been associated with the expression of the Tau protein.

### Example 8.1: Inhibition of Human Tau in SH-SY5Y Cells by 5-8-5 PMO-Gapmers

Antisense oligonucleotides targeting Tau were tested for their inhibitory effects on human Tau mRNA in vitro. Cultured SH-SY5Y cells were transfected using Endo-Porter with 10, 30 or 100 nM antisense oligonucleotide. After a treatment period of 2 days, RNA was isolated from the cells using Maxwell® RSC simply RNA Cells/Tissue Kit and cDNA was synthesized. Tau mRNA levels were measured by quantitative real-time PCR using TaqMan probes specific to Human MAPT (Assay ID Hs00902194_m1) and Human GAPDH (Assay ID HS99999905_m1). Tau mRNA levels were normalized to the levels of the endogenous reference gene GAPDH. Results are presented as relative expression of control cells treated with vehicle.

Seventy stereorandom 5-8-5 PMO-gapmers targeting MAPT gene transcripts where synthesized and their ability to reduce the expression of said transcripts was measured by determining the relative expression of the Tau mRNA normalized to the expression of the endogenous reference gene GAPDH. The in vitro activity of the 17 stereorandom 5-8-5 PMO-gapmers at concentrations of either 10 nM, 30 nM or 100 nM are shown below in Table 9:

TABLE 9

| | | Relative | | |
|---|---|---|---|---|
| | Compound | expression | | |
| | ID | (MAPT/GAPDH) | | |
| Sequence | (SEQ ID NO) | 10 nM | 30 nM | 100 nM |
| GGGGACTCG | 103 | 0.771 | 0.675 | 0.581 |
| CTGACATGG | (SEQ ID | | | |
| | NO: 1) | | | |
| TGGGTGTAG | 104 | 0.863 | 0.542 | 0.401 |
| CGAGAATCC | (SEQ ID | | | |
| | NO: 2) | | | |
| GGGTGCACT | 105 | 0.811 | 0.587 | 0.377 |
| AGTTTATAG | (SEQ ID | | | |
| | NO: 3) | | | |
| GGGGTCTTC | 106 | 0.619 | 0.410 | 0.291 |
| TAATATCCT | (SEQ ID | | | |
| | NO: 4) | | | |
| AGGTTCTCG | 107 | 0.850 | 0.628 | 0.357 |
| CTATATCGC | (SEQ ID | | | |
| | NO: 5) | | | |
| GAGTTAGAA | 108 | 0.801 | 0.480 | 0.378 |
| GCTTTGACT | (SEQ ID | | | |
| | NO: 6) | | | |
| GCAGATGAC | 109 | 0.866 | 0.587 | 0.373 |
| CCTTAGACA | (SEQ ID | | | |
| | NO: 7) | | | |
| CAAACCTGT | 110 | 0.898 | 0.785 | 0.547 |
| CACACCCGA | (SEQ ID | | | |
| | NO: 8) | | | |
| TTAAACCCC | 111 | 0.959 | 0.865 | 1.070 |
| ATAGACATA | (SEQ ID | | | |
| | NO: 9) | | | |
| GAGGCCCAA | 112 | 0.972 | 0.853 | 0.822 |
| ATGATCACA | (SEQ ID | | | |
| | NO: 10) | | | |
| TGGATTTAG | 113 | 0.896 | 0.710 | 0.441 |
| CAGTAGGGT | (SEQ ID | | | |
| | NO: 11) | | | |
| AGCAGATGA | 114 | 0.806 | 0.618 | 0.496 |
| CCCTTAGAC | (SEQ ID | | | |
| | NO: 12) | | | |
| AGCCGGCAT | 115 | 0.955 | 0.698 | 0.578 |
| ACAGTATAT | (SEQ ID | | | |
| | NO: 13) | | | |
| TGTGCTCTT | 116 | 0.764 | 0.632 | 0.435 |
| TATGGATGG | (SEQ ID | | | |
| | NO: 14) | | | |
| GGATTTAGC | 117 | 1.245 | 0.844 | 0.480 |
| AGTAGGGTG | (SEQ ID | | | |
| | NO: 15) | | | |
| CCCCATGAC | 118 | 0.893 | 0.747 | 0.442 |
| TACAGTGTG | (SEQ ID | | | |
| | NO: 16) | | | |
| GCTTTTGTG | 119 | 0.793 | 0.381 | 0.173 |
| ACCAGGGAC | (SEQ ID | | | |
| | NO: 17) | | | |

### Example 8.2: Inhibition of Human Tau in SH-SY5Y Cells by 4-10-4 PMO-Gapmers

Antisense oligonucleotides targeting Tau were tested for their inhibitory effects on human tau mRNA in vitro. Cultured SH-SY5Y cells were transfected using Endo-Porter with 30, 100 or 300 nM antisense oligonucleotide. After a treatment period of 2 days, RNA was isolated from the cells using Maxwell® RSC simply RNA Cells/Tissue Kit and cDNA was synthesized. Tau mRNA levels were measured by quantitative real-time PCR using TaqMan probes specific to Human MAPT (Assay ID Hs00902194_m1) and Human GAPDH (Assay ID HS99999905_m1). Tau mRNA levels were normalized to the levels of the endogenous reference gene GAPDH. Results for these 4-10-4 PMO-gapmers are shown in Table 10.

TABLE 10

| Compound No. | Relative expression (MAPT/GAPDH) | | |
|---|---|---|---|
| (SEQ ID NO:) | 30 nM | 100 nM | 300 nM |
| 120 | 0.613 | 0.492 | [not tested] |
| (SEQ ID NO: 11) | | | |
| 121 | 0.628 | 0.484 | 0.320 |
| (SEQ ID NO: 17) | | | |
| 122 | 0.646 | 0.520 | 0.429 |
| (SEQ ID NO: 5) | | | |
| 123 | 0.735 | 0.576 | 0.415 |
| (SEQ ID NO: 12) | | | |
| 124 | 0.812 | 0.663 | 0.541 |
| (SEQ ID NO: 14) | | | |
| 125 | 0.705 | 0.565 | 0.439 |
| (SEQ ID NO: 16) | | | |
| 126 | 0.600 | 0.450 | 0.324 |
| (SEQ ID NO: 2) | | | |
| 127 | 0.638 | 0.520 | 0.442 |
| (SEQ ID NO: 3) | | | |
| 128 | 0.556 | 0.423 | 0.311 |
| (SEQ ID NO: 4) | | | |
| 129 | 0.709 | 0.545 | 0.436 |
| (SEQ ID NO: 7) | | | |
| 130 | 0.747 | 0.528 | 0.486 |
| (SEQ ID NO: 10) | | | |
| 131 | 0.989 | 0.914 | 0.801 |
| (SEQ ID NO: 9) | | | |

The results from the in vitro evaluations of the stereorandom PMO-gapmers as reported in Examples 8.1 and 8.2 show that the disclosed PMO-gapmers are capable of binding to MAPT gene transcripts and inducing RNase H activity, thus reducing the expression of the MAPT mRNA.

### Example 8.3: MALDI-MASS Analysis

MALDI-MASS analysis was conducted for seventeen 5-8-5 PMO-gapmers and twelve 4-10-4 gapmers, with the results shown in Table 11 and Table 12, respectively. MASS spectra were obtained by negative mode on Autoflex MALDI-TOF-MS spectrometer calibrated by standard oligonucleotide (Bruker). 3-Hydroxypicolinic acid with the addition of Diammonium hydrogen citrate was used as matrix.

TABLE 11
MALDI-MASS for 5-8-5 PMO-Gapmers

| Compound | | | |
|---|---|---|---|
| No. | | | |
| (SEQ ID | | | |
| NO:) | Sequence (5′-3′) | Theoretical | Found |
| 103 | GGGGACTCGCTGACATGG | 5942.9 | 5944.0 |
| (SEQ ID | | | |
| NO: 1) | | | |
| 104 | TGGGTGTAGCGAGAATCC | 5942.0 | 5943.8 |
| (SEQ ID | | | |
| NO: 2) | | | |
| 105 | GGGTGCACTAGTTTATAG | 5931.9 | 5933.7 |
| (SEQ ID | | | |
| NO: 3) | | | |
| 106 | GGGGTCTTCTAATATCCT | 5842.9 | 5844.9 |
| (SEQ ID | | | |
| NO: 4) | | | |
| 107 | AGGTTCTCGCTATATCGC | 5827.9 | 5828.6 |
| (SEQ ID | | | |
| NO: 5) | | | |
| 108 | GAGTTAGAAGCTTTGACT | 5915.9 | 5916.0 |
| (SEQ ID | | | |
| NO: 6) | | | |
| 109 | GCAGATGACCCTTAGACA | 5854.9 | 5857.3 |
| (SEQ ID | | | |
| NO: 7) | | | |
| 110 | CAAACCTGTCACACCCGA | 5759.9 | 5759.7 |
| (SEQ ID | | | |
| NO: 8) | | | |
| 111 | TTAAACCCCATAGACATA | 5797.9 | 5800.8 |
| (SEQ ID | | | |
| NO: 9) | | | |
| 112 | GAGGCCCAAATGATCACA | 5863.9 | 5865.9 |
| (SEQ ID | | | |
| NO: 10) | | | |
| 113 | TGGATTTAGCAGTAGGGT | 5972.0 | 5972.8 |
| (SEQ ID | | | |
| NO: 11) | | | |
| 114 | AGCAGATGACCCTTAGAC | 5854.9 | 5853.3 |
| (SEQ ID | | | |
| NO: 12) | | | |
| 115 | AGCCGGCATACAGTATAT | 5869.9 | 5869.8 |
| (SEQ ID | | | |
| NO: 13) | | | |
| 116 | TGTGCTCTTTATGGATGG | 5913.9 | 5914.7 |
| (SEQ ID | | | |
| NO: 14) | | | |
| 117 | GGATTTAGCAGTAGGGTG | 5997.0 | 5996.2 |
| (SEQ ID | | | |
| NO: 15) | | | |
| 118 | CCCCATGACTACAGTGTG | 5821.9 | 5822.1 |
| (SEQ ID | | | |
| NO: 16) | | | |
| 119 | GCTTTTGTGACCAGGGAC | 5892.9 | 5892.7 |
| (SEQ ID | | | |
| NO: 17) | | | |

TABLE 12
MALDI-MASS for 4-10-4 PMO-Gapmers

| Compound | | | |
|---|---|---|---|
| No. | | | |
| (SEQ ID | | | |
| NO:) | Sequence (5′-3′) | Theoretical | Found |
| 120 | TGGATTTAGCAGTAGGGT | 5951.9 | 5952.9 |
| (SEQ ID | | | |
| NO: 11) | | | |
| 121 | GCTTTTGTGACCAGGGAC | 5872.9 | 5873.9 |
| (SEQ ID | | | |
| NO: 17) | | | |
| 122 | AGGTTCTCGCTATATCGC | 5807.8 | 5809.0 |
| (SEQ ID | | | |
| NO: 5) | | | |
| 123 | AGCAGATGACCCTTAGAC | 5834.9 | 5837.1 |
| (SEQ ID | | | |
| NO: 12) | | | |
| 124 | TGTGCTCTTTATGGATGG | 5893.9 | 5897.6 |
| (SEQ ID | | | |
| NO: 14) | | | |
| 125 | CCCCATGACTACAGTGTG | 5801.8 | 5803.9 |
| (SEQ ID | | | |
| NO: 16) | | | |
| 126 | TGGGTGTAGCGAGAATCC | 5921.9 | 5921.7 |
| (SEQ ID | | | |
| NO: 2) | | | |
| 127 | GGGTGCACTAGTTTATAG | 5911.9 | 5911.4 |
| (SEQ ID | | | |
| NO: 3) | | | |
| 128 | GGGGTCTTCTAATATCCT | 5822.8 | 5822.2 |
| (SEQ ID | | | |
| NO: 4) | | | |
| 129 | GCAGATGACCCTTAGACA | 5834.9 | 5835.6 |
| (SEQ ID | | | |
| NO: 7) | | | |
| 130 | GAGGCCCAAATGATCACA | 5843.9 | 5844.1 |
| (SEQ ID | | | |
| NO: 10) | | | |
| 131 | TTAAACCCCATAGACATA | 5777.8 | 5778.7 |
| (SEQ ID | | | |
| NO: 9) | | | |

### Example 9: In Vivo Knockdown of Human Tau by PMO-Gapmers

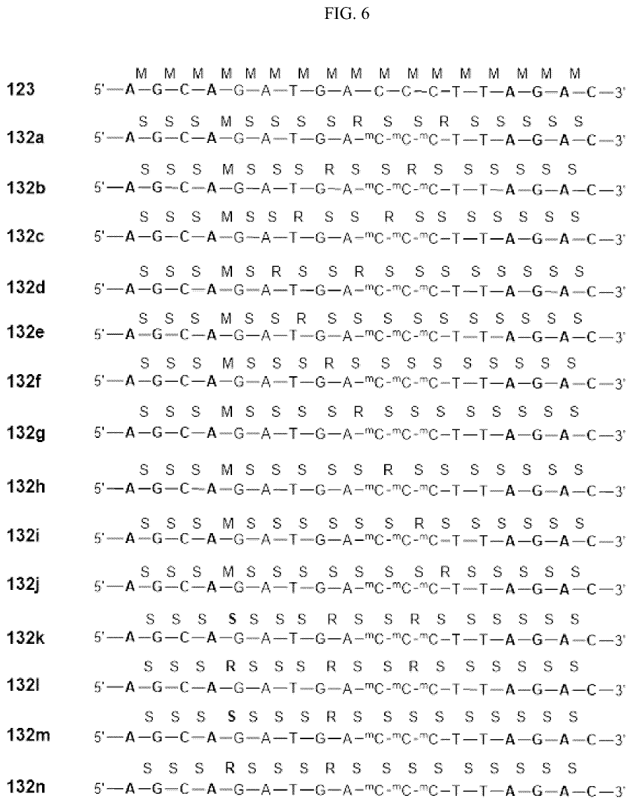

Selected antisense oligonucleotides using the chiralities referred to in FIG. 6 were tested in vivo. An antisense oligonucleotide having random chirality was also tested. Each of these was a 4-10-4 PMO-gapmer having (SEQ ID NO: 12). Groups of 4 human MAPT knock-in mice (Saito et al., J. Biol. Chem., 23; 294(34):12754-12765) were administered 60 or 100 μg of a selected antisense oligonucleotide by intracerebroventricular (ICV) bolus injection. A control group of 4 mice was similarly treated with saline. All procedures were performed under butorphanol, medetomidine and midazolam anesthesia and in accordance with IACUC regulations.

For ICV bolus injections, the antisense oligonucleotide was injected into the left lateral ventricle of human MAPT knock-in mice. Ten microliters of a saline solution containing 60 or 100 μg of oligonucleotide were injected. Tissues were collected 3 days after oligonucleotide administration. RNA was extracted from hippocampus and examined for human tau mRNA expression by real-time PCR analysis. Human tau mRNA levels were measured using TaqMan probes specific to Human MAPT and Mouse Gapdh. Results, shown in Table 13a and Table 13b, were calculated as inhibition of human tau mRNA expression normalized to Gapdh levels compared to the control mice.

TABLE 13a

| Compound | Dose | Relative expression level of MAPT |
|---|---|---|
| ID | (μg) | mRNA |
| 123 | 100 | 0.684 |
| 132a | 100 | 0.683 |
| 132b | 100 | 0.435 |
| 132c | 100 | 0.569 |
| 132d | 100 | 0.799 |
| 132e | 100 | 0.602 |
| 132f | 100 | 0.466 |
| 132g | 100 | 0.671 |
| 132h | 100 | 0.671 |
| 132i | 100 | 0.727 |
| 132j | 100 | 0.795 |
| 132k | 60 | 0.513 |
| 132l | 60 | 0.622 |
| 132m | 60 | 0.597 |
| 132n | 60 | 0.681 |

TABLE 13b

| | | | Chirality |
|---|---|---|---|
| Compound | | | of |
| ID | SEQ ID NO: | Sequence¹ | linkages |
| 123 | SEQ ID NO: 12 | AGCAGATGA | Stereorandom |
| | | CCCTTAGAC | |
| 132a | SEQ ID NO: 12 | AGCAGATGA | SSSMSSSSRS |
| | | ^{m}C^{m}C^{m}CTTA | SRSSSSS |
| | | GAC | |
| 132b | SEQ ID NO: 12 | AGCAGATGA | SSSMSSSRSS |
| | | ^{m}C^{m}C^{m}CTTA | RSSSSSS |
| | | GAC | |
| 132c | SEQ ID NO: 12 | AGCAGATGA | SSSMSSRSSR |
| | | ^{m}C^{m}C^{m}CTTA | SSSSSSS |
| | | GAC | |
| 132d | SEQ ID NO: 12 | AGCAGATGA | SSSMSRSSRS |
| | | ™C^{m}C^{m}CTTA | SSSSSSS |
| | | GAC | |
| 132e | SEQ ID NO: 12 | AGCAGATGA | SSSMSSRSSS |
| | | ^{m}C^{m}C^{m}CTTA | SSSSSSS |
| | | GAC | |
| 132f | SEQ ID NO: 12 | AGCAGATGA | SSSMSSSRSS |
| | | ^{m}C^{m}C^{m}CTTA | SSSSSSS |
| | | GAC | |
| 132g | SEQ ID NO: 12 | AGCAGATGA | SSSMSSSSRS |
| | | ^{m}C^{m}C^{m}CTTA | SSSSSSS |
| | | GAC | |
| 132h | SEQ ID NO: 12 | AGCAGATGA | SSSMSSSSSR |
| | | ^{m}C^{m}C^{m}CTTA | SSSSSSS |
| | | GAC | |
| 132i | SEQ ID NO: 12 | AGCAGATGA | SSSMSSSSSS |
| | | ^{m}C^{m}C^{m}CTTA | RSSSSSS |
| | | GAC | |
| 132j | SEQ ID NO: 12 | AGCAGATGA | SSSMSSSSSS |
| | | ^{m}C^{m}C^{m}CTTA | SRSSSSS |
| | | GAC | |
| 132k | SEQ ID NO: 12 | AGCAGATGA | SSSSSSSRSS |
| | | ^{m}CC^{m}CTTAG | RSSSSSS |
| | | AC | |
| 132l | SEQ ID NO: 12 | AGCAGATGA | SSSRSSSRSS |
| | | ^{m}C^{m}C^{m}CTTA | RSSSSSS |
| | | GAC | |
| 132m | SEQ ID NO: 12 | AGCAGATGA | SSSSSSSRSS |
| | | ^{m}C^{m}C^{m}CTTA | SSSSSSS |
| | | GAC | |
| 132n | SEQ ID NO: 12 | AGCAGATGA | SSSRSSSRSS |
| | | ^{m}C^{m}C^{m}CTTA | SSSSSSS |
| | | GAC | |

¹“C” means cytosine and “^{m}C” means 5-methylcytosine.

Although embodiments have been described in terms of specific exemplary embodiments and examples, the embodiments disclosed herein are for illustrative purposes only and various modifications and alterations might be made by those skilled in the art without departing from the spirit and scope of the invention as set forth in the following claims.

### CITED DOCUMENTS

All cited documents herein including those below are hereby incorporated by reference in their entirety.
1. WO2018057430A1.
2. U.S. Pat. No. 10,457,698.
3. U.S. Pat. No. 10,836,784
4. C. F. Bennett, Annu. Rev. Med. 2019, 70, 307.

1. A gapmer or pharmaceutically acceptable salt of the gapmer comprising:
a gap region, wherein the gap region contains deoxyribonucleosides linked to each other by phosphorothioate bonds;
a 5′ wing region positioned at the 5′ end of the gap region, wherein the 5′ wing region contains morpholino monomers linked to each other by phosphorodiamidate bonds; and
a 3′ wing region positioned at the 3′ end of the gap region, wherein the 3′ wing region contains morpholino monomers linked to each other by phosphorodiamidate bonds.

2. The gapmer or pharmaceutically acceptable salt of the gapmer according to claim 1, wherein the deoxyribonucleosides are comprised of the following structure: 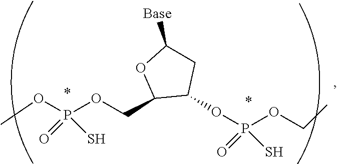
wherein P* represents a stereocenter that is either in the (R) or (S) configuration;
wherein the morpholino monomers are comprised of the following structure: 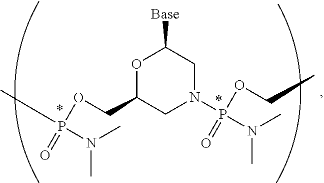
wherein P* represents a stereocenter that is either in the (R) or (S) configuration;
wherein the base in the deoxyribonucleoside and morpholino monomer structures is independently selected from the group consisting of: 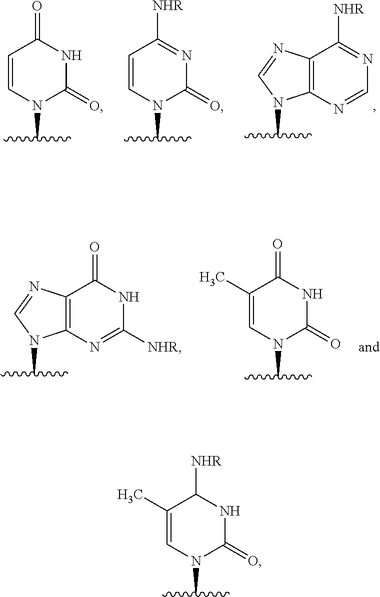
wherein R is selected from H, C(O)R₁ or C(O)OR₁,
wherein R₁ is selected from C₁-C₆ alkyl or aryl, and wherein the aryl is optionally substituted with a substituent selected from the group consisting of halogen, nitro and methoxy.

3. The gapmer or pharmaceutically acceptable salt of the gapmer according to claim 2, wherein the gapmer or pharmaceutically acceptable salt of the gapmer possesses the following structure: 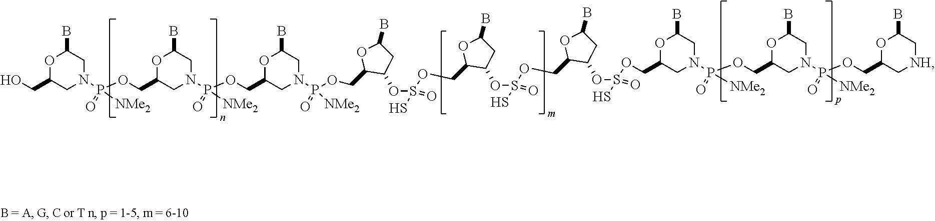
wherein n and p are an integer between 1 and 5,
m is an integer between 6 and 10; and
B is the base.

4. The gapmer or pharmaceutically acceptable salt of the gapmer according to claim 1, wherein the phosphorothioate and phosphorodiamidate bonds each possess a phosphorus that is independently in an R or S configuration, and wherein each R or S configuration is at least 90% pure.

5-8. (canceled)

9. The gapmer or pharmaceutically acceptable salt of the gapmer according to claim 1, wherein all the phosphorodiamidate bonds of the 5′ and 3′ wing regions possess a phosphorus atom having an S configuration, and wherein each S configuration is at least 90% pure.

10-12. (canceled)

13. The gapmer or pharmaceutically acceptable salt of the gapmer according to claim 1, wherein all the phosphorothioate bonds in the gap region possess a phosphorus atom having an S configuration, and wherein each S configuration is at least 95% pure.

14. (canceled)

15. The gapmer or pharmaceutically acceptable salt of the gapmer according to claim 1, wherein the phosphorothioate bonds in the gap region have a mix of R and S phosphorus configurations, and wherein each R and S configuration is at least 90% pure.

16-17. (canceled)

18. The gapmer or pharmaceutically acceptable salt of the gapmer according to claim 1, wherein the phosphorothioate bonds and the phosphorodiamidate bonds all possess phosphorus atoms that are stereorandom.

19. The gapmer or pharmaceutically acceptable salt of the gapmer according to claim 1, wherein the gapmers are conjugated to a lipid.

20-22. (canceled)

23. The gapmer or pharmaceutically acceptable salt of the gapmer according to claim 19, wherein all the phosphorodiamidate bonds of the 5′ and 3′ wing regions possess a phosphorus atom having an S configuration, and wherein each S configuration is at least 90% pure.

24. The gapmer or pharmaceutically acceptable salt of the gapmer according to claim 19, wherein all the phosphorothioate bonds in the gap region possess a phosphorus atom having an S configuration, and wherein each S configuration is at least 90% pure.

25. The gapmer or pharmaceutically acceptable salt of the gapmer according to claim 19, wherein the phosphorothioate bonds in the gap region have a mix of R and S phosphorus configurations, and wherein each R and S configuration is at least 90% pure.

26-27. (canceled)

28. A pharmaceutical composition comprising the gapmer or pharmaceutically acceptable salt of the gapmer of claim 1.

29-31. (canceled)

30-31. (canceled)

32. A method for preparing a stereorandom polymorpholino oligonucleotide (PMO) gapmer via solid-phase synthesis, wherein the method comprises:
attaching a morpholino monomer onto a solid support,
coupling a first morpholino-dimethylphosphoramidochloridate to the morpholino monomer on a solid support, thereby creating a 5′-wing region,
elongating the 5′-wing region to a first desired nucleotide length,
coupling a reverse DNA-phosphoramidite to the elongated 5′-wing region, thereby creating a DNA gap region,
elongating the DNA gap region to a second desired nucleotide length,
coupling a morpholino-phosphoramidate to the DNA gap region, thereby creating a 3′-wing region
elongating the 3′-wing region with morpholino-dimethylphosphoramidochloridates to a final desired nucleotide length, thereby forming a fully elongated stereorandom PMO-gapmer.

33. The method according to claim 32, wherein elongating the 5′-wing region, the DNA gap region and/or the 3′-wing region further comprises a detritylation step, wherein the detritylation step comprises treating the elongating 5′-wing region, the elongating DNA gap region and/or the elongating 3′-wing region in a mixture of 3 wt/v % trichloroacetic acid (TCA) in dichloromethane (CH₂Cl₂).

34. The method according to claim 32, wherein elongating the 5′-wing region and/or elongating the 3′-wing region further comprises neutralizing the elongating 5′-wing region and/or the elongating 3′-wing region, wherein the neutralization comprises washing the elongating 5′-wing region and/or the elongating 3′-wing region with a mixture of N,N-Diisopropylethylamine (iPr₂NEt), 1,3-Dimethyl-2-imidazolidinone (DMI) and CH₂Cl₂ in a ratio of 10:45:45.

35. The method according to claim 32, wherein elongating the 5′-wing region comprises coupling morpholino- or reverse DNA-dimethylphosphoramidochloridates to morpholino monomers of the elongating 5′-wing region in the presence of 1,2,2,6,6-pentamethylpiperidine (PMP) in DMI.

36-48. (canceled)

49. A method for preparing a stereodefined polymorpholino oligonucleotide (PMO) gapmer via a solution-phase synthesis process, wherein the method comprises:
synthesizing a stereodefined 5′-fragment of a first length,
synthesizing a stereodefined 3′-fragment of a second length,
coupling the stereodefined 5′-fragment and the stereodefined 3′-fragment in a solution to create an elongated stereospecific PMO-gapmer,
deprotecting the elongated stereospecific PMO-gapmer and
purifying the deprotected, elongated stereospecific PMO-gapmer.

50. The method according to claim 49, wherein synthesizing the stereodefined 5′-fragment further comprises performing a series of steps comprising a coupling step, a deprotection step, an activation step or combinations thereof until the stereodefined 5′-fragment of the first length is synthesized.

51. (canceled)

52. The method according to claim 50, wherein the coupling step of the series comprises mixing a morpholino- or a reverse DNA-dimethylphosphoramidochloridate in 1,3-dimethyl-2-imidazolidinone and in the presence of 1,2,2,6,6-pentamethylpiperidine (PMP).

53-80. (canceled)
